(19) United States
(12) Patent Application Publication
(10) Pub. No.: US 2025/0026853 A1
(43) Pub. Date: Jan. 23, 2025

(54) HUMANIZED ANTI-MUC1* ANTIBODIES

(71) Applicant: Minerva Biotechnologies Corporation, Waltham, MA (US)

(72) Inventors: Cynthia Bamdad, Boston, MA (US); Benoit Smagghe, Honolulu, HI (US)

(21) Appl. No.: 18/644,795

(22) Filed: Apr. 24, 2024

Related U.S. Application Data
(62) Division of application No. 17/817,525, filed on Aug. 4, 2022, now Pat. No. 12,006,371.
Application No. 17/817,525 is a continuation of application No. 15/549,942, filed on Aug. 9, 2017, now Pat. No. 11,746,159.
(60) Provisional application No. 62/114,526, filed on Feb. 10, 2015.

(51) Int. Cl. C07K 16/30; A61K 39/00

(52) U.S. Cl. CPC C07K 16/3092; A61K 39/4611; A61K 39/4631; A61K 39/4632; A61K 39/46447; A61K 2239/21; C07K 2317/24; C07K 2317/34; C07K 2317/52; C07K 2317/622; C07K 2319/02; C07K 2319/30

(57) ABSTRACT

The present application discloses humanized antibodies and antibody like proteins and fragments thereof.

### CROSS-REFERENCE TO RELATED APPLICATIONS

This application is a division of U.S, patent application Ser. No. 17/817,525, filed Aug. 4, 2022, which is a continuation of U.S, patent application Ser. No. 15/549,942, filed Aug. 9, 2017, now U.S. Pat. No. 11,746,159, which is a national stage entry of International Application No. PCT/US2016/017422, filed Feb. 10, 2016, which claims the benefit of U.S. Provisional Application No. 62/114,526, filed Feb. 10, 2015, which applications are incorporated herein by reference in their entirety.

### SEQUENCE LISTING

The instant application contains a Sequence Listing which has been submitted electronically in XML format and is hereby incorporated by reference in its entirety. Said XML copy, created on Apr. 19, 2024, is named 56699-731_401SL.xml and is 993,011 bytes in size.

### BACKGROUND OF THE INVENTION

### 1. Field of the Invention

The present application relates to humanized anti-MUC1* antibodies and methods of making and using them.

### 2. General Background and State of the Art

We previously discovered that a cleaved form of the MUC1 (SEQ ID NO:1) transmembrane protein is a growth factor receptor that drives the growth of over 75% of all human cancers. The cleaved form of MUC1, which we called MUC1* (pronounced muk 1 star), is a powerful growth factor receptor. Cleavage and release of the bulk of the extracellular domain of MUC1 unmasks a binding site for activating ligands dimeric NME1, NME6 or NME7. It is an ideal target for cancer drugs as it is aberrantly expressed on over 75% of all cancers and is likely overexpressed on an even higher percentage of metastatic cancers (Fessler SP, Wotkowicz MT, Mahanta SK and Bamdad C. (2009). MUC1* is a determinant of trastuzumab (Herceptin) resistance in breast cancer cells.Breast Cancer Res Treat. 118 (1): 113-124). After MUC1 cleavage most of its extracellular domain is shed from the cell surface. The remaining portion has a truncated extracellular domain that at least comprises the primary growth factor receptor sequence, PSMGFR (SEQ ID NO:2).

Antibodies are increasingly used to treat human diseases. Antibodies generated in non-human species have historically been used as therapeutics in humans, such as horse antibodies. More recently, antibodies are engineered or selected so that they contain mostly human sequences in order to avoid a generalized rejection of the foreign antibody. The process of engineering recognition fragments of a non-human antibody into a human antibody is generally called ‘humanizing’. The amount of non-human sequences that are used to replace the human antibody sequences determines whether they are called chimeric, humanized or fully human.

Alternative technologies exist that enable generation of humanized or fully human antibodies. These strategies involve screening libraries of human antibodies or antibody fragments and identifying those that bind to the target antigen, rather than immunizing an animal with the antigen. Another approach is to engineer the variable region(s) of an antibody into an antibody-like molecule. The present invention is intended to also encompass these approaches for use with recognition fragments of antibodies that the inventors have determined bind to the extracellular domain of MUC1*.

In addition to treating patients with an antibody, cancer immunotherapies have recently been shown to be effective in the treatment of cancers. T-cell based cancer immunotherapy is an attractive approach to overcome the cancer cells evasion from the immune system. A first immunotherapy, called CAR T (chimeric antigen receptor T cell) therapy relies on the expression of a CAR on the surface of the patient T cells for adoptive T-cell therapy (Dai H, Wang Y, Lu X, Han W. (2016) Chimeric Antigen Receptors Modified T-Cells for Cancer Therapy. J Natl Cancer Inst. 108 (7): djv439). Such receptor is composed of an anti cancer scFv linked to a T cell transmembrane and signaling domains. Upon binding of the receptor to a cancer associated antigen, a signal is transmitted resulting in T-cell activation, propagation and the targeted killing of the cancer cells. In practice, a patient's T cells are isolated and transduced with a CAR, expanded and then injected back into the patient. When the patient's CAR T cells bind to the antigen on a cancer cell, the CAR T cells expand and attack the cancer cells. A drawback of this method is the risk of activating the patient's immune system to destroy cells bearing the target antigen, when most cancer antigens are expressed on some healthy tissues, but overexpressed on cancerous tissues. To minimize the risk of off-tumor/on-target effects, the cancer antigen should be minimally expressed on healthy tissues.

A second cancer immunotherapy involves BiTEs (Bi-specific T cell Engagers). The BiTE approach attempts to eliminate the CAR T associated risk of off-tumor/on-target effects. Unlike CAR T, BiTEs are bispecific antibodies that should not pose any greater risk than regular antibody-based therapies. However, unlike typical anti-cancer antibodies that bind to and block a cancer antigen, BiTEs are designed to bind to an antigen on the tumor cell and simultaneously bind to an antigen on an immune cell, such as a T cell. In this way, a BiTE recruits the T cell to the tumor. BiTEs are engineered proteins that simultaneously bind to a cancer associated antigen and a T-cell surface protein such as CD3-epsilon. BiTEs are antibodies made by genetically linking the scFv's of an antibody that binds to a T cell antigen, like anti-CD3-epsilon to a scFv of a therapeutic monoclonal antibody that binds to a cancer antigen (Patrick A. Baeuerle, and Carsten Reinhardt (2009) Bispecific T-cell engaging antibodies for cancer therapy. Cancer Res. 69 (12): 4941-4944).

### SUMMARY OF THE INVENTION

In one aspect, the present invention is directed to a human or humanized anti-MUC1* antibody or antibody fragment or antibody-like protein that binds to a region on extracellular domain of MUC1 isoform or cleavage product that is devoid of the tandem repeat domains. The human or humanized anti-MUC1* antibody or antibody fragment or antibody-like protein may specifically bind to
(i) PSMGFR region of MUC1;
(ii) PSMGFR peptide;
(iii) a peptide having amino acid sequence of

(SEQ ID NO: 620)
SNIKFRPGSVVVQLTLAFREGTINVHDVETQFNQYKTEAASRY;

(iv) a peptide having amino acid sequence of

(SEQ ID NO: 621)
SVVVQLTLAFREGTINVHDVETQFNQYKTEAASRY;

(v) a peptide having amino acid sequence of

(SEQ ID NO: 622)
VQLTLAFREGTINVHDVETQFNQY;

(vi) a peptide having amino acid sequence of SNIKFRPGSVVVQLTLAFREGTIN (SEQ ID NO:623).

The human or humanized antibody may be IgG1, IgG2, IgG3, IgG4 or IgM. The human or humanized antibody fragment or antibody-like protein may be scFv or scFv-Fc.

The human or humanized antibody, antibody fragment or antibody-like protein as in above may comprise a heavy chain variable region and light chain variable region which is derived from mouse monoclonal MN-E6 antibody, and has at least 80%, 90% or 95% or 98% sequence identity to the mouse monoclonal MN-E6 antibody. The heavy chain variable region may have at least 90% or 95% or 98% sequence identity to SEQ ID NO:13 and the light chain variable region may have at least 90% or 95% or 98% sequence identity to SEQ ID NO: 66.

The human or humanized antibody, antibody fragment or antibody-like protein according to above may include complementarity determining regions (CDRs) in the heavy chain variable region and light chain variable region having at least 90% or 95% or 98% sequence identity to CDR1, CDR2 or CDR3 regions having sequence as follows:
CDR1 heavy chain SEQ ID NO:17
CDR1 light chain SEQ ID NO:70,
CDR2 heavy chain SEQ ID NO:21
CDR2 light chain SEQ ID NO:74,
CDR3 heavy chain SEQ ID NO:25
CDR3 light chain SEQ ID NO:78.

The human or humanized antibody, antibody fragment or antibody-like protein described above may include a heavy chain variable region and light chain variable region which is derived from mouse monoclonal MN-C2 antibody, and has at least 80%, 90% or 95% or 98% sequence identity to the mouse monoclonal MN-C2 antibody. The heavy chain variable region may have at least 90% or 95% or 98% sequence identity to SEQ ID NO:119 and the light chain variable region has at least 90% or 95% or 98% sequence identity to SEQ ID NO: 169. The complementarity determining regions (CDRs) in the heavy chain variable region and light chain variable region may have at least 90% or 95% or 98% sequence identity to CDR1, CDR2 or CDR3 regions having sequence as follows:
CDR1 heavy chain SEQ ID NO:123
CDR1 light chain SEQ ID NO:173,
CDR2 heavy chain SEQ ID NO:127
CDR2 light chain SEQ ID NO:177,
CDR3 heavy chain SEQ ID NO: 131
CDR3 light chain SEQ ID NO: 181.

The human or humanized antibody, antibody fragment or antibody-like protein as in above may include a heavy chain variable region and light chain variable region which is derived from mouse monoclonal MN-C3 antibody, and may have at least 80%, 90% or 95% or 98% sequence identity to the mouse monoclonal MN-C3 antibody. The heavy chain variable region may have at least 90% or 95% or 98% sequence identity to SEQ ID NO:414 and the light chain variable region may have at least 90% or 95% or 98% sequence identity to SEQ ID NO:459. The complementarity determining regions (CDRs) in the heavy chain variable region and light chain variable region may have at least 90% or 95% or 98% sequence identity to CDR1, CDR2 or CDR3 regions having sequence as follows:
CDR1 heavy chain SEQ ID NO:418
CDR1 light chain SEQ ID NO:463,
CDR2 heavy chain SEQ ID NO:422
CDR2 light chain SEQ ID NO:467,
CDR3 heavy chain SEQ ID NO:426,
CDR3 light chain SEQ ID NO:471.

The human or humanized antibody, antibody fragment or antibody-like protein described above may include a heavy chain variable region and light chain variable region which is derived from mouse monoclonal MN-C8 antibody, and has at least 80%, 90% or 95% or 98% sequence identity to the mouse monoclonal MN-C8 antibody. The heavy chain variable region may have at least 90% or 95% or 98% sequence identity to SEQ ID NO:506 and the light chain variable region may have at least 90% or 95% or 98% sequence identity to SEQ ID NO:544. The complementarity determining regions (CDRs) in the heavy chain variable region and light chain variable region may have at least 90% or 95% or 98% sequence identity to CDR1, CDR2 or CDR3 regions having sequence as follows:
CDR1 heavy chain SEQ ID NO:508
CDR1 light chain SEQ ID NO:546,
CDR2 heavy chain SEQ ID NO:510
CDR2 light chain SEQ ID NO:548,
CDR3 heavy chain SEQ ID NO:512,
CDR3 light chain SEQ ID NO:550.

In another aspect, the present invention is directed to an anti-MUC1* extracellular domain antibody comprised of sequences of a humanized MN-E6 represented by humanized IgG2 heavy chain, or humanized IgG1 heavy chain, paired with humanized Kappa light chain, or humanized Lambda light chain. The humanized IgG2 heavy chain may be SEQ ID NOS: 53, humanized IgG1 heavy chain may be SEQ ID NO:57, humanized Kappa light chain may be SEQ ID NO: 108, and humanized Lambda light chain may be SEQ ID NO: 112, or a sequence having 90%, 95% or 98% sequence identity thereof.

In another aspect, the invention is directed to an anti-MUC1* extracellular domain antibody comprised of sequences of a humanized MN-C2 represented by humanized IgG1 heavy chain, humanized IgG2 heavy chain, paired with humanized Lambda light chain, and humanized Kappa light chain. The humanized IgG1 heavy chain MN-C2 may be SEQ ID NOS: 159 or IgG2 heavy chain may be SEQ ID NOS: 164 paired with Lambda light chain (SEQ ID NO:219) or Kappa light chain (SEQ ID NO:213), or a sequence having 90%, 95% or 98% sequence identity thereof.

In another aspect, the invention is directed to an anti-MUC1* extracellular domain antibody comprised of sequences of a humanized MN-C3 represented by humanized IgG1 heavy chain or humanized IgG2 heavy chain paired with humanized Lambda light chain or humanized Kappa light chain. The humanized MN-C3 IgG1 heavy chain may be SEQ ID NOS: 454, IgG2 heavy chain may be SEQ ID NOS: 456, Lambda light chain may be SEQ ID NO: 501, and Kappa light chain may be SEQ ID NO:503, or a sequence having 90%, 95% or 98% sequence identity thereof.

In another aspect, the invention is directed to an anti-MUC1* extracellular domain antibody comprised of sequences of a humanized MN-C8 represented by humanized IgG1 heavy chain or humanized IgG2 heavy chain paired with humanized Lambda light chain or humanized Kappa light chain. The humanized MN-C8 IgG1 heavy chain may be SEQ ID NOS: 540, IgG2 heavy chain may be SEQ ID NOS: 542, Lambda light chain may be SEQ ID NO: 580 and Kappa light chain may be SEQ ID NO:582, or a sequence having 90%, 95% or 98% sequence identity thereof.

In another aspect, the invention is directed to a human or humanized anti-MUC1* antibody or antibody fragment or antibody-like protein according to above, which inhibits the binding of NME protein to MUC1*. The NME may be NME1, NME6, NME7AB, NME7 or NME8.

In yet another aspect, the invention is directed to a single chain variable fragment (scFv) comprising a heavy and light chain variable regions connected via a linker, further comprising CDRs of antibodies that bind to MUC1* extracellular domain. The CDRs may be derived from MN-E6, MN-C2, MN-C3 or MN-C8 antibodies or humanized antibodies thereof. The scFv may be one that possesses the SEQ ID NOS: 233, 235 and 237 (E6): SEQ ID NOS: 239, 241, and 243 (C2): SEQ ID NOS: 245, 247, and 249 (C3): or SEQ ID NOS: 251, 253, and 255 (C8).

In still another aspect, the invention is directed to a chimeric antigen receptor (CAR) comprising a scFv or a humanized variable region that binds to the extracellular domain of a MUC1 that is devoid of tandem repeats, a linker molecule, a transmembrane domain and a cytoplasmic domain. The single chain antibody fragment may bind to
(i) PSMGFR region of MUC1,
(ii) PSMGFR peptide,
(iii) a peptide having amino acid sequence

(SEQ ID NO: 620)
SNIKFRPGSVVVQLTLAFREGTINVHDVETQFNQYKTEAASRY;

(iv) a peptide having amino acid sequence of

(SEQ ID NO: 621)
SVVVQLTLAFREGTINVHDVETQFNQYKTEAASRY;

(v) a peptide having amino acid sequence of

(SEQ ID NO: 622)
VQLTLAFREGTINVHDVETQFNQY;

(vi) a peptide having amino acid sequence of

(SEQ ID NO: 623)
SNIKFRPGSVVVQLTLAFREGTIN.

In the CAR as describe above, portions of any of the variable regions set forth and described above, or combination thereof may be used in the extracellular domain, a transmembrane region and a cytoplasmic tail that comprises sequence motifs that signal immune system activation. The extracellular domain may be comprised of humanized single chain antibody fragments of an MN-E6 scFv, MN-C2 scFv, MN-C3 scFv or MN-C8 scFv.

In the CAR as described above, the extracellular domain include humanized single chain antibody fragments of an MN-E6 scFv set forth as SEQ ID NOS: 233, 235, or 237), MN-C2 scFv (SEQ ID NOS: 239, 241, or 243), MN-C3 scFv (SEQ ID NOS: 245, 247, or 249) or MN-C8 scFv (SEQ ID NOS: 251, 253, or 255).

In any of the CAR described above, the cytoplasmic tail may be comprised of one or more of signaling sequence motifs CD3-zeta, CD27, CD28, 4-1BB, OX40, CD30, CD40, PD-1, ICAm-1, LFA-1, ICOS, CD2, CD5, or CD7.

In any of the CAR described above, the sequence may be CARMN-E6 CD3z (SEQ ID NOS: 295), CARMN-E6 CD28/CD3z (SEQ ID NOS: 298): CARMN-E6 4-1BB/CD3z (SEQ ID NOS: 301); CARMN-E6 OX40/CD3z (SEQ ID NOS: 617): CARMN-E6 CD28/4-1BB/CD3z (SEQ ID NOS: 304): CARMN-E6 CD28/0X40/CD3z (SEQ ID NOS: 619): CAR MN-C2 CD3z (SEQ ID NOS: 607): CAR MN-C2 CD28/CD3z (SEQ ID NOS: 609): CAR MN-C2 4-1BB/CD3z (SEQ ID NOS: 611); CAR MN-C2 OX40/CD3z (SEQ ID NOS: 613): CAR MN-C2 CD28/4-1BB/CD3z (SEQ ID NOS: 307); or CAR MN-C2 CD28/OX40/CD3z (SEQ ID NOS: 615).

In another aspect, the CAR may have an extracellular domain unit that recognizes a peptide. The peptide may be PSMGFR (SEQ ID NO:2). The peptide may be a peptide derived from NME7. The peptide may be

NME7A peptide 1 (A domain):
(SEQ ID NO: 7)
MLSRKEALDFHVDHQS;
NME7A peptide 2 (A domain):
(SEQ ID NO: 8)
SGVARTDASES;
NME7B peptide 1 (B domain):
(SEQ ID NO: 9)
DAGFEISAMQMFNMDRVNVE;
NME7B peptide 2 (B domain):
(SEQ ID NO: 10)
EVYKGVVTEYHDMVTE;
or
NME7B peptide 3 (B domain):
(SEQ ID NO: 11)
AIFGKTKIQNAVHCTDLPEDGLLEVQYFF.

In another aspect, the invention is directed a composition that includes at least two CARs with different extracellular domain units transfected into the same cell.

The at least two CARs may have one CAR that does not have a targeting recognition unit and the other CAR does have a targeting recognition unit. Or, one of the extracellular domain recognition units may bind to MUC1* extracellular domain. Or, one of the extracellular domain recognition units may bind PD-1. Or, one of the extracellular domain recognition units is an antibody fragment and the other is a peptide. Or, one is an anti-MUC1* scFv chosen from the group consisting of scFv of MN-E6 antibody, scFv of MN-C2 antibody, scFv of MN-C3 antibody or scFv of MN-C8 antibody and the other is a peptide derived from NME7 or chosen from the group consisting of

NME7A peptide 1 (A domain):
(SEQ ID NO: 7)
MLSRKEALDFHVDHQS;
NME7A peptide 2 (A domain):
(SEQ ID NO: 8)
SGVARTDASES;
NME7B peptide 1 (B domain):
(SEQ ID NO: 9)
DAGFEISAMQMFNMDRVNVE;
NME7B peptide 2 (B domain):
(SEQ ID NO: 10)
EVYKGVVTEYHDMVTE;
and
NME7B peptide 3 (B domain):
(SEQ ID NO: 11)
AIFGKTKIQNAVHCTDLPEDGLLEVQYFF.

In another aspect, the invention is directed to a cell comprising a CAR with an extracellular domain that binds to MUC1* transfected or transduced cell. The cell that includes the CAR may be an immune system cell, preferably a T cell or dendritic cell or mast cell.

In another aspect, the invention is directed to an engineered antibody-like protein.

In another aspect, the invention is directed to a method of screening a library of antibodies or antibody fragments that are human, for those that bind to
(i) PSMGFR peptide;
(ii) a peptide having amino acid sequence

(SEQ ID NO: 620)
SNIKFRPGSVVVQLTLAFREGTINVHDVETQFNQYKTEAASRY;

(iii) a peptide having amino acid sequence of

(SEQ ID NO: 621)
SVVVQLTLAFREGTINVHDVETQFNQYKTEAASRY;

(iv) a peptide having amino acid sequence of

(SEQ ID NO: 622)
VQLTLAFREGTINVHDVETQFNQY;

(v) a peptide having amino acid sequence of SNIKFRPGSVVVQLTLAFREGTIN (SEQ ID NO:623);
(vi) NME7 protein: or
(vii) a peptide fragment of NME7 protein.

In another aspect, the invention is directed to a method for treating a disease in a subject comprising administering an antibody according to any claim above, to a person suffering from the disease, wherein the subject expresses MUC1 aberrantly. The disease may be cancer, such as breast cancer, lung cancer, colon cancer, gastric cancer.

In another aspect, the invention is directed to a method for treating a disease in a subject comprising administering an NME peptide, to a person suffering from the disease, wherein the subject expresses MUC1 aberrantly.

In another aspect, the invention is directed to a method of proliferating or expanding stem cell population comprising contacting the cells with the antibody according to any method or composition described above.

In another aspect, the invention is directed to amethod of facilitating stem cell attachment to a surface comprising coating the surface with a humanized MN-C3 or MN-C8 antibody, antibody fragment or single chain antibody thereof and contacting stem cell to the surface.

In another aspect, the invention is directed to a method of delivering stem cell in vitro or in vivo comprising the steps of coating a surface with a humanized MN-C3 or MN-C8 antibody, antibody fragment or single chain antibody thereof, contacting the stem cell to the surface and delivering the stem cell to a specific location.

In another aspect, the invention is directed to a method of isolating stem cell comprising the steps of coating a surface with a humanized MN-C3 or MN-C8 antibody, antibody fragment or single chain antibody thereof, and contacting a mixed population of cells to the surface and isolating stem cell.

In another aspect, the invention is directed to a scFv comprising variable domain fragments derived from an antibody that binds to a extracellular domain of MUC1 isoform or cleavage product that is devoid of the tandem repeat domains. The variable domain fragments may be derived from mouse monoclonal antibody MN-E6 (SEQ ID NO:13 and 66) or from the humanized MN-E6 (SEQ ID NO: 39 and 94), or from MN-E6 scFv (SEQ ID NO: 233, 235 and 237). Or, the variable domain fragments may be derived from mouse monoclonal antibody MN-C2 (SEQ ID NO: 119 and 169) or from the humanized MN-C2 (SEQ ID NO: 145 and 195), or from MN-C2 scFv (SEQ ID NO: 239, 241 and 243). Or, the variable domain fragments may be derived from mouse monoclonal antibody MN-C3 (SEQ ID NO: 414 and 459) or from the humanized MN-C3 (SEQ ID NO: 440 and 487), or from MN-C3 scFv (SEQ ID NO: 245, 247 and 249). Or, the variable domain fragments may be derived from mouse monoclonal antibody MN-C8 (SEQ ID NO: 505 and 544) or from the humanized MN-C8 (SEQ ID NO: 526 and 566), or from MN-C8 scFv (SEQ ID NO: 251, 253, 255).

In another aspect, the invention is directed to a method for the treatment of a person diagnosed with, suspected of having or at risk of developing a MUC1 Or MUC1* positive cancer involving administering to the person an effective amount of the scFv described above.

In another aspect, the invention is directed to a scFv-Fc construct comprising the scFv as described above. The scFv-Fc may be dimerized. Or, the Fc component may be mutated so that scFv-Fc is monomeric. The mutation may include mutating or deleting hinge region on Fc, making F405Q, Y407R, T366W/L368W, and T364R/L368R mutation or combinations thereof on the Fc represented by SEQ ID NO: 281, 279, 285 and 287.

In another aspect, the invention is directed to a polypeptide comprising at least two different scFv sequences, wherein one of the scFv sequences is a sequence that binds to extracellular domain of MUC1 isoform or cleavage product that is devoid of the tandem repeat domains. The polypeptide may bind to
(i) PSMGFR region of MUC1;
(ii) PSMGFR peptide;
(iii) peptide having amino acid sequence of

(SEQ ID NO: 620)
SNIKFRPGSVVVQLTLAFREGTINVHDVETQFNQYKTEAASRY;

(iv) a peptide having amino acid sequence of

(SEQ ID NO: 621)
VQLTLAFREGTINVHDVETQFNQYKTEAASRY;

(v) a peptide having amino acid sequence of

(SEQ ID NO: 622)
VQLTLAFREGTINVHDVETQFNQY;

(vi) a peptide having amino acid sequence of SNIKFRPGSVVVQLTLAFREGTIN (SEQ ID NO:623).

The polypeptide may bind to a receptor on an immune cell, such as T cell, and in particular, CD3 on T-cell.

In another aspect, the invention is directed to a method of detecting presence of a cell that expresses MUC1* aberrantly, comprising contacting a sample of cells with the scFv-Fc described above and detecting for the presence of the binding of scFv-Fc to the cell. The cell may be cancer cell.

In another aspect, the invention is directed to a method for testing a subject's cancer for suitability of treatment with a composition comprising portions of the variable regions of MN-E6, MN-C2, MN-C3 or MN-C8, comprising the steps of contacting a bodily specimen from the patient with the corresponding MN-E6 scFv-Fc, MN-C3 scFv-Fc, MN-C3 scFv-Fc or MN-C8 scFv-Fc.

In another aspect, the invention is directed to a method of treating a subject suffering from a disease comprising, exposing T cells from the subject to MUC1* peptides wherein through various rounds of maturation, T cells develop MUC1* specific receptors, creating adapted T cells, and expanding and administering the adapted T cells to the donor patient who is diagnosed with, suspected of having, or is at risk of developing a MUC1* positive cancer.

These and other objects of the invention will be more fully understood from the following description of the invention, the referenced drawings attached hereto and the claims appended hereto.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present invention will become more fully understood from the detailed description given herein below, and the accompanying drawings which are given by way of illustration only, and thus are not limitative of the present invention, and wherein:

FIG. 1 shows cell growth assay graphs of MUC1* positive cells treated with either bivalent by′ anti-MUC1* antibody, monovalent my′ or Fab. NM23-H1 dimers or NME7-AB. Bivalent anti-MUC1* antibodies stimulate growth of cancer cells whereas the monovalent Fab inhibits growth (A. B). Classic bell-shaped curve indicates ligand induced dimerization stimulates growth. Dimeric NM23-H1, aka NME1, stimulates growth of MUC1* positive cancer cells but siRNA to suppress MUC1 expression eliminate its effect (C). NME7-AB also stimulates the growth of MUC1* positive cells (D).

FIG. 2 shows results of ELISA assays. MUC1* peptides PSMGFR. PSMGFR minus 10 amino acids from the N-terminus aka N-10, or PSMGFR minus 10 amino acids from the C-terminus, aka C-10 are immobilized on the plate and the following are assayed for binding: NME7-AB (A). MN-C2 monoclonal antibody (B). MN-E6 monoclonal antibody (C), or dimeric NME1 (D). These assays show that NME1. NME7-AB and monoclonal antibodies MN-C2 and MN-E6 all require the first membrane proximal 10 amino acids of the MUC1* extracellular domain to bind. MUC1* peptides PSMGFR minus 10 amino acids from the N-terminus aka N-10, or PSMGFR minus 10 amino acids from the C-terminus, aka C-10, are immobilized on the plate and the following are assayed for binding: MN-C3 (E) and MN-C8 (F).

FIG. 3 shows results of competitive ELISA assays. The PSMGFR MUC1* peptide is immobilized on the plate and dimeric NM23-H1, aka NME1, is added either alone or after the MN-E6 antibody has been added (A). The same experiment was performed wherein NM23-H7. NME7-AB, is added alone or after MN-E6 has been added (B). Results show that MN-E6 competitively inhibits the binding of MUC1* activating ligands NME1 and NME7. In a similar experiment (C). PSMGFR or PSMGFR minus 10 amino acids from the N-terminus, aka N-10, is immobilized on the plate. Dimeric NM23-H1 is then added. Anti-MUC1* antibodies MN-E6, MN-C2, MN-C3 or MN-C8 are then tested for their ability to compete off the NM23-H1. Results show that although all three antibodies bind to the PSMGFR peptides. MN-E6 and MN-C2 competitively inhibit binding of the MUC1* activating ligands.

FIG. 4 shows FACS scans of anti-MUC1* antibodies binding specifically to MUC1* positive cancer cells and MUC1* transfected cells but not MUC1* or MUC1 negative cells. ZR-75-1, aka 1500, MUC1* positive breast cancer cells were stained with 1:2 or 1:10 dilutions of the 1.5 ug/ml humanized MN-C2. After two washes, cells were stained with secondary antibody. Anti-Penta-His antibody at conjugated to Alexa 488 (Qiagen) dilutions of 1:200 (A). 1:50 (B), or 1:10 (C) to detect the 6× His tag on the huMN-C2 scFv. Flow cytometric analysis revealed a concentration-dependent shift of a subset of cells, indicating specific binding, which is unseen in the absence of the MN-C2 scFv (A-C). In another case. MN-E6 was used to stain MUC1 negative HCT-116 colon cancer cells transfected with the empty vector, single cell clone #8 (D). HCT-116 colon cancer cells transfected with MUC1* single cell clone #10) (E), or ZR-75-1, aka 1500. MUC1* positive breast cancer cells. As the FACS scans show, both MN-C2 and MN-E6 only stain MUC1* positive cells and not MUC1 or MUC1* negative cells.

FIG. 5 shows a graph of an ELISA in which surface is coated with either the MUC1* PSMGFR peptide or a control peptide. Humanized MN-C2 scFv is then incubated with the surface, washed and detected according to standard methods. The ELISA shows that the huMN-C2 scFv binds to the MUC1* peptide with an EC-50 of about 333 nM.

FIG. 6 shows graphs of cancer cell growth inhibition by MUC1* antibody variable region fragment humanized MN-C2 scFv, hMN-C2 scFv potently inhibited the growth of ZR-75-1, aka 1500. MUC1* positive breast cancer cells (A) and T47D MUC1* positive breast cancer cells (B) with approximately the same EC-50 as the in vitro ELISAs.

FIG. 7 shows graphs of tumor growth in immune compromised mice that have been implanted with human tumors then treated with anti-MUC1* antibody MN-E6 Fab or mock treatment. Female nu/nu mice implanted with 90-day estrogen pellets were implanted with 6 million T47D human breast cancer cells that had been mixed 50/50 with Matrigel. Mice bearing tumors that were at least 150 mm3 and had three successive increases in tumor volume were selected for treatment. Animals were injected sub cutaneously twice per week with 80 mg/kg MN-E6 Fab and an equal number of mice fitting the same selection criteria were injected with vehicle alone (A). Male NOD/SCID mice were implanted with 6 million DU-145 human prostate cancer cells that had been mixed 50/50 with Matrigel. Mice bearing tumors that were at least 150 mm3 and had three successive increases in tumor volume were selected for treatment. Animals were injected sub-cutaneously every 48 hours with 160 mg/kg MN-E6 Fab and an equal number of mice fitting the same selection criteria were injected with vehicle alone (B). Tumors were measured independently by two researchers twice per week and recorded. Statistics were blindly calculated by independent statistician, giving a P value of 0.0001 for each. Anti-MUC1* Fab inhibited breast cancer growth and prostate cancer growth. Treatment had no effect on weight, bone marrow cell type or number.

FIG. 8 is a graph of an ELISA assay showing differing levels of expression of humanizedMN-E6 anti-MUC1* antibody depending on whether the light chain was kappa or lambda and whether the variable portion was fused to a human IgG1 or IgG2.

FIG. 9 is a graph of an ELISA assay comparing the binding of the parent mouseMN-E6 antibody to the humanized versions of theMN-E6 antibody to a surface presenting the PSMGFR peptide derived from the MUC1* extracellular domain.

FIG. 10 is a graph of an ELISA assay showing differing levels of expression of humanized MN-C2 anti-MUC1* antibody depending on whether the light chain was kappa or lambda and whether the variable portion was fused to a human IgG1 or IgG2.

FIG. 11 is a graph of an ELISA assay comparing the binding of the parent mouse MN-C2 antibody to the humanized versions of the MN-C2 antibody to a surface presenting the PSMGFR peptide derived from the MUC1* extracellular domain.

FIG. 12 is a graph of an ELISA assay showing binding of humanized single chain (scFv) MN-C2 andMN-E6 antibodies binding to a surface presenting the PSMGFR peptide derived from the MUC1* extracellular domain.

FIG. 13 shows FPLC traces of the purification of MN-E6 scFv-Fc fusion protein that was grown in low IgG FBS over a Protein A affinity column. A) is the trace of the flow through. B) is the trace of the elution. C) shows the purified protein on a reducing or non-reducing gel.

FIG. 14 shows photographs of SDS-PAGE characterization of purified MN-E6 scFv-Fc fusion proteins on a non-reducing gels, wherein the Fc portion that was fused to the MN-E6 was either wild type (wt) or mutated as follows: A) F405Q. Y407R. T394D: B) T366W/L368W. T364R/L368R. T366W/L368W or T364R/L368R. Fc mutants F405Q. Y407R. T366W/L368W. T364R/L368R. T366W/L368W and T364R/L368R all favored monomer over dimer formation. The reference construct amino acid sequence for the indicated mutations is SEQ ID NO:273.

FIGS. 15A-15B show FPLC traces of the purification of MN-E6 scFv-Fc Y407Q fusion protein that was grown in low IgG FBS over a Protein A affinity column. FIG. 15A is the trace of the flow through. FIG. 15B is the trace of the elution. FIG. 15C shows the protein was further purified by size exclusion over an S200 column. FIG. 15D is a photograph of an SDS-PAGE gel showing which fractions had a predominance of monomer. The reference construct amino acid sequence for the indicated mutations is SEQ ID NO: 273.

FIG. 16 shows a photograph of SDS-PAGE characterization of purified MN-E6 scFv-Fc-mutant fusion proteins on a non-reducing gel, wherein the Fc portion that was fused to the MN-E6 scFv was either wild type (wt) or mutated by elimination of the hinge region, ‘DHinge’, of the Fc or elimination of the hinge region of the Fc and also bearing the Y407R mutation. All the Fc mutants favored monomer over dimer formation. The reference construct amino acid sequence for the indicated mutations is SEQ ID NO:273.

FIG. 17A and FIG. 17B show photograph of non-reducing SDS-PAGE characterization of large scale expression and purification of MN-E6 scFv-Fc hingeless mutant, showing that it is a monomer. FIG. 17C shows FPLC characterization and purification of MN-E6 scFv-Fc hingeless mutant.

FIGS. 18A-18C show photographs of the SDS-PAGE characterization of the purified MN-C3 scFv-Fc fusion protein on a non-reducing gel (FIG. 18A) or a reducing gel (FIG. 18B). The protein was purified by size exclusion. The FPLC trace is shown (FIG. 18C).

FIG. 19 shows photographs of Native gels of MN-C3 or MN-E6 Fabs, scFv, scFv-Fc, wherein the Fc portion is wild type or mutants that prefer or are exclusively monomers. Native gels show that the Y407R Fc mutation (A) and the double mutant Y407R and a deleted hinge (B) favor monomer over dimer the best. Note that proteins are loaded onto a gel at much higher concentrations than typical use concentrations. The dimer formation of other Fc mutants may only reflect the fact that loading concentration is very high.

FIG. 20 shows a graph of an ELISA wherein the surface was immobilized with either PSMGFR peptide, PSMGFR minus 10 amino acids from the N-terminus or minus 10 amino acids from the C-terminus. The hu MN-E6 scFv-Fc bound to the PSMGFR peptide and to the PSMGFR N-10 peptide but not to the PSMGFR C-10 peptide. The parent MN-E6 antibody and the humanized MN-E6 require the C-terminal 10 amino acids of PSMGFR for binding.

FIG. 21 shows an ELISA graph of several anti-MUC1* scFv-Fc fusion proteins wherein the Fc region has been eliminated or mutated. Shown are hu MN-E6 scFv-Fc-wt, hu MN-E6 scFv-Fc-Y407R, hu MN-E6 scFv-Fc-hingeless, and hu MN-E6 scFv-Fc-Y407R-hingeless. All mutants bind to the PSMGFR peptide of the MUC1* extracellular domain (A). An ELISA graph of several anti-MUC1* scFv-Fc fusion proteins wherein the Fc region is either wild type or mutated. Shown are hu MN-E6 scFv-Fc-wt, hu MN-E6 scFv-Fc-hingeless, and hu MN-C3 scFv-Fc is shown (B). All bind to the PSMGFR peptide of the MUC1* extracellular domain.

FIG. 22 shows graphs of ELISAs wherein the assay plate surface was immobilized with either PSMGFR peptide, PSMGFR minus 10 amino acids from the N-terminus or minus 10 amino acids from the C-terminus. The MN-C3 antibody variants were then assayed for binding to the various MUC1* peptides. A) Purified mouse monoclonal MN-C3 antibody: B) Impure humanized MN-C3 antibody; and C) the humanized MN-C3 scFv-Fc. ELISAs show binding to the PSMGFR peptide as well as to certain deletion peptides.

FIG. 23 shows a graph of an ELISA assay that quantifies the binding of humanized MN-E6 scFv-Fc-delta hinge, aka Dhinge or hingeless, and humanized MN-E6 scFv to the MUC1* peptide PSMGFR.

FIG. 24 shows photographs of immunofluorescence experiments in which humanized MN-C2 scFv or MN-E6 scFv specifically binds to MUC1* positive breast cancer cells in an identical concentration dependent manner. A-G: hu MN-C2 scFv binding to T47D breast cancer cells at concentrations indicated. H-N shows the fluorescently labeled scFv and DAPI. O-U: hu MN-E6 scFv binding to T47D breast cancer cells at concentrations indicated. V-B′ shows the fluorescently labeled scFv and DAPI. C′ is the secondary antibody control.

FIG. 25 shows photographs of 1500 MUC1* positive breast cancer cells that have been cultured in normal medium or in the presence of humanized MN-E6 scFv. A-D are bright field images taken at 4× magnification. E-H are calcein fluorescent images taken at 4× magnification. I-L are calcein fluorescent images taken at 10× magnification. A,E, I show control cells cultured in normal RPMI medium. B.F. J show control cells cultured in normal RPMI medium plus a volume of PBS equal to the volume of MN-E6 scFv in PBS that is added to experimental wells. C. G. K show cells cultured in normal RPMI medium plus 500 ug/mL MN-E6 scFv. D. H. L show cells cultured in normal RPMI medium plus 5 ug/mL MN-E6 scFv. The photographs show killing and/or growth inhibition of MUC1* positive cells by MN-E6 scFv at 5 ug/mL and an even greater effect at 500 ug/mL. Images were taken at 96 hours post addition of test molecule.

FIG. 26 shows photographs of 1500 MUC1* positive breast cancer cells that have been cultured in normal medium or in the presence of humanized MN-E6 scFv-Fc Dhinge, which is a hingeless or delta hinge mutant. A-F are bright field images taken at 20× magnification. G-L are calcein fluorescent images taken at 4× magnification. A. G show control cells cultured in normal RPMI medium. B. H show cells cultured in normal RPMI medium plus 100 ug/mL hMN-E6 scFv-Fc Dhinge. C. I show cells cultured in normal RPMI medium plus 50 ug/mL hMN-E6 scFv-Fc Dhinge. D. J show cells cultured in normal RPMI medium plus 5 ug/mL hMN-E6 scFv-Fc Dhinge. E. K show cells cultured in normal RPMI medium plus 0.5 ug/mL hMN-E6 scFv-Fc Dhinge. F. L show cells cultured in normal RPMI medium plus 500 ug/mL of MN-E6 Fab. The photographs show killing and/or growth inhibition of MUC1* positive cells by hMN-E6 scFv-Fc Dhinge 5 ug/mL, an even greater effect at 50 ug/mL and yet an even greater effect at 100 ug/mL. Comparing cell morphology to the control cells, cancer cells grown in MN-E6 Fab or in an effective amount of hMN-E6 scFv-Fc Dhinge, show rounding up of the cells which morphology change occurs before cell death. Images were taken at 96 hours post addition of test molecule.

FIG. 27 shows a graph of the image analysis of the fluorescent images of FIGS. 25 and 26. Image J was used to quantify the number of cells remaining after 96 hours treatment in humanized MN-E6scFv or MN-E6 scFv-Fc-delta hinge, aka Dhinge. The analysis software uses pixel counting and pixel fluorescence intensity to quantify the number of cells in each photograph. Analysis was performed over the entire image 512×512 pixels. 8-bit image. For comparison, the inhibition of mouse monoclonal MN-E6 Fab is also analyzed.

FIG. 28 shows schematics of CAR sequence components.

FIG. 29 is a graph of an experiment measuring IL-2 cytokine secretion by Jurkat T cells that were transduced with a panel of CARs, including MN-E6-CD8-32, MN-E6-CD8-CD28-3z, MN-E6-CD8-41BB-37, MN-E6-CD4-CD28-3z and MN-E6-CD4-CD28-41BB-3z, when the CAR T cells were exposed to K562-wt cells or K562 cells that had been transfected with MUC1*.

FIG. 30 is a graph of an experiment measuring IL-2 cytokine secretion by Jurkat T cells that were transduced with a panel of CARs, including MN-E6-CD8-CD28-37. MN-E6-CD8-41BB-32, MN-E6-CD4-CD28-37 and MN-E6-CD4-41BB-3z, when the CAR T cells were exposed to K562-wt cells or K562 cells that had been transfected with MUC1*.

FIG. 31 is a graph of an experiment measuring IL-2 cytokine secretion by primary human T cells, isolated from a blood sample, that were transduced with a panel of CARs, including MN-E6-CD8-CD28-3z, MN-E6-CD8-41BB-3z and MN-E6-CD4-41BB-37, when the CAR T cells were exposed to K562-wt cells or K562 cells that had been transfected with MUC1*.

FIG. 32 is a graph of an experiment measuring interferon-gamma (IFN-g) cytokine secretion by primary human T cells, isolated from a blood sample, that were transduced with a panel of CARs, including MN-E6-CD8-CD28-3z, and MN-E6-CD4-41BB-3z, when the CAR T cells were exposed to K562-wt cells or K562 cells that had been transfected with MUC1*.

FIG. 33 is a graph of an experiment measuring interferon-gamma (IFN-g) cytokine secretion by primary human T cells, isolated from a blood sample, that were transduced with a panel of CARs, including MN-E6-CD8-CD28-37, MN-E6-CD8-41BB-3z and MN-E6-CD8-CD28-41BB-32, when the CAR T cells were exposed to K562-wt cells, K562 cells that had been transfected with MUC1*, or MUC1* positive cancer cells of prostate cancer, breast cancer or pancreatic cancer.

FIG. 34 is a graph of an experiment measuring target cell death when primary human T cells, isolated from a blood sample, that were transduced with a panel of CARs, including MN-E6-CD8-CD28-3z, MN-E6-CD8-41BB-32 and MN-E6-CD4-41BB-3z, when the CAR T cells were exposed to K562-wt cells or K562 cells that had been transfected with MUC1*. The ratio of T cells to target cells was 1:1 and the cells were co-cultured for 24 hours.

FIG. 35 is a graph of FACS measuring a time course of target cell survival from Day 1 to Day 3. Primary human T cells, isolated from a blood sample, were transduced with a panel of CARs, including humanized MN-E6-CD8-3z, MN-E6-CD8-CD28-3z, MN-E6-CD8-41BB-3z and MN-E6-CD8-CD28-41BB-3z. The CAR T cells were then exposed to K562-wt cells that naturally express low levels of MUC1*, or K562 cells that had been transfected with MUC1* high. The ratio of MUC1* targeting CAR T cells to target cells was either 1:1. 10:1, or 20:1. Surviving cells were detected and measured at Day 1 (A) or Day 3 (B).

FIG. 36 is a graph of FACS measurements of target cell survival at Day 3 of co-culture experiment. Primary human T cells were transduced with a panel of CARs, including humanized MN-E6-CD8-37, MN-E6-CD8-CD28-37, MN-E6-CD8-41BB-3z and MN-E6-CD8-CD28-41BB-32. The CAR T cells were then exposed to MUC1* positive T47D breast cancer cells or MUC1* positive 1500 aka ZR-75-1 breast cancer cells. The ratio of MUC1* targeting CAR T cells to target cells was either 1:1 or 10:1. As can be seen from the graph, T cells transduced with a MUC1* targeting CAR have a much greater killing effect on MUC1* cancer cells than the untransduced control T cells. In addition, the killing effect is much greater when the ratio of T cells: target cells is increased.

FIG. 37 is a graph of FACS measurements of target cell survival at Day 1 of co-culture experiment. Primary human T cells were transduced with a panel of CARs, including humanized MN-E6-CD8-41BB-3z, MN-E6-CD4-41BB-32, and MN-E6-CD8-CD28-41BB-37. The CAR T cells were then exposed to the following MUC1* positive cancer cells: T47D breast cancer: capan2 pancreatic cancer: or DU-145 prostate cancer. The ratio of MUC1* targeting CAR T cells to target cells was 5:1. As can be seen from the graph, T cells transduced with a MUC1* targeting CAR have a much greater killing effect on MUC1* cancer cells than the untransduced control T cells. Note that the measurements were taken after 24 hours with only a 5:1 T cell to target cell ratio. Also note that MUC1* targeting CARs that have a CD4 extracellular domain-transmembrane-cytoplasmic tail work equally well as CD8 constructs.

FIG. 38 is a graph of FACS measurements of target cell survival at Day 3 of co-culture experiment. Primary human T cells were transduced with a panel of CARs, including humanized MN-E6-CD8-41BB-3z, MN-E6-CD4-41BB-3z, and MN-E6-CD8-CD28-41BB-3z. The CAR T cells were then exposed to the following MUC1* positive cancer cells: K562 leukemia cells transfected with MUC1*: T47D breast cancer; 1500 aka ZR-75-1 breast cancer cells: or CAPAN-2 pancreatic cancer cells. In addition to the untransduced T cell controls, the assay was performed on PC3 MUC1* negative prostate cancer cells. The ratio of MUC1* targeting CAR T cells to target cells was 1:1. As can be seen from the graph, T cells transduced with a MUC1* targeting CAR have a much greater killing effect on MUC1* cancer cells than the untransduced control T cells. In addition, the killing effect is specific for MUC1* positive cells. Note that MUC1* targeting CARs that have a CD4 extracellular domain-transmembrane-cytoplasmic tail work equally well as CD8 constructs.

FIG. 39 is a graph of FACS measurements of CAR T cell expansion over 24 hours in co-culture with target cells at a ratio of 5:1 CAR T cells to target cells. The primary human T cells were transduced with a panel of CARs, including humanized MN-E6-CD8-41BB-3z, MN-E6-CD4-41BB-3z, and MN-E6-CD8-CD28-41BB-37. The CAR T cells were co-cultured with MUC1* positive T47D breast cancer cells, MUC1* positive Capan pancreatic cancer cells, and MUC1-negative cells HCT-116 colon cancer cells and HEK-293 human embryonic kidney cells. As can be seen from the graph, the CAR T population is increased in the presence of MUC1* positive cells.

FIG. 40 shows a photograph of a Western blot of MUC1* targeting CARs. From 1 to 9 are:
1.E6scFv-Fc-8-41BB-CD3z (Human Fc as hinge region with CD8 TM):
2: E6scFv-FcH-8-41BB-CD3z (Human Fc hingeless as hinge region with CD8 TM)
3: E6scFv-Fc-4-41BB-CD3z (Human Fc as hinge region with CD4 TM)
4: E6scFv-FcH-4-41BB-CD3z (Human Fc as hingeless hinge region with CD4 TM)
5: E6scFv-IgD-8-41BB-CD3z (hinge region from human IgD with CD8 TM)
6: E6scFv-IgD-4-41BB-CD3z (hinge region from human IgD with CD4 TM)
7: E6scFv-X4-8-41BB-CD3z (Long flexible linker as hinge region with CD8 TM)
8: E6scFv-X4-4-41BB-CD3z (Long flexible linker as hinge region with CD4 TM)
9: E6scFv-8-4-41BB-CD3z (Hinge region from CD8 and CD4 a with CD4 TM).

FIG. 41 shows graphs of FACS scans of T47D breast cancer cells co-cultured with human T cells that were transduced with MN-E6scFv-Fc-8-41BB-CD3z, MN-E6scFv-FcH-8-41BB-CD3z, MN-E6scFv-Fc-4-41BB-CD3z, MN-E6scFv-IgD-8-41BB-CD37, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

FIG. 42 shows graphs of FACS scans of T47D breast cancer cells. Capan-2 pancreatic cancer cells, K562-MUC1* transfected cells, and K562-wt cells that were co-cultured with human T cells that were transduced with MN-E6scFv-Fc-8-41BB-CD3z, MN-E6scFv-FcH-8-41BB-CD32, MN-E6scFv-Fc-4-41BB-CD37, MN-E6scFv-IgD-8-41BB-CD3z, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

FIG. 43 shows photographs of breast cancer tissue arrays. A) was stained with VU4H5 which recognizes MUC1-FL (full length): B) was stained with mouse monoclonal antibody MN-C2 which recognizes cancerous MUC1*. Following automated staining (Clarient Diagnostics), the tissue staining was scored using Allred scoring method which combines an intensity score and a distribution score. C,D,E,F are color coded graphs showing the score calculated for MUC1 full-length staining for each patient's tissue. G,H,I,J are color coded graphs showing the score calculated for MUC1* staining for each patient's tissue.

FIG. 44 shows photographs of breast cancer tissue arrays. A) was stained with VU4H5 which recognizes MUC1-FL (full length): B) was stained with mouse monoclonal antibody MN-C2 which recognizes cancerous MUC1*. Following automated staining (Clarient Diagnostics), the tissue staining was scored using Allred scoring method which combines an intensity score and a distribution score. C,D,E,F are color coded graphs showing the score calculated for MUC1 full-length staining for each patient's tissue. G,H,I,J are color coded graphs showing the score calculated for MUC1* staining for each patient's tissue.

FIG. 45 shows photographs of normal breast and breast cancer tissues stained with humanized MN-E6-scFv-Fc biotinylated anti-MUC1* antibody at 2.5 ug/mL, then stained with a secondary streptavidin HRP antibody. A) is a normal breast tissue. B-D are breast cancer tissues from patients as denoted in the figure. E-H are photographs of the corresponding serial sections that were stained with the secondary antibody alone.

FIG. 46 shows photographs of normal breast and breast cancer tissues stained with humanized MN-E6-scFv-Fc biotinylated anti-MUC1* antibody at 2.5 ug/mL, then stained with a secondary streptavidin HRP antibody. A) is a normal breast tissue. B-C are breast cancer tissues from patients as denoted in the figure. D-F are photographs of the corresponding serial sections that were stained with the secondary antibody alone.

FIG. 47 shows photographs of breast cancer tissues stained with MN-E6 anti-MUC1* antibody at 10 ug/mL, then stained with a rabbit anti mouse secondary HRP antibody. A-D are breast cancer tissues from patient #300. E-H are breast cancer tissues from metastatic patient #291.

FIG. 48 shows photographs of normal lung and lung cancer tissues stained with humanized MN-E6-scFv-Fc biotinylated anti-MUC1* antibody at 2.5 ug/mL, then stained with a secondary streptavidin HRP antibody. A) is a normal lung tissue. B,C are lung cancer tissues from patients as denoted in the figure. D-F are photographs of the corresponding serial sections that were stained with the secondary antibody alone.

FIG. 49 shows photographs of normal lung and lung cancer tissues stained with humanized MN-E6-scFv-Fc biotinylated anti-MUC1* antibody at 2.5 ug/mL, then stained with a secondary streptavidin HRP antibody. A) is a normal lung tissue. B.C are lung cancer tissues from patients as denoted in the figure. D-F are photographs of the corresponding serial sections that were stained with the secondary antibody alone.

FIG. 50 shows photographs of normal lung and lung cancer tissues stained with humanized MN-E6-scFv-Fc biotinylated anti-MUC1* antibody at 25 ug/mL, then stained with a secondary streptavidin HRP antibody. A) is a normal lung tissue. B.C are lung cancer tissues from patients as denoted in the figure. D-F are photographs of the corresponding serial sections that were stained with the secondary antibody alone.

FIG. 51 shows photographs of normal lung and lung cancer tissues stained with humanized MN-E6-scFv-Fc biotinylated anti-MUC1* antibody at 25 ug/mL, then stained with a secondary streptavidin HRP antibody. A) is a normal lung tissue. B,C are lung cancer tissues from patients as denoted in the figure. D-F are photographs of the corresponding serial sections that were stained with the secondary antibody alone.

FIG. 52 shows photographs of normal small intestine and cancerous small intestine tissues stained with humanized MN-E6-scFv-Fc biotinylated anti-MUC1* antibody at 5 ug/mL, then stained with a secondary streptavidin HRP antibody. A) is a normal small intestine tissue. B) is small intestine cancer from patient as denoted in the figure. C,D are photographs of the corresponding serial sections that were stained with the secondary antibody alone.

FIG. 53 shows photographs of normal small intestine tissues stained with humanized MN-E6-scFv-Fc anti-MUC1* antibody at 50 ug/mL, then stained with a secondary goat-anti-human HRP antibody. A-D are normal small intestine tissue. E-H are photographs of the corresponding serial sections that were stained with the secondary antibody alone.

FIG. 54 shows photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone.

FIG. 55 shows photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone.

FIG. 56 shows photographs of normal colon tissues stained with humanized MN-E6-scFv-Fc anti-MUC1* antibody at 50 ug/mL, then stained with a secondary goat-anti-human HRP antibody. A-D are normal colon. E-H are photographs of the corresponding serial sections that were stained with the secondary antibody alone.

FIG. 57 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone.

FIG. 58 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a Grade 2 patient as denoted in figure. E-H are photographs of the corresponding serial sections that were stained with the secondary antibody alone.

FIG. 59 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone.

FIG. 60 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

FIG. 61 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

FIG. 62 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

### DETAILED DESCRIPTION OF THE PREFERRED EMBODIMENTS

In the present application, “a” and “an” are used to refer to both single and a plurality of objects.

As used herein, “h” or “hu” placed before an antibody construct is short-hand for humanized.

As used herein, the term “antibody-like” means a molecule that may be engineered such that it contains portions of antibodies but is not an antibody that would naturally occur in nature. Examples include but are not limited to CAR (chimeric antigen receptor) T cell technology and the Ylanthia® technology. The CAR technology uses an antibody epitope fused to a portion of a T cell so that the body's immune system is directed to attack a specific target protein or cell. The Ylanthia® technology consists of an “antibody-like” library that is a collection of synthetic human Fabs that are then screened for binding to peptide epitopes from target proteins. The selected Fab regions can then be engineered into a scaffold or framework so that they resemble antibodies.

As used herein, the antibodies MN-C2, MN-E6, MN-C3 and MN-C8, may also be referred to as C2, E6, C3 and C8, respectively.

As used herein, “PSMGFR” is abbreviation for Primary Sequence of the MUC1 Growth Factor Receptor which is identified by SEQ ID NO:2, and thus is not to be confused with a six amino acid sequence. “PSMGFR peptide” or “PSMGFR region” refers to a peptide or region that incorporates the Primary Sequence of the MUC1 Growth Factor Receptor (SEQ ID NO:2).

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:2)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

Other clipped amino acid sequences may include SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

As used herein, the term “PSMGFR” is an acronym for Primary Sequence of MUC1 Growth Factor Receptor as as set forth GTINVHDVETQFNQYKTEAASRYNLTISDVSVSDVPFPFSAQSGA (SEQ ID NO:2). In this regard, the “N-number” as in “N-10 PSMGFR”. “N-15 PSMGFR”, or “N-20 PSMGFR” refers to the number of amino acid residues that have been deleted at the N-terminal end of PSMGFR. Likewise “C-number” as in “C-10 PSMGFR”. “C-15 PSMGFR”, or “C-20 PSMGFR” refers to the number of amino acid residues that have been deleted at the C-terminal end of PSMGFR.

As used herein, the “extracellular domain of MUC1*” refers to the extracellular portion of a MUC1 protein that is devoid of the tandem repeat domain. In most cases. MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein “sequence identity” means homology in sequence of a particular polypeptide or nucleic acid to a reference sequence of nucleic acid or amino acid such that the function of the homologous peptide is the same as the reference peptide or nucleic acid. Such homology can be so close with the reference peptide such that at times the two sequences may be 90%, 95% or 98% identical yet possess the same function in binding or other biological activities.

MUC1* antibodies (anti-PSMGFR) for treatment or prevention of cancers

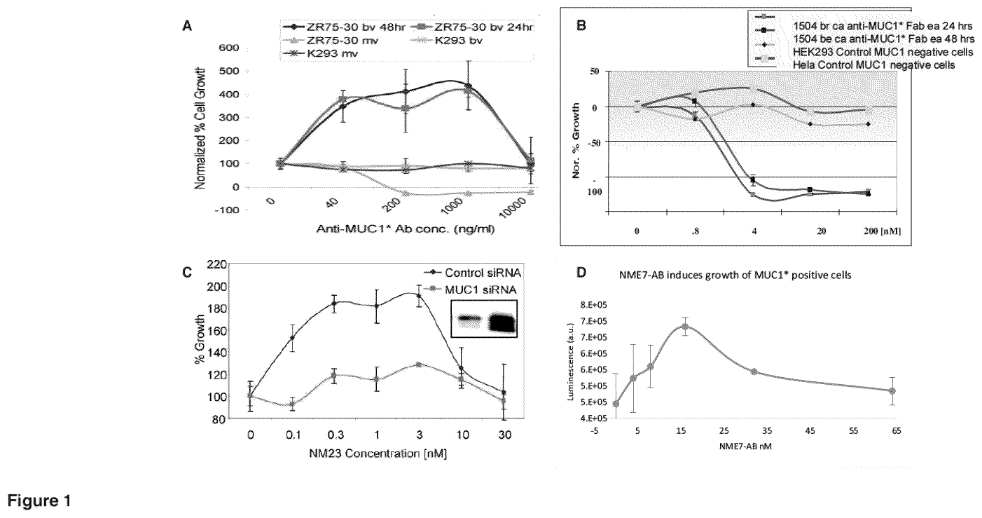

We discovered that a cleaved form of the MUC1 (SEQ ID NO:1) transmembrane protein is a growth factor receptor that drives the growth of over 75% of all human cancers. The cleaved form of MUC1, which we called MUC1* (pronounced muk 1 star), is a powerful growth factor receptor. Enzymatic cleavage releases the bulk of the MUC1 extracellular domain. It is the remaining portion comprising a truncated extracellular domain, transmembrane and cytoplasmic tail that is called MUC1*. Cleavage and release of the bulk of the extracellular domain of MUC1 unmasks a binding site for activating ligands dimeric NME1, NME6, NME8, NME7-AB or NME7. Cell growth assays show that it is ligand-induced dimerization of the MUC1* extracellular domain that promotes growth (FIG. 1A-D). MUC1* positive cells treated with either bivalent ‘bv’ anti-MUC1* antibody, monovalent ‘my’ or Fab. NM23-H1 dimers or NME7-AB. Bivalent anti-MUC1* antibodies stimulate growth of cancer cells whereas the monovalent Fab inhibits growth. Classic bell-shaped curve indicates ligand induced dimerization stimulates growth. Dimeric NM23-H1, aka NME1, stimulates growth of MUC1* positive cancer cells but siRNA to suppress MUC1 expression eliminate its effect (C). NME7-AB also stimulates the growth of MUC1* positive cells (D).

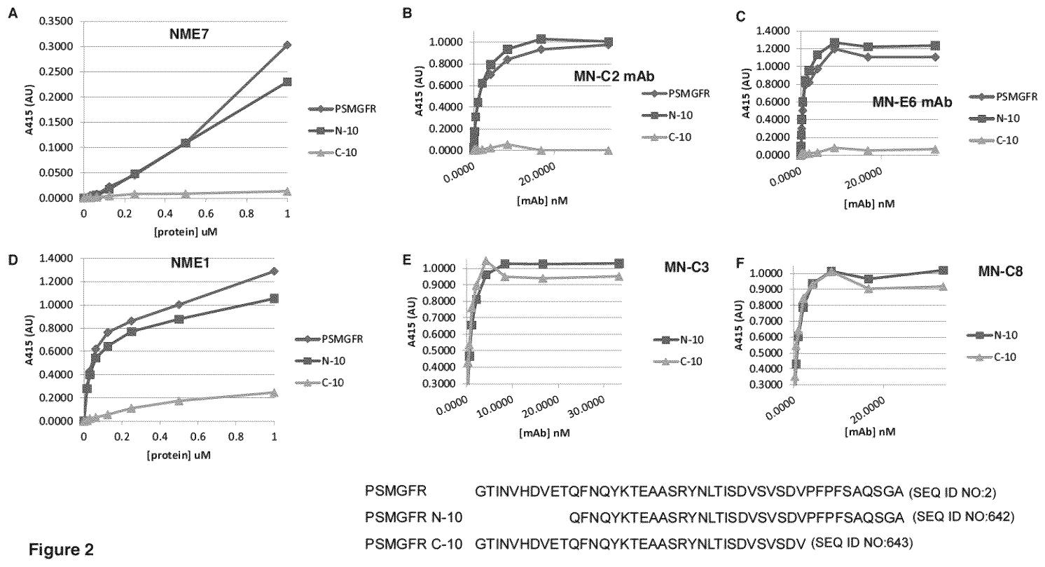

MUC1* is an ideal target for cancer drugs as it is aberrantly expressed on over 75% of all cancers and is likely overexpressed on an even higher percentage of metastatic cancers (Fessler SP. Wotkowicz MT. Mahanta SK and Bamdad C. (2009). MUC1* is a determinant of trastuzumab (Herceptin) resistance in breast cancer cells. Breast Cancer Res Treat. 118 (1): 113-124). After MUC1 cleavage most of its extracellular domain is shed from the cell surface. The remaining portion has a truncated extracellular domain that at least comprises the primary growth factor receptor sequence. PSMGFR (SEQ ID NO:2). Antibodies that bind to the PSMGFR sequence and especially those that competitively inhibit the binding of activating ligands such as NME proteins, including NME1, NME6, NME8 and NME7, are ideal therapeutics and can be used to treat or prevent MUC1 positive or MUC1* positive cancers, as stand-alone antibodies, antibody fragments or variable region fragments thereof incorporated into bispecific antibodies, or chimeric antigen receptors also called CARs. Therapeutics anti-MUC1* antibodies can be monoclonal, polyclonal, antibody mimics, engineered antibody-like molecules, full antibodies or antibody fragments. Examples of antibody fragments include but are not limited to Fabs, scFv, and scFv-Fc. Human or humanized antibodies are preferred for use in the treatment or prevention of cancers. In any of these antibody-like molecules, mutations can be introduced to prevent or minimize dimer formation. Anti-MUC1* antibodies that are monovalent or bispecific are preferred because MUC1* function is activated by ligand induced dimerization. Typical binding assays show that NME1 and NME7 bind to the PSMGFR peptide portion of MUC1* (FIG. 2A, D). Further, they show that these activating growth factors bind to the membrane proximal portion of MUC1*, as they do not bind to the PSMGFR peptide if the 10 C-terminal amino acids are missing. Similarly, anti-MUC1* antibodies MN-C2 and MN-E6 bind to the PSMGFR peptide if an only if the 10 C-terminal amino acids are present (FIG. 2B, C). Antibodies MN-C3 and MN-C8 bind to epitopes that are different from MN-C2 and MN-E6, as they do not depend on the presence of the 10 C-terminal amino acids of the PSMGFR peptide (FIG. 2E, F). Antibodies MN-C2, MN-E6, MN-C3 or MN-C8, or fragments derived from them, as stand-alone antibodies or incorporated into bispecific antibodies. BiTEs or chimeric antigen receptors also called CARs expressed by immune cells are all potent anti-cancer therapeutics.

Therapeutic anti-MUC1* antibodies for use as a stand alone antibody therapeutic or for integration into a BiTE or a CAR can be selected based on specific criteria. The parent antibody can be generated using typical methods for generating monoclonal antibodies in animals. Alternatively, they can be selected by screening antibody and antibody fragment libraries for their ability to bind to a MUC1* peptide, which can be the PSMGFR peptide (SEQ ID NO:2). SNIKFRPGSVVVQLTLAFREGTINVHDVETQFNQYKTEAASRY (SEQ ID NO: 620): orSVVVQLTLAFREGTINVHDVETQFNQYKTEAASRY (SEQ ID NO:621).

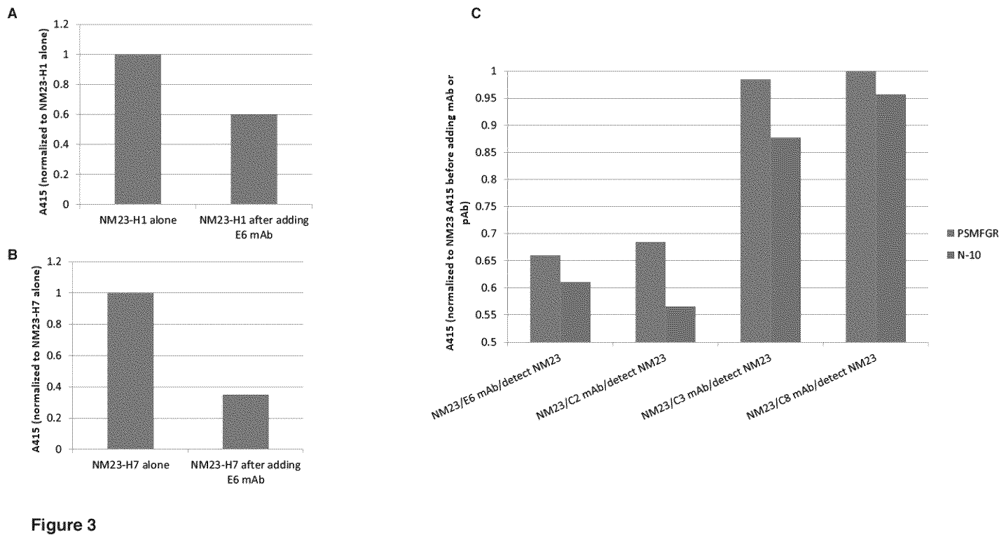

Resultant antibodies or antibody fragments generated or selected in this way can then be further selected by passing additional screens. For example, antibodies or antibody fragments become more preferred based on their ability to bind to MUC1* positive cancer cells or tissues but not to MUC1 negative cancer cells or tissues. Further, anti-MUC1* antibodies or antibody fragments may be de-selected as anti-cancer therapeutics if they bind to stem or progenitor cells. Anti-MUC1* antibodies or antibody fragments become more preferred if they have the ability to competitively inhibit the binding of activating ligands to MUC1*. FIG. 3A-C shows that MN-E6 and MN-C2 competitively inhibit the binding of activating ligands NME1 and NME7 to MUC1*. A process for selecting anti-MUC1* antibodies for use in treating a patient diagnosed with a MUC1 positive cancer, at risk of developing a MUC1 positive cancer or suspected of having a MUC1 positive cancer comprises one or more of the following steps of selecting antibodies or antibody fragments that 1) bind to the PSMGFR peptide: 2) bind to the N-10 PSMGFR peptide: 3) bind to cancer cells; 4) do not bind to stem or progenitor cells; and 5) competitively inhibited the binding of dimeric NME1 or NME7-AB to the PSMGFR peptide. For example. FIG. 3A-C show that monoclonals MN-E6 and MN-C2 satisfy all five criteria, while monoclonals MN-C3 and MN-C8 do not competitively inhibit the binding of activating ligands NME1 and NME7 (Figure M3 C). However, antibodies or antibody fragments derived from MN-C3 and MN-C8 are equally potent as anti-cancer agents when integrated into a BiTE or a CAR as in these methods, the killing effect of the immune cells is more important than the ability to inhibit the binding of activating ligands. In addition, toxic agents conjugated to MN-E6, MN-C2, MN-C3 or MN-C8 are potent anti-cancer therapeutics. Recall that the MUC1* growth factor receptor is activated by ligand-induced dimerization of its extracellular domain. Therefore the ideal antibody therapeutic should not dimerize the MUC1* extracellular domain. Preferably, suitable antibodies in this regard include monovalent antibodies such as those generated in lamas and camels, Fabs, scFv's, single domain antibodies (sdAb), scFv-Fc as long as the Fc portion is constructed such that it does not homo-dimerize.

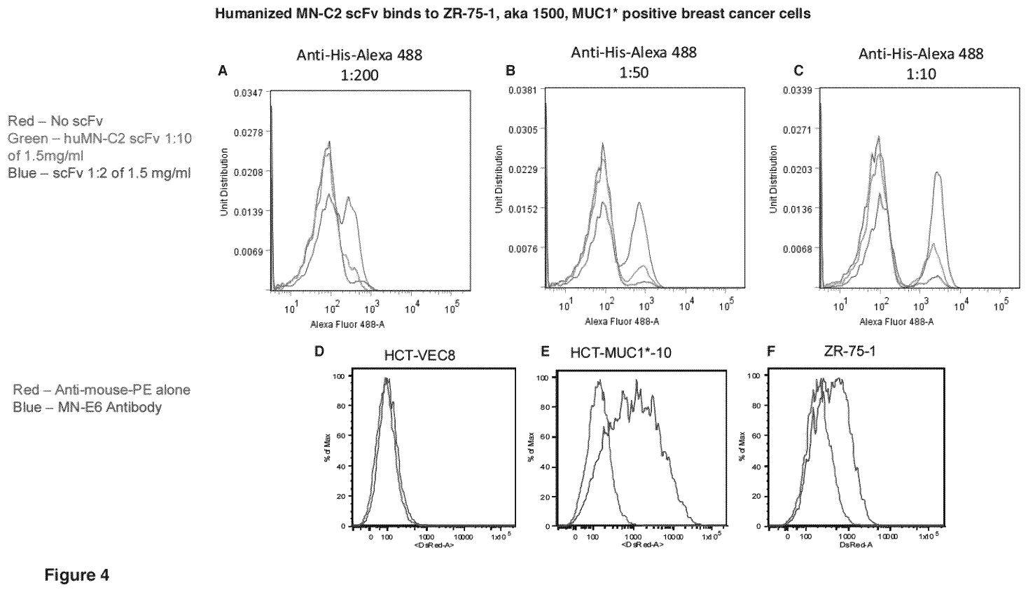

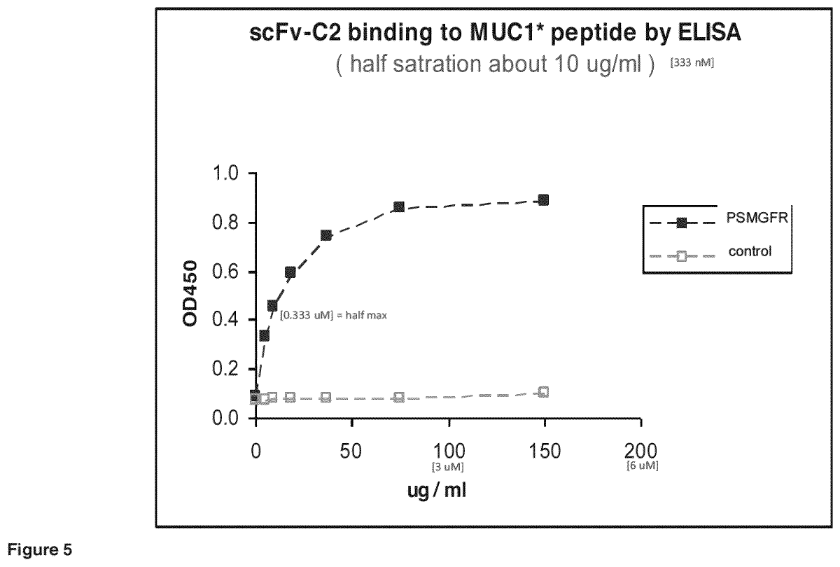

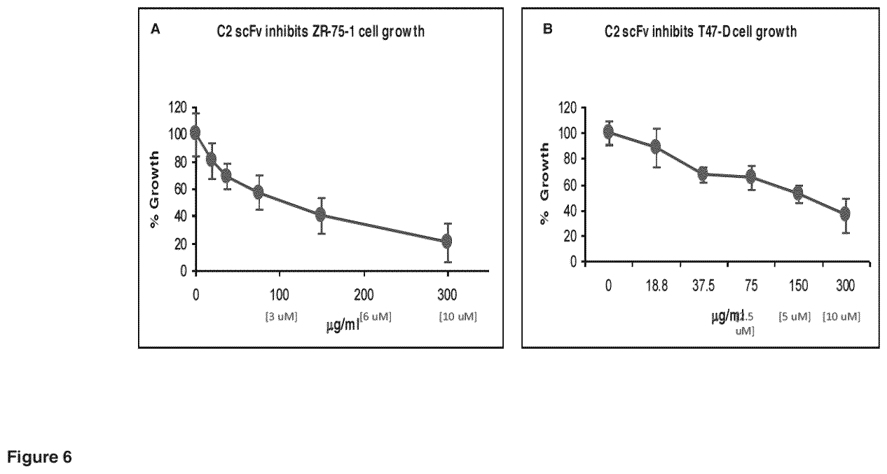

FACS scans show that anti-MUC1* antibodies MN-C2, MN-E6, MN-C3 and MN-C8 specifically bind to MUC1* positive cancer cells and MUC1* transfected cells but not MUC1* or MUC1 negative cells. In one example, a humanized MN-C2 scFv is shown to bind to ZR-75-1, aka 1500, MUC1* positive breast cancer cells (FIG. 4A-C). MN-E6 was shown to bind to MUC1 negative HCT-116 colon cancer cells if an only if they were transfected with MUC1*, MN-E6 also bound to MUC1* positive cancer cells such as ZR-75-1, aka 1500, MUC1* positive breast cancer cells (FIG. 4D-F). Binding assays such as ELISAs, immunofluorescence, and the like all confirm that MN-C2 and MN-E6 bind to the PSMGFR peptide and to live MUC1 positive cancer cells. Humanized anti-MUC1* antibodies are selected based on their ability to also bind to the PSMGFR peptide or to MUC1 positive cancer cells. FIG. 5 shows that humanized MN-C2 scFv binds with high affinity to the MUC1* peptide PSMGFR with an EC-50 of about 333 nM. Humanized MN-C2 scFv, like Fabs, potently inhibits the growth of MUC1* positive cancer cells as is shown in one example in FIG. 6A. B.

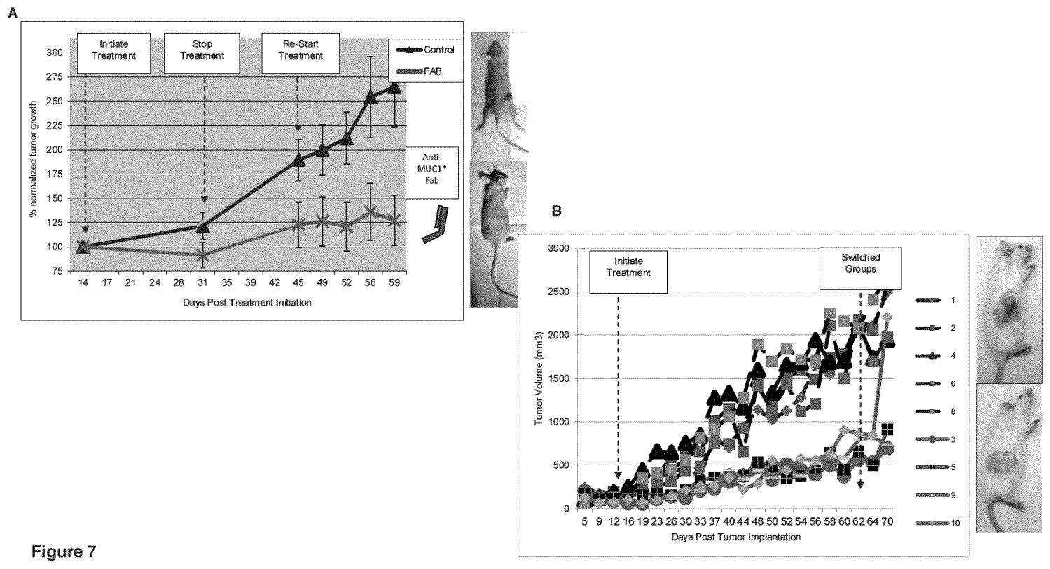

The Fabs of MN-E6 and MN-C2 or the comparable single chain variable regions derived from them potently inhibit the growth of MUC1* positive cancers in vitro and in vivo. In several examples, the Fabs of Anti-MUC1* antibodies inhibited the growth of human MUC1* positive cancers in vivo. In one case, immune-compromised mice were implanted with human breast tumors then treated with MN-E6 Fab after tumor engraftment. FIG. 7A shows that MN-E6 Fab potently inhibited the growth of MUC1* positive breast cancers. Female nu/nu mice implanted with 90-day estrogen pellets were implanted with 6 million T47D human breast cancer cells that had been mixed 50/50 with Matrigel. Mice bearing tumors that were at least 150 mm\hat{ }3 and had three successive increases in tumor volume were selected for treatment. Animals were injected sub cutaneously twice per week with 80 mg/kg MN-E6 Fab and an equal number of mice fitting the same selection criteria were injected with vehicle alone (A). In another aspect. MN_E6 was shown to halt the growth of prostate cancer. FIG. 7B shows that MN-E6 Fab potently inhibited the growth of MUC1* positive prostate cancers. Male NOD/SCID mice were implanted with 6 million DU-145 human prostate cancer cells that had been mixed 50/50 with Matrigel. Mice bearing tumors that were at least 150 mm\hat{ }3 and had three successive increases in tumor volume were selected for treatment. Animals were injected sub-cutaneously every 48 hours with 160 mg/kg MN-E6 Fab and an equal number of mice fitting the same selection criteria were injected with vehicle alone (B). Tumors were measured independently by two researchers twice per week and recorded. Statistics were blindly calculated by independent statistician, giving a P value of 0.0001 for each. Anti-MUC1* Fab inhibited breast cancer growth and prostate cancer growth. Treatment had no effect on weight, bone marrow cell type or number. The MN-E6 Fab effectively inhibited the growth of the tumors, while the control group's tumors continued to grow until sacrifice. No adverse effects of treatment were observed or detected.

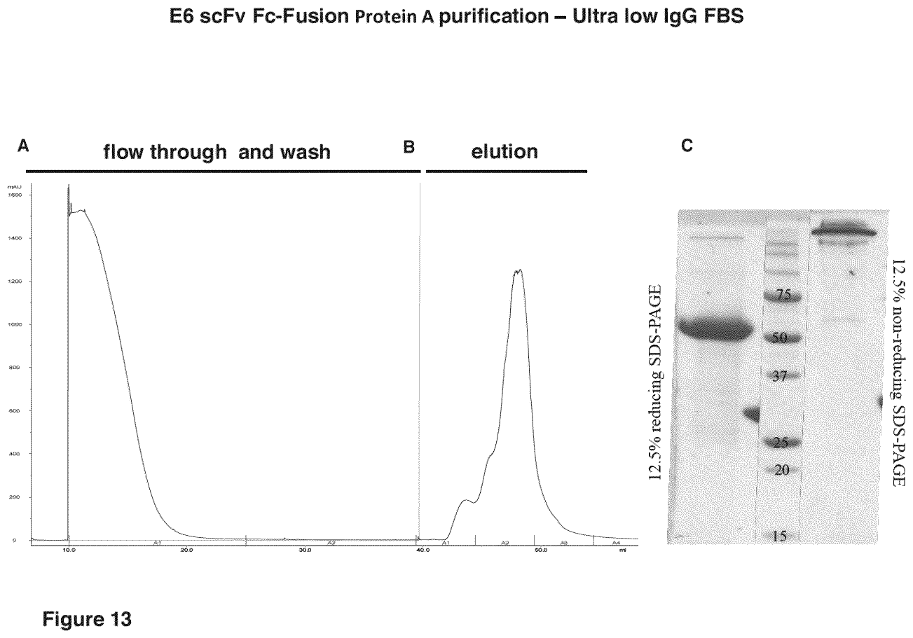

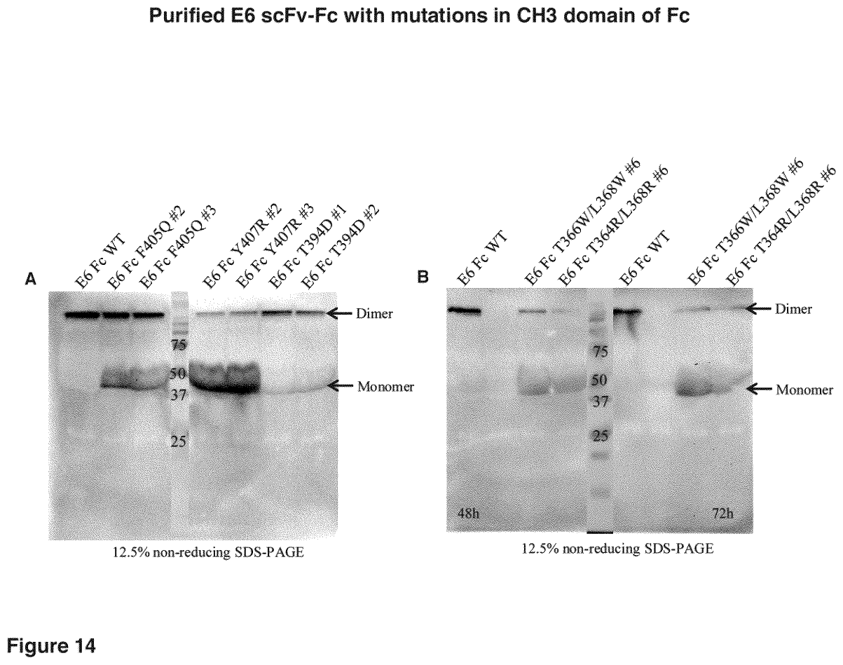

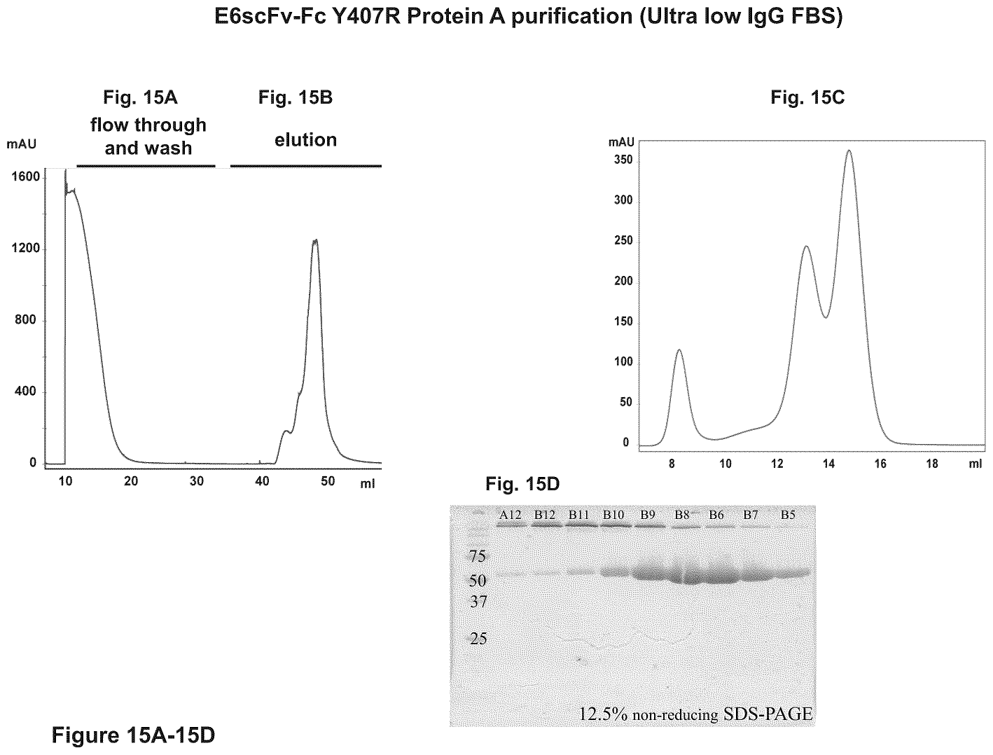

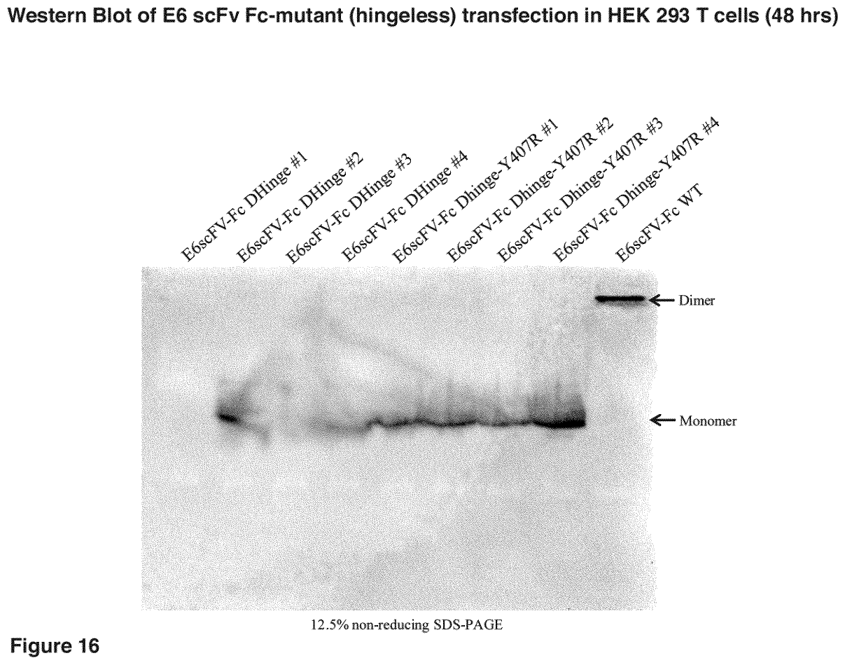

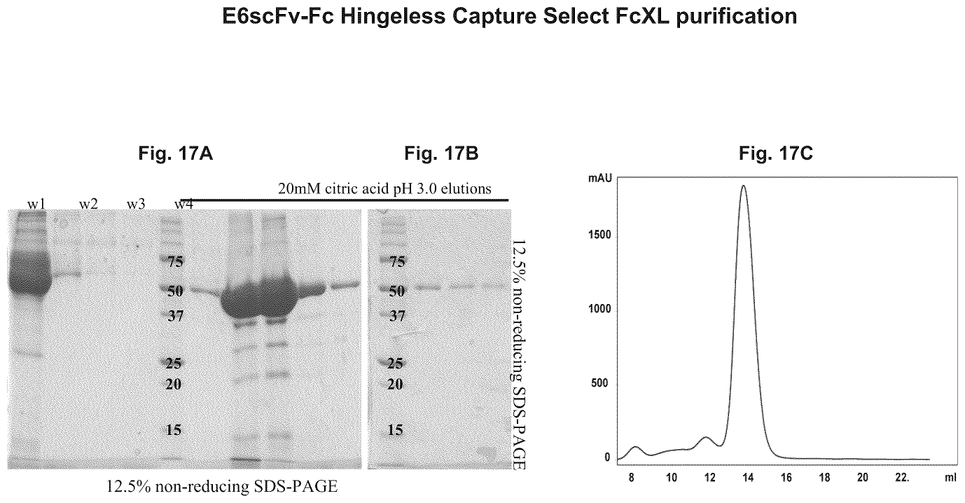

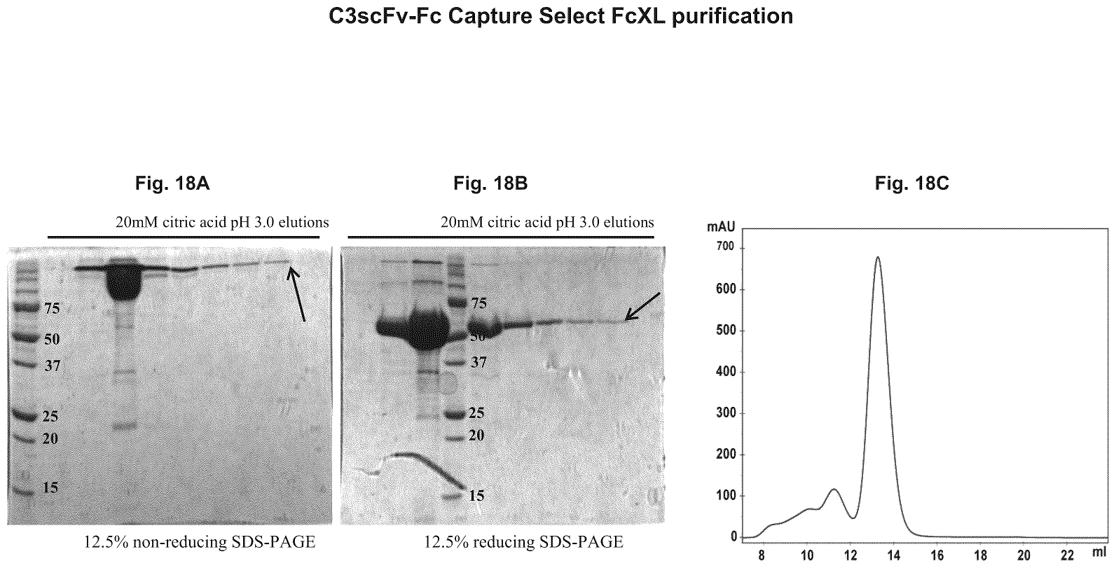

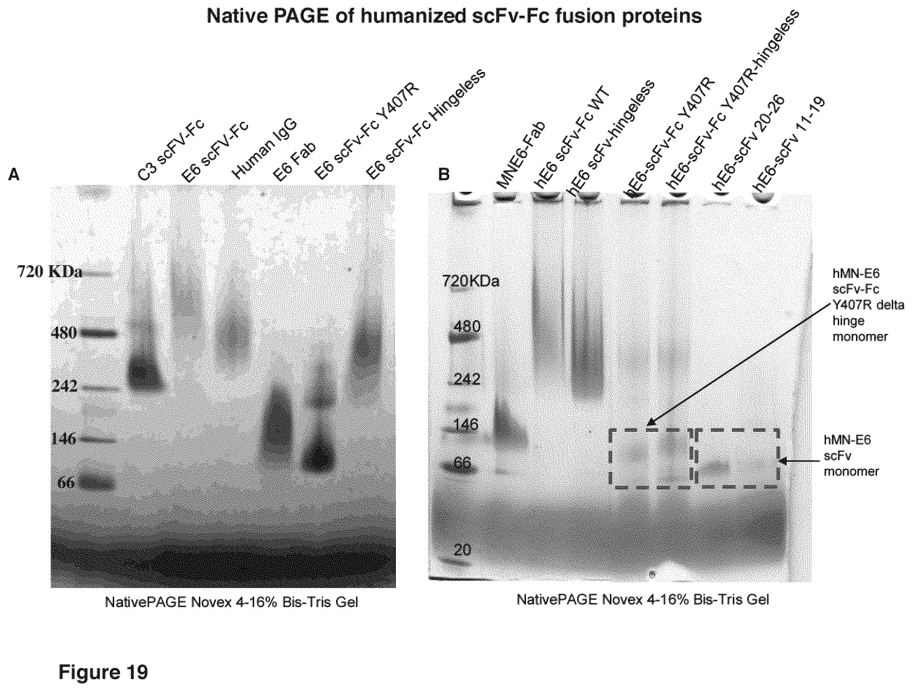

A recombinant MN-E6 was constructed that like the Fab is monomeric. In this case. MN-E6 was humanized. There are a number of methods known to those skilled in the art for humanizing antibodies. In addition to humanizing, libraries of human antibodies can be screened to identify other fully human antibodies that bind to the PSMGFR. A single chain of the humanized MN-E6 variable region, called an scFv, was genetically engineered such that it was connected to the Fc portion of the antibody (SEQ ID NO:256 and 257). Fc regions impart certain benefits to antibody fragments for use as therapeutics. The Fc portion of an antibody recruits complement, which in general means it can recruit other aspects of the immune system and thus amplify the anti-tumor response beyond just inhibiting the target. The addition of the Fc portion also increases the half-life of the antibody fragment (Czajkowsky DM. Hu J. Shao Z and Pleass RJ. (2012) Fc-fusion proteins: new developments and future perspectives. EMBO Mol Med. 4 (10): 1015-1028). However, the Fc portion of an antibody homo-dimerizes, which in the case of anti-MUC1* antibody based therapeutics is not optimal since ligand-induced dimerization of the MUC1* receptor stimulates growth. As can be seen in FIG. 13 B, humanized MN-E6 scFv-Fc is a dimer, in part due to disulfide bonding. Therefore, mutations in the Fc region that resist dimer formation are preferred for anti-MUC1* anti-cancer therapeutics. Deletion of the hinge region (hingeless also called delta hinge or Dhinge in some figures and examples SEQ ID NO: 288 and 289) and other mutations in the Fc region that make the Fc-mutant resistant to dimerization were made. The following mutations were made in the CH3 domain to create a monomeric scFv-Fc fusion protein: Y407R (SEQ ID NO: 278 and 279). F405Q (SEQ ID NO: 280 and 281). T394D (SEQ ID NO: 282 and 283). T366W/L368W (SEQ ID NO: 284 and 285). T364R/L368R (SE ID NO: 286 and 287). FIG. 14 shows photographs of SDS-PAGE characterization of purified MN-E6 scFv-Fc fusion proteins on a non-reducing gels, wherein the Fc portion that was fused to the MN-E6 was either wild type (wt) or mutated as follows: A) F405Q. Y407R. T394D; B) T366W/L368W. T364R/L368R. T366W/L368W or T364R/L368R. Fc mutants F405Q. Y407R. T366W/L368W. T364R/L368R. T366W/L368W and T364R/L368R all favored monomer over dimer formation. FIG. 15 shows FPLC traces of the purification of MN-E6 scFv-Fc Y407Q fusion protein that was grown in low IgG FBS over a Protein A affinity column. A) is the trace of the flow through. B) is the trace of the elution. The protein was further purified by size exclusion over an S200 column (C). (D) is a photograph of an SDS-PAGE gel showing which fractions had a predominance of monomer. FIG. 16 shows a photograph of SDS-PAGE characterization of purified MN-E6 scFv-Fc-mutant fusion proteins on a non-reducing gel, wherein the Fc portion that was fused to the MN-E6 scFv was either wild type (wt) or mutated by elimination of the hinge region. “DHinge′, of the Fc or elimination of the hinge region of the Fc and also bearing the Y407R mutation. All the Fc mutants favored monomer over dimer formation. The reference construct amino acid sequence for the indicated mutation is SEQ ID NO:273. Other relevant sequences are SEQ ID NOS: 289 and 279. FIGS. 17A-C. A and B show photograph of non-reducing SDS-PAGE characterization of large scale expression and purification of MN-E6 scFv-Fc hingeless mutant, showing that it is a monomer. FPLC characterization and purification of MN-E6 scFv-Fc hingeless mutant is shown (C). FIG. 18A-C shows photographs of the SDS-PAGE characterization of the purified MN-C3 scFv-Fc fusion protein on a non-reducing gel (A) or a reducing gel (B). The protein was purified by size exclusion. The FPLC trace is shown (C). FIG. 19A-B shows photographs of Native gels of MN-C3 or MN-E6 Fabs, scFv, scFv-Fc, wherein the Fc portion is wild type or mutants that prefer or are exclusively monomers. Native gels show that the Y407R Fc mutation (A) and the double mutant Y407R and a deleted hinge (B) favor monomer over dimer the best. Note that proteins are loaded onto a gel at much higher concentrations than typical use concentrations. The dimer formation of other Fc mutants may only reflect the fact that loading concentration is very high.

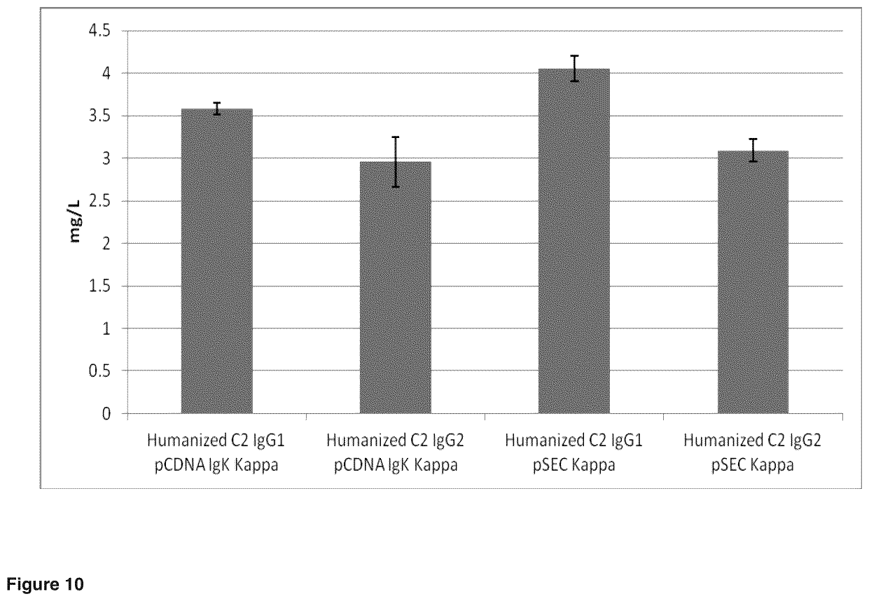

Some mutations or deletions were so effective that, even when loaded onto a gel at high concentrations, they resist dimer formation (FIG. 14A. B). The Y407R mutation results in a nearly pure population of dimeric scFv-Fc (FIG. 10). Similarly deletions of the hinge region of the Fc result in fusion proteins that are monomers rather than dimers. Combinations of mutations can result in even more effective resistance of dimer formation (FIGS. 16 and 17). These and other mutations and combinations thereof were introduced into CH2-CH3 (SEQ ID NO:274 and 275) and CH3 (SEQ ID NO:276 and 277) fusion proteins such as scFv or in the hingeless Fc-fusion proteins such as scFv and were shown to eliminate or minimize dimerization.

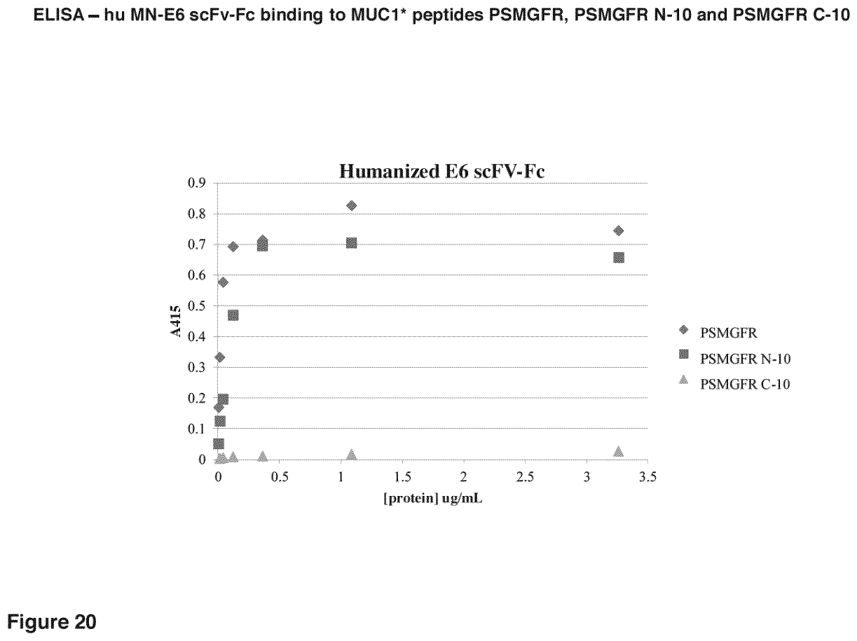

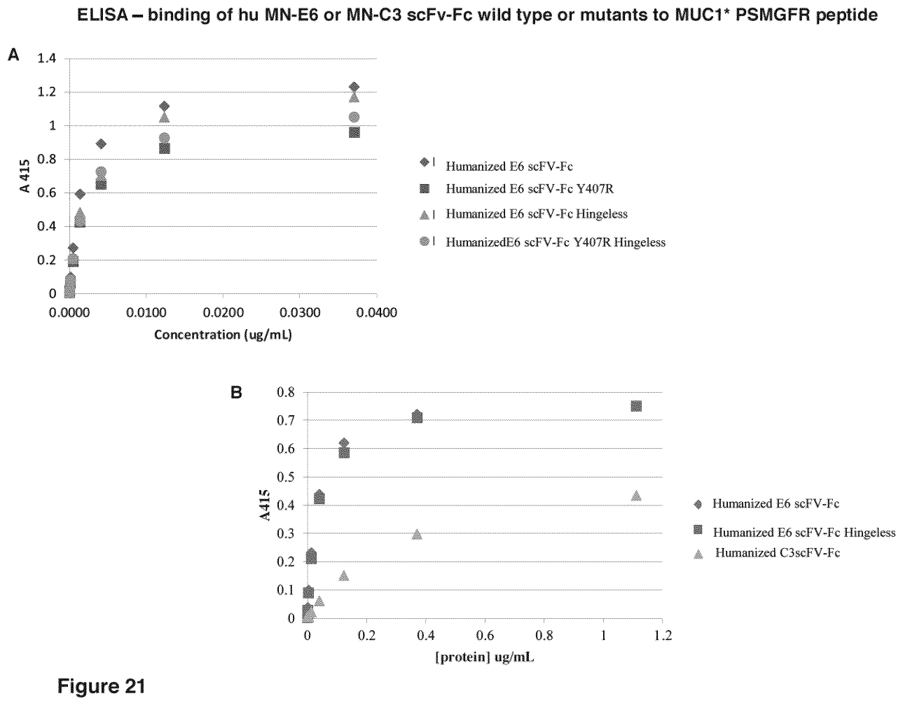

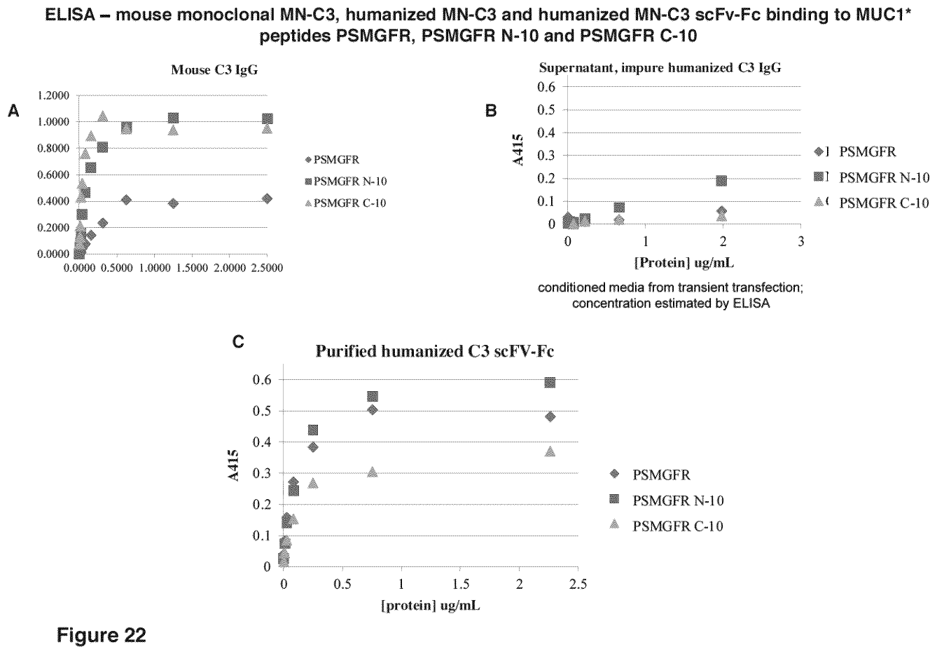

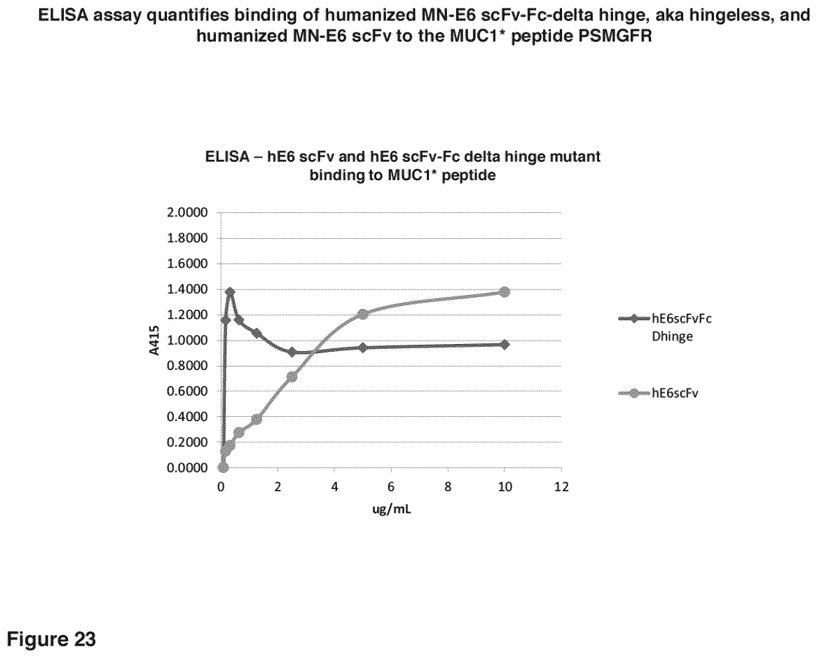

Like the parent mouse monoclonal antibodies, human or humanized antibodies as well as single chain constructs, scFv's, scFv-Fc fusions or scFv-Fc-mutants specifically bind to the synthetic MUC1* peptides (FIGS. 20-22). Figure E23 shows a graph of an ELISA assay that quantifies the binding of humanized MN-E6 scFv-Fc-delta hinge, aka Dhinge or hingeless, and humanized MN-E6 scFv to the MUC1* peptide PSMGFR.

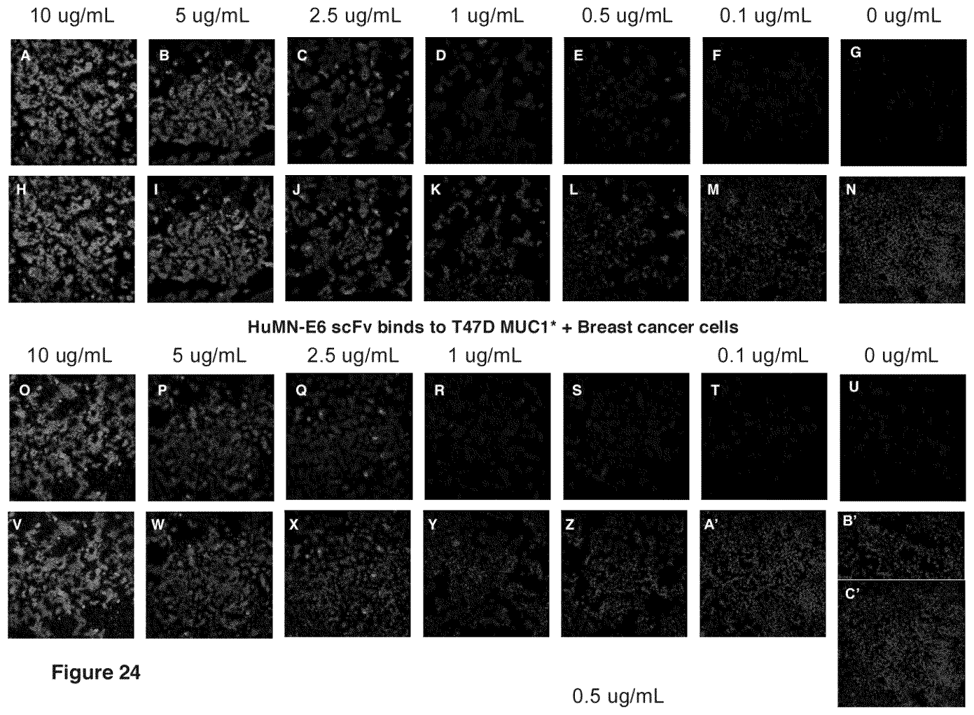

The human or humanized anti-MUC1* antibody fragments described here specifically bind to MUC1 and MUC1* positive cancer cells. FIG. 24 shows photographs of immunofluorescence experiments in which humanized MN-C2 scFv or MN-E6 scFv specifically binds to MUC1* positive breast cancer cells in an identical concentration dependent manner. A-G: hu MN-C2 scFv binding to T47D breast cancer cells at concentrations indicated. H-N shows the fluorescently labeled scFv and DAPI. O-U: hu MN-E6 scFv binding to T47D breast cancer cells at concentrations indicated. V-B′ shows the fluorescently labeled scFv and DAPI. C′ is the secondary antibody control.

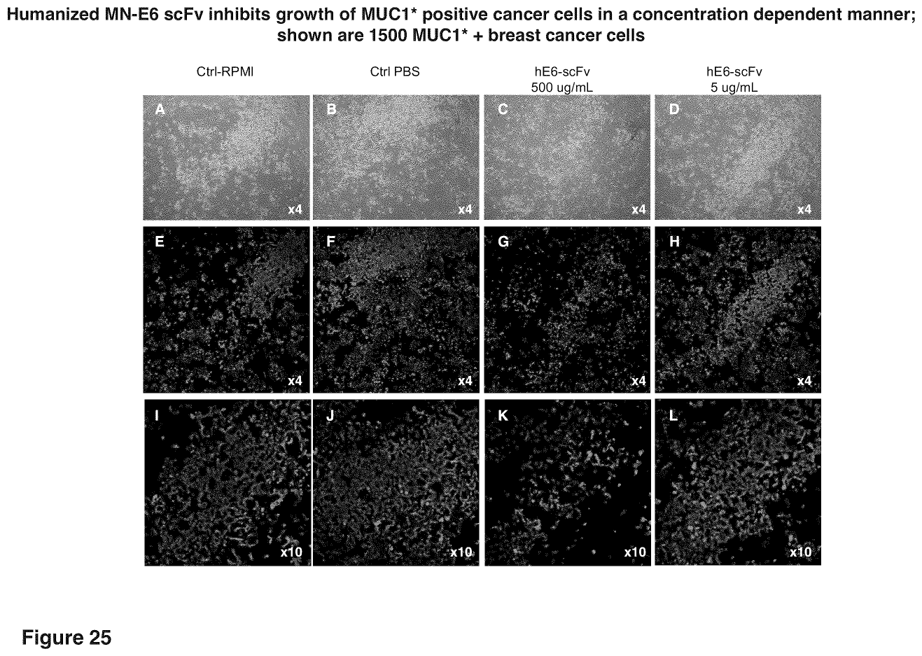

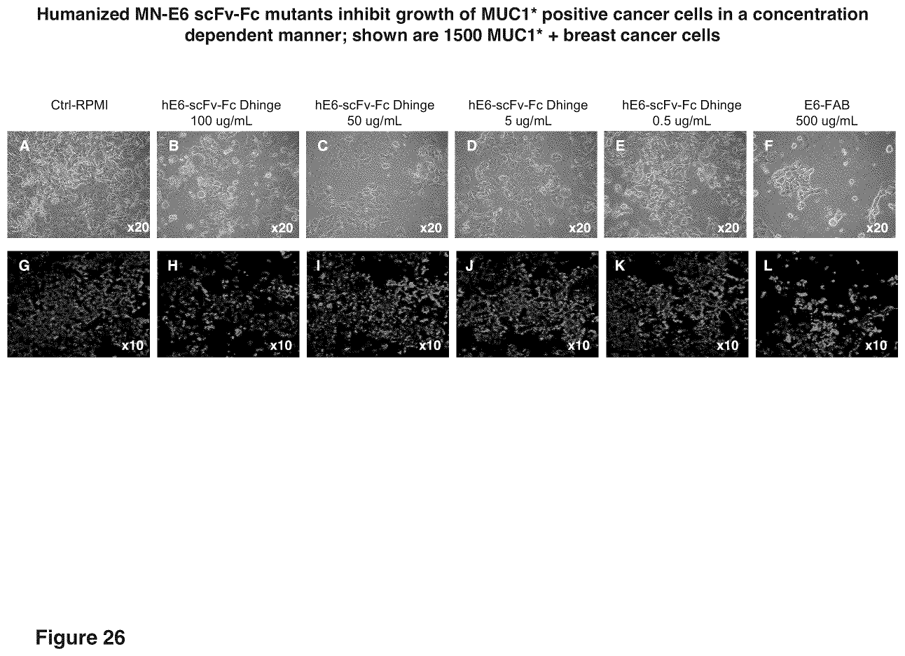

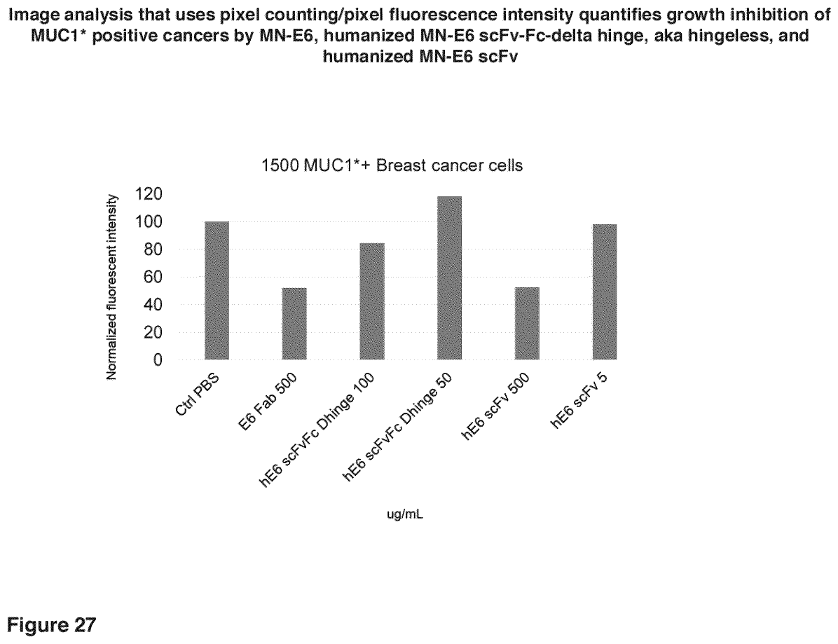

In addition to binding to MUC1* positive cancer cells, the anti-MUC1* antibody variable region fragments, scFv's, scFv-Fc's and scFv-Fc-mutants inhibited growth of MUC1-positive cancer cells. FIG. 25A-L shows photographs of MUC1* positive breast cancer cells that have been cultured in normal medium or in the presence of humanized MN-E6 scFv. The photographs show killing and/or growth inhibition of MUC1* positive cells by MN-E6 scFv at 5 ug/mL and an even greater effect at 500 ug/mL. FIG. 26A-L shows photographs of MUC1* positive breast cancer cells that have been cultured in normal medium or in the presence of humanized MN-E6 scFv-Fc Dhinge, which is a hingeless or delta hinge mutant. The photographs show killing and/or growth inhibition of MUC1* positive cells by hMN-E6 scFv-Fc Dhinge 5 ug/mL, an even greater effect at 50 ug/mL and yet an even greater effect at 100 ug/mL. FIG. 27 shows a graph of the image analysis of the fluorescent images of FIGS. 25 and 26. Image J was used to quantify the number of cells remaining after 96 hours treatment in humanized MN-E6scFv or MN-E6 scFv-Fc-delta hinge, aka Dhinge. The analysis software uses pixel counting and pixel fluorescence intensity to quantify the number of cells in each photograph. Analysis was performed over the entire image 512×512 pixels, 8-bit image. For comparison, the inhibition of mouse monoclonal MN-E6 Fab is also analyzed.

These data show that a human or humanized MN-E6 antibody or antibody fragment, Fab, MN-E6 scFv or hu MN-E6 scFv-FCmut are effective anti-cancer agents that can be administered to a person diagnosed with a MUC1 or MUC1* positive cancer, suspected of having a MUC1 or MUC1* positive cancer or is at risk of developing a MUC1 or MUC1* positive cancer.

In these specific examples, the dimer resistant Fc that was fused onto an antibody fragment or scFv is hu MN-E7 scFv. However, any of these Fc region mutations or combinations thereof that eliminate or minimize dimerization can be fused onto variable region fragments or single chain constructs of MN-E6, MN-C2, MN-C3 or MN-C8 or other antibodies identified that selectively bind to MUC1* as it exists on cancer cells or tissues. In addition, the Fabs of these antibodies can be used as an anti-cancer therapeutic. In one aspect of the invention, a person diagnosed with, suspected of having or is at risk of developing a MUC1* or MUC1 positive cancer is treated with an effective amount of human or humanized MN-E6 scFv, MN-C2 scFv, MN-C3 scFv, or MN-C8 scFv. In another aspect of the invention, a person diagnosed with, suspected of having or is at risk of developing a MUC1* or MUC1 positive cancer is treated with an effective amount of human or humanized MN-E6 SCFV-FC_{Y407R}, MN-C2 ScFv-FC_{Y407R}, MN-C3 ScFy-FC_{Y407R}, or MN-C8 scFv-FC_{Y407R}. In another aspect of the invention, a person diagnosed with, suspected of having or is at risk of developing a MUC1* or MUC1 positive cancer is treated with an effective amount of human or humanized MN-E6 scFv-Fc mutantDhinge, MN-C2 scFv-Fc mutantDhinge, MN-C3 scFv-Fc mutantDhinge, or MN-C8 scFv-Fc mutantDhinge. In yet another aspect of the invention, a person diagnosed with, suspected of having or is at risk of developing a MUC1* or MUC1 positive cancer is treated with an effective amount of human or humanized MN-E6 scFv-Fc mutant_{Y407R-Dhinge}, MN-C2 scFv-Fc mutant_{Y407R-Dhinge}, MN-C3 scFv-Fc mutant_{Y407R-Dhinge}, or MN-C8 scFv-Fc mutant_{Y407R-Dhinge}. One aspect of the invention is a method for treating a patient diagnosed with, suspected of having, or at risk of developing a MUC1 positive or MUC1* positive cancer, wherein the patient is administered an effective amount of a monomeric MN-E6 scFv, MN-C2 scFv, MN-C3 scFv, MN-C8 scFv, or MN-E6 scFv-Fc, MN-C2 scFv-Fc, MN-C3 scFv-Fc, MN-C8 scFv-Fc, wherein the Fc portion of the antibody-like protein has been mutated such that it resists dimer formation.

### Humanizing

Humanized antibodies or antibody fragments or fully human antibodies that bind to the extracellular domain of −MUC1* are preferred for therapeutic use. The techniques described herein for humanizing antibodies are but a few of a variety of methods known to those skilled in the art. The invention is not meant to be limited by the technique used to humanize the antibody.

Humanization is the process of replacing the non-human regions of a therapeutic antibody (usually mouse monoclonal antibody) by human one without changing its binding specificity and affinity. The main goal of humanization is to reduce immunogenicity of the therapeutic monoclonal antibody when administered to human. Three distinct types of humanization are possible. First, a chimeric antibody is made by replacing the non-human constant region of the antibody by the human constant region. Such antibody will contain the mouse Fab region and will contain about 80-90% of human sequence. Second, a humanized antibody is made by grafting of the mouse CDR regions (responsible of the binding specificity) onto the variable region of a human antibody, replacing the human CDR (CDR-grafting method). Such antibody will contain about 90-95% of human sequence. Third and last, a full human antibody (100% human sequence) can be created by phage display, where a library of human antibodies is screened to select antigen specific human antibody or by immunizing transgenic mice expressing human antibody.

A general technique for humanizing an antibody is practiced approximately as follows. Monoclonal antibodies are generated in a host animal, typically in mice. Monoclonal antibodies are then screened for affinity and specificity of binding to the target. Once a monoclonal antibody that has the desired effect and desired characteristics is identified, it is sequenced. The sequence of the animal-generated antibody is then aligned with the sequences of many human antibodies in order to find human antibodies with sequences that are the most homologous to the animal antibody. Biochemistry techniques are employed to paste together the human antibody sequences and the animal antibody sequences. Typically, the non-human CDRs are grafted into the human antibodies that have the highest homology to the non-human antibody. This process can generate many candidate humanized antibodies that need to be tested to identify which antibody or antibodies has the desired affinity and specificity.

Once a human antibody or a humanized antibody has been generated it can be further modified for use as an Fab fragment, as a full antibody, or as an antibody-like entity such as a single chain molecule containing the variable regions, such as scFv or an scFv-Fc. In some cases it is desirable to have Fc region of the antibody or antibody-like molecule mutated such that it does not dimerize.

In addition to methods that introduce human sequences into antibodies generated in non-human species, fully human antibodies can be obtained by screening human antibody libraries with a peptide fragment of an antigen. A fully human antibody that functions like MN-E6 or MN-C2 is generated by screening a human antibody library with a peptide having the sequence of the PSMGFR N-10 peptide. A fully human antibody that functions like MN-C3 or MN-C8 is generated by screening a human antibody library with a peptide having the sequence of the PSMGFR C-10 peptide.

Humanized anti-MUC1* antibodies were generated based on the sequences of the mouse monoclonal antibodies MN-E6, MN-C2, MN-C3 and MN-C8. In one aspect of the invention, a patient diagnosed with a MUC1* positive cancer is treated with an effective amount of humanized MN-E6, MN-C2, MN-C3 or MN-C8. In a preferred embodiment, a patient diagnosed with a MUC1* positive cancer is treated with an effective amount of humanized MN-E6 or MN-C2. In another aspect of the invention, a patient diagnosed with a MUC1* positive cancer is treated with an effective amount of humanized monovalent MN-E6, MN-C2, MN-C3 or MN-C8, wherein monovalent means the corresponding Fab fragment, the corresponding scFv or the corresponding scFv-Fc fusion. In a preferred embodiment, a patient diagnosed with a MUC1* positive cancer is treated with an effective amount of a humanized scFv or monomeric humanized scFv-Fc of MN-E6 or MN-C2. Since the MUC1* growth factor receptor is activated by ligand induced dimerization of its extracellular domain, and because the Fc portion of an antibody homo-dimerizes, it is preferable that a construct that includes an Fc portion uses a mutated Fc region that prevents or minimizes dimerization.

Antibodies that bind to PSMGFR (SEQ ID NO:2) peptide of the extracellular domain of the MUC1* receptor are potent anti-cancer therapeutics that are effective for the treatment or prevention of MUC1* positive cancers. They have been shown to inhibit the binding of activating ligands dimeric NME1 (SEQ ID NOS: 3 and 4) and NME7 (SEQ ID NOS: 5 and 6) to the extracellular domain of MUC1*. Anti-MUC1* antibodies that bind to the PSMGFR sequence inhibit the growth of MUC1*-positive cancer cells, specifically if they inhibit ligand-induced receptor dimerization. Fabs of anti-MUC1* antibodies have been demonstrated to block tumor growth in animals. Thus, antibodies or antibody fragments that bind to the extracellular domain of MUC1* would be beneficial for the treatment of cancers wherein the cancerous tissues express MUC1*.

Antibodies that bind to PSMGFR region of MUC1* or bind to a synthetic PSMGFR peptide are preferred. We have identified several monoclonal antibodies that bind to the extracellular domain of MUC1*. Among this group are mouse monoclonal antibodies MN-E6, MN-C2, MN-C3 and MN-C8, the variable regions of which were sequenced and are given as for MN-E6 SEQ ID NOS: 12-13 and 65-66, for MN-C2 SEQ ID NOS: 118-119 and 168-169, for MN-C3 SEQ ID NOS: 413-414 and 458-459 and for MN-C8 SEQ ID NOS: 505-506 and 543-554. The CDRs of these antibodies make up the recognition units of the antibodies and are the most important parts of the mouse antibody that should be retained when grafting into a human antibody. The sequences of the CDRs for each mouse monoclonal are as follows, heavy chain sequence followed by light chain: MN-E6 CDR1 (SEQ ID NO:16-17 and 69-70) CDR2 (SEQ ID NO:20-21 and 73-74) CDR3 (SEQ ID NO: 24-25 and 77-78), MN-C2 CDR1 (SEQ ID NO:122-123 and 172-173) CDR2 (SEQ ID NO: 126-127 and 176-177) CDR3 (SEQ ID NO:130-131 and 180-181), MN-C3 CDR1 (SEQ ID NO: 417-418 and 462-463) CDR2 (SEQ ID NO:421-422 and 466-467) CDR3 (SEQ ID NO: 425-426 and 470-471). MN-C8 CDR1 (SEQ ID NO:507-508 and 545-546) CDR2 (SEQ ID NO: 509-510 and 547-548) CDR3 (SEQ ID NO:511-512 and 549-550)). In some cases, portions of the framework regions that by modeling are thought to be important for the 3, dimensional structure of the CDRs, are also imported from the mouse sequence.

Monoclonal antibodies MN-E6 and MN-C2 have greater affinity for MUC1* as it appears on cancer cells. Monoclonal antibodies MN-C3 and MN-C8 have greater affinity for MUC1* as it appears on stem cells. By sequence alignment the following human antibodies were chosen as being sufficiently homologous to the mouse antibody that substitution of the mouse CDRs would result in an antibody that retained ability to recognize the target. Mouse MN-E6 heavy chain variable region was homologous to human IGHV3-21*03 heavy chain variable region (SEQ ID NO: 26-27) and the light chain variable region was homologous to human IGKV3-11*02 light chain variable region (SEQ ID NO: 79-80). Mouse MN-C2 heavy chain variable region was homologous to human IGHV3-21*04 heavy chain variable region (SEQ ID NO: 132-133) and the light chain variable region was homologous to human IGKV7-3*01 light chain variable region (SEQ ID NO: 182-183). Mouse MN-C3 heavy chain variable region was homologous to human IGHV1-18*04 heavy chain variable region (SEQ ID NO: 427-428) and the light chain variable region was homologous to human IGKV2-29*03 light chain variable region (SEQ ID NO:472-473). Mouse MN-C8 heavy chain variable region was homologous to human IGHV3-21*04 heavy chain variable region (SEQ ID NO: 513-514) and the light chain variable region was homologous to human Z00023 light chain variable region (SEQ ID NO:551-552).

All four antibodies have been humanized, which process has resulted in several humanized forms of each antibody. CDRs derived from the variable regions of the mouse antibodies were biochemically grafted into a homologous human antibody variable region sequence. Humanized variable regions of MN-E6 (SEQ ID NOS: 38-39 and 93-94), MN-C2 (SEQ ID NOS: 144-145 and 194-195). MN-C3 (SEQ ID NOS: 439-440 and 486-487) and MN-C8 (SEQ ID NOS: 525-526 and 543-544) were generated by grafting the mouse CDRs into the variable region of a homologous human antibody. The humanized heavy chain variable constructs were then fused into constant regions of either human IgG1 heavy chain constant region (SEQ ID NOS: 58-59) or human IgG2 heavy chain constant region (SEQ ID NO: 54-55), which are then paired with either humanized light chain variable constructs fused to a human kappa chain (SEQ ID NO: 109-110) or human lambda chain (SEQ ID NO: 113-114) constant region. Other IgG isotypes could be used as constant region including IgG3 or IgG4.

Examples of humanized MN-E6 variable region into an IgG2 heavy chain (SEQ ID NOS: 52-53) and into an IgG1 heavy chain (SEQ ID NOS: 56-57), humanized MN-C2 variable into an IgG1 heavy chain (SEQ ID NOS: 158-159) or into an IgG2 heavy chain (SEQ ID NOS: 163-164) paired with either Lambda light chain (SEQ ID NO: 111-112 and 216-219) or Kappa chain (SEQ ID NO: 107-108 and 210-213) and, humanized MN-C3 (SEQ ID NOS: 455-456. 453-454 and 500-501. 502-503) and MN-C8 (SEQ ID NOS: 541-542. 539-540 and 579-580, 581-582) antibodies were generated. Which IgG constant region is fused to the humanized variable region depends on the desired effect since each isotype has its own characteristic activity. The isotype of the human constant region is selected on the basis of things such as whether antibody dependent cell cytotoxicity (ADCC) or complement dependent cytotoxicity (CDC) is desired but can also depend on the yield of antibody that is generated in cell-based protein expression systems. In a preferred embodiment, humanized anti-MUC1* antibodies or antibody fragments are administered to a person diagnosed with or at risk of developing a MUC1-positive cancer.

One method for testing and selecting the humanized anti-MUC1* antibodies that would be most useful for the treatment of persons with cancer or at risk of developing cancers is to test them for their ability to inhibit the binding of activating ligands to the MUC1* extracellular domain. Dimeric NME1 can bind to and dimerize the MUC1* extracellular domain and in so doing stimulates cancer cell growth. Antibodies and antibody fragments that compete with NME1 for binding to the MUC1* extracellular domain are therefore anti-cancer agents. NME7 is another activating ligand of MUC1*. In some cases, it is preferable to identify antibodies that block the binding of NME7, or an NME7 truncation or cleavage product, to the MUC1* extracellular domain. Antibodies and antibody fragments that compete with NME7 and NME7 variants for binding to the MUC1* extracellular domain are effective as anti-cancer therapeutics. These antibodies include but are not limited to MN-E6. MN-C2, MN-C3, MN-C8 as well as single chain versions, such as scFv, of these antibodies and humanized version thereof. Other NME proteins also bind to MUC1 or MUC1* including NME6 and NME8. Antibodies that compete with these proteins for binding to MUC1* may also be useful as therapeutics. In a preferred embodiment, humanized anti-MUC1* antibodies or antibody fragments are administered to a person diagnosed with or at risk of developing a MUC1-positive cancer. In a more preferred embodiment, single chain antibody fragments, or monomeric scFv-Fc fusions, derived from humanized sequences of MN-E6 and MN-C2 are administered to a person diagnosed with or at risk of developing a MUC1-positive cancer.

Single chain variable fragments, scFv, or other forms that result in a monovalent antibody or antibody-like protein are also useful. In some cases it is desired to prevent dimerization of the MUC1* extracellular domain. Single chain variable fragments. Fabs and other monovalent antibody-like proteins have been shown to be effective in binding to the extracellular domain of MUC1* and blocking MUC1* dimerization. These single chain variable fragments. Fabs and other monovalent antibody-like molecules effectively blocked cancer growth in vitro and in animals xenografted with human MUC1-positive cancer cells. Thus, humanized single chain variable fragments or monovalent anti-MUC1* antibodies or antibody-like molecules would be very effective as an anti-cancer therapeutic. Such humanized single chain antibodies. Fabs and other monovalent antibody-like molecules that bind to the MUC1* extracellular domain or to a PSMGFR peptide are therefore useful as anti-cancer therapeutics. Anti-MUC1* single chain variable fragments are generated by grafting non-human CDRs of antibodies, which bind to extracellular domain of MUC1* or bind to PSMGFR peptide, into a framework of a homologous variable region human antibody. The resultant humanized heavy and light chain variable regions are then connected to each other via a suitable linker, wherein the linker should be flexible and of length that it allows heavy chain binding to light chain but discourages heavy chain of one molecule binding to the light chain of another. For example a linker of about 10-15 residues. Preferably, the linker includes [(Glycine)₄ (Serine)_{]}[₃ (SEQ ID NOS: 40]-402), but is not limited to this sequence as other sequences are possible.

In one aspect, the humanized variable regions of MN-E6 (SEQ ID NOS: 38-39 and 93-94). MN-C2 (SEQ ID NOS: 144-145 and 194-195), MN-C3 (SEQ ID NOS: 439-440) and 486-487) and MN-C8 (SEQ ID NOS: 525-526 and 565-566) are biochemically grafted into a construct that connects heavy and light chains via a linker. Examples of humanized single chain anti-MUC1* antibodies comprising humanized sequences from the variable regions of MN-E6, MN-C2, MN-C3 and MN-C8 were generated. Several humanized MN-E6 single chain proteins were generated (SEQ ID NOS: 232-237). Several humanized MN-C2 single chain proteins were generated (SEQ ID NOS: 238-243). Several humanized MN-C3 single chain proteins were generated (SEQ ID NOS: 244-249). Several humanized MN-C8 single chain proteins were generated (SEQ ID NOS: 250-255). In a preferred embodiment, humanized anti-MUC1* antibody fragments, including variable fragments, scFv antibody fragments MN-E6 scFv, MN-C2 scFv, MN-C3 scFv, or MN-C8 scFv are administered to a person diagnosed with or at risk of developing a MUC1-positive cancer. In a more preferred embodiment, single chain antibody fragments, such as variable fragments derived from humanized sequences of MN-E6 and MN-C2, are administered to a person diagnosed with or at risk of developing a MUC1-positive cancer.

In another aspect, the humanized variable regions of MN-E6 (SEQ ID NOS: 38-39 and 93-94), MN-C2 (SEQ ID NOS: 144-145 and 194-195). MN-C3 (SEQ ID NOS: 439-440) and 486-487) and MN-C8 (SEQ ID NOS: 525-526 and 565-566) are biochemically grafted into a single chain variable fragment, scFv, that also contains an Fc portion of an antibody. Examples of humanized single chain variable fragment of MN-E6, MN-C2, MN-C3 and MN-C8 fused to a Fc region of an antibody were generated (SEQ ID NOS: 256-257. 260-261. 264-265 and 268-269). Inclusion of an Fc region serves several purposes. It increases the molecular weight of the antibody fragment, which slows degradation and increases half-life. An Fc region also recruits immune system complement to the tumor site. Additionally, the addition of an antibody Fc region makes the scFv a convenient diagnostic tool, as the secondary antibodies detect and label the Fc portion. However, the Fc portion homo-dimerizes. Thus an scFv-Fc would be bivalent and could dimerize and activate the MUC1* growth factor receptor. In order to get the benefits of having an Fc attached to an anti-MUC1* scFv, without the drawback of inducing MUC1* dimerization, the Fc region was mutated to minimize or eliminate Fc homo-dimerization. The following mutations were made in the CH3 domain to create a monomeric scFv-Fc fusion protein: Y407R (SEQ ID NOS: 278 and 279), F405Q (SEQ ID NOS: 280 and 281). T394D (SEQ ID NOS: 282 and 283). T366W/L368W (SEQ ID NOD: 284 and 285). T364R/L368R (SEQ ID NOS: 286 and 285). Any combinations of those mutations can be tested and could be introduced into Fc (SEQ ID NOS: 272-273). CH2-CH3 (SEQ ID NOS: 274-275) or CH3 (SEQ ID NOS: 276-277) fusion proteins or in the hingeless Fc-fusion proteins (SEQ ID NOS: 288-289).

One aspect of the invention is a method for treating a patient diagnosed with, suspected of having, or at risk of developing a MUC1 positive or MUC1* positive cancer, wherein the patient is administered an effective amount of a monomeric MN-E6 scFv, MN-C2 scFv, MN-C3 scFv, MN-C8 scFv, or MN-E6 scFv-Fc. MN-C2 scFv-Fc. MN-C3 scFv-Fc. MN-C8 scFv-Fc, wherein the antibody variable fragment portions are human or have been humanized and wherein the Fc portion of the antibody-like protein has been mutated such that it resists dimer formation.

CAR T and cancer immuno therapy techniques

In another aspect of the invention, some or all of the single chain portions of anti-MUC1* antibody fragments are biochemically fused onto immune system molecules, using several different chimeric antigen receptor. “CAR′ strategies. The idea is to fuse the recognition portion of an antibody, typically as a single chain variable fragment, to an immune system molecule that has a transmembrane domain and a cytoplasmic tail that is able to transmit signals that activate the immune system. The recognition unit can be an antibody fragment, a single chain variable fragment, scFv, or a peptide. In one aspect, the recognition portion of the extracellular domain of the CAR is comprised of sequences from the humanized variable region of MN-E6 (SEQ ID NOS: 38-39 and 93-94). MN-C2 (SEQ ID NOS: 144-145 and 194-195), MN-C3 (SEQ ID NOS: 439-440 and 486-487) and MN-C8 (SEQ ID NOS: 525-526 and 565-566). In another aspect, it is comprised of sequences from a single chain variable fragment. Examples of single chain constructs are given. Several humanized MN-E6 single chain proteins, scFv, were generated (SEQ ID NOS: 232-237). Several humanized MN-C2 single chain proteins, scFv, were generated (SEQ ID NOS: 238-243). Several humanized MN-C3 single chain proteins, scFv, were generated (SEQ ID NOS: 244-249). Several humanized MN-C8 single chain proteins, scFv, were generated (SEQ ID NOS: 250-255). The transmembrane region of the CAR can be derived from CD8, CD4, antibody domains or other transmembrane region, including the transmembrane region of the proximal cytoplasmic co-stimulatory domain. The cytoplasmic tail of the CAR can be comprised of one or more motifs that signal immune system activation. This group of cytoplasmic signaling motifs, sometimes referred to as, co-stimulatory cytoplasmic domains, includes but is not limited to CD3-zeta, CD27, CD28. 4-1BB, OX40, CD30, CD40, PD-1, ICAm-1, LFA-1, ICOS, CD2, CD5, CD7 and Fc receptor gamma domain. A minimal CAR may have the CD3-zeta or an Fc receptor gamma domain then one or two of the above domains in tandem on the cytoplasmic tail. In one aspect, the cytoplasmic tail comprises CD3-zeta, CD28. 4-1BB and/or OX40. Several examples of humanized MN-E6 CARs were generated: CAR MN-E6 CD3z (SEQ ID NOS: 294-295): CAR MN-E6 CD28/CD3z (SEQ ID NOS: 297-298): CAR MN-E6 4-1BB/CD3% (SEQ ID NOS: 300-301): CAR MN-E6 OX40/CD3z (SEQ ID NOS: 616-617); CAR MN-E6 CD28/OX40/CD3z (SEQ ID NOS: 618-619): CAR MN-E6 CD28/4-1BB/CD3% (SEQ ID NOS: 303-304). Several examples of humanized MN-C2 CARs were generated: CAR MN-C2 CD3% (SEQ ID NOS: 606-607); CAR MN-C2 CD28/CD3% (SEQ ID NOS: 608-609): CAR MN-C2 4-1BB/CD3z (SEQ ID NOS: 610-611): CAR MN-C2 OX40/CD3z (SEQ ID NOS: 612-613): CAR MN-C2 CD28/4-1BB/CD3z (SEQ ID NOS: 306-307); CAR MN-C2 CD28/0X40/CD3z (SEQ ID NOS: 614-615). Humanized MN-C3 CAR was generated: CAR MN-C3 4-1BB/CD3z (SEQ ID NOS: 600-601).

Several examples of humanized MN-E6 CARs with different hinge regions (SEQ ID NOS: 345-360) were generated: CAR MN-E6-Fc/8/41BB/CD3z (SEQ ID NOS: 310-311): CAR MN-E6 FcH/8/41BB/CD3z (SEQ ID NOS: 315-316): CAR MN-E6 Fc/4/41BB/CD3Z (SEQ ID NOS: 318-319); CAR MN-E6 FcH/4/41BB/CD3% (SEQ ID NOS: 321-322): : CAR MN-E6 IgD/8/41BB/CD3z (SEQ ID NOS: 323-324): CAR MN-E6 IgD/4/41BB/CD3z (SEQ ID NOS: 327-328): CAR MN-E6 X4/8/41BB/CD3% (SEQ ID NOS: 330-331); CAR MN-E6 X4/4/41BB/CD3% (SEQ ID NOS: 333-334): CAR MN-E6 8+4/4/41BB/CD3Z (SEQ ID NOS: 336-337). In addition, several humanized MN-C3 single chain variable fragment and humanized MN-C8 single chain variable fragments were also generated.

The extracellular domain recognition unit of a MUC1* targeting CAR can comprise the variable regions of humanized MN-E6, MN-C2, MN-C3 or MN-C8 or other antibody that binds to the PSMGFR portion of MUC1* or a PSMGFR peptide. In one aspect, the extracellular domain recognition unit of a CAR is comprised essentially of a humanized MN-E6, MN-C2, MN-C3 or MN-C8 single chain variable fragment scFv. The transmembrane region of the CAR can be derived from CD8 (SEQ ID NOS: 363-364), or can be the transmembrane domain of CD3-zeta, CD28, 41bb, OX40) or other transmembrane region (SEQ ID NOS: 361-372) and the cytoplasmic domain of a CAR with antibody fragment targeting MUC1* extracellular domain can be comprised of one or more selected from the group comprising an immune system co-stimulatory cytoplasmic domain. The group of immune system co-stimulatory domains includes but is not limited to CD3-zeta, CD27. CD28. 4-1BB. OX40, CD30, CD40. PD-1. ICAm-1. LFA-1. ICOS. CD2, CD5, CD7 and Fc receptor gamma domain (SEQ ID NOS: 373-382). Alternatively, the recognition unit portion of a CAR can comprise a peptide wherein the peptide binds to the target. NME7 binds to and activates MUC1*. In one aspect of the invention, the recognition unit of a CAR is a peptide derived from NME7 (SEQ ID NOS: 5-6) or a peptide derived from NME7, including but not limited to NME7 peptide A1 (SEQ ID NO: 7). NME7 peptide A2 (SEQ ID NO: 8). NME7 peptide B1 (SEQ ID NO: 9). NME7 peptide B2 (SEQ ID NO: 10) and NME7 peptide B3 (SEQ ID NO: 11).

Some strategies for generating CARs include a portion of the molecule that dimerizes with itself. In some cases, dimerization of the target is not desirable. Therefore CARs can be constructed such that they heterodimerize. In one case the recognition unit of the first CAR binds to a first target while the recognition unit of the second CAR binds to a second target. Both recognition units can be antibody fragments, both can be peptides or one can be an antibody fragment and the other a peptide. A first target of the CAR can be the extracellular domain of MUC1*. The recognition unit of the CAR would be comprised of an antibody fragment that binds to MUC1* extracellular domain or to a PSMGFR peptide. Alternatively, the recognition unit of the CAR would be comprised of a peptide that binds to MUC1* extracellular domain, such peptides include peptides derived from an NME protein such as NME1 or NME7, more particularly NME7 derived peptides listed as SEQ ID NOS: 7-11. A second target of a heterodimeric CAR may be a peptide or antibody fragment that binds to NME7. Alternatively, a second target of a heterodimeric CAR may be a peptide or antibody fragment that binds to PDI or other target on a MUC1*-presenting cell. A second target may be a peptide or antibody fragment that binds to NME1. Because it is desirable to prevent dimerization of MUC1 induced by a CAR, heterodimeric CARs can be constructed so that only the extracellular domain of one molecule has an extracellular recognition unit that binds to a target (SEQ ID NOS: 584-587). The other molecule can have a truncated extracellular domain that is devoid of a target recognition unit or antibody fragment (SEQ ID NOS: 588-599). The CARs described can be transfected or transduced into a cell of the immune system. In a preferred embodiment, a MUC1* targeting CAR is transfected or transduced into a T cell. In one aspect the T cell is a CD3+/CD28+T cell. In another case it is a dendritic cell. In another case it is a B cell. In another case it is a mast cell. The recipient cell can be from a patient or from a donor. If from a donor, it can be engineered to remove molecules that would trigger rejection. Cells transfected or transduced with a CAR of the invention can be expanded ex vivo or in vitro then administered to a patient. Administrative routes are chosen from a group containing but not limited to bone marrow transplant, intravenous injection, in situ injection or transplant. In a preferred embodiment, the MUC1* targeting CAR is administered to a person diagnosed with or at risk of developing a MUC1-positive cancer.

There are many possible anti-MUC1* CAR constructs that can be transduced into T cells or other immune cells for the treatment or prevention of MUC1* positive cancers. CARs are made up of modules and the identity of some of the modules is relatively unimportant, while the identity of other modules is critically important.

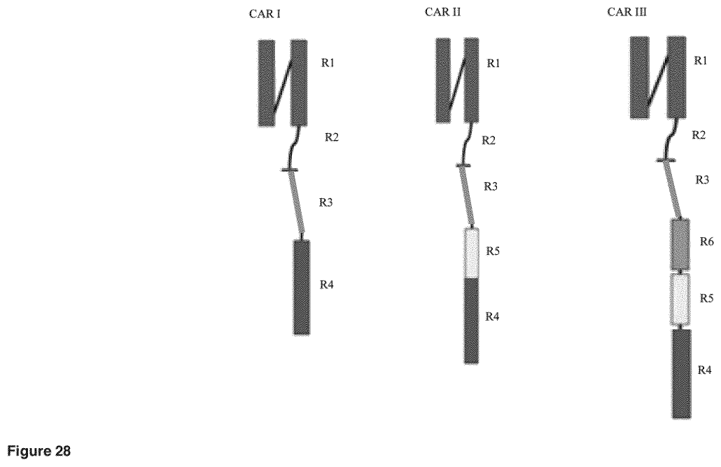

Our experiments demonstrate that the antibody recognition fragment at the outermost portion of the CAR is critically important because it targets the immune cell bearing the CAR to the tumor site. The intracellular signaling motifs are also very important but can be interchanged. FIG. 28 shows a schematic of the components of CAR and the various sequences that may be included in a CAR. Referring to FIG. 28.

R1 is: nothing: or
a ligand or a fragment of a ligand of a cancer associated antigen: or
a ligand or a fragment of a ligand of MUC1 or MUC1*: or
an antibody or antibody fragment wherein the antibody or antibody fragment binds to MUC1 or MUC1*: or an antibody or antibody fragment wherein the antibody or antibody fragment binds to PSMGFR*, wherein the antibody may be human or humanized: or an antibody or antibody fragment of MN-E6, MN-C2, MN-C3 or MN-C8 or humanized MN-E6, MN-C2, MN-C3 or MN-C8: or a single chain variable fragment of an antibody, scFv, that binds to a cleaved MUC1 or MUC1*: or a scFv of MN-E6, MN-C2, MN-C3 or MN-C8, which may be humanized: or a peptide that binds to MUC1* or PSMGFR peptide: or is an antibody fragment, a scFv, or a peptide that binds the PSMGFR portion of MUC1*: or is comprised of sequence from the humanized variable region of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 565-566). In one aspect R1 is a scFv that binds the PSMGFR portion of MUC1* comprised of sequence from humanized MN-E6 scFv (SEQ ID NOS: 232-237), humanized MN-C2 scFv (SEQ ID NOS: 238-243), humanized MN-C3 scFv (SEQ ID NOS: 244-249) or humanized MN-C8 scFv (SEQ ID NOS: 250-255). In another aspect R1 is a scFv that binds the PSMGFR portion of MUC1* comprised of sequence from humanized MN-E6 scFv (SEQ ID NOS: 232-237) or humanized MN-C2 scFv (SEQ ID NOS: 238-243). In one example R1 is a scFv that binds the PSMGFR portion of MUC1* comprised of sequence from humanized MN-E6 scFv (SEQ ID NOS: 232-237)

R2 is a polypeptide flexible linker that connects the recognition portion to the transmembrane domain of the CAR. In one aspect. R2 can be a polypeptide linker of different length from 5 to 250 amino acids. In another aspect. R2 is a polypeptide linker of human origin. In one aspect R2 can be made of or a modification of the Fc region of a human immunoglobulin (IgG. IgA. IgE. IgM or IgD). I another aspect. R2 can be the hinge region or a modification of the hinge region of a human immunoglobulin (IgG. IgA. IgE. IgM or IgD). In one aspect. R2 can be the hinge region or a modification of the hinge region of a T-cell receptor (CD8a. CD28 or CD4). In one example R2 is the hinge region of CD8a, the hinge region of human IgD or the Fc domain of human IgG1.

R3 is a transmembrane domain. In one aspect. R3 can be a transmembrane domain or a modification of a transmembrane domain of any transmembrane human proteins. In another aspect. R3 can be a transmembrane domain or a modification of a transmembrane domain from human cell receptor. In one aspect. R3 can be a transmembrane domain or a modification of a transmembrane domain of a T-cell receptor (CD8a. CD4, CD28, CD3%. OX40 or 41-BB). In another aspect. R3 is a transmembrane domain from the first cytoplasmic co-stimulatory domain of the CAR. In one aspect. R3 can be a transmembrane domain or a modification of a transmembrane domain of a T-cell receptor extended with 1.2.3.4 or 5 amino acids of the cytoplasmic domain associated to the transmembrane domain. In another aspect. R3 can be a transmembrane domain or a modification of a transmembrane domain of a T-cell receptor extended with 1.2.3.4 or five amino acids of the cytoplasmic domain associated to the transmembrane domain followed by a cystein for disulfide bond formation. In one example R3 is the transmembrane domain of CD8a or CD4.

R4 is a signaling domain from a T-cell receptor. In one aspect. R4 can be the cytoplasmic signaling domain of CD3-zeta, CD27, CD28. 4-1BB. OX40, CD30, CD40, PD-1. ICAm-1. LFA-1. ICOS. CD2, CD5, CD7 and Fc receptor gamma domain. In one example R4 is the cytoplasmic domain of CD3-zeta. Several examples of humanized CAR with single signaling domain (CAR I) were regenerated: CAR MN-E6 CD3% (SEQ ID NOS: 294-295): CAR MN-C2 CD3% (SEQ ID NOS: 606-607)

R5 is a co-stimulatory domain from a T-cell receptor. In one aspect. R5 can be the cytoplasmic signaling domain of CD27, CD28, 4-1BB. OX40, CD30, CD40. PD-1. ICAm-1. LFA-1. ICOS. CD2, CD5, CD7 and Fc receptor gamma domain. R5 will be different from R4 and R6. In one example R5 is the cytoplasmic domain of CD28. 4-1BB or OX40. Several examples of humanized CAR with two signaling domain (CAR II) were regenerated: CAR MN-E6 CD28/CD3% (SEQ ID NOS: 297-298): CAR MN-E6 4-1BB/CD3% (SEQ ID NOS: 300-301); CAR MN-E6 OX40/CD3% (SEQ ID NOS: 616-617): CAR MN-C2 CD28/CD3% (SEQ ID NOS: 608-609); CAR MN-C2 4-1BB/CD3% (SEQ ID NOS: 610-611); CAR MN-C2 OX40/CD3% (SEQ ID NOS: 612-613): MN-C3 4-1BB/CD3% (SEQ ID NOS: 600-601): CAR MN-E6-Fc/8/41BB/CD3% (SEQ ID NOS: 310-311): CAR MN-E6 FcH/8/41BB/CD3% (SEQ ID NOS: 315-316): CAR MN-E6 Fc/4/41BB/CD3% (SEQ ID NOS: 318-319); CAR MN-E6 FcH/4/41BB/CD3% (SEQ ID NOS: 321-322): : CAR MN-E6 IgD/8/41BB/CD3z (SEQ ID NOS: 323-324): CAR MN-E6 IgD/4/41BB/CD3% (SEQ ID NOS: 327-328): CAR MN-E6 X4/8/41BB/CD3% (SEQ ID NOS: 330-331); CAR MN-E6 X4/4/41BB/CD3% (SEQ ID NOS: 333-334): CAR MN-E6 8+4/4/41BB/CD3% (SEQ ID NOS: 336-337).

R6 is a co-stimulatory domain from a T-cell receptor. In one aspect. R6 can be the cytoplasmic signaling domain of CD27, CD28. 4-1BB. OX40, CD30, CD40, PD-1. ICAm-1. LFA-1. ICOS. CD2, CD5, CD7 and Fc receptor gamma domain. R6 will be different from R4 and R5. In one example R5 is the cytoplasmic domain of CD28. Several examples of humanized CAR with two signaling domain (CAR III) were regenerated: CAR MN-E6 CD28/OX40/CD3% (SEQ ID NOS: 618-619); CAR MN-E6 CD28/4-1BB/CD3% (SEQ ID NOS: 303-304): CAR MN-C2 CD28/4-1BB/CD3% (SEQ ID NOS: 306-307): CAR MN-C2 CD28/OX40/CD3% (SEQ ID NOS: 614-615)

We and others (Pule MA. Straathof KC. Dotti G. Heslop HE. Rooney CM and Brenner MK. (2005) A chimeric T cell antigen receptor that augments cytokine release and supports clonal expansion of primary human T cells. Mol Ther. 12 (5): 933-941: Hombach AA. Heiders J. Foppe M. Chmielewski M and Abken H. (2012) OX40 costimulation by a chimeric antigen receptor abrogates CD28 and IL-2 induced IL-10 secretion by redirected CD4 (+) T cells. Oncoimmunology. 1 (4): 458-466; Kowolik CM. Topp MS. Gonzalez S. Pfeiffer T. Olivares S. Gonzalez N. Smith DD. Forman SJ. Jensen MC and Cooper LJ. (2006) CD28 costimulation provided through a CD19-specific chimeric antigen receptor enhances in vivo persistence and antitumor efficacy of adoptively transferred T cells. Cancer Res. 66 (22): 10995-11004: Loskog A. Giandomenico V. Rossig C. Pule M. Dotti G and Brenner MK. (2006) Addition of the CD28 signaling domain to chimeric T-cell receptors enhances chimeric T-cell resistance to T regulatory cells. Leukemia. 20 (10): 1819-1828; Milone MC. Fish JD. Carpenito C. Carroll RG. Binder GK. Teachey D. Samanta M. Lakhal M. Gloss B. Danet-Desnoyers G. Campana D. Riley JL. Grupp SA and June CH. (2009) Chimeric receptors containing CD137 signal transduction domains mediate enhanced survival of T cells and increased antileukemic efficacy in vivo. Mol Ther. 17 (8): 1453-1464: Song DG. Ye Q. Carpenito C. Poussin M. Wang LP. Ji C. Figini M. June CH. Coukos G. Powell DJ Jr. (2011) In vivo persistence, tumor localization, and antitumor activity of CAR-engineered T cells is enhanced by costimulatory signaling through CD137 (4-1BB). Cancer Res. 71 (13): 4617-4627) have shown that intracellular signaling modules, such as CD3-zeta (SEQ ID NOS: 373-376). CD28 (SEQ ID NOS: 377-378) and 41BB (SEQ ID NOS: 379-380), alone or in combinations stimulate immune cell expansion, cytokine secretion and immune cell mediated killing of the targeted tumor cells. Less important is the identity of the short extracellular piece that presents the antibody fragment, the transmembrane domain, and the short cytoplasmic tail that comes before the intracellular signaling motifs.

The identity of the recognition antibody fragment that targets the CAR to a tumor is critically important. For the treatment of MUC1 positive or MUC1* positive cancers, that antibody recognition fragment must bind to the extracellular domain of portion of MUC1 that remains after cleavage and shedding of the bulk of the extracellular domain, which contains the tandem repeat domains. In one aspect of the invention, the portion that remains comprises the PSMGFR sequence. In another aspect of the invention, the portion of MUC1 that remains after cleavage and shedding contains the PSMGFR sequence plus nine (9) more amino acids extended at the N-terminus. In another aspect of the invention, the portion of MUC1 that remains after cleavage and shedding contains the PSMGFR sequence plus twenty one (21) more amino acids extended at the N-terminus. In one aspect the antibody recognition fragment binds to a PSMGFR peptide. In another aspect of the invention, the antibody recognition fragment a binds to peptide comprising the sequence SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621) As a demonstration, a single chain antibody fragment that included the variable domain of the monoclonal anti-MUC1* antibodies called MN-E6 or MN-C2 were engineered into a panel of CARs. The MUC1* targeting CARs were then transduced, separately or in combinations, into immune cells. When challenged with surfaces presenting a MUC1* peptide, an antigen presenting cell transfected with MUC1*, or MUC1* positive cancer cells, the immune cells that were transduced with MUC1* targeting CARs elicited immune responses, including cytokine release, killing of the targeted cells and expansion of the immune cells. In one case, human jurkat cells were transduced with MUC1*-targeting CARs and upon exposure to a surface presenting the PSMGFR peptide. K562 antigen presenting cells that had been transfected with MUC1* or MUC1* positive cancer cells, the jurkhat cells secreted IL-2. In another case, purified human T cells were transduced with MUC1*-targeting CARs and upon exposure to a surface presenting the PSMGFR peptide. K562 antigen presenting cells that had been transfected with MUC1* or MUC1* positive cancer cells, the T cells secreted IL-2, interferon gamma, and killed the targeted antigen presenting cells and cancer cells, while the T cells expanded. As demonstrated. CARs that comprise an antibody fragment, wherein the antibody fragment is able to bind to the PSMGFR peptide, a transmembrane domain and a cytoplasmic tail bearing co-stimulatory domains, elicit an immune system anti-tumor cell response when said CARs are transduced into immune cells, which include T cells. Therefore, other antibodies, antibody fragments or antibody mimics that are able to bind to the PSMGFR peptide will perform similarly and can be used to treat or prevent cancers. Those skilled in the art will recognize that there are a number of technologies available for transfecting or transducing cells with CARs and the invention is not limited by the method used for making the immune cell express a MUC1*-targeting CAR. For example, retroviruses, adeno viruses. lenti viruses and the like can be used. Similarly, the identity of molecules that make up the non-targeting, portions of the CAR such as the extracellular domain, transmembrane domain and membrane proximal portion of the cytoplasmic domain, are not essential to the function of a MUC1*-targeting CAR. For example, the extracellular domain, transmembrane domain and membrane proximal portion of the cytoplasmic domain can be comprised of portions of CD8, CD4, CD28, or generic antibody domains such as Fc. CH2CH3, or CH3. Further, the non-targeting portions of a CAR can be a composite of portions of one or more of these molecules or other family members.

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR. In another aspect of the invention, the immune cells are T cells isolated from a patient, which are then transduced with CARs wherein the targeting head of the CAR binds to MUC1*, and after expansion of transduced T cells, the CAR T cells are administered in an effective amount to the patient. In yet another aspect of the invention, the immune cells are T cells isolated from a patient, which are then transduced with CARs wherein the targeting head of the CAR comprises portions of huMN-E6, huMN-C2, huMN-C3 or huMN-C8, and after optional expansion of transduced T cells, the CAR T cells are administered in an effective amount to the patient.

Specifics of CARs made and tested

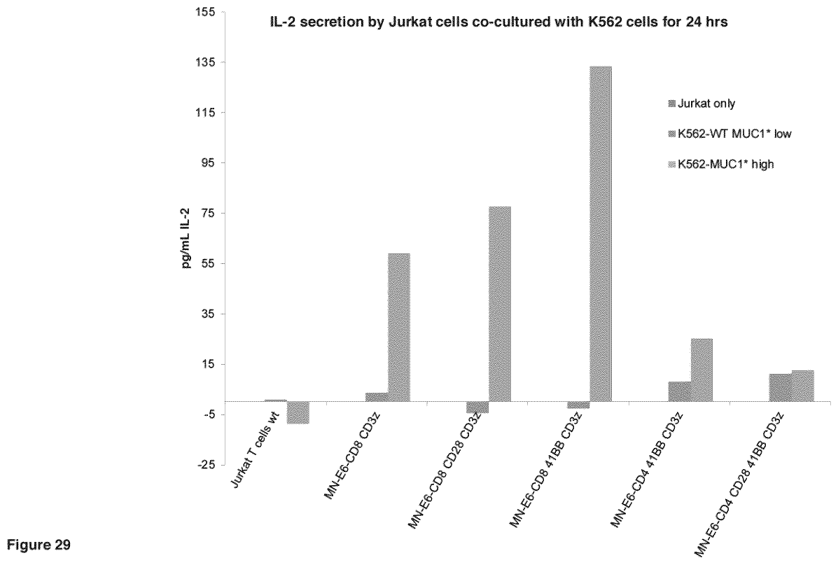

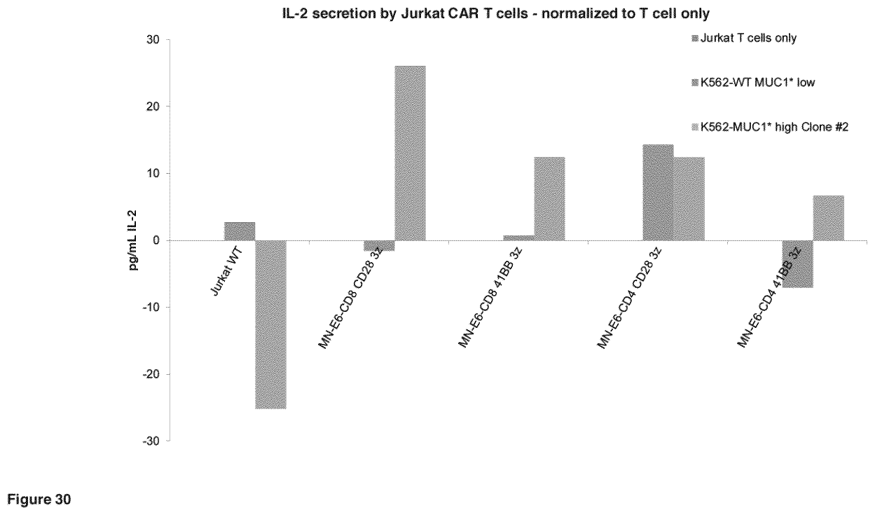

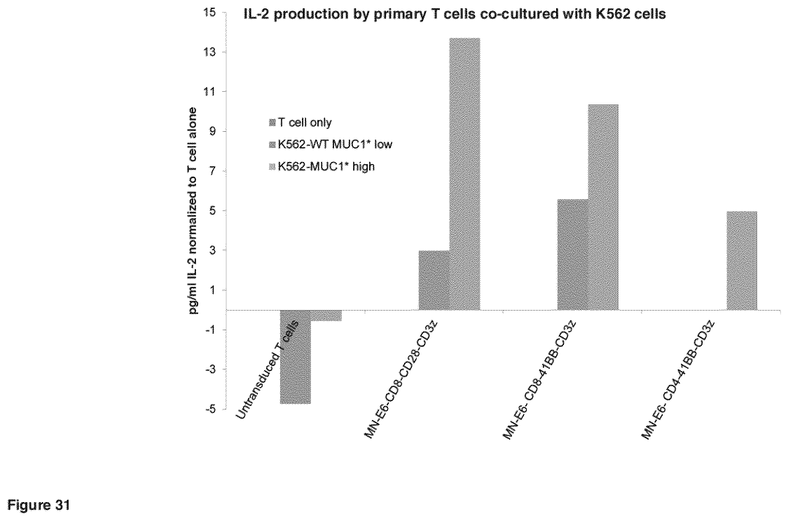

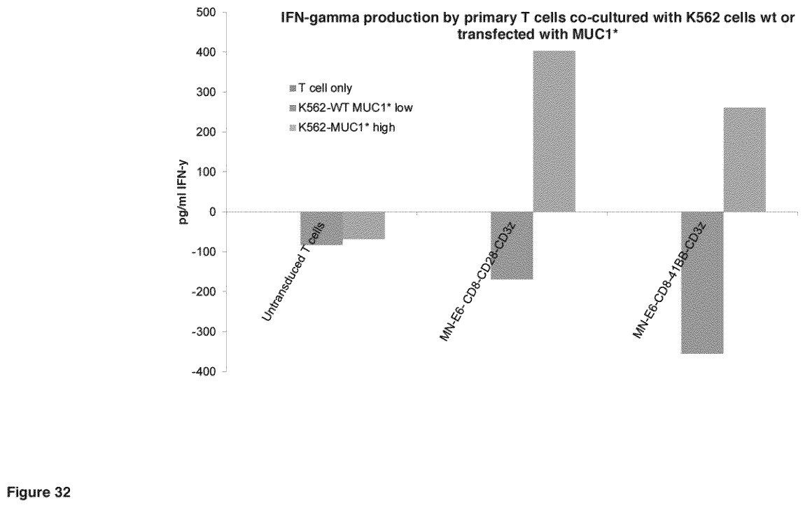

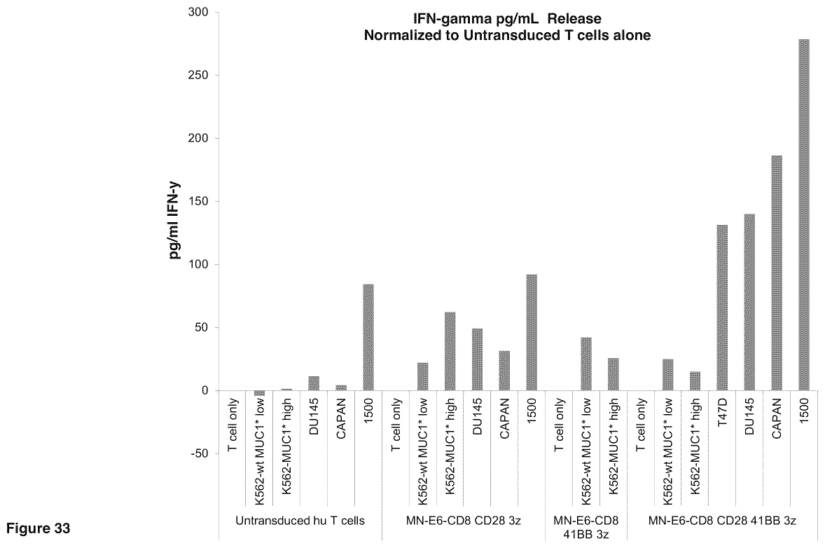

Many MUC1* targeting CARs were generated wherein the targeting antibody fragment at the distal end of the CAR was either MN-E6, MN-C2, MN-C3 or MN-C8. The DNA of each CAR was sequenced to verify that cloning was correctly done. Each construct was then shuffled into an expression plasmid, transfected into cells and then verified that the construct had successfully inserted by Western blot. Surface expression was verified by FACS. The MUC1* targeting CARs were then virally transduced into immune cells. In one aspect they were transduced into Jurkat cells. In another aspect they were transduced into primary human T cells that were purified from blood. A series of functional assays were performed and verified that the CARs were functional. Functional assays showed that both Jurkat cells and primary T cells transduced with MUC1* targeting CAR secreted the cytokine IL-2 when challenged with cells presenting MUC1*. FIG. 29 is a graph of an experiment measuring IL-2 cytokine secretion by Jurkat cells that were transduced with a panel of CARS, including MN-E6 CD8/CD3z, MN-E6 CD8/CD28/CD3z, MN-E6 CD8/41BB/CD3z, MN-E6 CD4/CD28/CD3z and MN-E6 CD4/CD28/41BB/CD3z, IL-2 was secreted only when the CAR Jurkat cells were exposed to K562-wt cells or K562 cells that had been transfected with MUC1*. It should be noted that the parent K562-wt cells express very low levels of MUC1*. Another group of CARs transfected into Jurkat cells was similarly tested for cytokine secretion. FIG. 30 shows IL-2 secretion by Jurkat T cells that were transduced with MN-E6 CD8/CD28/CD3z, MN-E6 CD8/41BB/CD3z, MN-E6 CD4/CD28/CD3z or MN-E6 CD4/41BB/CD3z, when the CAR T cells were exposed to K562-wt cells or K562 cells that had been transfected with MUC1*. Similarly. FIG. 31 shows IL-2 cytokine secretion by primary human T cells that were transduced with MN-E6 CD8/CD28/CD3z, MN-E6 CD8/41BB/CD3z or MN-E6 CD4/41BB/CD3z. Cytokine secretion only occurred when the MUC1* targeting CAR T cells were exposed to K562-wt cells or K562 cells that had been transfected with MUC1*. Another cytokine that is secreted by activated T cells when they see a target cell is interferon-gamma (IFN-g). FIG. 32 shows that interferon-gamma was secreted by primary human T cells that were transduced with a panel of CARs, including MN-E6 CD8/CD28/CD3% and MN-E6 CD4/41BB/CD3z, when the CAR T cells were exposed to K562-wt cells or K562 cells that had been transfected with MUC1*. Interferon-gamma was similarly secreted by primary human T cells that were transduced with a panel of CARs, including MN-E6 CD8/CD28/CD3%. MN-E6 CD8/41BB/CD3% and MN-E6 CD8/CD28/41BB/CD3%, when the MUC1* targeting CAR T cells were exposed to K562-wt cells. K562 cells that had been transfected with MUC1*, or MUC1* positive cancer cells of prostate cancer (DU145), breast cancer (1500) or pancreatic cancer (Capan) (FIG. 33).

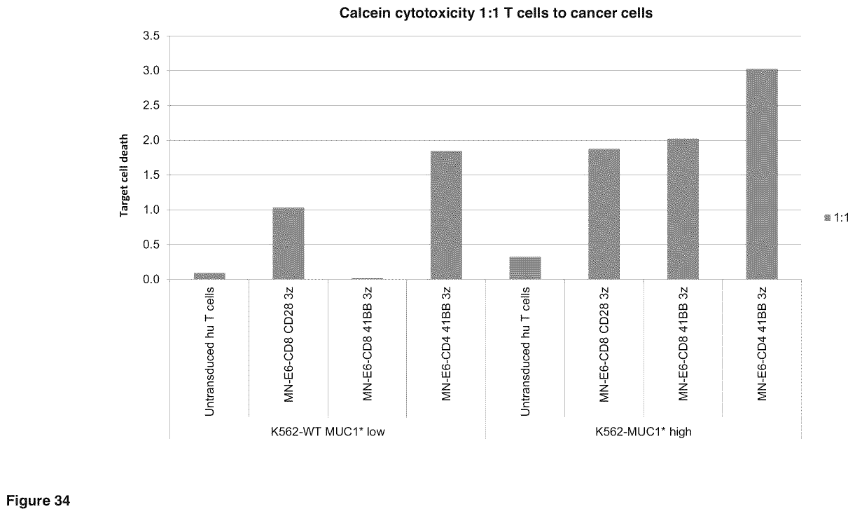

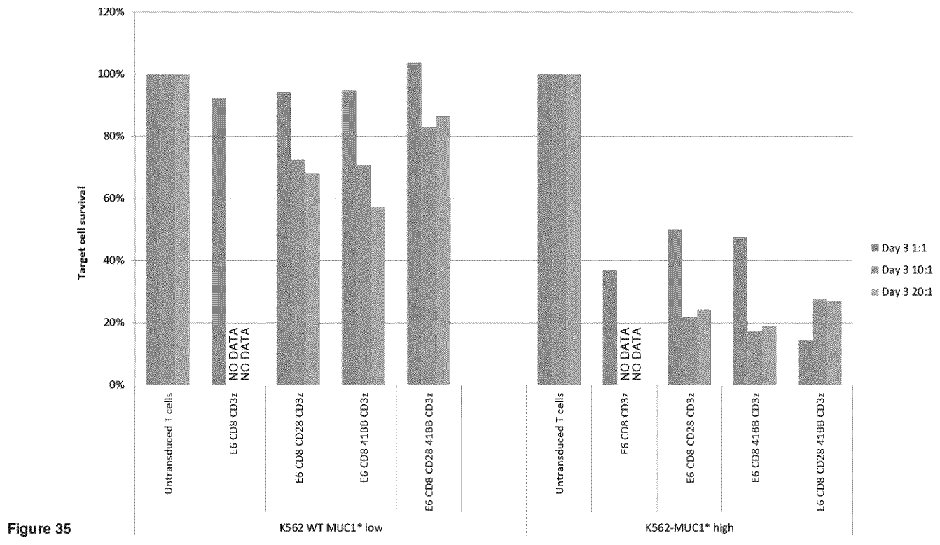

Another measure of function of CAR T cells is whether or not they induce killing of the targeted cells. T cells transfected with a variety of CARs comprising antibody fragments that bind to the PSMGFR sequence of MUC1* killed MUC1* expressing cells in co-culture assays. In one assay, target MUC1* expressing cells are incubated with calcein. When they are mixed with CAR T cells wherein the CAR comprises an antibody fragment such as MN-E6, MN-C2, MN-C3 or MN-C8 the CAR T cells kill the MUC1* presenting cells which causes the target cells to lyse and releases calcein into the supernatant. FIG. 34 is a graph of an experiment measuring target cell death when primary human T cells, isolated from a blood sample, that were transduced with a panel of CARs, including MN-E6 CD8/CD28/CD3z, MN-E6 CD8/41BB/CD3z and MN-E6 CD4/41BB/CD3z, when the CAR T cells were exposed to K562-wt cells or K562 cells that had been transfected with MUC1*. The ratio of T cells to target cells was 1:1 and the cells were co-cultured for 24 hours. FIG. 35 A-B is a graph of FACS measuring a time course of target cell survival from Day 1 to Day 3. Primary human T cells, isolated from a blood sample, were transduced with a panel of CARs, including humanized MN-E6-CD8-3%. MN-E6-CD8-CD28-3z, MN-E6-CD8-41BB-3z, and MN-E6-CD8-CD28-41BB-3%. The CAR T cells were then exposed to K562-wt cells that naturally express low levels of MUC1*, or K562 cells that had been transfected with MUC1* high. The ratio of MUC1* targeting CAR T cells to target cells was either 1:1. 10:1, or 20:1. Surviving cells were detected and measured at Day 1 (A) or Day 3 (B).

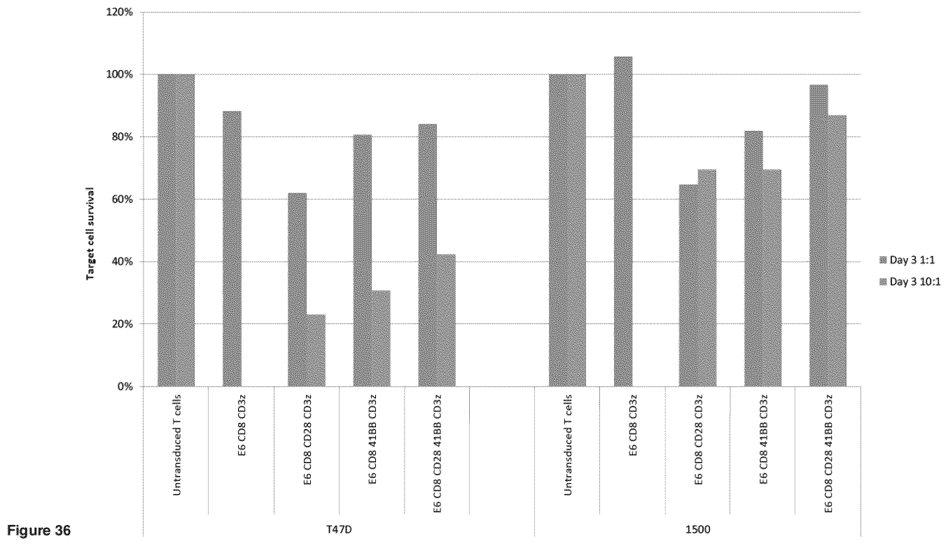

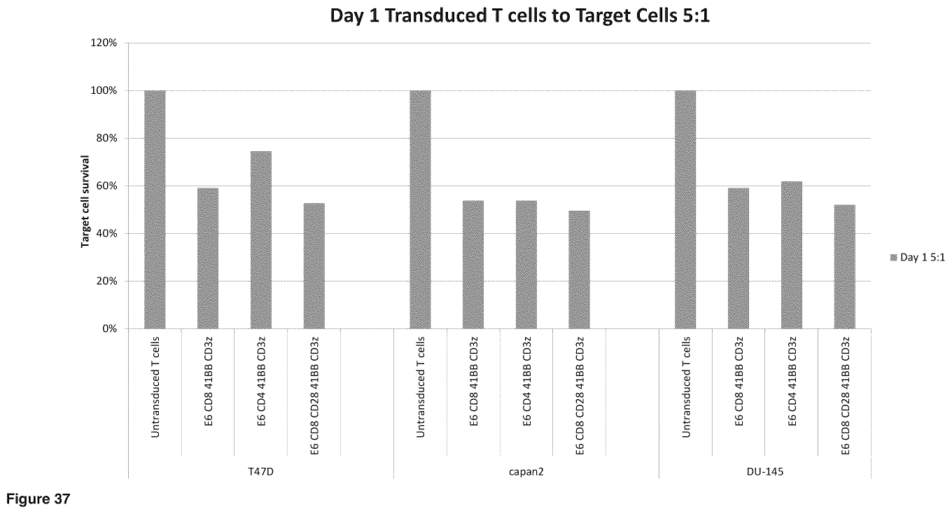

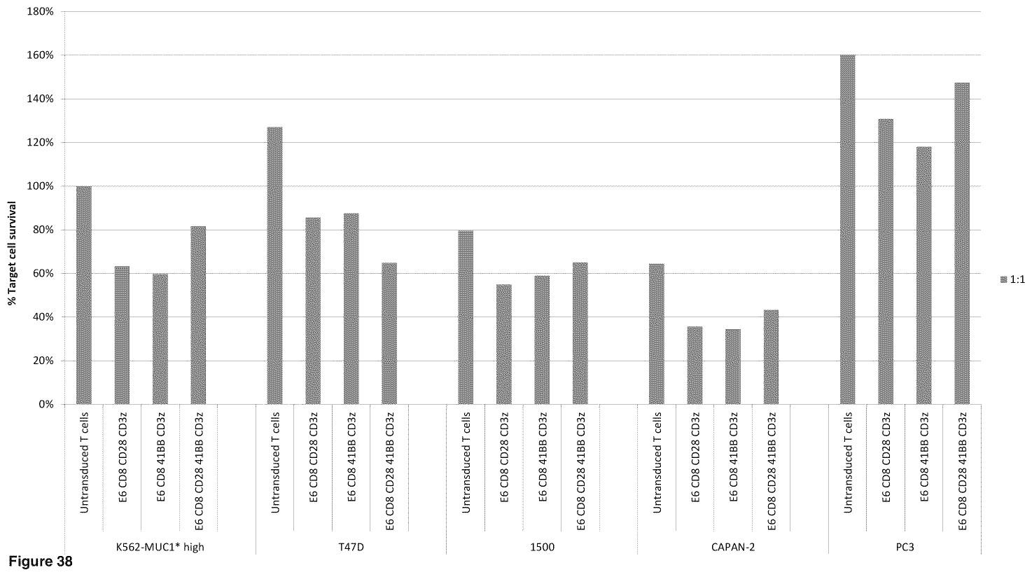

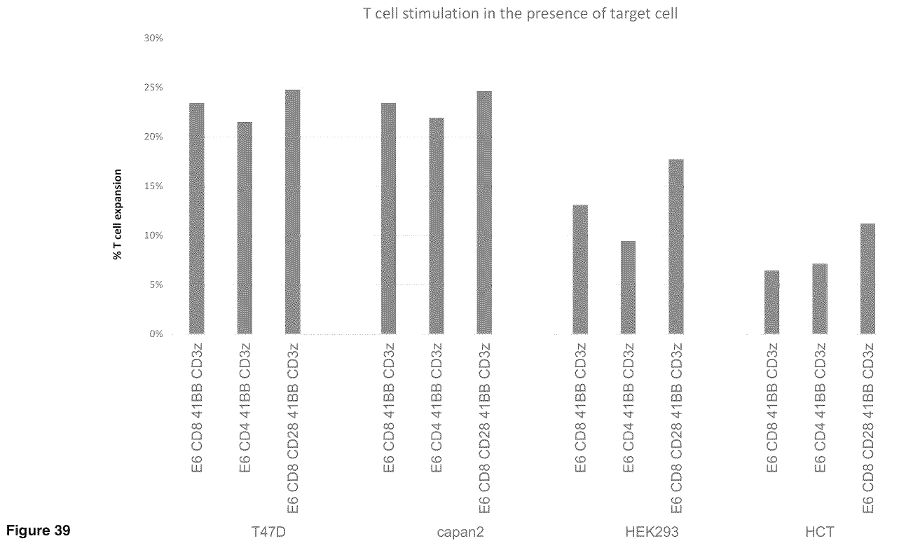

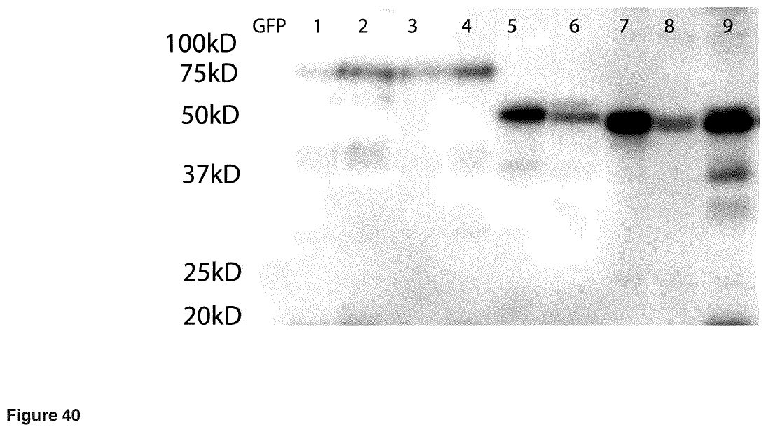

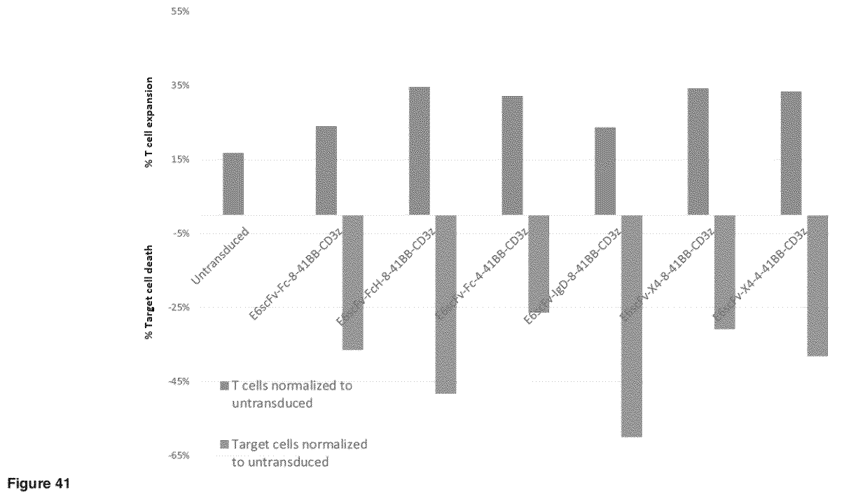

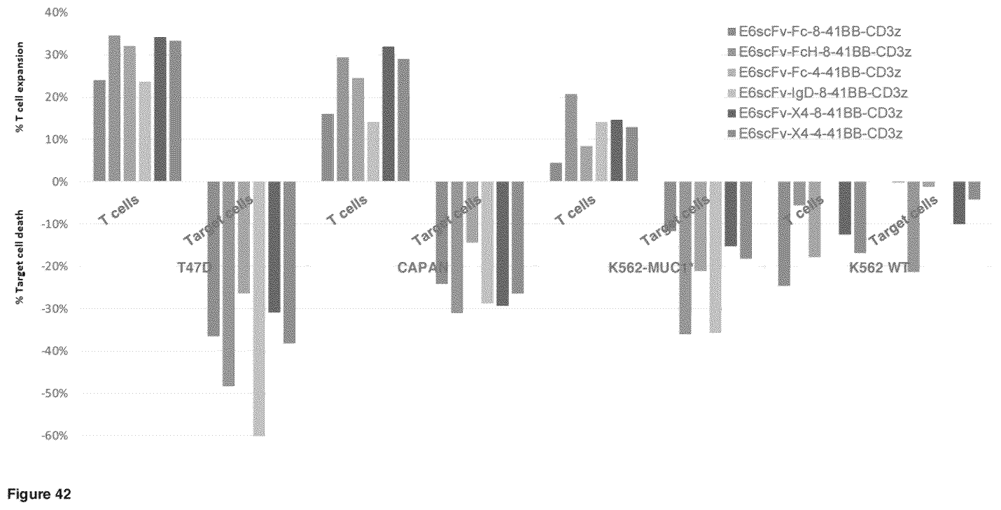

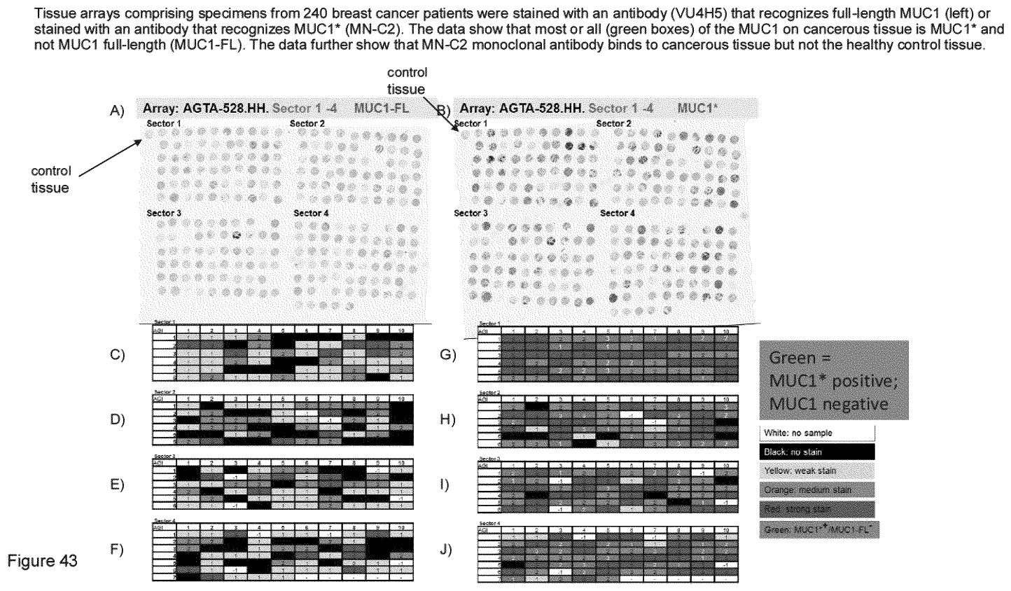

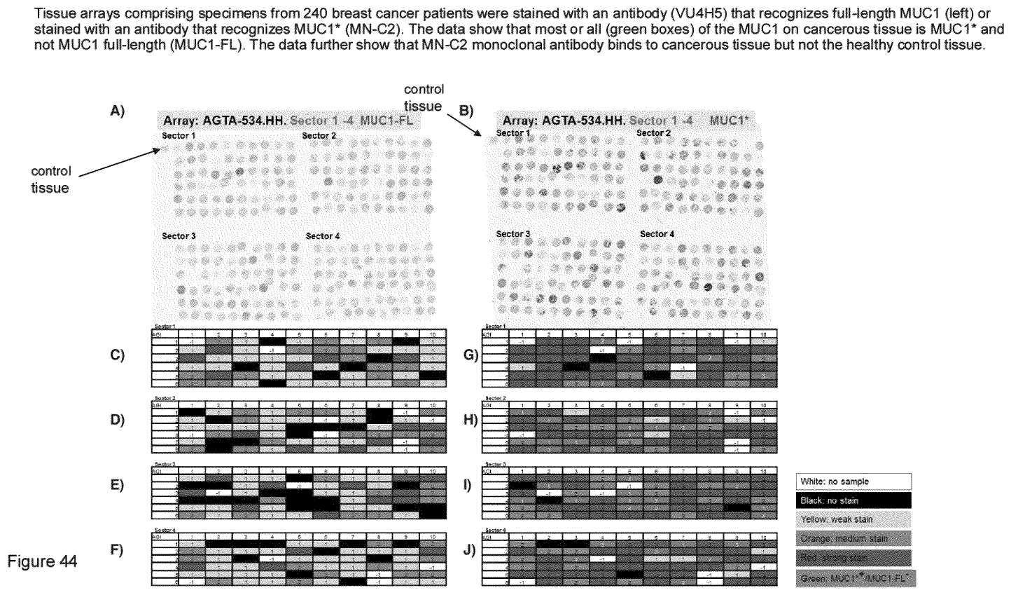

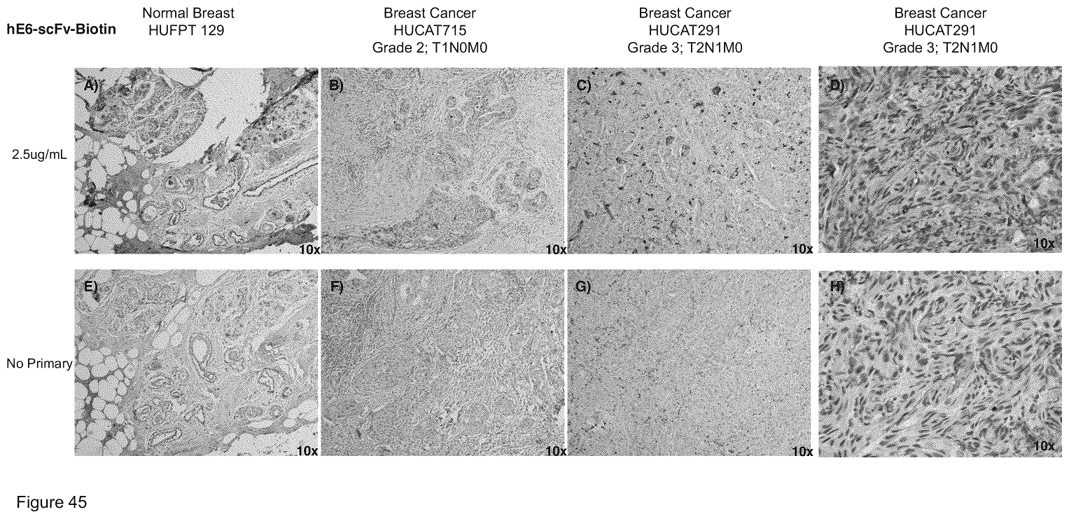

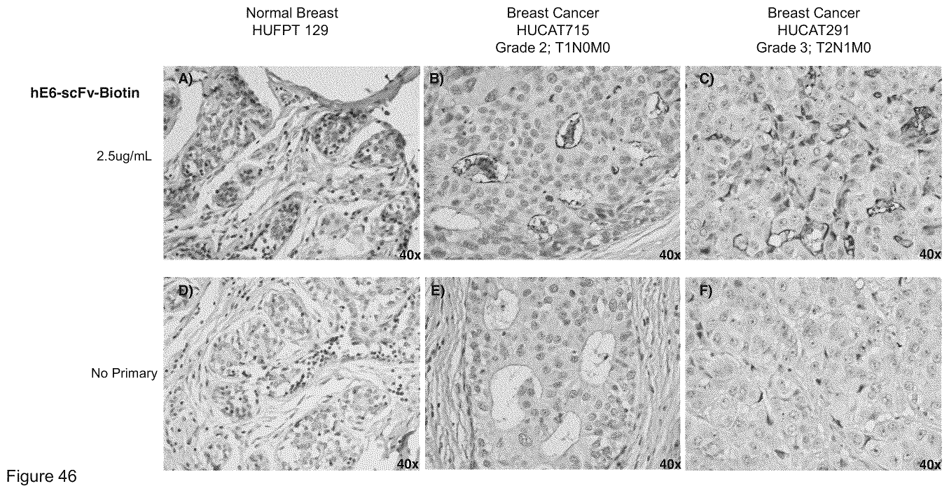

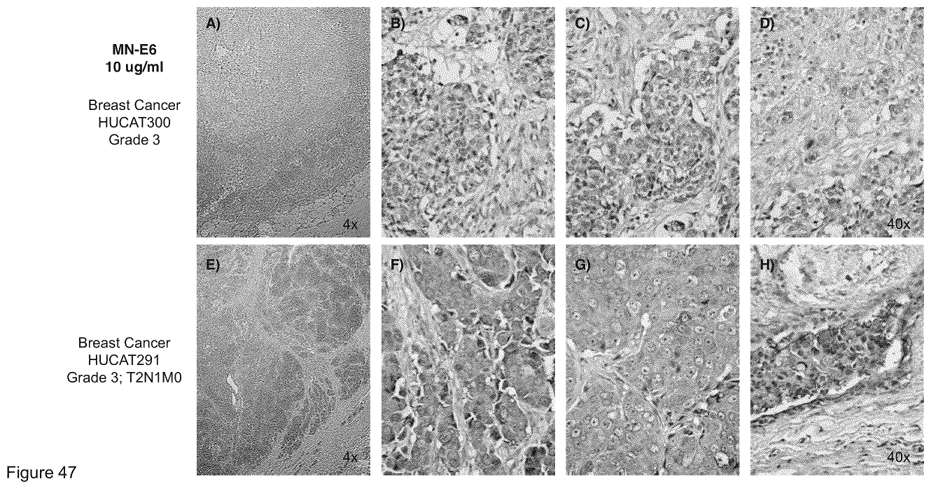

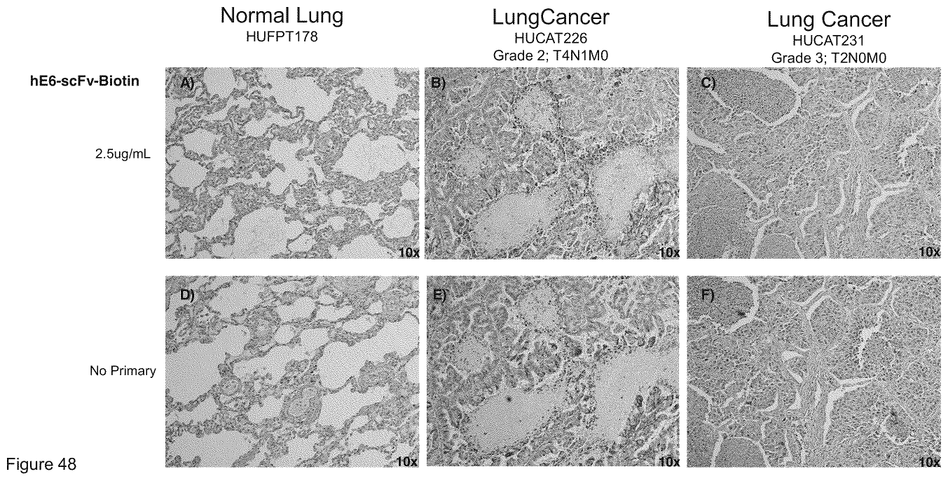

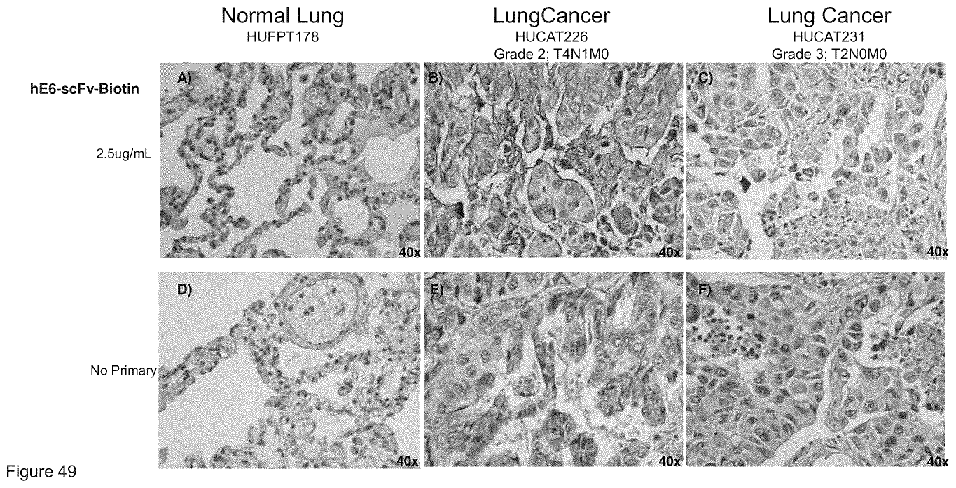

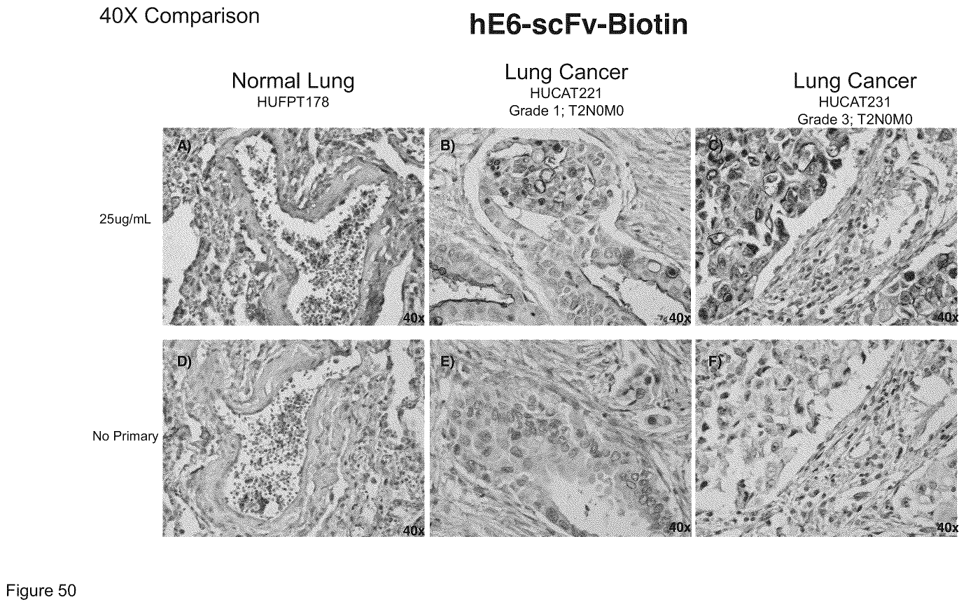

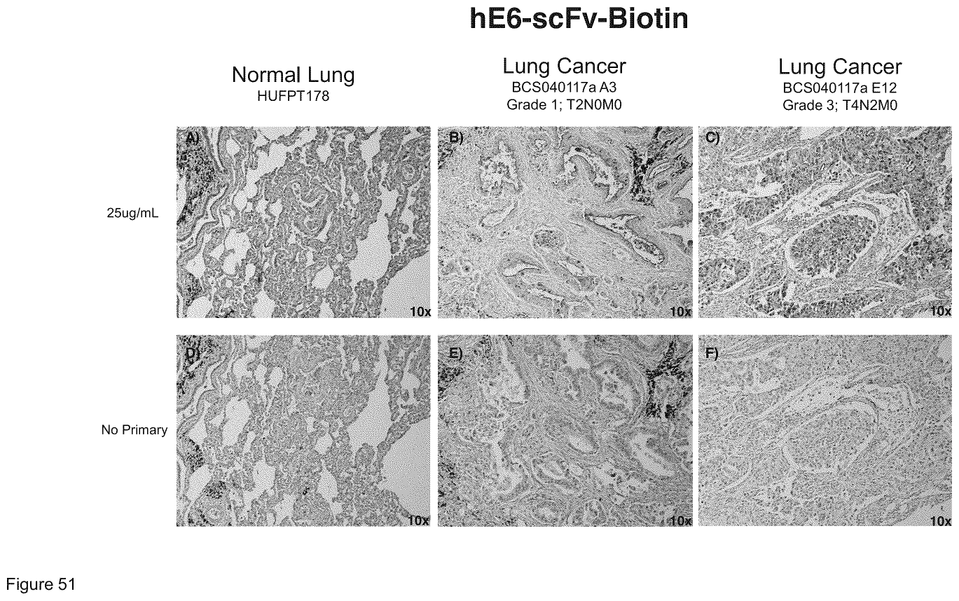

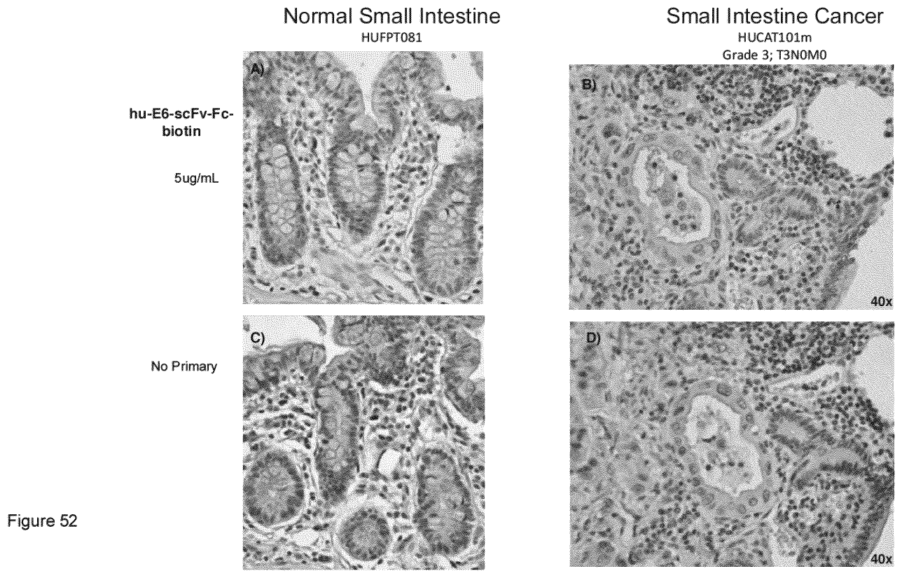

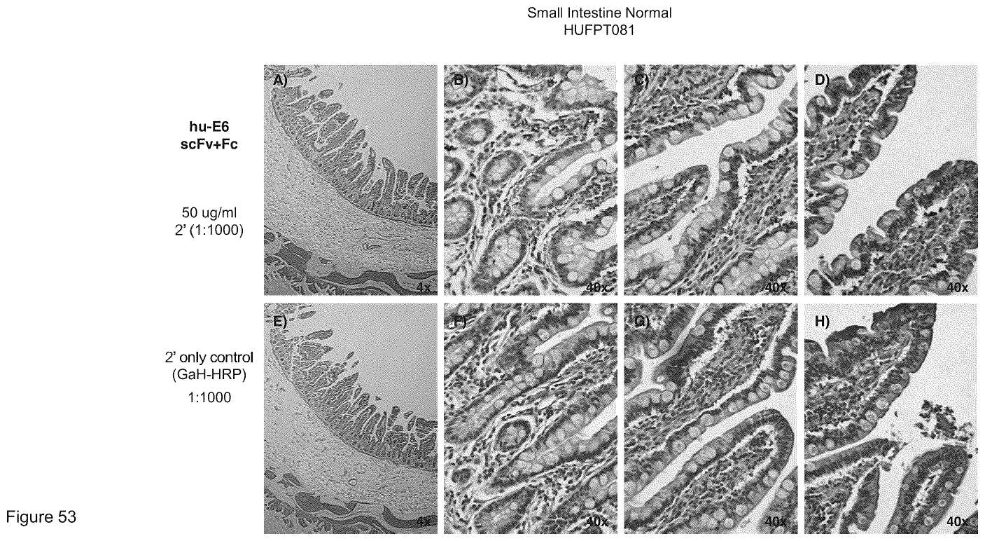

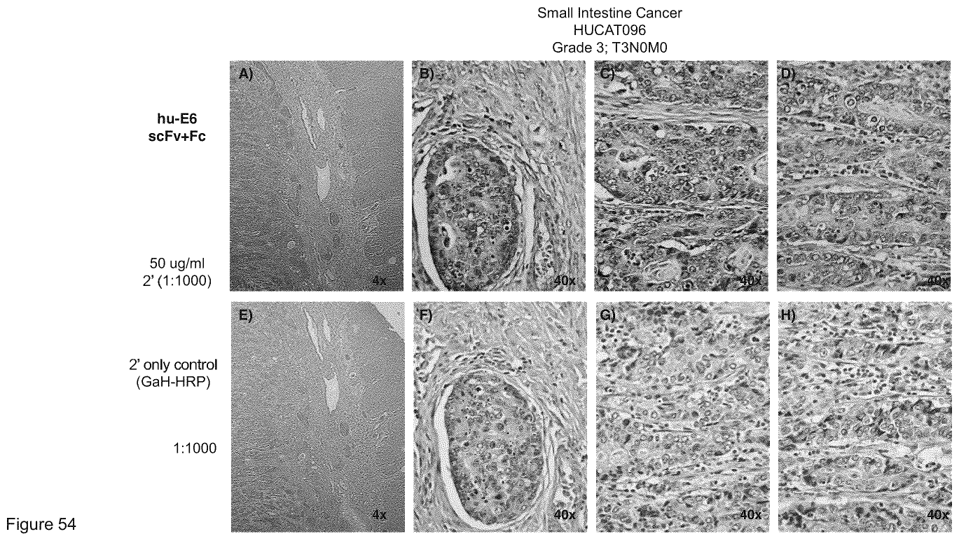

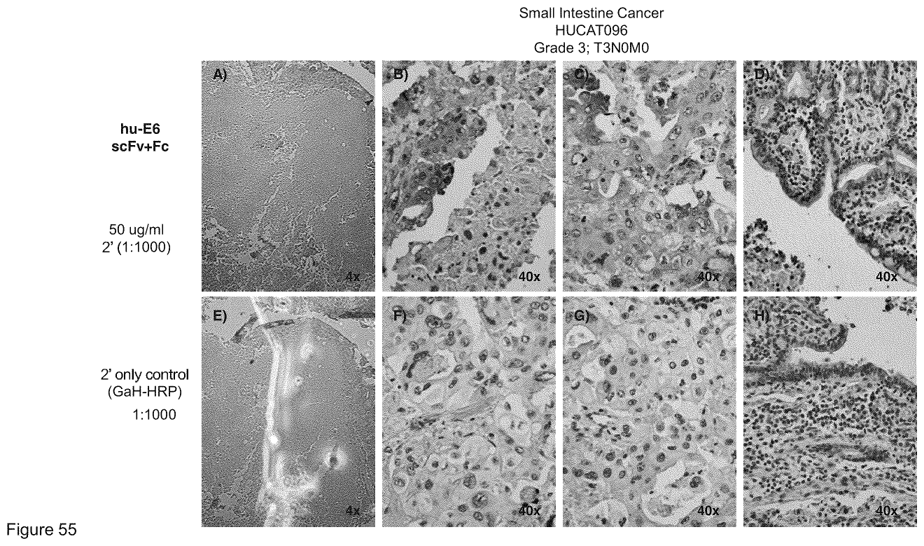

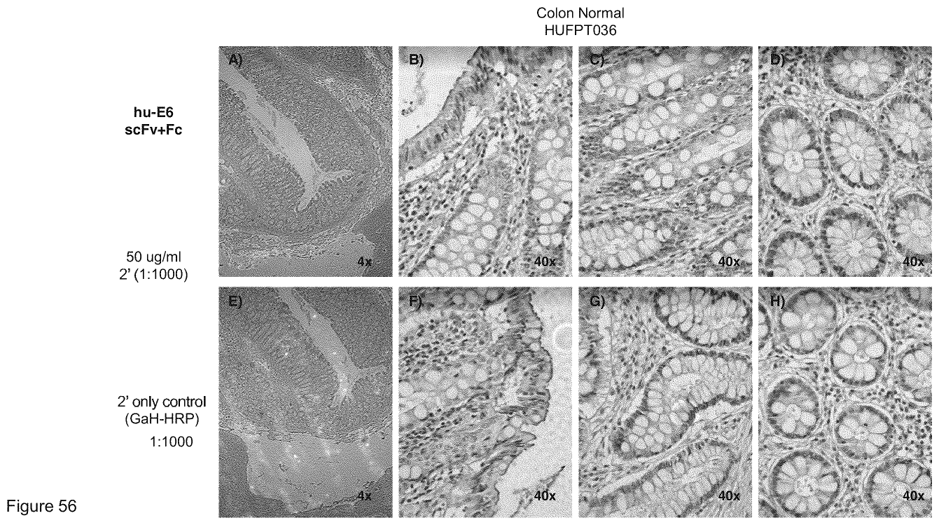

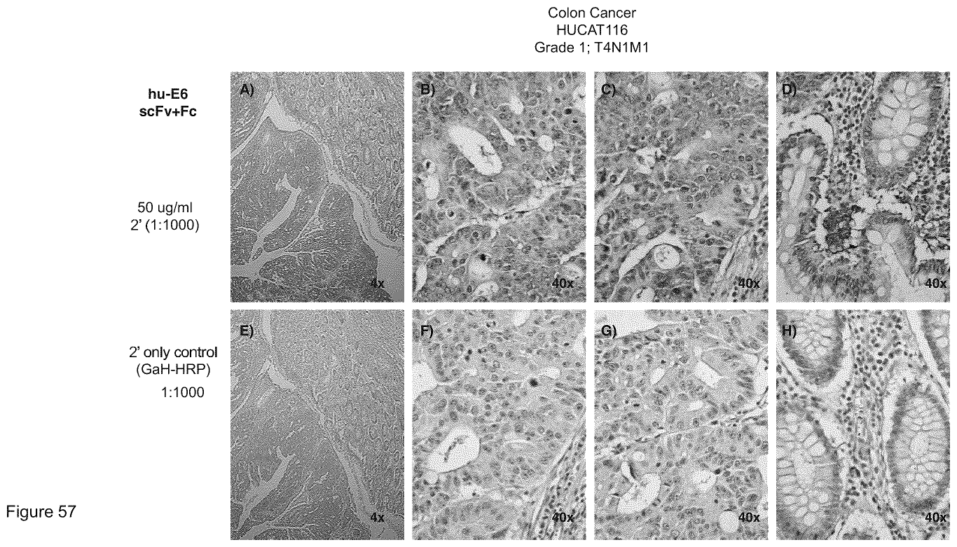

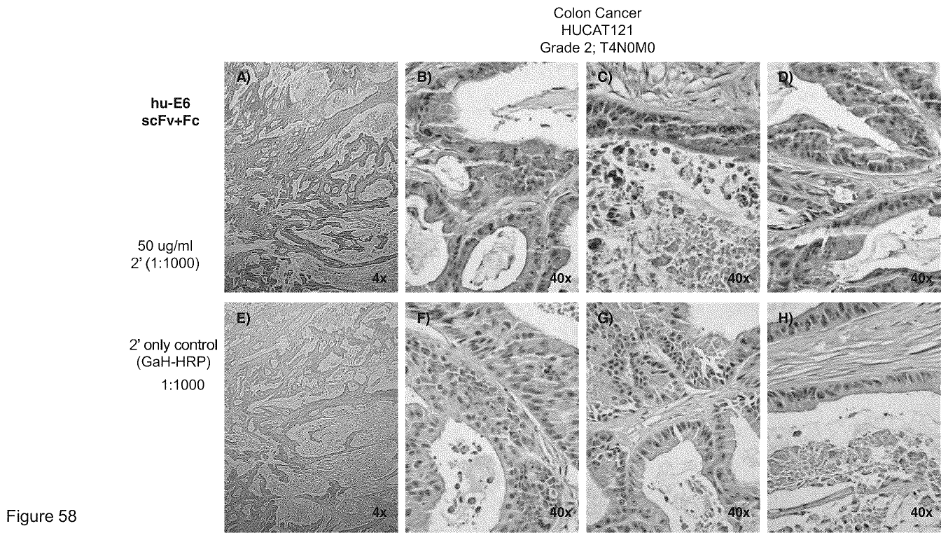

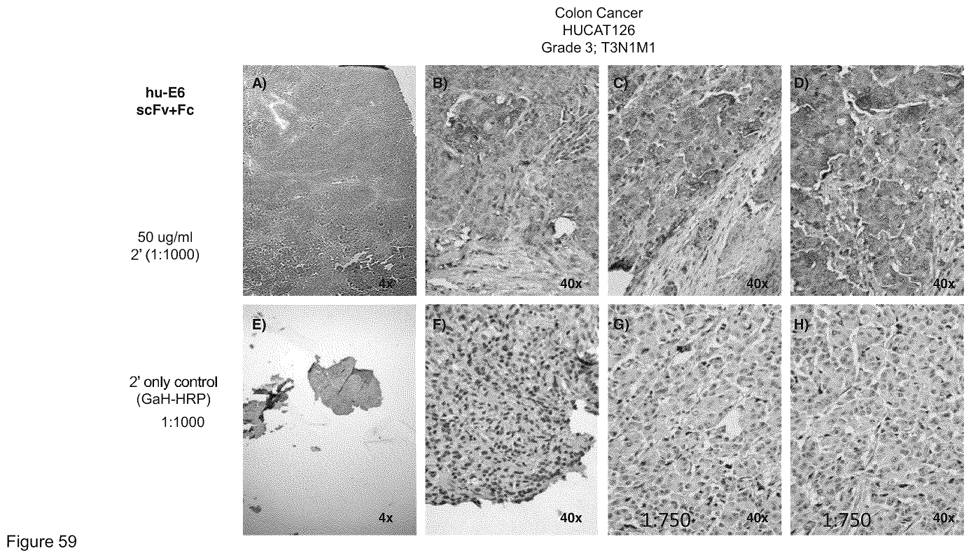

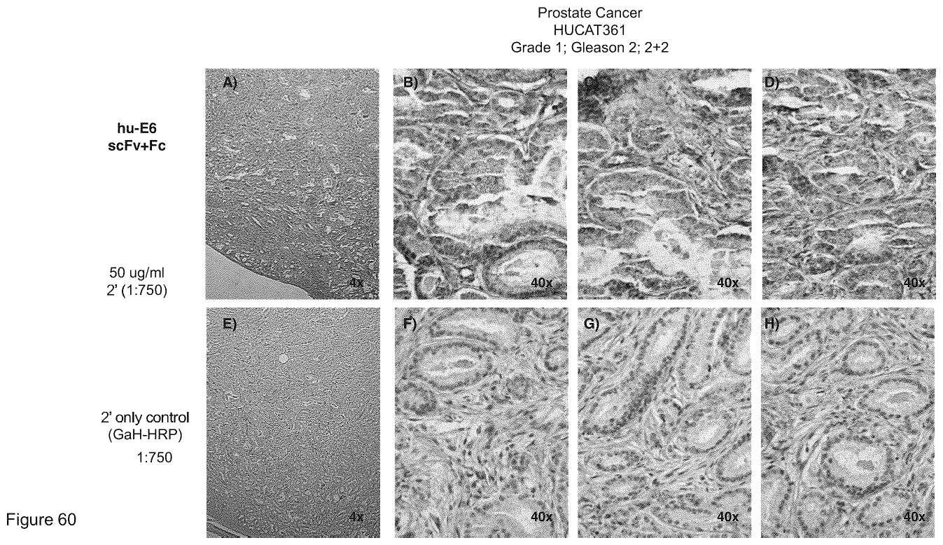

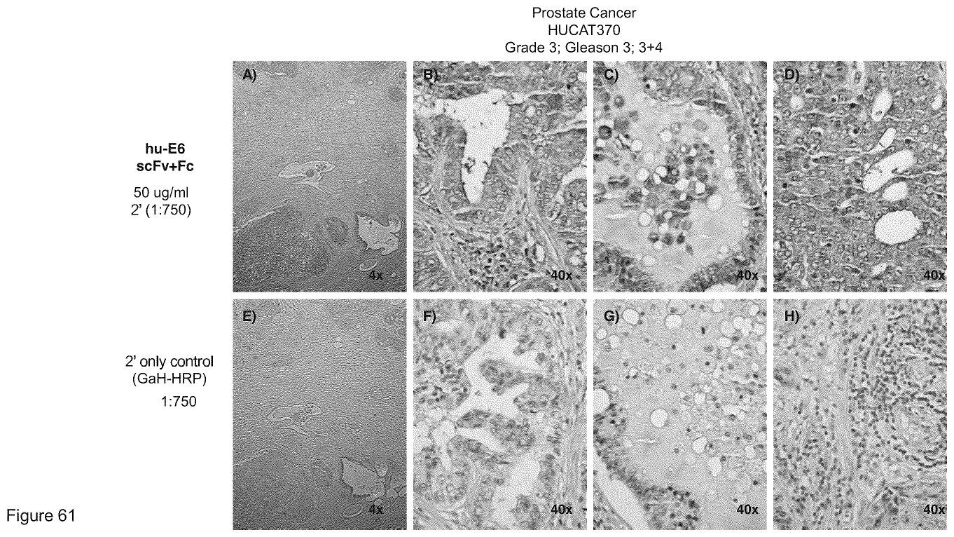

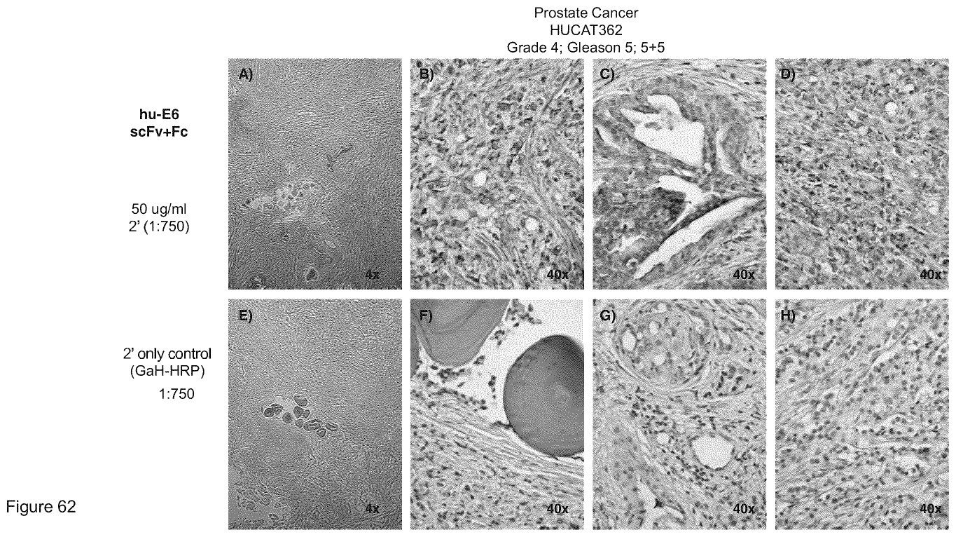

FIG. 36 is a graph of FACS measurements of target cell survival at Day 3 of co-culture experiment. Primary human T cells were transduced with a panel of CARs, including humanized MN-E6-CD8-3%. MN-E6-CD8-CD28-32, MN-E6-CD8-41BB-3z and MN-E6-CD8-CD28-41BB-3z. The CAR T cells were then exposed to MUC1* positive T47D breast cancer cells or MUC1* positive 1500 aka ZR-75-1 breast cancer cells. The ratio of MUC1* targeting CAR T cells to target cells was either 1:1 or 10:1. As can be seen from the graph. T cells transduced with a MUC1* targeting CAR have a much greater killing effect on MUC1* cancer cells than the untransduced control T cells. In addition, the killing effect is much greater when the ratio of T cells: target cells is increased. FIG. 37 is a graph of FACS measurements of target cell survival at Day 1 of co-culture experiment. Primary human T cells were transduced with a panel of CARs, including humanized MN-E6-CD8-41BB-37. MN-E6-CD4-41BB-32, and MN-E6-CD8-CD28-41BB-37. The CAR T cells were then exposed to the following MUC1* positive cancer cells: T47D breast cancer: capan2 pancreatic cancer: or DU-145 prostate cancer. The ratio of MUC1* targeting CAR T cells to target cells was 5:1. As can be seen from the graph. T cells transduced with a MUC1* targeting CAR have a much greater killing effect on MUC1* cancer cells than the untransduced control T cells. Note that the measurements were taken after 24 hours with only a 5:1 T cell to target cell ratio. Also note that MUC1* targeting CARs that have a CD4 extracellular domain-transmembrane-cytoplasmic tail work equally well as CD8 constructs. FIG. 38 is a graph of FACS measurements of target cell survival at Day 3 of co-culture experiment. Primary human T cells were transduced with a panel of CARs, including humanized MN-E6-CD8-41BB-37, MN-E6-CD4-41BB-37, and MN-E6-CD8-CD28-41BB-3z. The CAR T cells were then exposed to the following MUC1* positive cancer cells: K562 leukemia cells transfected with MUC1*: T47D breast cancer: 1500 aka ZR-75-1 breast cancer cells: or CAPAN-2 pancreatic cancer cells. In addition to the untransduced T cell controls, the assay was performed on PC3 MUC1* negative prostate cancer cells. The ratio of MUC1* targeting CAR T cells to target cells was 1:1. As can be seen from the graph. T cells transduced with a MUC1* targeting CAR have a much greater killing effect on MUC1* cancer cells than the untransduced control T cells. In addition, the killing effect is specific for MUC1* positive cells. Note that MUC1* targeting CARs that have a CD4 extracellular domain-transmembrane-cytoplasmic tail work equally well as CD8 constructs. FIG. 39 is a graph of FACS measurements of CAR T cell expansion over 24 hours in co-culture with target cells at a ratio of 5:1 CAR T cells to target cells. The primary human T cells were transduced with a panel of CARs, including humanized MN-E6-CD8-41BB-3z, MN-E6-CD4-41BB-3z, and MN-E6-CD8-CD28-41BB-3z. The CAR T cells were co-cultured with MUC1* positive T47D breast cancer cells, MUC1* positive Capan pancreatic cancer cells, and MUC1-negative cells HCT-116 colon cancer cells and HEK-293 human embryonic kidney cells. As can be seen from the graph, the CAR T population is increased in the presence of MUC1* positive cells. FIG. 40 shows a photograph of a Western blot of MUC1* targeting CARs. From 1 to 9 are: 1, MN-E6scFv-Fc-8-41BB-CD3z (Human Fc as hinge region with CD8 TM): 2: MN-E6scFv-FcH-8-41BB-CD3z (Human Fc hingeless as hinge region with CD8 TM): 3: MN-E6scFv-Fc-4-41BB-CD3z (Human Fc as hinge region with CD4 TM): 4: MN-E6scFv-FcH-4-41BB-CD3z (Human Fc as hingeless hinge region with CD4 TM): 5: MN-E6scFv-IgD-8-41BB-CD3z (hinge region from human IgD with CD8 TM): 6: MN-E6scFv-IgD-4-41BB-CD3% (hinge region from human IgD with CD4 TM): 7: MN-E6scFv-X4-8-41BB-CD3z (Long flexible linker as hinge region with CD8 TM): 8: MN-E6scFv-X4-4-41BB-CD3% (Long flexible linker as hinge region with CD4 TM): 9: MN-E6scFv-8-4-41BB-CD3z (Hinge region from CD8 and CD4 a with CD4 TM).

FIG. 41 shows graphs of FACS scans of T47D breast cancer cells co-cultured with human T cells that were transduced with MN-E6scFv-Fc-8-41BB-CD3z, MN-E6scFv-FcH-8-41BB-CD3% (hingeless). MN-E6scFv-Fc-4-41BB-CD3z, MN-E6scFv-IgD-8-41BB-CD3z, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

FIG. 42 shows graphs of FACS scans of T47D breast cancer cells. Capan-2 pancreatic cancer cells. K562-MUC1* transfected cells, and K562-wt cells that were co-cultured with human T cells that were transduced with MN-E6scFv-Fc-8-41BB-CD3z, MN-E6scFv-FcH-8-41BB-CD3Z. MN-E6scFv-Fc-4-41BB-CD3%. MN-E6scFv-IgD-8-41BB-CD3z, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

As these experiments demonstrate, the critical portion of a CAR is the antibody fragment that directs the immune cell to the tumor cell. As we will show in the following section. MN-E6 and MN-C2 are specific for the form of MUC1* that is expressed on tumor cells. The next most important part of a CAR is the cytoplasmic tail bearing immune system co-stimulatory domains. The identity of these domains modulates the degree of immune response but in no way effect the specificity. As shown, the identity of the transmembrane portion of a CAR is the least important. It appears that as long as the transmembrane portion has some flexibility and is long enough to allow the antibody fragment to reach its cognate receptor on the tumor cell, it will suffice. This is demonstrated in FIGS. 40-42. CARs comprising the MN-E6 targeting antibody fragment, and intracellular co-stimulatory domains 41BB and CD3-zeta but having a variety of different extracellular, transmembrane and short cytoplasmic tail all worked in that they specifically killed the targeted cells while stimulating the expansion of the host T cells. These CARs with variable mid-sections are: MN-E6scFv-Fc-8-41BB-CD3z (Human Fc as hinge region with CD8 TM): 2: MN-E6scFv-FcH-8-41BB-CD3% (Human Fc hingeless as hinge region with CD8 TM): 3: MN-E6scFv-Fc-4-41BB-CD3% (Human Fc as hinge region with CD4 TM): 4: MN-E6scFv-FcH-4-41BB-CD3% (Human Fc as hingeless hinge region with CD4 TM): 5: MN-E6scFv-IgD-8-41BB-CD37 (hinge region from human IgD with CD8 TM): 6: MN-E6scFv-IgD-4-41BB-CD3z (hinge region from human IgD with CD4 TM): 7: MN-E6scFv-X4-8-41BB-CD3z (Long flexible linker as hinge region with CD8 TM): 8: MN-E6scFv-X4-4-41BB-CD3z. (Long flexible linker as hinge region with CD4 TM): 9: MN-E6scFv-8-4-41BB-CD3z (Hinge region from CD8 and CD4 a with CD4 TM).

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR, wherein the CAR is chosen from among the group consisting of MN-E6-CD8-3z: MN-E6-CD4-3z: MN-E6-CD8-CD28-3z: MN-E6-CD4-CD28-3z: MN-E6-CD8-41BB-3z: MN-E6-CD4-41BB-3z: MN-E6-CD8-CD28-41BB-32: MN-E6-CD4-CD28-41BB-3z: MN-E6scFv-Fc-8-41BB-CD3z: MN-E6scFv-FcH-8-41BB-CD3z: MN-E6scFv-Fc-4-41BB-CD3z: MN-E6scFv-FcH-4-41BB-CD3z: MN-E6scFv-IgD-8-41BB-CD3z: MN-E6scFv-IgD-4-41BB-CD3z: MN-E6scFv-X4-8-41BB-CD3z: MN-E6scFv-X4-4-41BB-CD3z: MN-E6scFv-8-4-41BB-CD3z, or any of the aforementioned CARs wherein the MN-E6 is replaced by MN-C2, MN-C3 or MN-C8. Another aspect of the invention is a method for treating a patient diagnosed with, suspected of having, or at risk of developing a cancer, wherein the patient is administered an effective amount of immune cells that have been transduced with one of the aforementioned CARs wherein the MN-E6 is replaced by a peptide comprising antibody variable domain fragments that are specific for a cancer antigen. In any of the above methods, the immune cell may be a T cell and may further be isolated from the patient to be treated.

Specificity of anti-MUC1* targeting antibodies

The most accurate way of demonstrating antibody specificity is testing the antibody on normal human tissue specimens compared to cancerous tissue specimens. MN-C2 and MN-E6 were shown to specifically bind to MUC1 or MUC1* positive cancer cells. Several breast tumor arrays were assayed using several anti-MUC1 or MUC1* antibodies. Essentially the studies involving serial sections of breast cancer tissue specimens from over 1.200 different breast cancer patients showed that very little full-length MUC1 remains on breast cancer tissues. The vast majority of the MUC1 expressed is MUC1* and is stained by MN-C2. The analysis was performed by Clarient Diagnostics and tissue staining was scored using the Allred method. For example. FIG. 43 shows serial sections of breast cancer tissue arrays that were stained with either VU4H5, a commercially available anti-MUC1 antibody that binds to the tandem repeats, or MN-C2 that binds to MUC1*. FIGS. 43 and 44 are photographs of breast cancer tissue arrays stained with either VU4H5 which recognizes MUC1-FL (full length) or MN-C2 which recognizes cancerous MUC1*. Tissue staining was scored using Allred scoring method which combines an intensity score and a distribution score. Below the photographs of the tissue arrays are color-coded graphs displaying the results. As can be seen, the arrays stained with VU4H5 are very light and many tissues do not stain at all despite the published reports that MUC1 is aberrantly expressed on over 96% of all breast cancers as evidenced by nucleic acid based diagnostics. In contrast, the arrays stained with MN-C2 are very dark (red versus yellow or white in graph). Additionally, many tissues did not stain at all with anti-full-length MUC1 but stained very dark with MN-C2. (see green boxes in graph). Similarly, we stained normal or cancerous breast tissues with humanized MN-E6 scFv-Fc. The antibody fragment was biotinylated so it could be visualized by a secondary streptavidin based secondary. As can be seen in FIG. 45, hMN-E6 scFv-Fc does not stain normal breast tissue but stains cancerous breast tissue. Further, the intensity and homogeneity of staining increases with tumor grade and/or metastatic grade of the patient (FIGS. 45 and 46). Similarly, hMN-E6 scFv-Fc did not stain normal lung tissue but did stain lung cancer tissue (FIGS. 47-51) and the intensity and distribution of staining increased as tumor grade or metastatic grade increased. FIG. 52 shows photographs of normal small intestine and cancerous small intestine tissues stained with humanized MN-E6-scFv-Fc biotinylated anti-MUC1* antibody at 5 ug/mL, then stained with a secondary streptavidin HRP antibody. A) is a normal small intestine tissue. B) is small intestine cancer from patient as denoted in the figure. C,D are photographs of the corresponding serial sections that were stained with the secondary antibody alone. FIG. 53 shows photographs of normal small intestine tissues stained with humanized MN-E6-scFv-Fc anti-MUC1* antibody at 50 ug/mL, then stained with a secondary goat-anti-human HRP antibody. A-D are normal small intestine tissue. E-H are photographs of the corresponding serial sections that were stained with the secondary antibody alone. FIG. 54 shows photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone. FIG. 55 shows photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone. FIG. 56 shows photographs of normal colon tissues stained with humanized MN-E6-scFv-Fc anti-MUC1* antibody at 50 ug/mL, then stained with a secondary goat-anti-human HRP antibody. A-D are normal colon. E-H are photographs of the corresponding serial sections that were stained with the secondary antibody alone. FIG. 57 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone. FIG. 58 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a Grade 2 patient as denoted in figure. E-H are photographs of the corresponding serial sections that were stained with the secondary antibody alone. FIG. 59 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone. FIG. 60 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone. FIG. 61 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone. FIG. 62 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

One aspect of the invention is a method for treating a patient diagnosed with, suspected of having, or at risk of developing a MUC1 positive or MUC1* positive cancer, wherein a specimen is obtained from the patient's cancer and is tested for reactivity with an antibody that binds to PSMGFR SEQ ID NO:2, SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621). The patient is then treated with an scFv, scFv-Fc or CAR T that comprises antibody variable fragments from the antibody that reacted with their cancer specimen. Another aspect of the invention is a method for treating a patient diagnosed with, suspected of having, or at risk of developing a MUC1 positive or MUC1* positive cancer, wherein a specimen is obtained from the patient's cancer and is tested for reactivity with MN-E6-scFv, MN-C2-scFv, MN-C3-scFv or MN-C8-scFv: the patient is then treated with the scFv, scFv-Fc-mut or CAR T that comprises portions of the antibody that reacted with their cancer specimen.

### BiTEs

Divalent (or bivalent) single-chain variable fragments (di-scfvs, bi-scFvs) can be engineered by linking two scFvs. This can be done by producing a single peptide chain with two V_{H} and two V_{L} regions, yielding tandem scFvs. Another possibility is the creation of scFvs with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Consequently, diabody drugs could be dosed much lower than other therapeutic antibodies and are capable of highly specific targeting of tumors in vivo. Still shorter linkers (one or two amino acids) lead the formation of trimers, so-called triabodies or tribodies. Tetrabodies have also been produced. They exhibit an even higher affinity to their targets than diabodies.

All of these formats can be composed from variable fragments with specificity for two different antigens, in which case they are types of bispecific antibodies. The furthest developed of these are bispecific tandem di-scFvs, known as bi-specific T-cell engagers (BiTE antibody constructs). BiTEs are fusion proteins consisting of two scFvs of different antibodies, on a single peptide chain of about 55 kilodaltons. One of the scFvs may bind to T cells such as via the CD3 receptor, and the other to a tumor cell via a tumor specific molecule, such aberrantly expressed MUC1*.

Another aspect of the invention is a method for treating a patient diagnosed with, suspected of having, or at risk of developing a MUC1 positive or MUC1* positive cancer, wherein the patient is administered an effective amount of a BiTE wherein one antibody variable fragment of the BiTE binds to a T cell surface antigen and the other antibody variable fragment of the BiTE binds to PSMGFR SEQ ID NO:2, SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621). In one case, the antibody variable fragment of the BiTE that binds to MUC1* comprises portions of huMN-E6, huMN-C2, huMN-C3, or huMN-C8.

In another aspect of the invention, MUC1* peptides including PSMGFR SEQ ID NO: 2, most or all of SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO: 621) are used in adoptive T cell approaches. In this case, a patient's T cells are exposed to the MUC1* peptides and through various rounds of maturation, the T cells develop MUC1* specific receptors. The adapted T cells are then expanded and administered to the donor patient who is diagnosed with, suspected of having, or is at risk of developing a MUC1* positive cancer.

### Other MUC1 Cleavage Sites

However, MUC1 is cleaved to the growth factor receptor form, MUC1*, on some healthy cells in addition to cancer cells. For example, MUC1 is cleaved to MUC1* on healthy stem and progenitor cells. A large percentage of bone marrow cells are MUC1* positive. Portions of the intestine are MUC1* positive.

The inventors have discovered that MUC1 can be cleaved at different positions that are relatively close to each other but the location of cleavage changes the fold of the remaining portion of the extracellular domain. As a result, monoclonal antibodies can be identified that bind to MUC1* cleaved at a first position but do not bind to MUC1* that has been cleaved at a second position. This discovery is disclosed in WO2014/028668, filed August 14. 2013, the contents of which are incorporated by reference herein its entirety. We identified a set of anti-MUC1* monoclonal antibodies that bind to a MUC1* as it appears on cancer cells but do not bind to MUC1* as it appears on stem and progenitor cells. Conversely, we identified a second set of monoclonal antibodies that bind to stem and progenitor cells but do not bind to cancer cells. One method used to identify stem specific antibodies is as follows: supernatants from monoclonal hybridomas were separately adsorbed onto 2 multi-well plates. Stem cells, which are non-adherent cells, were put into one plate and cancer cells which are adherent were put into an identical plate. After an incubation period, the plates were rinsed and inverted. If the non-adherent stem cells stuck to the plate, then the monoclonal in that particular well recognizes stem cells and will not recognize cancer cells. Antibodies that did not capture stem cells or antibodies that captured cancer cells were identified as cancer specific stem cells. FACS analysis has confirmed this method works. Antibodies MN-E6 and MN-C2 are examples of cancer-specific antibodies. Antibodies MN-C3 and MN-C8 are examples of stem-specific antibodies. Although both sets of antibodies are able to bind to a peptide having the PSMGFR sequence. FACS analysis shows that the anti-MUC1* polyclonal antibody and MN-C3 bind to MUC1* positive bone marrow cells but MN-E6 does not. The MUC1* polyclonal antibody was generated by immunizing a rabbit with the PSMGFR peptide. Similarly. MN-C3 binds to stem cells of the intestinal crypts but MN-E6 does not. Conversely. MN-E6 antibody binds to cancerous tissue while the stem-specific MN-C3 does not. Competition ELISA experiments indicate that the C-terminal 10 amino acids of the PSMGFR peptide are required for MN-E6 and MN-C2 binding, but not for MN-C3 and MN-C8. Therefore, another method for identifying antibodies that are cancer specific is to immunize with a peptide having the sequence of the PSMGFR peptide minus the 10 N-terminal amino acids or use that peptide to screen for antibodies or antibody fragments that will be cancer specific. Antibodies that bind to a peptide with a sequence of PSMGFR peptide minus the N-terminal 10 amino acids but do not bind to a peptide with a sequence of PSMGFR peptide minus the C-terminal 10 amino acids are cancer specific antibodies for use in the treatment or prevention of cancers.

The extracellular domain of MUC1 is also cleaved on stem cells and some progenitor cells, where activation of cleaved MUC1 by ligands NME1 in dimer form or NME7 promotes growth and pluripotency and inhibits differentiation. The transmembrane portion of MUC1 that remains after cleavage is called MUC1* and the extracellular domain is comprised essentially of the Primary Sequence of MUC1 Growth Factor Receptor (PSMGFR) sequence. However, the exact site of cleavage can vary depending on cell type, tissue type, or which cleavage enzyme a particular person expresses or overexpresses. In addition to the cleavage site that we previously identified which leaves the transmembrane portion of MUC1* comprising most or all of the PSMGFR SEQ ID NO:2, other cleavage sites result in an extended MUC1* comprised of most or all of SNIKFRPGSVVVQLTLAFREGTINVHDVETQFNQYKTEAASRY (SEQ ID NO:620): or SVVVQLTLAFREGTINVHDVETQFNQYKTEAASRY (SEQ ID NO:621). The site of MUC1 cleavage affects how the remaining extracellular domain folds. We have identified monoclonal antibodies that bind to cleaved MUC1* on cancer cells but do not bind to cleaved MUC1* as it exists on healthy stem and progenitor cells.

Whereas an anti-MUC1* antibody or antibody-like molecule may be most effective if it competitively inhibits the binding of NME1. NME6. NME8 or NME7 or NME7-AB to MUC1*, for example an antibody that binds to the PSMGFR sequence especially if said antibody is unable to bind to a PSMGFR peptide if the 10 C-terminal amino acids are missing, antibodies or antibody-like molecules that carry a payload need not competitively inhibit the binding of MUC1* ligands to be effective as anti-cancer agents. For example antibodies or antibody-like molecules that are conjugated to a toxin could be effective at killing target cancer cells without necessarily inhibiting binding of the activating ligands. For example, antibodies or antibody-like molecules such as CARs or BiTEs which recruit the patient's immune system to the tumor can be effective as anti-cancer agents even if the antibody fragment targets a portion of MUC1* such that antibody fragment binding does not competitively inhibit the binding of NME1. NME6, NME8, NME7-AB or NME7. In a preferred embodiment the antibody fragment incorporated into a CAR, an adaptive T cell receptor or a BiTE does competitively inhibit the binding of NME1. NME6. NME8. NME7-AB or NME7 to MUC1*.

Antibodies that are able to bind to the extracellular domain of the remaining transmembrane portion block the interaction between the MUC1* extracellular domain and activating ligands and in this way can be used as therapeutic agents, for example for the treatment of cancers. Anti-MUC1* antibodies are also useful for the growth, delivery, identification or isolation of stem cells both in vitro and in vivo.

General Strategy for using antibodies, antibody fragments and CARs that target the extracellular domain of MUC1*

Monoclonal antibodies MN-C3 and MN-C8 have a greater binding affinity for stem cells than cancer cells. Humanized antibodies and antibody fragments containing sequences derived from the variable regions of MN-C3 and MN-C8 can be used as an adhesion surface coating for human stem cells.

Alternatively, humanized antibodies and antibody fragments containing sequences derived from the variable regions of MN-C3 and MN-C8 can be used to deliver stem cells to a specific location such as for in situ human therapeutics. In one case, a substrate coated with humanized MN-C3 or MN-C8 derived antibodies or antibody fragments is loaded with stem cells then inserted into a patient. In another case, a substrate coated with humanized MN-C3 or MN-C8 derived antibodies or antibody fragments is inserted into a patient in order to recruit the patient's own stem cells to a specific area for therapy. Human therapies in which antibodies that bind to human stem cells will be of therapeutic use include spinal cord repair. Substrates coated with humanized MN-C3 or MN-C8 derived antibodies or antibody fragments are also used to identify or isolate human antibodies. Humanized MN-C3 or MN-C8 derived antibodies can also be used to stimulate the growth of stem cells.

Sequence Listing Free Text: xml text file named “56699-731_302SL” having byte size of 993,008, created Aug. 3, 2022 is incorporated by reference herein.

As regards the use of nucleotide symbols other than a, g, c, t, they follow the convention set forth in WIPO Standard ST.25, Appendix 2, Table 1, wherein k represents t or g: n represents a, c, t or g; m represents a or c; r represents a or g: s represents c or g; w represents a or t and y represents c or t.

MUC1 Receptor
(Mucin 1 precursor, Genbank Accession number: P15941)
(SEQ ID NO: 1)
MTPGTQSPFFLLLLLTVLTVVTGSGHASSTPGGEKETSATQRSSVPSSTEKNAVSMTSSVLSSHSPGSGSSTT
QGQDVTLAPATEPASGSAATWGQDVTSVPVTRPALGSTTPPAHDVTSAPDNKPAPGSTAPPAHGVTSAPDTRP
APGSTAPPAHGVTSAPDTRPAPGSTAPPAHGVTSAPDTRPAPGSTAPPAHGVTSAPDTRPAPGSTAPPAHGVT
SAPDTRPAPGSTAPPAHGVTSAPDTRPAPGSTAPPAHGVTSAPDTRPAPGSTAPPAHGVTSAPDTRPAPGSTA
PPAHGVTSAPDTRPAPGSTAPPAHGVTSAPDTRPAPGSTAPPAHGVTSAPDTRPAPGSTAPPAHGVTSAPDTR
PAPGSTAPPAHGVTSAPDTRPAPGSTAPPAHGVTSAPDTRPAPGSTAPPAHGVTSAPDTRPAPGSTAPPAHGV
TSAPDTRPAPGSTAPPAHGVTSAPDTRPAPGSTAPPAHGVTSAPDTRPAPGSTAPPAHGVTSAPDTRPAPGST
APPAHGVTSAPDTRPAPGSTAPPAHGVTSAPDTRPAPGSTAPPAHGVTSAPDTRPAPGSTAPPAHGVTSAPDT
RPAPGSTAPPAHGVTSAPDTRPAPGSTAPPAHGVTSAPDTRPAPGSTAPPAHGVTSAPDTRPAPGSTAPPAHG
VTSAPDTRPAPGSTAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGSTAPPAHGVTSAPD
TRPAPGSTAPPAHGVTSAPDTRPAPGSTAPPAHGVTSAPDTRPAPGSTAPPAHGVTSAPDTRPAPGSTAPPAH
GVTSAPDTRPAPGSTAPPAHGVTSAPDTRPAPGSTAPPAHGVTSAPDTRPAPGSTAPPAHGVTSAPDNRPALG
STAPPVHNVTSASGSASGSASTLVHNGTSARATTTPASKSTPFSIPSHHSDTPTTLASHSTKTDASSTHHSSV
PPLTSSNHSTSPQLSTGVSFFFLSFHISNLQFNSSLEDPSTDYYQELQRDISEMFLQIYKQGGFLGLSNIKFR
PGSVVVQLTLAFREGTINVHDVETQFNQYKTEAASRYNLTISDVSVSDVPFPFSAQSGAGVPGWGIALLVLVC
VLVALAIVYLIALAVCQCRRKNYGQLDIFPARDTYHPMSEYPTYHTHGRYVPPSSTDRSPYEKVSAGNGGSSL
SYTNPAVAAASANL
PSMGFR
(SEQ ID NO: 2)
GTINVHDVETQFNQYKTEAASRYNLTISDVSVSDVPFPFSAQSGA
Human NME1
(DNA)
(SEQ ID NO: 3)
atggccaactgtgagcgtaccttcattgcgatcaaaccagatggggtccagcggggtcttgtgggagagatta
tcaagcgttttgagcagaaaggattccgccttgttggtctgaaattcatgcaagcttccgaagatcttctcaa
ggaacactacgttgacctgaaggaccgtccattctttgccggcctggtgaaatacatgcactcagggccggta
gttgccatggtctgggaggggctgaatgtggtgaagacgggccgagtcatgctcggggagaccaaccctgcag
actccaagcctgggaccatccgtggagacttctgcatacaagttggcaggaacattatacatggcagtgattc
tgtggagagtgcagagaaggagatcggcttgtggtttcaccctgaggaactggtagattacacgagctgtgct
cagaactggatctatgaatga
(amino acids)
(SEQ ID NO: 4)
MANCERTFIAIKPDGVQRGLVGEIIKRFEQKGFRLVGLKFMQASEDLLKEHYVDLKDRPFFAGLVKYMHSGPV
VAMVWEGLNVVKTGRVMLGETNPADSKPGTIRGDFCIQVGRNIIHGSDSVESAEKEIGLWFHPEELVDYTSCA
QNWIYE-
Human NME7
(DNA)
(SEQ ID NO: 5)
atgaatcatagtgaaagattcgttttcattgcagagtggtatgatccaaatgcttcacttcttcgacgttatg
agcttttattttacccaggggatggatctgttgaaatgcatgatgtaaagaatcatcgcacctttttaaagcg
gaccaaatatgataacctgcacttggaagatttatttataggcaacaaagtgaatgtcttttctcgacaactg
gtattaattgactatggggatcaatatacagctcgccagctgggcagtaggaaagaaaaaacgctagccctaa
ttaaaccagatgcaatatcaaaggctggagaaataattgaaataataaacaaagctggatttactataaccaa
actcaaaatgatgatgctttcaaggaaagaagcattggattttcatgtagatcaccagtcaagaccctttttc
aatgagctgatccagtttattacaactggtcctattattgccatggagattttaagagatgatgctatatgtg
aatggaaaagactgctgggacctgcaaactctggagtggcacgcacagatgcttctgaaagcattagagccct
ctttggaacagatggcataagaaatgcagcgcatggccctgattcttttgcttctgcggccagagaaatggag
ttgttttttccttcaagtggaggttgtgggccggcaaacactgctaaatttactaattgtacctgttgcattg
ttaaaccccatgctgtcagtgaaggactgttgggaaagatcctgatggctatccgagatgcaggttttgaaat
ctcagctatgcagatgttcaatatggatcgggttaatgttgaggaattctatgaagtttataaaggagtagtg
accgaatatcatgacatggtgacagaaatgtattctggcccttgtgtagcaatggagattcaacagaataatg
ctacaaagacatttcgagaattttgtggacctgctgatcctgaaattgcccggcatttacgccctggaactct
cagagcaatctttggtaaaactaagatccagaatgctgttcactgtactgatctgccagaggatggcctatta
gaggttcaatacttcttcaagatcttggataattag
(amino acids)
(SEQ ID NO: 6)
MNHSERFVFIAEWYDPNASLLRRYELLFYPGDGSVEMHDVKNHRTFLKRTKYDNLHLEDLFIGNKVNVFSRQL
VLIDYGDQYTARQLGSRKEKTLALIKPDAISKAGEIIEIINKAGFTITKLKMMMLSRKEALDFHVDHQSRPFF
NELIQFITTGPIIAMEILRDDAICEWKRLLGPANSGVARTDASESIRALFGTDGIRNAAHGPDSFASAAREME
LFFPSSGGCGPANTAKFTNCTCCIVKPHAVSEGLLGKILMAIRDAGFEISAMQMFNMDRVNVEEFYEVYKGVV
TEYHDMVTEMYSGPCVAMEIQQNNATKTFREFCGPADPEIARHLRPGTLRAIFGKTKIQNAVHCTDLPEDGLL
EVQYFFKILDN-
NME7 peptides
NME7A peptide 1 (A domain):
(SEQ ID NO: 7)
MLSRKEALDFHVDHQS
NME7A peptide 2 (A domain):
(SEQ ID NO: 8)
SGVARTDASES
NME7B peptide 1 (B domain):
(SEQ ID NO: 9)
DAGFEISAMQMFNMDRVNVE
NME7B peptide 2 (B domain):
(SEQ ID NO: 10)
EVYKGVVTEYHDMVTE
NME7B peptide 3 (B domain):
(SEQ ID NO: 11)
AIFGKTKIQNAVHCTDLPEDGLLEVQYFF
Mouse E6 Heavy chain variable region sequence:
(DNA)
(SEQ ID NO: 12)
gaggtgaaggtggtggagtctgggggagacttagtgaagcctggagggtccctgaaactctcctgtgtagtct
ctggattcactttcagtagatatggcatgtcttgggttcgccagactccaggcaagaggctggagtgggtcgc
aaccattagtggtggcggtacttacatctactatccagacagtgtgaaggggcgattcaccatctccagagac
aatgccaagaacaccctgtacctgcaaatgagcagtctgaagtctgaggacacagccatgtatcactgtacaa
gggataactacggtaggaactacgactacggtatggactactggggtcaaggaacctcagtcaccgtctcctc
a
(amino acids)
(SEQ ID NO: 13)
EVKVVESGGDLVKPGGSLKLSCVVSGFTFSRYGMSWVRQTPGKRLEWVATISGGGTYIYYPDSVKGRFTISRD
NAKNTLYLQMSSLKSEDTAMYHCTRDNYGRNYDYGMDYWGQGTSVTVSS
Mouse E6 heavy chain variable framework region 1 (FWR1) sequence:
(DNA)
(SEQ ID NO: 14)
gaggtgaaggtggtggagtctgggggagacttagtgaagcctggagggtccctgaaactctcctgtgtagtct
ct
(amino acids)
(SEQ ID NO: 15)
EVKVVESGGDLVKPGGSLKLSCVVSGFTFS
Mouse E6 heavy chain variable complementarity determining regions 1
(CDR1) sequence:
(DNA)
(SEQ ID NO: 16)
ggattcactttcagtagatatggcatgtct
(amino acids)
(SEQ ID NO: 17)
RYGMS
Mouse E6 heavy chain variable framework region 2 (FWR2) sequence:
(DNA)
(SEQ ID NO: 18)
tgggttcgccagactccaggcaagaggctggagtgggtcgca
(amino acids)
(SEQ ID NO: 19)
WVRQTPGKRLEWVA
Mouse E6 heavy chain variable complementarity determining regions 2
(CDR2) sequence:
(DNA)
(SEQ ID NO: 20)
accattagtggtggcggtacttacatctactatccagacagtgtgaagggg
(amino acids)
(SEQ ID NO: 21)
TISGGGTYIYYPDSVKG
Mouse E6 heavy chain variable framework region 3 (FWR3) acid sequence:
(DNA)
(SEQ ID NO: 22)
cgattcaccatctccagagacaatgccaagaacaccctgtacctgcaaatgagcagtctgaagtctgaggaca
cagccatgtatcactgtacaagg
(amino acids)
(SEQ ID NO: 23)
RFTISRDNAKNTLYLQMSSLKSEDTAMYHCTR
Mouse E6 heavy chain variable complementarity determining regions 3
(CDR3) sequence:
(DNA)
(SEQ ID NO: 24)
gataactacggtaggaactacgactacggtatggactac
(amino acids)
(SEQ ID NO: 25)
DNYGRNYDYGMDY
IGHV3-21*03 heavy chain variable region sequence:
(DNA)
(SEQ ID NO: 26)
gaggtgcagctggtggagtctgggggaggcctggtcaagcctggggggtccctgagactctcctgtgcagcct
ctggattcaccttcagtagctatagcatgaactgggtccgccaggctccagggaaggggctggagtgggtctc
atccattagtagtagtagtagttacatatactacgcagactcagtgaagggccgattcaccatctccagagac
aacgccaagaactcactgtatctgcaaatgaacagcctgagagccgaggacacggctgtgtattactgtgcga
ga
(amino acids)
(SEQ ID NO: 27)
EVQLVESGGGLVKPGGSLRLSCAASGFTFSSYSMNWVRQAPGKGLEWVSSISSSSSYIYYADSVKGRFTISRD
NAKNSLYLQMNSLRAEDTAVYYCAR
IGHV3-21*01 heavy chain variable framework region 1 (FWR1) sequence:
(DNA)
(SEQ ID NO: 28)
gaggtgcagctggtggagtctgggggaggcctggtcaagcctggggggtccctgagactctcctgtgcagcct
ctggattcaccttcagt
(amino acids)
(SEQ ID NO: 29)
EVQLVESGGGLVKPGGSLRLSCAASGFTFS
IGHV3-21*01 heavy chain variable complementarity determining regions 1
(CDR1) sequence:
(DNA)
(SEQ ID NO: 30)
agctatagcatgaac
(amino acids)
(SEQ ID NO: 31)
SYSMN
IGHV3-21*01 heavy chain variable framework region 2 (FWR2) sequence:
(DNA)
(SEQ ID NO: 32)
tgggtccgccaggctccagggaaggggctggagtgggtctca
(amino acids)
(SEQ ID NO: 33)
WVRQAPGKGLEWVS
IGHV3-21*01 heavy chain variable complementarity determining regions 2
(CDR2) sequence:
(DNA)
(SEQ ID NO: 34)
tccattagtagtagtagtagttacatatactacgcagactcagtgaagggc
(amino acids)
(SEQ ID NO: 35)
SISSSSSYIYYADSVKG
IGHV3-21*01 heavy chain variable framework region 3 (FWR3) sequence:
(DNA)
(SEQ ID NO: 36)
cgattcaccatctccagagacaacgccaagaactcactgtatctgcaaatgaacagcctgagagccgaggaca
cggctgtgtattactgtgcgaga
(amino acids)
(SEQ ID NO: 37)
RFTISRDNAKNSLYLQMNSLRAEDTAVYYCAR
Humanized E6 heavy chain variable region sequence:
(DNA)
(SEQ ID NO: 38)
gaggtgcagctggtggagtctgggggaggcctggtcaagcctggggggtccctgagactctcctgtgcagcct
ctggattcaccttcagtaggtatggcatgagctgggtccgccaggctccagggaagaggctggagtgggtctc
aaccattagtggcggaggcacctacatatactacccagactcagtgaagggccgattcaccatctccagagac
aacgccaagaacaccctgtatctgcaaatgaacagcctgagagccgaggacacggctgtgtattactgtacca
gagataactatggccgcaactatgattatggcatggattattggggccagggcaccctggtgaccgtgagcag
c
(amino acids)
(SEQ ID NO: 39)
EVQLVESGGGLVKPGGSLRLSCAASGFTFSRYGMSWVRQAPGKRLEWVSTISGGGTYIYYPDSVKGRFTISRD
NAKNTLYLQMNSLRAEDTAVYYCTRDNYGRNYDYGMDYWGQGTLVTVSS
Humanized E6 heavy chain variable framework region 1 (FWR1) acid
sequence:
(DNA)
(SEQ ID NO: 40)
gaggtgcagctggtggagtctgggggaggcctggtcaagcctggggggtccctgagactctcctgtgcagcct
ctggattcaccttcagt
(amino acids)
(SEQ ID NO: 41)
EVQLVESGGGLVKPGGSLRLSCAASGFTFS
Humanized E6 heavy chain variable complementarity determining regions 1
(CDR1) sequence:
(DNA)
(SEQ ID NO: 42)
aggtatggcatgagc
(amino acids)
(SEQ ID NO: 43)
RYGMS
Humanized E6 heavy chain variable framework region 2 (FWR2) acid
sequence:
(DNA)
(SEQ ID NO: 44)
tgggtccgccaggctccagggaagaggctggagtgggtctca
(amino acids)
(SEQ ID NO: 45)
WVRQAPGKRLEWVS
Humanized E6 heavy chain variable complementarity determining regions 2
(CDR2) sequence:
(DNA)
(SEQ ID NO: 46)
accattagtggcggaggcacctacatatactacccagactcagtgaagggc
(amino acids)
(SEQ ID NO: 47)
TISGGGTYIYYPDSVKG
Humanized E6 heavy chain variable framework region 3 (FWR3) acid
sequence:
(DNA)
(SEQ ID NO: 48)
cgattcaccatctccagagacaacgccaagaacaccctgtatctgcaaatgaacagcctgagagccgaggaca
cggctgtgtattactgtaccaga
(amino acids)
(SEQ ID NO: 49)
RFTISRDNAKNTLYLQMNSLRAEDTAVYYCTR
Humanized E6 heavy chain variable complementarity determining regions 3
(CDR3) sequence:
(DNA)
(SEQ ID NO: 50)
gataactatggccgcaactatgattatggcatggattat
(amino acids)
(SEQ ID NO: 51)
DNYGRNYDYGMDY
Humanized E6 IgG2 heavy chain synthesized by Genescript:
(DNA)
(SEQ ID NO: 52)
gaattctaagcttgggccaccatggaactggggctccgctgggttttccttgttgctattttagaaggtgtcc
agtgtgaggtgcagctggtggagtctgggggaggcctggtcaagcctggggggtccctgagactctcctgtgc
agcctctggattcaccttcagtaggtatggcatgagctgggtccgccaggctccagggaagaggctggagtgg
gtctcaaccattagtggcggaggcacctacatatactacccagactcagtgaagggccgattcaccatctcca
gagacaacgccaagaacaccctgtatctgcaaatgaacagcctgagagccgaggacacggctgtgtattactg
taccagagataactatggccgcaactatgattatggcatggattattggggccagggcaccctggtgaccgtg
agcagcgcctccaccaagggcccatcggtcttccccctggcgccctgctccaggagcacctccgagagcacag
ccgccctgggctgcctggtcaaggactacttccccgaaccggtgacggtgtcgtggaactcaggcgctctgac
cagcggcgtgcacaccttcccagctgtcctacagtcctcaggactctactccctcagcagcgtggtgaccgtg
ccctccagcaacttcggcacccagacctacacctgcaacgtagatcacaagcccagcaacaccaaggtggaca
agacagttgagcgcaaatgttgtgtcgagtgcccaccgtgcccagcaccacctgtggcaggaccgtcagtctt
cctcttccccccaaaacccaaggacaccctcatgatctcccggacccctgaggtcacgtgcgtggtggtggac
gtgagccacgaagaccccgaggtccagttcaactggtacgtggacggcgtggaggtgcataatgccaagacaa
agccacgggaggagcagttcaacagcacgttccgtgtggtcagcgtcctcaccgttgtgcaccaggactggct
gaacggcaaggagtacaagtgcaaggtctccaacaaaggcctcccagcccccatcgagaaaaccatctccaaa
accaaagggcagccccgagaaccacaggtgtacaccctgcccccatcccgggaggagatgaccaagaaccagg
tcagcctgacctgcctggtcaaaggcttctaccccagcgacatcgccgtggagtgggagagcaatgggcagcc
ggagaacaactacaagaccacacctcccatgctggactccgacggctccttcttcctctacagcaagctcacc
gtggacaagagcaggtggcagcaggggaacgtcttctcatgctccgtgatgcatgaggctctgcacaaccact
acacgcagaagagcctctccctgtctccgggtaaatagtaagtttaaactctaga
(amino acids)
(SEQ ID NO: 53)
EF*AWATMELGLRWVFLVAILEGVQCEVQLVESGGGLVKPGGSLRLSCAASGFTFSRYGMSWVRQAPGKRLEW
VSTISGGGTYIYYPDSVKGRFTISRDNAKNTLYLQMNSLRAEDTAVYYCTRDNYGRNYDYGMDYWGQGTLVTV
SSASTKGPSVFPLAPCSRSTSESTAALGCLVKDYFPEPVTVSWNSGALTSGVHTFPAVLQSSGLYSLSSVVTV
PSSNFGTQTYTCNVDHKPSNTKVDKTVERKCCVECPPCPAPPVAGPSVFLFPPKPKDTLMISRTPEVTCVVVD
VSHEDPEVQFNWYVDGVEVHNAKTKPREEQFNSTFRVVSVLTVVHQDWLNGKEYKCKVSNKGLPAPIEKTISK
TKGQPREPQVYTLPPSREEMTKNQVSLTCLVKGFYPSDIAVEWESNGQPENNYKTTPPMLDSDGSFFLYSKLT
VDKSRWQQGNVFSCSVMHEALHNHYTQKSLSLSPGK**V*TLX
Human IgG2 heavy chain constant region sequence:
(DNA)
(SEQ ID NO: 54)
gcctccaccaagggcccatcggtcttccccctggcgccctgctccaggagcacctccgagagcacagccgccc
tgggctgcctggtcaaggactacttccccgaaccggtgacggtgtcgtggaactcaggcgctctgaccagcgg
cgtgcacaccttcccagctgtcctacagtcctcaggactctactccctcagcagcgtggtgaccgtgccctcc
agcaacttcggcacccagacctacacctgcaacgtagatcacaagcccagcaacaccaaggtggacaagacag
ttgagcgcaaatgttgtgtcgagtgcccaccgtgcccagcaccacctgtggcaggaccgtcagtcttcctctt
ccccccaaaacccaaggacaccctcatgatctcccggacccctgaggtcacgtgcgtggtggtggacgtgagc
cacgaagaccccgaggtccagttcaactggtacgtggacggcgtggaggtgcataatgccaagacaaagccac
gggaggagcagttcaacagcacgttccgtgtggtcagcgtcctcaccgttgtgcaccaggactggctgaacgg
caaggagtacaagtgcaaggtctccaacaaaggcctcccagcccccatcgagaaaaccatctccaaaaccaaa
gggcagccccgagaaccacaggtgtacaccctgcccccatcccgggaggagatgaccaagaaccaggtcagcc
tgacctgcctggtcaaaggcttctaccccagcgacatcgccgtggagtgggagagcaatgggcagccggagaa
caactacaagaccacacctcccatgctggactccgacggctccttcttcctctacagcaagctcaccgtggac
aagagcaggtggcagcaggggaacgtcttctcatgctccgtgatgcatgaggctctgcacaaccactacacgc
agaagagcctctccctgtctccgggtaaatag
(amino acids)
(SEQ ID NO: 55)
ASTKGPSVFPLAPCSRSTSESTAALGCLVKDYFPEPVTVSWNSGALTSGVHTFPAVLQSSGLYSLSSVVTVPS
SNFGTQTYTCNVDHKPSNTKVDKTVERKCCVECPPCPAPPVAGPSVFLFPPKPKDTLMISRTPEVTCVVVDVS
HEDPEVQFNWYVDGVEVHNAKTKPREEQFNSTFRVVSVLTVVHQDWLNGKEYKCKVSNKGLPAPIEKTISKTK
GQPREPQVYTLPPSREEMTKNQVSLTCLVKGFYPSDIAVEWESNGQPENNYKTTPPMLDSDGSFFLYSKLTVD
KSRWQQGNVFSCSVMHEALHNHYTQKSLSLSPGK
Humanized E6 IgG1 heavy chain sequence:
(DNA)
(SEQ ID NO: 56)
gaggtgcagctggtggagtctgggggaggcctggtcaagcctggggggtccctgagactctcctgtgcagcct
ctggattcaccttcagtaggtatggcatgagctgggtccgccaggctccagggaagaggctggagtgggtctc
aaccattagtggcggaggcacctacatatactacccagactcagtgaagggccgattcaccatctccagagac
aacgccaagaacccactgtatctgcaaatgaacagcctgagagccgaggacacggctgtgtattactgtccca
gagataactatggccgcaactatgattatggcatggattattggggccagggcaccctggtgaccgtgagcag
cgctagcaccaagggcccatcggtcttccccctggcaccctcctccaagagcacctctgggggcacagcggcc
ctgggctgcctggtcaaggactacttccccgaaccggtgacggtgtcgtggaactcaggcgccctgaccagcg
gcgtgcacaccttcccggctgtcctacagtcctcaggactctactccctcagcagcgtggtgacagtgccctc
cagcagcttgggcacccagacctacatctgcaacgtgaatcacaagcccagcaacaccaaggtggacaagaaa
gttgagcccaaatcttgtgacaaaactcacacatgcccaccgtgcccagcacctgaactcctggggggaccgt
cagtcttcctcttccccccaaaacccaaggacaccctcatgatctcccggacccctgaggtcacatgcgtggt
ggtggacgtgagccacgaagaccctgaggtcaagttcaactggtacgtggacggcgtggaggtgcataatgcc
aagacaaagccgcgggaggagcagtacaacagcacgtaccgtgtggtcagcgtcctcaccgtcctgcaccagg
actggctgaatggcaaggagtacaagtgcaaggtctccaacaaagccctcccagcccccatcgagaaaaccat
ctccaaagccaaagggcagccccgagaaccacaggtgtacaccctgcccccatcccgggaggagatgaccaag
aaccaggtcagcctgacctgcctggtcaaaggcttctatcccagcgacatcgccgtggagtgggagagcaatg
ggcagccggagaacaactacaagaccacgcctcccgtgctggactccgacggctccttcttcctctacagcaa
gctcaccgtggacaagagcaggtggcagcaggggaacgtcttctcatgctccgtgatgcatgaggctctgcac
aaccactacacgcagaagagcctctccctgtctccgggtaaatgataa
(amino acids)
(SEQ ID NO: 57)
EVQLVESGGGLVKPGGSLRLSCAASGFTFSRYGMSWVRQAPGKRLEWVSTISGGGTYIYYPDSVKGRFTISRD
NAKNPLYLQMNSLRAEDTAVYYCPRDNYGRNYDYGMDYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHEALH
NHYTQKSLSLSPGK**
Human IgG1 heavy chain constant region sequence:
(DNA)
(SEQ ID NO: 58)
gctagcaccaagggcccatcggtcttccccctggcaccctcctccaagagcacctctgggggcacagcggccc
tgggctgcctggtcaaggactacttccccgaaccggtgacggtgtcgtggaactcaggcgccctgaccagcgg
cgtgcacaccttcccggctgtcctacagtcctcaggactctactccctcagcagcgtggtgacagtgccctcc
agcagcttgggcacccagacctacatctgcaacgtgaatcacaagcccagcaacaccaaggtggacaagaaag
ttgagcccaaatcttgtgacaaaactcacacatgcccaccgtgcccagcacctgaactcctggggggaccgtc
agtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgcca
agacaaagccgcgggaggagcagtacaacagcacgtaccgtgtggtcagcgtcctcaccgtcctgcaccagga
ctggctgaatggcaaggagtacaagtgcaaggtctccaacaaagccctcccagcccccatcgagaaaaccatc
tccaaagccaaagggcagccccgagaaccacaggtgtacaccctgcccccatcccgggaggagatgaccaaga
accaggtcagcctgacctgcctggtcaaaggcttctatcccagcgacatcgccgtggagtgggagagcaatgg
gcagccggagaacaactacaagaccacgcctcccgtgctggactccgacggctccttcttcctctacagcaag
ctcaccgtggacaagagcaggtggcagcaggggaacgtcttctcatgctccgtgatgcatgaggctctgcaca
accactacacgcagaagagcctctccctgtctccgggtaaatgataa
(amino acids)
(SEQ ID NO: 59)
ASTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK**
Human IgG1 heavy chain constant region gBLOCK#1 sequence:
(DNA)
(SEQ ID NO: 60)
atggcatggattattggggccagggcaccctggtgaccgtgagcagcgctagcaccaagggcccatcggtctt
ccccctggcaccctcctccaagagcacctctgggggcacagcggccctgggctgcctggtcaaggactacttc
cccgaaccggtgacggtgtcgtggaactcaggcgccctgaccagcggcgtgcacaccttcccggctgtcctac
agtcctcaggactctactccctcagcagcgtggtgacagtgccctccagcagcttgggcacccagacctacat
ctgcaacgtgaatcacaagcccagcaacaccaaggtggacaagaaagttgagcccaaatcttgtgacaaaact
cacacatgcccaccgtgcccagcacctgaactcctggggggaccgtcagtcttcctcttccccccaaaaccca
aggacaccctcatgatctcccggacccctgaggtcacatgcgtggtggtggacgtgagccacgaagaccctga
ggtcaagttcaactggtacgtggacggcgtggaggtgcataatgccaag
Human IgG1 heavy chain constant region gBLOCK#2 sequence:
(DNA)
(SEQ ID NO: 61)
tacgtggacggcgtggaggtgcataatgccaagacaaagccgcgggaggagcagtacaacagcacgtaccgtg
tggtcagcgtcctcaccgtcctgcaccaggactggctgaatggcaaggagtacaagtgcaaggtctccaacaa
agccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatccca
gcgacatcgccgtggagtgggagagcaatgggcagccggagaacaactacaagaccacgcctcccgtgctgga
ctccgacggctccttcttcctctacagcaagctcaccgtggacaagagcaggtggcagcaggggaacgtcttc
tcatgctccgtgatgcatgaggctctgcacaaccactacacgcagaagagcctctccctgtctccgggtaaat
gataagtttaaacccgctgatcagcctcgactgtgccttctagttg
E6 heavy chain variable region overlapping sequence:
(DNA)
(SEQ ID NO: 62)
atggcatggattattggggccagggcaccct
IgG1 heavy chain constant region overlapping region sequence:
(DNA)
(SEQ ID NO: 63)
tacgtggacggcgtggaggtgcataatgccaag
pCDNA3.1 V5 and pSECTag2 overlapping sequence:
(DNA)
(SEQ ID NO: 64)
ccgctgatcagcctcgactgtgccttctagttg
Mouse E6 Light Chain variable region sequence:
(DNA)
(SEQ ID NO: 65)
caaattgttctcacccagtctccagcaatcatgtctgcatctccaggggaggaggtcaccctaacct
gcagtgccacctcaagtgtaagttacatacactggttccagcagaggccaggcacttctcccaaact
ctggatttatagcacatccaacctggcttctggagtccctgttcgcttcagtggcagtggatatggg
acctcttactctctcacaatcagccgaatggaggctgaagatgctgccacttattactgccagcaaa
ggagtagttccccattcacgttcggctcggggacaaagttggaaataaaa
(amino acids)
(SEQ ID NO: 66)
QIVLTQSPAIMSASPGEEVTLTCSATSSVSYIHWFQQRPGTSPKLWIYSTSNLASGVPVRFSGSGYGTSYSLT
ISRMEAEDAATYYCQQRSSSPFTFGSGTKLEIK
Mouse E6 light chain variable framework region 1 (FWR1) sequence:
(DNA)
(SEQ ID NO: 67)
caaattgttctcacccagtctccagcaatcatgtctgcatctccaggggaggaggtcaccctaacctgc
(amino acids)
(SEQ ID NO: 68)
QIVLTQSPAIMSASPGEEVTLTC
Mouse E6 light chain variable complementarity determining regions 1
(CDR1) sequence:
(DNA)
(SEQ ID NO: 69)
AGTGCCACCTCAAGTGTAAGTTACATACAC
(amino acids)
(SEQ ID NO: 70)
SATSSVSYIH
Mouse E6 light chain variable framework region 2 (FWR2) sequence:
(DNA)
(SEQ ID NO: 71)
tggttccagcagaggccaggcacttctcccaaactctggatttat
(amino acids)
(SEQ ID NO: 72)
WFQQRPGTSPKLWIY
Mouse E6 light chain variable complementarity determining regions 2
(CDR2) sequence:
(DNA)
(SEQ ID NO: 73)
agcacatccaacctggcttct
(amino acids)
(SEQ ID NO: 74)
STSNLAS
Mouse E6 light chain variable framework region 3 (FWR3) sequence:
(DNA)
(SEQ ID NO: 75)
ggagtccctgttcgcttcagtggcagtggatatgggacctcttactctctcacaatcagccgaatggaggctg
aagatgctgccacttattactgc
(amino acids)
(SEQ ID NO: 76)
GVPVRFSGSGYGTSYSLTISRMEAEDAATYYC
Mouse E6 light chain variable complementarity determining regions 3
(CDR3) sequence:
(DNA)
(SEQ ID NO: 77)
cagcaaaggagtagttccccattcacg
(amino acids)
(SEQ ID NO: 78)
QQRSSSPFT
IGKV3-11*02 light chain variable region sequence:
(DNA)
(SEQ ID NO: 79)
gaaattgtgttgacacagtctccagccaccctgtctttgtctccaggggaaagagccaccctctcctgcaggg
ccagtcagagtgttagcagctacttagcctggtaccaacagaaacctggccaggctcccaggctcctcatcta
tgatgcatccaacagggccactggcatcccagccaggttcagtggcagtgggtctgggagagacttcactctc
accatcagcagcctagagcctgaagattttgcagtttattactgtcagcagcgtagcaactggcctcc
(amino acids)
(SEQ ID NO: 80)
EIVLTQSPATLSLSPGERATLSCRASQSVSSYLAWYQQKPGQAPRLLIYDASNRATGIPA
RFSGSGSGRDFTLTISSLEPEDFAVYYCQQRSNWPP
IGKV3-11*02 light chain variable framework region 1 (FWR1) acid sequence:
(DNA)
(SEQ ID NO: 81)
gaaattgtgttgacacagtctccagccaccctgtctttgtctccaggggaaagagccaccctctcctgc
(amino acids)
(SEQ ID NO: 82)
EIVLTQSPATLSLSPGERATLSC
IGKV3-11*02 light chain variable complementarity determining regions 1
(CDR1) sequence:
(DNA)
(SEQ ID NO: 83)
agggccagtcagagtgttagcagctacttagcc
(amino acids)
(SEQ ID NO: 84)
RASQSVSSYLA
IGKV3-11*02 light chain variable framework region 2 (FWR2) sequence:
(DNA)
(SEQ ID NO: 85)
tggtaccaacagaaacctggccaggctcccaggctcctcatctat
(amino acids)
(SEQ ID NO: 86)
WYQQKPGQAPRLLIY
IGKV3-11*02 light chain variable complementarity determining regions 2
(CDR2) sequence:
(DNA)
(SEQ ID NO: 87)
gatgcatccaacagggccact
(amino acids)
(SEQ ID NO: 88)
DASNRAT
IGKV3-11*02 light chain variable framework region 3 (FWR3) sequence:
(DNA)
(SEQ ID NO: 89)
ggcatcccagccaggttcagtggcagtgggtctgggagagacttcactctcaccatcagcagcctagagcctg
aagattttgcagtttattactgt
(amino acids)
(SEQ ID NO: 90)
GIPARFSGSGSGTDFTLTISSLEPEDFAVYYC
IGKV3-11*02 light chain variable complementarity determining regions3
(CDR3) sequence:
(DNA)
(SEQ ID NO: 91)
cagcagcgtagcaactggcctcc
(amino acids)
(SEQ ID NO: 92)
QQRSNWPP
Humanized E6 light chain variable region sequence:
(DNA)
(SEQ ID NO: 93)
gaaattgtgttgacacagtctccagccaccctgtctttgtctccaggggaaagagccaccctcacctgcagcg
ccaccagcagtgttagctacatccactggtaccaacagaggcctggccagagccccaggctcctcatctatag
cacctccaacctggccagcggcatcccagccaggttcagtggcagtgggtctgggagcgactacactctcacc
atcagcagcctagagcctgaagattttgcagtttattactgtcagcagcgtagcagctcccctttcacctttg
gcagcggcaccaaagtggaaattaaa
(amino acids)
(SEQ ID NO: 94)
EIVLTQSPATLSLSPGERATLTCSATSSVSYIHWYQQRPGQSPRLLIYSTSNLASGIPARFSGSGSGSDYTLT
ISSLEPEDFAVYYCQQRSSSPFTFGSGTKVEIK
Humanized E6 light chain variable framework region 1 (FWR1) acid
sequence:
(DNA)
(SEQ ID NO: 95)
gaaattgtgttgacacagtctccagccaccctgtctttgtctccaggggaaagagccaccctcacctgc
(amino acids)
(SEQ ID NO: 96)
EIVLTQSPATLSLSPGERATLTC
Humanized E6 light chain variable complementarity determining regions 1
(CDR1) sequence:
(DNA)
(SEQ ID NO: 97)
agcgccaccagcagtgttagctacatccac
(amino acids)
(SEQ ID NO: 98)
SATSSVSYIH
Humanized E6 heavy light variable framework region 2 (FWR2) acid
sequence:
(DNA)
(SEQ ID NO: 99)
tggtaccaacagaggcctggccagagccccaggctcctcatctat
(amino acids)
(SEQ ID NO: 100)
WYQQRPGQSPRLLIY
Humanized E6 light chain variable complementarity determining regions 2
(CDR2) sequence:
(DNA)
(SEQ ID NO: 101)
agcacctccaacctggccagc
(amino acids)
(SEQ ID NO: 102)
STSNLAS
Humanized E6 light chain variable framework region 3 (FWR3) acid
sequence:
(DNA)
(SEQ ID NO: 103)
ggcatcccagccaggttcagtggcagtgggtctgggagcgactacactctcaccatcagcagcctagagcctg
aactattttgcagtttattactgt
(amino acids)
(SEQ ID NO: 104)
GIPARFSGSGSGSDYTLTISSLEPEDFAVYYC
Humanized E6 light chain variable complementarity determining regions 3
(CDR3) sequence:
(DNA)
(SEQ ID NO: 105)
cagcagcgtagcagctcccctttcacc
(amino acids)
(SEQ ID NO: 106)
QQRSSSPFT
Humanized E6 Kappa light chain synthesized by Genescript:
(DNA)
(SEQ ID NO: 107)
gaattctaagcttgggccaccatggaagccccagcgcagcttctcttcctcctgctactctggctcccagata
ccactggagaaattgtgttgacacagtctccagccaccctgtctttgtctccaggggaaagagccaccctcac
ctgcagcgccaccagcagtgttagctacatccactggtaccaacagaggcctggccagagccccaggctcctc
atctatagcacctccaacctggccagcggcatcccagccaggttcagtggcagtgggtctgggagcgactaca
ctctcaccatcagcagcctagagcctgaagattttgcagtttattactgtcagcagcgtagcagctccccttt
cacctttggcagcggcaccaaagtggaaattaaaaggacggtggctgcaccatctgtcttcatcttcccgcca
tctgatgagcagttgaaatctggaactgcctctgttgtgtgcctgctgaataacttctatcccagagaggcca
aagtacagtggaaggtggataacgccctccaatcgggtaactcccaggagagtgtcacagagcaggacagcaa
ggacagcacctacagcctcagcagcaccctgacgctgagcaaagcagactacgagaaacacaaagtctacgcc
tgcgaagtcacccatcagggcctgagctcgcccgtcacaaagagcttcaacaggggagagtgttagtaagttt
aaactctaga
(amino acids)
(SEQ ID NO: 108)
EF*AWATMEAPAQLLFLLLLWLPDTTGEIVLTQSPATLSLSPGERATLTCSATSSVSYIHWYQQRPGQSPRLL
IYSTSNLASGIPARFSGSGSGSDYTLTISSLEPEDFAVYYCQQRSSSPFTFGSGTKVEIKRTVAAPSVFIFPP
SDEQLKSGTASVVCLLNNFYPREAKVQWKVDNALQSGNSQESVTEQDSKDSTYSLSSTLTLSKADYEKHKVYA
CEVTHQGLSSPVTKSFNRGEC**V*TLX
Human Kappa light chain constant region sequence:
(DNA)
(SEQ ID NO: 109)
aggacggtggctgcaccatctgtcttcatcttcccgccatctgatgagcagttgaaatctggaactgcctctg
ttgtgtgcctgctgaataacttctatcccagagaggccaaagtacagtggaaggtggataacgccctccaatc
gggtaactcccaggagagtgtcacagagcaggacagcaaggacagcacctacagcctcagcagcaccctgacg
ctgagcaaagcagactacgagaaacacaaagtctacgcctgcgaagtcacccatcagggcctgagctcgcccg
tcacaaagagcttcaacaggggagagtgttag
(amino acids)
(SEQ ID NO: 110)
RTVAAPSVFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Humanized E6 lambda light chain sequence:
(DNA)
(SEQ ID NO: 111)
gaaattgtgttgacacagtctccagccaccctgtctttgtctccaggggaaagagccaccctcacctgcagcg
ccaccagcagtgttagctacatccactggtaccaacagaggcctggccagagccccaggctcctcatctatag
cacctccaacctggccagcggcatcccagccaggttcagtggcagtgggtctgggagcgactacactctcacc
atcagcagcctagagcctgaagattttgcagtttattactgtcagcagcgtagcagctcccctttcacctttg
gcagcggcaccaaagtggaaattaaaggtcagcccaaggctgccccctcggtcactctgttcccgccctcctc
tgaggagcttcaagccaacaaggccacactggtgtgtctcataagtgacttctacccgggagccgtgacagtg
gcctggaaggcagatagcagccccgtcaaggcgggagtggagaccaccacaccctccaaacaaagcaacaaca
agtacgcggccagcagctatctgagcctgacgcctgagcagtggaagtcccacagaagctacagctgccaggt
cacgcatgaagggagcaccgtggagaagacagtggcccctacagaatgttcatagtaa
(amino acids)
(SEQ ID NO: 112)
EIVLTQSPATLSLSPGERATLTCSATSSVSYIHWYQQRPGQSPRLLIYSTSNLASGIPARFSGSGSGSDYTLT
ISSLEPEDFAVYYCQQRSSSPFTFGSGTKVEIKGQPKAAPSVTLFPPSSEELQANKATLVCLISDFYPGAVTV
AWKADSSPVKAGVETTTPSKQSNNKYAASSYLSLTPEQWKSHRSYSCQVTHEGSTVEKTVAPTECS**
Humanized lambda light chain constant region sequence:
(DNA)
(SEQ ID NO: 113)
ggtcagcccaaggctgccccctcggtcactctgttcccgccctcctctgaggagcttcaagccaacaaggcca
cactggtgtgtctcataagtgacttctacccgggagccgtgacagtggcctggaaggcagatagcagccccgt
caaggcgggagtggagaccaccacaccctccaaacaaagcaacaacaagtacgcggccagcagctatctgagc
ctgacgcctgagcagtggaagtcccacagaagctacagctgccaggtcacgcatgaagggagcaccgtggaga
agacagtggcccctacagaatgttcatagtaa
(amino acids)
(SEQ ID NO: 114)
GQPKAAPSVTLFPPSSEELQANKATLVCLISDFYPGAVTVAWKADSSPVKAGVETTTPSKQSNNKYAASSYLS
LTPEQWKSHRSYSCQVTHEGSTVEKTVAPTECS**
Human lambda light chain constant region gBLOCK#3 sequence:
(DNA)
(SEQ ID NO: 115)
agcgccaccagcagtgttagctacatccactggtaccaacagaggcctggccagagccccaggctcctcatct
atagcacctccaacctggccagcggcatcccagccaggttcagtggcagtgggtctgggagcgactacactct
caccatcagcagcctagagcctgaagattttgcagtttattactgtcagcagcgtagcagctcccctttcacc
tttggcagcggcaccaaagtggaaattaaaggtcagcccaaggctgccccctcggtcactctgttcccgccct
cctctgaggagcttcaagccaacaaggccacactggtgtgtctcataagtgacttctacccgggagccgtgac
agtggcctggaaggcagatagcagccccgtcaaggcgggagtggagaccaccacaccctccaaacaaagcaac
aacaagtacgcggccagcagctatctgagcctgacgcctgagcagtggaagtcccacagaagctacagctgcc
aggtcacgcatgaagggagcaccgtggagaagacagtggcccctacagaatgttcatagtaagtttaaacccg
ctgatcagcctcgactgtgccttctagttg
E6 light chain variable region overlapping sequence:
(DNA)
(SEQ ID NO: 116)
agcgccaccagcagtgttagctacatccact
pCDNA3.1 V5 and pSECTag2 overlapping sequence:
(DNA)
(SEQ ID NO: 117)
ccgctgatcagcctcgactgtgccttctagttg
Mouse C2 heavy chain variable region sequence:
(DNA)
(SEQ ID NO: 118)
gaggtccagctggaggagtcagggggaggcttagtgaagcctggagggtccctgaaactctcctgtgcagcct
ctggattcactttcagtggctatgccatgtcttgggttcgccagactccggagaagaggctggagtgggtcgc
aaccattagtagtggtggtacttatatctactatccagacagtgtgaaggggcgattcaccatctccagagac
aatgccaagaacaccctgtacctgcaaatgagcagtctgaggtctgaggacacggccatgtattactgtgcaa
gacttgggggggataattactacgaatacttcgatgtctggggcgcagggaccacggtcaccgtctcctccgc
caaaacgacacccccatctgtctat
(amino acids)
(SEQ ID NO: 119)
EVQLEESGGGLVKPGGSLKLSCAASGFTFSGYAMSWVRQTPEKRLEWVATISSGGTYIYYPDSVKGRFTISRD
NAKNTLYLQMSSLRSEDTAMYYCARLGGDNYYEYFDVWGAGTTVTVSSAKTTPPSVY
Mouse C2 heavy chain variable framework region 1 (FWR1) sequence:
(DNA)
(SEQ ID NO: 120)
gaggtccagctggaggagtcagggggaggcttagtgaagcctggagggtccctgaaactctcctgtgcagcct
ctggattcactttcagt
(amino acids)
(SEQ ID NO: 121)
EVQLEESGGGLVKPGGSLKLSCAASGFTFS
Mouse C2 heavy chain variable complementarity determining regions 1
(CDR1) sequence:
(DNA)
(SEQ ID NO: 122)
ggctatgccatgtct
(amino acids)
(SEQ ID NO: 123)
GYAMS
Mouse C2 heavy chain variable framework region 2 (FWR2) sequence:
(DNA)
(SEQ ID NO: 124)
tgggttcgccagactccggagaagaggctggagtgggtcgca
(amino acids)
(SEQ ID NO: 125)
WVRQTPEKRLEWVA
Mouse C2 heavy chain variable complementarity determining regions 2
(CDR2) sequence:
(DNA)
(SEQ ID NO: 126)
accattagtagtggtggtacttatatctactatccagacagtgtgaagggg
(amino acids)
(SEQ ID NO: 127)
TISSGGTYIYYPDSVKG
Mouse C2 heavy chain variable framework region 3 (FWR3) sequence:
(DNA)
(SEQ ID NO: 128)
cgattcaccatctccagagacaatgccaagaacaccctgtacctgcaaatgagcagtctgaggtctgaggaca
cggccatgtattactgtgcaaga
(amino acids)
(SEQ ID NO: 129)
RFTISRDNAKNTLYLQMSSLRSEDTAMYYCAR
Mouse C2 heavy chain variable complementarity determining regions 3
(CDR3) sequence:
(DNA)
(SEQ ID NO: 130)
cttgggggggataattactacgaatacttcgatgtc
(amino acids)
(SEQ ID NO: 131)
LGGDNYYEYFDV
IGHV3-21*04 heavy chain variable region sequence:
(DNA)
(SEQ ID NO: 132)
gaggtgcagctggtggagtctgggggaggcctggtcaagcctggggggtccctgagactctcctgtgcagcct
ctggattcaccttcagtagctatagcatgaactgggtccgccaggctccagggaaggggctggagtgggtctc
atccattagtagtagtagtagttacatatactacgcagactcagtgaagggccgattcaccatctccagagac
aacgccaagaactcactgtatctgcaaatgaacagcctgagagccgaggacacggccgtgtattactgtgcga
ga
(amino acids)
(SEQ ID NO: 133)
EVQLVESGGGLVKPGGSLRLSCAASGFTFSSYSMNWVRQAPGKGLEWVSSISSSSSYIYYADSVKGRFTISRD
NAKNSLYLQMNSLRAEDTAVYYCAR
IGHV3-21*04 heavy chain variable framework region 1 (FWR1) sequence:
(DNA)
(SEQ ID NO: 134)
gaggtgcagctggtggagtctgggggaggcctggtcaagcctggggggtccctgagactctcctgtgcagcct
ctggattcaccttcagt
(amino acids)
(SEQ ID NO: 135)
EVQLVESGGGLVKPGGSLRLSCAASGFTFS
IGHV3-21*04 heavy chain variable complementarity determining regions 1
(CDR1) sequence:
(DNA)
(SEQ ID NO: 136)
agctatagcatgaac
(amino acids)
(SEQ ID NO: 137)
SYSMN
IGHV3-21*04 heavy chain variable framework region 2 (FWR2) sequence:
(DNA)
(SEQ ID NO: 138)
gggtccgccaggctccagggaaggggctggagtgggtctca
(amino acids)
(SEQ ID NO: 139)
WVRQAPGKGLEWVS
IGHV3-21*04 heavy chain variable complementarity determining regions 2
(CDR2) sequence:
(DNA)
(SEQ ID NO: 140)
tccattagtagtagtagtagttacatatactacgcagactcagtgaagggc
(amino acids)
(SEQ ID NO: 141)
SISSSSSYIYYADSVKG
IGHV3-21*04 heavy chain variable framework region 3 (FWR3) sequence:
(DNA)
(SEQ ID NO: 142)
cgattcaccatctccagagacaacgccaagaactcactgtatctgcaaatgaacagcctgagagccgaggaca
cggccgtgtattactgtgcgaga
(amino acids)
(SEQ ID NO: 143)
RFTISRDNAKNSLYLQMNSLRAEDTAVYYCAR
Humanized C2 heavy chain variable region sequence:
(DNA)
(SEQ ID NO: 144)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccccgactcagtgaagggccgattcaccatctccagagac
aacgccaagaactcactgtatctgcaaatgaacagcctgagagccgaggacacggccgtgtattactgtgcga
gacttgggggggataattactacgaatacttcgatgtctggggcaaagggaccacggtcaccgtctcctcc
(amino acids)
(SEQ ID NO: 145)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSS
Humanized C2 heavy chain variable framework region 1 (FWR1) sequence:
(DNA)
(SEQ ID NO: 146)
gaggtgcagctggtggagtctgggggaggcctggtcaagcctggggggtccctgagactctcctgtgcagcct
ctggattcaccttcagt
(amino acids)
(SEQ ID NO: 147)
EVQLVESGGGLVKPGGSLRLSCAASGFTFS
Humanized C2 heavy chain variable complementarity determining regions 1
(CDR1) sequence:
(DNA)
(SEQ ID NO: 148)
ggctatgccatgagc
(amino acids)
(SEQ ID NO: 149)
GYAMS
Humanized C2 heavy chain variable framework region 2 (FWR2) sequence:
(DNA)
(SEQ ID NO: 150)
tgggtccgccaggctccagggaaggggctggagtgggtctcaa
(amino acids)
(SEQ ID NO: 151)
WVRQAPGKGLEWVS
Humanized C2 heavy chain variable complementarity determining regions 2
(CDR2) sequence:
(DNA)
(SEQ ID NO: 152)
accattagtagtggcggaacctacatatactaccccgactcagtgaagggc
(amino acids)
(SEQ ID NO: 153)
TISSGGTYIYYPDSVKG
Humanized C2 heavy chain variable framework region 3 (FWR3) sequence:
(DNA)
(SEQ ID NO: 154)
cgattcaccatctccagagacaacgccaagaactcactgtatctgcaaatgaacagcctgagagccgaggaca
cggccgtgtattactgtgcgaga
(amino acids)
(SEQ ID NO: 155)
RFTISRDNAKNSLYLQMNSLRAEDTAVYYCAR
Humanized C2 heavy chain variable complementarity determining regions 3
(CDR3) sequence:
(DNA)
(SEQ ID NO: 156)
cttgggggggataattactacgaatacttcgatgtc
(amino acids)
(SEQ ID NO: 157)
LGGDNYYEYFDV
Humanized C2 IgG1 heavy chain sequence
(DNA)
(SEQ ID NO: 157)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccccgactcagtgaagggccgattcaccatctccagagac
aacgccaagaactcactgtatctgcaaatgaacagcctgagagccgaggacacggccgtgtattactgtgcga
gacttgggggggataattactacgaatacttcgatgtctggggcaaagggaccacggtcaccgtctcctccgc
tagcaccaagggcccatcggtcttccccctggcaccctcctccaagagcacctctgggggcacagcggccctg
ggctgcctggtcaaggactacttccccgaaccggtgacggtgtcgtggaactcaggcgccctgaccagcggcg
tgcacaccttcccggctgtcctacagtcctcaggactctactccctcagcagcgtggtgacagtgccctccag
cagcttgggcacccagacctacatctgcaacgtgaatcacaagcccagcaacaccaaggtggacaagaaagtt
gagcccaaatcttgtgacaaaactcacacatgcccaccgtgcccagcacctgaactcctggggggaccgtcag
tcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctc
caaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggc
agccggagaacaactacaagaccacgcctcccgtgctggactccgacggctccttcttcctctacagcaagct
caccgtggacaagagcaggtggcagcaggggaacgtcttctcatgctccgtgatgcatgaggctctgcacaac
cactacacgcagaagagcctctccctgtctccgggtaaatgataa
(amino acids)
(SEQ ID NO: 158)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGNVFSCSVMHEALHN
HYTQKSLSLSPGK**
Humanized C2 gBLOCK#4 sequence:
(DNA)
(SEQ ID NO: 160)
actcactatagggagacccaagctggctagttaagcttgggccaccatggagacagacacactcctgctatgg
gtactgctgctctgggttccaggttccactggtgacgaggtgcagctggtggagtctgggggaggcctggtca
agcctggggggtccctgagactctcctgtgcagcctctggattcaccttcagtggctatgccatgagctgggt
ccgccaggctccagggaaggggctggagtgggtctcaaccattagtagtggcggaacctacatatactacccc
gactcagtgaagggccgattcaccatctccagagacaacgccaagaactcactgtatctgcaaatgaacagcc
tgagagccgaggacacggccgtgtattactgtgcgagacttgggggggataattactacgaatacttcgatgt
ctggggcaaagggaccacggtcaccgtctcctccgctagcaccaagggcccatcggtcttccccctggcaccc
tcctccaagagcacctctgggggcacagcggccctgggctgcctggtcaaggactacttccccgaaccggtga
cggtgtcgtggaactcaggcgccctgaccagc
pCDNA3.1 V5 overlapping sequence:
(DNA)
(SEQ ID NO: 161)
actcactatagggagacccaagctggctagtt
Human IgG1 constant region overlapping sequence:
(DNA)
(SEQ ID NO: 162)
gacggtgtcgtggaactcaggcgccctgaccagc
Humanized C2 IgG2 heavy chain sequence
(DNA)
(SEQ ID NO: 163)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccccgactcagtgaagggccgattcaccatctccagagac
aacgccaagaactcactgtatctgcaaatgaacagcctgagagccgaggacacggccgtgtattactgtgcga
gacttgggggggataattactacgaatacttcgatgtctggggcaaagggaccacggtcaccgtctcctccgc
ctccaccaagggcccatcggtcttccccctggcgccctgctccaggagcacctccgagagcacagccgccctg
ggctgcctggtcaaggactacttccccgaaccggtgacggtgtcgtggaactcaggcgctctgaccagcggcg
tgcacaccttcccagctgtcctacagtcctcaggactctactccctcagcagcgtggtgaccgtgccctccag
caacttcggcacccagacctacacctgcaacgtagatcacaagcccagcaacaccaaggtggacaagacagtt
gagcgcaaatgttgtgtcgagtgcccaccgtgcccagcaccacctgtggcaggaccgtcagtcttcctcttcc
ccccaaaacccaaggacaccctcatgatctcccggacccctgaggtcacgtgcgtggtggtggacgtgagcca
cgaagaccccgaggtccagttcaactggtacgtggacggcgtggaggtgcataatgccaagacaaagccacgg
gaggagcagttcaacagcacgttccgtgtggtcagcgtcctcaccgttgtgcaccaggactggctgaacggca
aggagtacaagtgcaaggtctccaacaaaggcctcccagcccccatcgagaaaaccatctccaaaaccaaagg
gcagccccgagaaccacaggtgtacaccctgcccccatcccgggaggagatgaccaagaaccaggtcagcctg
acctgcctggtcaaaggcttctaccccagcgacatcgccgtggagtgggagagcaatgggcagccggagaaca
actacaagaccacacctcccatgctggactccgacggctccttcttcctctacagcaagctcaccgtggacaa
gagcaggtggcagcaggggaacgtcttctcatgctccgtgatgcatgaggctctgcacaaccactacacgcag
aagagcctctccctgtctccgggtaaatagtaa
(amino acids)
(SEQ ID NO: 164)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSSASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMHEALHNHYTQ
KSLSLSPGK**
Humanized C2 gBLOCK#5 sequence:
(DNA)
(SEQ ID NO: 165)
tgctctgggttccaggttccactggtgacgcggcccagccggccgaggtgcagctggtggagtctgggggagg
cctggtcaagcctggggggtccctgagactctcctgtgcagcctctggattcaccttcagtggctatgccatg
agctgggtccgccaggctccagggaaggggctggagtgggtctcaaccattagtagtggcggaacctacatat
actaccccgactcagtgaagggccgattcaccatctccagagacaacgccaagaactcactgtatctgcaaat
gaacagcctgagagccgaggacacggccgtgtattactgtgcgagacttgggggggataattactacgaatac
ttcgatgtctggggcaaagggaccacggtcaccgtctcctccgcctccaccaagggcccatcggtcttccccc
tggcgccctgctccaggagcacctccgagagcacagccgccctgggctgcctggtcaaggactacttccccga
accggtgacggtgtcgtggaactcaggcgctctgacca
pSEC Tag2 overlapping sequence:
(DNA)
(SEQ ID NO: 166)
tgctctgggttccaggttccactggtgacgc
Human IgG2 constant region overlapping sequence:
(DNA)
(SEQ ID NO: 167)
gacggtgtcgtggaactcaggcgctctgacca
Mouse C2 light chain variable region sequence:
(DNA)
(SEQ ID NO: 168)
gacattgtgatcacacagtctacagcttccttaggtgtatctctggggcagagggccaccatctcatgcaggg
ccagcaaaagtgtcagtacatctggctatagttatatgcactggtaccaacagagaccaggacagccacccaa
actcctcatctatcttgcatccaacctagaatctggggtccctgccaggttcagtggcagtgggtctgggaca
gacttcaccctcaacatccatcctgtggaggaggaggatgctgcaacctattactgtcagcacagtagggagc
ttccgttcacgttcggaggggggaccaagctggagataaaacgggctgatgctgcaccaactgtatcc
(amino acids)
(SEQ ID NO: 169)
DIVITQSTASLGVSLGQRATISCRASKSVSTSGYSYMHWYQQRPGQPPKLLIYLASNLESGVPARFSGSGSGT
DFTLNIHPVEEEDAATYYCQHSRELPFTFGGGTKLEIKRADAAPTVS
Mouse C2 light chain variable framework region 1 (FWR1) sequence:
(DNA)
(SEQ ID NO: 170)
gacattgtgatcacacagtctacagcttccttaggtgtatctctggggcagagggccaccatctcatgc
(amino acids)
(SEQ ID NO: 171)
DIVITQSTASLGVSLGQRATISC
Mouse C2 light chain variable complementarity determining regions 1
(CDR1) sequence:
(DNA)
(SEQ ID NO: 172)
agggccagcaaaagtgtcagtacatctggctatagttatatgcac
(amino acids)
(SEQ ID NO: 173)
RASKSVSTSGYSYMH
Mouse C2 light chain variable framework region 2 (FWR2) sequence:
(DNA)
(SEQ ID NO: 174)
tggtaccaacagagaccaggacagccacccaaactcctcatctat
(amino acids)
(SEQ ID NO: 175)
WYQQRPGQPPKLLIY
Mouse C2 light chain variable complementarity determining regions 2
(CDR2) sequence:
(DNA)
(SEQ ID NO: 176)
cttgcatccaacctagaatc
(amino acids)
(SEQ ID NO: 177)
LASNLES
Mouse C2 light chain variable framework region 3 (FWR3) sequence:
(DNA)
(SEQ ID NO: 178)
tggggtccctgccaggttcagtggcagtgggtctgggacagacttcaccctcaacatccatcctgtggaggag
gaggatgctgcaacctattactgt
(amino acids)
(SEQ ID NO: 179)
GVPARFSGSGSGTDFTLNIHPVEEEDAATYYC
Mouse C2 light chain variable complementarity determining regions 3
(CDR3) sequence:
(DNA)
(SEQ ID NO: 180)
cagcacagtagggagcttccgttcacg
(amino acids)
(SEQ ID NO: 181)
QHSRELPFT
IGKV7-3*01 light chain variable region sequence:
(DNA)
(SEQ ID NO: 182)
gacattgtgctgacccagtctccagcctccttggccgtgtctccaggacagagggccaccatcacctgcagag
ccagtgagagtgtcagtttcttgggaataaacttaattcactggtatcagcagaaaccaggacaacctcctaa
actcctgatttaccaagcatccaataaagacactggggtcccagccaggttcagcggcagtgggtctgggacc
gatttcaccctcacaattaatcctgtggaagctaatgatactgcaaattattactgtctgcagagtaagaatt
ttcctcccaca
(amino acid)
(SEQ ID NO: 183)
DIVLTQSPASLAVSPGQRATITCRASESVSFLGINLIHWYQQKPGQPPKLLIYQASNKDTGVPARFSGSGSGT
DFTLTINPVEANDTANYYCLQSKNFPPT
IGKV7-3*01 light chain variable framework region 1 (FWR1) sequence:
(DNA)
(SEQ ID NO:184)
gacattgtgctgacccagtctccagcctccttggccgtgtctccaggacagagggccaccatcacctgc
(amino acids)
(SEQ ID NO: 185)
DIVLTQSPASLAVSPGQRATITC
IGKV7-3*01 light chain variable complementarity determining regions 1
(CDR1) sequence:
(DNA)
(SEQ ID NO: 186)
agagccagtgagagtgtcagtttcttgggaataaacttaattcac
(amino acids)
(SEQ ID NO:187)
RASESVSFLGINLIH
IGKV7-3*01 light chain variable framework region 2 (FWR2) sequence:
(DNA)
(SEQ ID NO: 188)
tggtatcagcagaaaccaggacaacctcctaaactcctgatttac
(amino acids)
(SEQ ID NO: 189)
WYQQKPGQPPKLLIY
IGKV7-3*01 light chain variable complementarity determining regions 2
(CDR2) sequence:
(DNA)
(SEQ ID NO: 190)
caagcatccaataaagacact
(amino acids)
(SEQ ID NO: 191)
QASNKDT
IGKV7-3*01 light chain variable framework region 3 (FWR3) sequence:
(DNA)
(SEQ ID NO: 192)
ggggtcccagccaggttcagcggcagtgggtctgggaccgatttcaccctcacaattaatcctgtggaagcta
atgatactgcaaattattactgt
(amino acids)
(SEQ ID NO: 193)
GVPARFSGSGSGTDFTLTINPVEANDTANYYC
Humanized C2 light chain variable region sequence:
(DNA)
(SEQ ID NO: 194)
gacattgtgctgacccagtctccagcctccttggccgtgtctccaggacagagggccaccatcacctgcagag
ccagtaagagtgtcagtaccagcggatactcctacatgcactggtatcagcagaaaccaggacaacctcctaa
actcctgatttacctggcatccaatctggagagcggggtcccagccaggttcagcggcagtgggtctgggacc
gatttcaccctcacaattaatcctgtggaagctaatgatactgcaaattattactgtcagcacagtagggagc
tgcctttcacattcggcggagggaccaaggtggagatcaaacgaact
(amino acids)
(SEQ ID NO: 195)
DIVLTQSPASLAVSPGQRATITCRASKSVSTSGYSYMHWYQQKPGQPPKLLIYLASNLESGVPARFSGSGSGT
DFTLTINPVEANDTANYYCQHSRELPFTFGGGTKVEIKRT
Humanized C2 light chain variable framework region 1 (FWR1) acid
sequence:
(DNA)
(SEQ ID NO: 196)
gacattgtgctgacccagtctccagcctccttggccgtgtctccaggacagagggccaccatcacctgc
(amino acids)
(SEQ ID NO: 197)
DIVLTQSPASLAVSPGQRATITC
Humanized C2 light chain variable complementarity determining regions 1
(CDR1) sequence:
(DNA)
(SEQ ID NO: 198)
agagccagtaagagtgtcagtaccagcggatactcctacatgcac
(amino acids)
(SEQ ID NO: 199)
RASKSVSTSGYSYMH
Humanized C2 heavy light variable framework region 2 (FWR2) acid
sequence:
(DNA)
(SEQ ID NO: 200)
tggtatcagcagaaaccaggacaacctcctaaactcctgatttac
(amino acids)
(SEQ ID NO: 201)
WYQQKPGQPPKLLIY
Humanized C2 light chain variable complementarity determining regions 2
(CDR2) sequence:
(DNA)
(SEQ ID NO: 202)
ctggcatccaatctggagagc
(amino acids)
(SEQ ID NO: 203)
LASNLES
Humanized C2 light chain variable framework region 3 (FWR3) acid
sequence:
(DNA)
(SEQ ID NO: 204)
ggggtcccagccaggttcagcggcagtgggtctgggaccgatttcaccctcacaattaatcctgtggaagcta
atgatactgcaaattattactgt
(amino acids)
(SEQ ID NO: 205)
GVPARFSGSGSGTDFTLTINPVEANDTANYYC
Humanized C2 light chain variable complementarity determining regions 3
(CDR3) sequence:
(DNA)
(SEQ ID NO: 206)
cagcacagtagggagctgcctttcaca
(amino acids)
(SEQ ID NO: 207)
QHSRELPFT
Humanized C2 light chain variable complementarity determining regions 3
(CDR3) sequence:
(DNA)
(SEQ ID NO: 208)
ctgcagagtaagaattttcctcccaca
(amino acids)
(SEQ ID NO: 209)
LQSKNFPPT
Humanized C2 gBLOCK#6 sequence (Kappa light chain in pCDNA3.1 V5):
(DNA)
(SEQ ID NO: 210)
actcactatagggagacccaagctggctagttaagcttgggccaccatggagacagacacactcctgctatgg
gtactgctgctctgggttccaggttccactggtgacgacattgtgctgacccagtctccagcctccttggccg
tgtctccaggacagagggccaccatcacctgcagagccagtaagagtgtcagtaccagcggatactcctacat
gcactggtatcagcagaaaccaggacaacctcctaaactcctgatttacctggcatccaatctggagagcggg
gtcccagccaggttcagcggcagtgggtctgggaccgatttcaccctcacaattaatcctgtggaagctaatg
atactgcaaattattactgtcagcacagtagggagctgcctttcacattcggcggagggaccaaggtggagat
caaacgaactacggtggctgcaccatctgtcttcatcttcccgccatctgatgagcagttgaaatctggaact
gcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcac
cctgacgctgagcaaagcagactacgagaaacacaaagtctacgcctgcgaagtcacccatcagggcctgagc
tcgcccgtcacaaagagcttcaacaggggagagtgttagtaagtttaaacccgctgatcagcctcgactgtgc
cttctagttg
pCDNA3.1 V5 5′ overlapping sequence:
(DNA)
(SEQ ID NO: 211)
actcactatagggagacccaagctggctagtt
pCDNA3.1 V5 3′ overlapping sequence:
(DNA)
(SEQ ID NO: 212)
ccgctgatcagcctcgactgtgccttctagttg
Humanized C2 gBLOCK#7 sequence (Kappa light chain in pSEC Tag2):
(DNA)
(SEQ ID NO: 213)
tgctctgggttccaggttccactggtgacgcggcccagccggccgacattgtgctgacccagtctccagcctc
cttggccgtgtctccaggacagagggccaccatcacctgcagagccagtaagagtgtcagtaccagcggatac
tcctacatgcactggtatcagcagaaaccaggacaacctcctaaactcctgatttacctggcatccaatctgg
agagcggggtcccagccaggttcagcggcagtgggtctgggaccgatttcaccctcacaattaatcctgtgga
agctaatgatactgcaaattattactgtcagcacagtagggagctgcctttcacattcggcggagggaccaag
gtggagatcaaacgaactacggtggctgcaccatctgtcttcatcttcccgccatctgatgagcagttgaaat
ctggaactgcctctgttgtgtgcctgctgaataacttctatcccagagaggccaaagtacagtggaaggtgga
taacgccctccaatcgggtaactcccaggagagtgtcacagagcaggacagcaaggacagcacctacagcctc
agcagcaccctgacgctgagcaaagcagactacgagaaacacaaagtctacgcctgcgaagtcacccatcagg
gcctgagctcgcccgtcacaaagagcttcaacaggggagagtgttagtaagtttaaacccgctgatcagcctc
gactgtgccttctagttg
pSEC Tag2 5′ overlapping sequence:
(DNA)
(SEQ ID NO: 214)
tgctctgggttccaggttccactggtgacgc
pSEC Tag2 3′ overlapping sequence:
(DNA)
(SEQ ID NO: 215)
ccgctgatcagcctcgactgtgccttctagttg
Humanized C2 gBLOCK#8 sequence (lambda light chain in pCDNA3.1 V5):
(DNA)
(SEQ ID NO: 216)
actcactatagggagacccaagctggctagttaagcttgggccaccatggagacagacacactcctgctatgg
gtactgctgctctgggttccaggttccactggtgacgacattgtgctgacccagtctccagcctccttggccg
tgtctccaggacagagggccaccatcacctgcagagccagtaagagtgtcagtaccagcggatactcctacat
gcactggtatcagcagaaaccaggacaacctcctaaactcctgatttacctggcatccaatctggagagcggg
gtcccagccaggttcagcggcagtgggtctgggaccgatttcaccctcacaattaatcctgtggaagctaatg
atactgcaaattattactgtcagcacagtagggagctgcctttcacattcggcggagggaccaaggtggagat
caaacgaactggtcagcccaaggctgccccctcggtcactctgttcccgccctcctctgaggagcttcaagcc
aacaaggccacactggtgtgtctcataagtgacttctacccgggagccgtgacagtggcctggaaggcagata
gcagccccgtcaaggcgggagtggagaccaccacaccctccaaacaaagcaacaacaagtacgcggccagcag
ctatctgagcctgacgcctgagcagtggaagtcccacagaagctacagctgccaggtcacgcatgaagggagc
accgtggagaagacagtggcccctacagaatgttcatagtaagtttaaacccgctgatcagcctcgactgtgc
cttctagttg
pCDNA3.1 V5 5′ overlapping sequence:
(DNA)
(SEQ ID NO: 217)
actcactatagggagacccaagctggctagtt
pCDNA3.1 V5 3′ overlapping sequence:
(DNA)
(SEQ ID NO: 218)
ccgctgatcagcctcgactgtgccttctagttg
Humanized C2 gBLOCK#9 sequence (lambda light chain in pSEC Tag2):
(DNA)
(SEQ ID NO: 219)
tgctctgggttccaggttccactggtgacgcggcccagccggccgacattgtgctgacccagtctccagcctc
cttggccgtgtctccaggacagagggccaccatcacctgcagagccagtaagagtgtcagtaccagcggatac
tcctacatgcactggtatcagcagaaaccaggacaacctcctaaactcctgatttacctggcatccaatctgg
agagcggggtcccagccaggttcagcggcagtgggtctgggaccgatttcaccctcacaattaatcctgtgga
agctaatgatactgcaaattattactgtcagcacagtagggagctgcctttcacattcggcggagggaccaag
gtggagatcaaacgaactggtcagcccaaggctgccccctcggtcactctgttcccgccctcctctgaggagc
ttcaagccaacaaggccacactggtgtgtctcataagtgacttctacccgggagccgtgacagtggcctggaa
ggcagatagcagccccgtcaaggcgggagtggagaccaccacaccctccaaacaaagcaacaacaagtacgcg
gccagcagctatctgagcctgacgcctgagcagtggaagtcccacagaagctacagctgccaggtcacgcatg
aagggagcaccgtggagaagacagtggcccctacagaatgttcatagtaagtttaaacccgctgatcagcctc
gactgtgccttctagttg
pSEC Tag2 5′ overlapping sequence:
(DNA)
(SEQ ID NO: 220)
tgctctgggttccaggttccactggtgacgc
pSEC Tag2 3′ overlapping sequence:
(DNA)
(SEQ ID NO: 221)
ccgctgatcagcctcgactgtgccttctagttg
Murine Ig kappa chain leader sequence
(DNA)
(SEQ ID NO: 222)
atggagacagacacactcctgctatgggtactgctgctctgggttccaggttccactggtgac
(amino acids)
(SEQ ID NO: 223)
METDTLLLWVLLLWVPGSTGD
Interleukin-2 (IL-2) leader sequence
(DNA)
(SEQ ID NO: 224)
atgtacaggatgcaactcctgtcttgcattgcactaagtcttgcacttgtcacaaacagt
(amino acids)
(SEQ ID NO: 225)
MYRMQLLSCIALSLALVTNS
CD33 leader sequence
(DNA)
(SEQ ID NO: 226)
atgcctcttctgcttctgcttcctctgctttgggctggagctcttgct
(amino acids)
(SEQ ID NO: 227)
MPLLLLLPLLWAGALA
IGHV3-21*03 leader sequence
(DNA)
(SEQ ID NO: 228)
atggaactggggctccgctgggttttccttgttgctattttagaaggtgtccagtgt
(amino acids)
(SEQ ID NO: 229)
MELGLRWVFLVAILEGVQC
IGHV3-11*02 leader sequence
(DNA)
(SEQ ID NO: 230)
atggaagccccagcgcagcttctcttcctcctgctactctggctcccagataccactgga
(amino acids)
(SEQ ID NO: 231)
MEAPAQLLFLLLLWLPDTTG
Humanized E6 single chain GS3
(DNA)
(SEQ ID NO: 232)
gaggtgcagctggtggagtctgggggaggcctggtcaagcctggggggtccctgagactctcctgtg
cagcctctggattcaccttcagtaggtatggcatgagctgggtccgccaggctccagggaagaggct
ggagtgggtctcaaccattagtggcggaggcacctacatatactacccagactcagtgaagggccga
ttcaccatctccagagacaacgccaagaacaccctgtatctgcaaatgaacagcctgagagccgagg
acacggctgtgtattactgtaccagagataactatggccgcaactatgattatggcatggattattg
gggccagggcaccctggtgaccgtgagcagcggcggtggcggatccggcggtggcggatccggcggt
ggcggatccgaaattgtgttgacacagtctccagccaccctgtctttgtctccaggggaaagagcca
ccctcacctgcagcgccaccagcagtgttagctacatccactggtaccaacagaggcctggccagag
ccccaggctcctcatctatagcacctccaacctggccagcggcatcccagccaggttcagtggcagt
gggtctgggagcgactacactctcaccatcagcagcctagagcctgaagattttgcagtttattact
gtcagcagcgtagcagctcccctttcacctttggcagcggcaccaaagtggaaattaaa
(amino acids)
(SEQ ID NO: 233)
EVQLVESGGGLVKPGGSLRLSCAASGFIFSRYGMSWVRQAPGKRLEWVSTISGGGIYIYYPDSVKGRFTISRD
NAKNTLYLQMNSLRAEDTAVYYCIRDNYGRNYDYGMDYWGQGTLVIVSSGGGGSGGGGSGGGGSEIVLIQSPA
ILSLSPGERATLICSATSSVSYIHWYQQRPGQSPRLLIYSTSNLASGIPARFSGSGSGSDYILTISSLEPEDF
AVYYCQQRSSSPFTFGSGTKVEIK
Humanized E6 single chain IgG1noC
(DNA)
(SEQ ID NO: 234)
gaggtgcagctggtggagtctgggggaggcctggtcaagcctggggggtccctgagactctcctgtgcagcct
ctggattcaccttcagtaggtatggcatgagctgggtccgccaggctccagggaagaggctggagtgggtctc
aaccattagtggcggaggcacctacatatactacccagactcagtgaagggccgattcaccatctccagagac
aacgccaagaacaccctgtatctgcaaatgaacagcctgagagccgaggacacggctgtgtattactgtacca
gagataactatggccgcaactatgattatggcatggattattggggccagggcaccctggtgaccgtgagcag
cgataaaacccatactaaaccgccaaaaccggcgccggaactgctgggtggtcctggtaccggtgaaattgtg
ttgacacagtctccagccaccctgtctttgtctccaggggaaagagccaccctcacctgcagcgccaccagca
gtgttagctacatccactggtaccaacagaggcctggccagagccccaggctcctcatctatagcacctccaa
cctggccagcggcatcccagccaggttcagtggcagtgggtctgggagcgactacactctcaccatcagcagc
ctagagcctgaagattttgcagtttattactgtcagcagcgtagcagctcccctttcacctttggcagcggca
ccaaagtggaaattaaa
(amino acids)
(SEQ ID NO: 235)
EVQLVESGGGLVKPGGSLRLSCAASGFTFSRYGMSWVRQAPGKRLEWVSTISGGGTYIYYPDSVKGRFTISRD
NAKNTLYLQMNSLRAEDTAVYYCTRDNYGRNYDYGMDYWGQGTLVTVSSDKTHTKPPKPAPELLGGPGTGEIV
LTQSPATLSLSPGERATLTCSATSSVSYIHWYQQRPGQSPRLLIYSTSNLASGIPARFSGSGSGSDYTLTISS
LEPEDFAVYYCQQRSSSPFTFGSGTKVEIK
Humanized E6 single chain X4 (linker is IgG1 and IgG2 modified hinge
region)
(DNA)
(SEQ ID NO: 236)
gaggtgcagctggtggagtctgggggaggcctggtcaagcctggggggtccctgagactctcctgtgcagcct
ctggattcaccttcagtaggtatggcatgagctgggtccgccaggctccagggaagaggctggagtgggtctc
aaccattagtggcggaggcacctacatatactacccagactcagtgaagggccgattcaccatctccagagac
aacgccaagaacaccctgtatctgcaaatgaacagcctgagagccgaggacacggctgtgtattactgtacca
gagataactatggccgcaactatgattatggcatggattattggggccagggcaccctggtgaccgtgagcag
cgataaaacccatactaaaccgccaaaaccggcgccggaactgctgggtggtcctggtaccggtactggtggt
ccgactattaaacctccgaaacctccgaaacctgctccgaacctgctgggtggtccggaaattgtgttgacac
agtctccagccaccctgtctttgtctccaggggaaagagccaccctcacctgcagcgccaccagcagtgttag
ctacatccactggtaccaacagaggcctggccagagccccaggctcctcatctatagcacctccaacctggcc
agcggcatcccagccaggttcagtggcagtgggtctgggagcgactacactctcaccatcagcagcctagagc
ctgaagattttgcagtttattactgtcagcagcgtagcagctcccctttcacctttggcagcggcaccaaagt
ggaaattaaa
(amino acids)
(SEQ ID NO: 237)
EVQLVESGGGLVKPGGSLRLSCAASGFTFSRYGMSWVRQAPGKRLEWVSTISGGGTYIYYPDSVKGRFTISRD
NAKNTLYLQMNSLRAEDTAVYYCTRDNYGRNYDYGMDYWGQGTLVTVSSDKTHTKPPKPAPELLGGPGTGTGC
PTIKPPKPPKPAPNLLGGPEIVLTQSPATLSLSPGERATLTCSATSSVSYIHWYQQRPGQSPRLLIYSTSNLA
SGIPARFSGSGSGSDYTLTISSLEPEDFAVYYCQQRSSSPFTFGSGTKVEIK
Humanized C2 single chain GS3
(DNA)
(SEQ ID NO: 238)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccccgactcagtgaagggccgattcaccatctccagagac
aacgccaagaactcactgtatctgcaaatgaacagcctgagagccgaggacacggccgtgtattactgtgcga
gacttgggggggataattactacgaatacttcgatgtctggggcaaagggaccacggtcaccgtctcctccgg
cggtggcggatccggcggtggcggatccggcggtggcggatccgacattgtgctgacccagtctccagcctcc
ttggccgtgtctccaggacagagggccaccatcacctgcagagccagtaagagtgtcagtaccagcggatact
cctacatgcactggtatcagcagaaaccaggacaacctcctaaactcctgatttacctggcatccaatctgga
gagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaagg
tggagatcaaacgaact
(amino acids)
(SEQ ID NO: 239)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSSGGGGSGGGGSGGGGSDIVLTQSPAS
LAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRT
Humanized C2 single chain IgG (no Cysteine)
(DNA)
(SEQ ID NO: 240)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccccgactcagtgaagggccgattcaccatctccagagac
aacgccaagaactcactgtatctgcaaatgaacagcctgagagccgaggacacggccgtgtattactgtgcga
gacttgggggggataattactacgaatacttcgatgtctggggcaaagggaccacggtcaccgtctcctccga
taaaacccatactaaaccgccaaaaccggcgccggaactgctgggtggtcctggtaccggtgacattgtgctg
acccagtctccagcctccttggccgtgtctccaggacagagggccaccatcacctgcagagccagtaagagtg
tcagtaccagcggatactcctacatgcactggtatcagcagaaaccaggacaacctcctaaactcctgattta
cctggcatccaatctggagagcggggtcccagccaggttcagcggcagtgggtctgggaccgatttcaccctc
acaattaatcctgtggaagctaatgatactgcaaattattactgtcagcacagtagggagctgcctttcacat
tcggcggagggaccaaggtggagatcaaacgaact
(amino acids)
(SEQ ID NO: 241)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSSDKTHTKPPKPAPELLGGPGTGDIVL
TQSPASLAVSPGQRATITCRASKSVSTSGYSYMHWYQQKPGQPPKLLIYLASNLESGVPARFSGSGSGTDFTL
TINPVEANDTANYYCQHSRELPFTFGGGTKVEIKRT
Humanized C2 single chain X4 (linker is IgG1 and IgG2 modified hinge
region)
(DNA)
(SEQ ID NO: 242)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccccgactcagtgaagggccgattcaccatctccagagac
aacgccaagaactcactgtatctgcaaatgaacagcctgagagccgaggacacggccgtgtattactgtgcga
gacttgggggggataattactacgaatacttcgatgtctggggcaaagggaccacggtcaccgtctcctccga
taaaacccatactaaaccgccaaaaccggcgccggaactgctgggtggtcctggtaccggtactggtggtccg
actattaaacctccgaaacctccgaaacctgctccgaacctgctgggtggtccggacattgtgctgacccagt
ctccagcctccttggccgtgtctccaggacagagggccaccatcacctgcagagccagtaagagtgtcagtac
cagcggatactcctacatgcactggtatcagcagaaaccaggacaacctcctaaactcctgatttacctggca
tccaatctggagagcggggtcccagccaggttcagcggcagtgggtctgggaccgatttcaccctcacaatta
atcctgtggaagctaatgatactgcaaattattactgtcagcacagtagggagctgcctttcacattcggcgg
agggaccaaggtggagatcaaacgaact
(amino acids)
(SEQ ID NO: 243)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSSDKTHTKPPKPAPELLGGPGTGTGGP
TIKPPKPPKPAPNLLGGPDIVLTQSPASLAVSPGQRATITCRASKSVSTSGYSYMHWYQQKPGQPPKLLIYLA
SNLESGVPARFSGSGSGTDFTLTINPVEANDTANYYCQHSRELPFTFGGGTKVEIKRT
Humanized C3 single chain GS3
(DNA)
(SEQ ID NO: 244)
caggttcagctggtgcagtctggagctgaggtgaagaagcctggggcctcagtgaaggtctcctgcaaggctt
ctggttacacctttaccgactacgccatgaactgggtgcgacaggcccctggacaagggcttgagtggatggg
agtgatcagcaccttcagcggtaacacaaacttcaaccagaagttcaagggcagagtcaccatgaccacagac
acatccacgagcacagcctacatggagctgaggagcctgagatctgacgacacggccgtgtattactgtgcga
gaagcgactactacggcccatacttcgactactggggccagggcaccaccctgaccgtgtccagcggcggtgg
cggatccggcggtggcggatccggcggtggcggatccgatattgtgatgacccagactccactctctctgtcc
gtcacccctggacagccggcctccatctcctgcaggtctagtcagaccattgtccatagtaatggaaacacct
atttggagtggtacctgcagaagccaggccagtctccacagctcctgatctataaggtttccaaccggttctc
tggagtgccagataggttcagtggcagcgggtcagggacagatttcacactgaaaatcagccgggtggaggct
gaggatgttggggtttattactgcttccaaggtagccacgtgcctttcaccttcggcggagggaccaaggtgg
agatcaaacgaact
(amino acids)
(SEQ ID NO: 245)
QVQLVQSGAEVKKPGASVKVSCKASGYTFTDYAMNWVRQAPGQGLEWMGVISTFSGNTNFNQKFKGRVTMTTD
TSTSTAYMELRSLRSDDTAVYYCARSDYYGPYFDYWGQGTTLTVSSGGGGSGGGGSGGGGSDIVMTQTPLSLS
VTPGQPASISCRSSQTIVHSNGNTYLEWYLQKPGQSPQLLIYKVSNRFSGVPDRFSGSGSGTDFTLKISRVEA
EDVGVYYCFQGSHVPFTFGGGTKVEIKRT
Humanized C3 single chain IgG1 (no Cysteine)
(DNA)
(SEQ ID NO: 246)
caggttcagctggtgcagtctggagctgaggtgaagaagcctggggcctcagtgaaggtctcctgcaaggctt
ctggttacacctttaccgactacgccatgaactgggtgcgacaggcccctggacaagggcttgagtggatggg
agtgatcagcaccttcagcggtaacacaaacttcaaccagaagttcaagggcagagtcaccatgaccacagac
acatccacgagcacagcctacatggagctgaggagcctgagatctgacgacacggccgtgtattactgtgcga
gaagcgactactacggcccatacttcgactactggggccagggcaccaccctgaccgtgtccagcgataaaac
ccatactaaaccgccaaaaccggcgccggaactgctgggtggtcctggtaccggtgatattgtgatgacccag
actccactctctctgtccgtcacccctggacagccggcctccatctcctgcaggtctagtcagaccattgtcc
atagtaatggaaacacctatttggagtggtacctgcagaagccaggccagtctccacagctcctgatctataa
ggtttccaaccggttctctggagtgccagataggttcagtggcagcgggtcagggacagatttcacactgaaa
atcagccgggtggaggctgaggatgttggggtttattactgcttccaaggtagccacgtgcctttcaccttcg
gcggagggaccaaggtggagatcaaacgaact
(amino acids)
(SEQ ID NO: 247)
QVQLVQSGAEVKKPGASVKVSCKASGYTFTDYAMNWVRQAPGQGLEWMGVISTFSGNTNFNQKFKGRVTMTTD
TSTSTAYMELRSLRSDDTAVYYCARSDYYGPYFDYWGQGTTLTVSSDKTHTKPPKPAPELLGGPGTGDIVMTQ
TPLSLSVTPGQPASISCRSSQTIVHSNGNTYLEWYLQKPGQSPQLLIYKVSNRFSGVPDRFSGSGSGTDFTLK
ISRVEAEDVGVYYCFQGSHVPFTFGGGTKVEIKRT
Humanized C3 single chain X4 (linker is IgG1 and IgG2 modified hinge
region)
(DNA)
(SEQ ID NO: 248)
caggttcagctggtgcagtctggagctgaggtgaagaagcctggggcctcagtgaaggtctcctgcaaggctt
ctggttacacctttaccgactacgccatgaactgggtgcgacaggcccctggacaagggcttgagtggatggg
agtgatcagcaccttcagcggtaacacaaacttcaaccagaagttcaagggcagagtcaccatgaccacagac
acatccacgagcacagcctacatggagctgaggagcctgagatctgacgacacggccgtgtattactgtgcga
gaagcgactactacggcccatacttcgactactggggccagggcaccaccctgaccgtgtccagcgataaaac
ccatactaaaccgccaaaaccggcgccggaactgctgggtggtcctggtaccggtactggtggtccgactatt
aaacctccgaaacctccgaaacctgctccgaacctgctgggtggtccggatattgtgatgacccagactccac
tctctctgtccgtcacccctggacagccggcctccatctcctgcaggtctagtcagaccattgtccatagtaa
tggaaacacctatttggagtggtacctgcagaagccaggccagtctccacagctcctgatctataaggtttcc
aaccggttctctggagtgccagataggttcagtggcagcgggtcagggacagatttcacactgaaaatcagcc
gggtggaggctgaggatgttggggtttattactgcttccaaggtagccacgtgcctttcaccttcggcggagg
gaccaaggtggagatcaaacgaact
(amino acids)
(SEQ ID NO: 249)
QVQLVQSGAEVKKPGASVKVSCKASGYTFTDYAMNWVRQAPGQGLEWMGVISTFSGNTNFNQKFKGRVTMTTD
TSTSTAYMELRSLRSDDTAVYYCARSDYYGPYFDYWGQGTTLTVSSDKTHTKPPKPAPELLGGPGTGTGGPTI
KPPKPPKPAPNLLGGPDIVMTQTPLSLSVTPGQPASISCRSSQTIVHSNGNTYLEWYLQKPGQSPQLLIYKVS
NRFSGVPDRFSGSGSGTDFTLKISRVEAEDVGVYYCFQGSHVPFTFGGGTKVEIKRT
Humanized C8 single chain GS3 (linker is [Gly₄Ser₁]₃)
(DNA)
(SEQ ID NO: 250)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccctgactcagtgaagggccgattcaccatctccagagac
aacgccaagaactcactgtatctgcaaatgaacagcctgagagccgaggacacggccgtgtattactgtgcga
gactgggcggcgataactattatgaatattggggcaaagggaccacggtcaccgtctcctccggcggtggcgg
atccggcggtggcggatccggcggtggcggatccgacatcgtgatgacccagtctccagactccctggctgtg
tctctgggcgagagggccaccatcaactgcagggccagcaagagtgttagcaccagcggctacagctacatgc
actggtaccagcagaaaccaggacagcctcctaagctgctcatttacctggtgtctaacctggaatccggggt
ccctgaccgattcagtggcagcgggtctgggacagatttcactctcaccatcagcagcctgcaggctgaagat
gtggcagtttattactgtcaacacattcgggaactgaccaggagtgaattcggcggagggaccaaggtggaga
tcaaacgaact
(amino acids)
(SEQ ID NO: 251)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYWGKGTTVTVSSGGGGSGGGGSGGGGSDIVMTQSPDSLAV
SLGERATINCRASKSVSTSGYSYMHWYQQKPGQPPKLLIYLVSNLESGVPDRFSGSGSGTDFTLTISSLQAED
VAVYYCQHIRELTRSEFGGGTKVEIKRT
Humanized C8 single chain IgG1 (no Cysteine)
(DNA)
(SEQ ID NO: 252)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccctgactcagtgaagggccgattcaccatctccagagac
aacgccaagaactcactgtatctgcaaatgaacagcctgagagccgaggacacggccgtgtattactgtgcga
gactgggcggcgataactattatgaatattggggcaaagggaccacggtcaccgtctcctccgataaaaccca
tactaaaccgccaaaaccggcgccggaactgctgggtggtcctggtaccggtgacatcgtgatgacccagtct
ccagactccctggctgtgtctctgggcgagagggccaccatcaactgcagggccagcaagagtgttagcacca
gcggctacagctacatgcactggtaccagcagaaaccaggacagcctcctaagctgctcatttacctggtgtc
taacctggaatccggggtccctgaccgattcagtggcagcgggtctgggacagatttcactctcaccatcagc
agcctgcaggctgaagatgtggcagtttattactgtcaacacattcgggaactgaccaggagtgaattcggcg
gagggaccaaggtggagatcaaacgaact
(amino acids)
(SEQ ID NO: 253)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYWGKGTTVTVSSDKTHTKPPKPAPELLGGPGTGDIVMTQS
PDSLAVSLGERATINCRASKSVSTSGYSYMHWYQQKPGQPPKLLIYLVSNLESGVPDRFSGSGSGTDFTLTIS
SLQAEDVAVYYCQHIRELTRSEFGGGTKVEIKRT
Humanized C8 single chain X4 (linker is IgG1 and IgG2 modified hinge
region)
(DNA)
(SEQ ID NO: 254)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactacccagactcagtgaagggccgattcaccatctccagagac
aacgccaagaactcactgtatctgcaaatgaacagcctgagagccgaggacacggccgtgtattactgtgcga
gactgggcggcgacaattactatgagtattggggcaaagggaccacggtcaccgtctcctccgataaaaccca
tactaaaccgccaaaaccggcgccggaactgctgggtggtcctggtaccggtactggtggtccgactattaaa
cctccgaaacctccgaaacctgctccgaacctgctgggtggtccggacatcgtgatgacccagtctccagact
ccctggctgtgtctctgggcgagagggccaccatcaactgcagggccagcaagagtgttagcaccagcggcta
cagctacatgcactggtaccagcagaaaccaggacagcctcctaagctgctcatttacctggtgtctaacctg
gaatccggggtccctgaccgattcagtggcagcgggtctgggacagatttcactctcaccatcagcagcctgc
aggctgaagatgtggcagtttattactgtcaacacattcgggaactgaccaggagtgaattcggcggagggac
caaggtggagatcaaacgaact
(amino acids)
(SEQ ID NO: 255)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYWGKGTTVTVSSDKTHTKPPKPAPELLGGPGTGTGGPTIK
PPKPPKPAPNLLGGPDIVMTQSPDSLAVSLGERATINCRASKSVSTSGYSYMHWYQQKPGQPPKLLIYLVSNL
ESGVPDRFSGSGSGTDFTLTISSLQAEDVAVYYCQHIRELTRSEFGGGTKVEIKRT
pSECTag2 E6 scFV-FC
(DNA)
(SEQ ID NO: 256)
atggagacagacacactcctgctatgggtactgctgctctgggttccaggttccactggtgacgcgg
cccagccggccgaggtgcagctggtggagtctgggggaggcctggtcaagcctggggggtccctgag
actctcctgtgcagcctctggattcaccttcagtaggtatggcatgagctgggtccgccaggctcca
gggaagaggctggagtgggtctcaaccattagtggcggaggcacctacatatactacccagactcag
tgaagggccgattcaccatctccagagacaacgccaagaacaccctgtatctgcaaatgaacagcct
gagagccgaggacacggctgtgtattactgtaccagagataactatggccgcaactatgattatggc
atggattattggggccagggcaccctggtgaccgtgagcagcggcggtggcggatccggcggtggcg
gatccggcggtggcggatccgaaattgtgttgacacagtctccagccaccctgtctttgtctccagg
ggaaagagccaccctcacctgcagcgccaccagcagtgttagctacatccactggtaccaacagagg
cctggccagagccccaggctcctcatctatagcacctccaacctggccagcggcatcccagccaggt
tcagtggcagtgggtctgggagcgactacactctcaccatcagcagcctagagcctgaagattttgc
agtttattactgtcagcagcgtagcagctcccctttcacctttggcagcggcaccaaagtggaaatt
aaagagcccaaatcttgtgacaaaactcacacatgcccaccgtgcccagcacctgaactcctggggg
gaccgtcagtcttcctcttccccccaaaacccaaggacaccctcatgatctcccggacccctgaggt
cacatgcgtggtggtggacgtgagccacgaagaccctgaggtcaagttcaactggtacgtggacggc
gtggaggtgcataatgccaagacaaagccgcgggaggagcagtacaacagcacgtaccgtgtggtca
gcgtcctcaccgtcctgcaccaggactggctgaatggcaaggagtacaagtgcaaggtctccaacaa
agccctcccagcccccatcgagaaaaccatctccaaagccaaagggcagccccgagaaccacaggtg
tacaccctgcccccatcccgggaggagatgaccaagaaccaggtcagcctgacctgcctggtcaaag
gcttctatcccagcgacatcgccgtggagtgggagagcaatgggcagccggagaacaactacaagac
cacgcctcccgtgctggactccgacggctccttcttcctctacagcaagctcaccgtggacaagagc
aggtggcagcaggggaacgtcttctcatgctccgtgatgcatgaggctctgcacaaccactacacgc
agaagagcctctccctgtctccgggtaaatgataa
(amino acids)
(SEQ ID NO: 257)
METDTLLLWVLLLWVPGSTGDAAQPAEVQLVESGGGLVKPGGSLRLSCAASGFTFSRYGMSWVRQAP
GKRLEWVSTISGGGTYIYYPDSVKGRFTISRDNAKNTLYLQMNSLRAEDTAVYYCTRDNYGRNYDYG
MDYWGQGTLVTVSSGGGGSGGGGSGGGGSEIVLTQSPATLSLSPGERATLTCSATSSVSYIHWYQQR
PGQSPRLLIYSTSNLASGIPARFSGSGSGSDYTLTISSLEPEDFAVYYCQQRSSSPFTFGSGTKVEI
KEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK**
E6 scFC-FC 1 gBLOCk sequence:
(SEQ ID NO: 258)
tgctctgggttccaggttccactggtgacgcggcccagccggccgaggtgcagctggtggagtctgg
gggaggcctggtcaagcctggggggtccctgagactctcctgtgcagcctctggattcaccttcagt
aggtatggcatgagctgggtccgccaggctccagggaagaggctggagtgggtctcaaccattagtg
gcggaggcacctacatatactacccagactcagtgaagggccgattcaccatctccagagacaacgc
caagaacaccctgtatctgcaaatgaacagcctgagagccgaggacacggctgtgtattactgtacc
agagataactatggccgcaactatgattatggcatggattattggggccagggcaccctggtgaccg
tgagcagcggcggtggcggatccggcggtggcggatccggcggtggcggatccgaaattgtgttgac
acagtctccagccaccctgtctttgtc
E6 scFC-FC 2 gBLOCk sequence:
(SEQ ID NO: 259)
aattgtgttgacacagtctccagccaccctgtctttgtctccaggggaaagagccaccctcacctgc
agcgccaccagcagtgttagctacatccactggtaccaacagaggcctggccagagccccaggctcc
tcatctatagcacctccaacctggccagcggcatcccagccaggttcagtggcagtgggtctgggag
cgactacactctcaccatcagcagcctagagcctgaagattttgcagtttattactgtcagcagcgt
agcagctcccctttcacctttggcagcggcaccaaagtggaaattaaagagcccaaatcttgtgaca
aaactcacacatgcccaccgtgcccagcacctgaactcctggggggaccgtcagtcttcctcttccc
cccaaaacccaaggacaccctcatgatctcccggacccctgaggtcacatgcgtggtggtggacgtg
agccacgaagaccctgaggtcaagttcaactggtacgtggacggcgtggaggtgcataatgccaaga
caaagccgcgggaggagcagtacaacagcacgtaccgtgtggtcagc
pSECTag2 C2 scFV-FC
(DNA)
(SEQ ID NO: 260)
atggagacagacacactcctgctatgggtactgctgctctgggttccaggttccactggtgacgcgg
cccagccggccgaggtgcagctggtggagtctgggggaggcctggtcaagcctggggggtccctgag
actctcctgtgcagcctctggattcaccttcagtggctatgccatgagctgggtccgccaggctcca
gggaaggggctggagtgggtctcaaccattagtagtggcggaacctacatatactaccccgactcag
tgaagggccgattcaccatctccagagacaacgccaagaactcactgtatctgcaaatgaacagcct
gagagccgaggacacggccgtgtattactgtgcgagacttgggggggataattactacgaatacttc
gatgtctggggcaaagggaccacggtcaccgtctcctccggcggtggcggatccggcggtggcggat
ccggcggtggcggatccgacattgtgctgacccagtctccagcctccttggccgtgtctccaggaca
gagggccaccatcacctgcagagccagtaagagtgtcagtaccagcggatactcctacatgcactgg
tatcagcagaaaccaggacaacctcctaaactcctgatttacctggcatccaatctggagagcgggg
tcccagccaggttcagcggcagtgggtctgggaccgatttcaccctcacaattaatcctgtggaagc
taatgatactgcaaattattactgtcagcacagtagggagctgcctttcacattcggcggagggacc
aaggtggagatcaaacgaactgagcccaaatcttgtgacaaaactcacacatgcccaccgtgcccag
cacctgaactcctggggggaccgtcagtcttcctcttccccccaaaacccaaggacaccctcatgat
ctcccggacccctgaggtcacatgcgtggtggtggacgtgagccacgaagaccctgaggtcaagttc
aactggtacgtggacggcgtggaggtgcataatgccaagacaaagccgcgggaggagcagtacaaca
gcacgtaccgtgtggtcagcgtcctcaccgtcctgcaccaggactggctgaatggcaaggagtacaa
gtgcaaggtctccaacaaagccctcccagcccccatcgagaaaaccatctccaaagccaaagggcag
ccccgagaaccacaggtgtacaccctgcccccatcccgggaggagatgaccaagaaccaggtcagcc
tgacctgcctggtcaaaggcttctatcccagcgacatcgccgtggagtgggagagcaatgggcagcc
ggagaacaactacaagaccacgcctcccgtgctggactccgacggctccttcttcctctacagcaag
ctcaccgtggacaagagcaggtggcagcaggggaacgtcttctcatgctccgtgatgcatgaggctc
tgcacaaccactacacgcagaagagcctctccctgtctccgggtaaatgataa
(amino acids)
(SEQ ID NO: 261)
METDTLLLWVLLLWVPGSTGDAAQPAEVQLVESGGGLVKPGGSLRLSCAASGFTFSGYAMSWVRQAP
GKGLEWVSTISSGGTYIYYPDSVKGRFTISRDNAKNSLYLQMNSLRAEDTAVYYCARLGGDNYYEYF
DVWGKGTTVTVSSGGGGSGGGGSGGGGSDIVLTQSPASLAVSPGQRATITCRASKSVSTSGYSYMHW
YQQKPGQPPKLLIYLASNLESGVPARFSGSGSGTDFTLTINPVEANDTANYYCQHSRELPFTFGGGT
KVEIKRTEPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSK
LTVDKSRWQQGNVESCSVMHEALHNHYTQKSLSLSPGK**
C2 scFV-FC 1 gBLOCk sequence:
(DNA)
(SEQ ID NO: 262)
tgctctgggttccaggttccactggtgacgcggcccagccggccgaggtgcagctggtggagtctgg
gggaggcctggtcaagcctggggggtccctgagactctcctgtgcagcctctggattcaccttcagt
ggctatgccatgagctgggtccgccaggctccagggaaggggctggagtgggtctcaaccattagta
gtggcggaacctacatatactaccccgactcagtgaagggccgattcaccatctccagagacaacgc
caagaactcactgtatctgcaaatgaacagcctgagagccgaggacacggccgtgtattactgtgcg
agacttgggggggataattactacgaatacttcgatgtctggggcaaagggaccacggtcaccgtct
cctccggcggtggcggatccggcggtggcggatccggcggtggcggatccgacattgtgctgaccca
gtctccagcctccttggc
C2 scFV-FC 2 gBLOCk sequence:
(DNA)
(SEQ ID NO: 263)
cattgtgctgacccagtctccagcctccttggccgtgtctccaggacagagggccaccatcacctgc
agagccagtaagagtgtcagtaccagcggatactcctacatgcactggtatcagcagaaaccaggac
aacctcctaaactcctgatttacctggcatccaatctggagagcggggtcccagccaggttcagcgg
cagtgggtctgggaccgatttcaccctcacaattaatcctgtggaagctaatgatactgcaaattat
tactgtcagcacagtagggagctgcctttcacattcggcggagggaccaaggtggagatcaaacgaa
ctgagcccaaatcttgtgacaaaactcacacatgcccaccgtgcccagcacctgaactcctgggggg
accgtcagtcttcctcttccccccaaaacccaaggacaccctcatgatctcccggacccctgaggtc
acatgcgtggtggtggacgtgagccacgaagaccctgaggtcaagttcaactggtacgtggacggcg
tggaggtgcataatgccaagacaaagccgcgggaggagcagtacaacagcacgtaccgtgtggtcag
c
pSECTag2 C3 scFV-FC
(DNA)
(SEQ ID NO: 264)
atggagacagacacactcctgctatgggtactgctgctctgggttccaggttccactggtgacgcgg
cccagccggcccaggttcagctggtgcagtctggagctgaggtgaagaagcctggggcctcagtgaa
ggtctcctgcaaggcttctggttacacctttaccgactacgccatgaactgggtgcgacaggcccct
ggacaagggcttgagtggatgggagtgatcagcaccttcagcggtaacacaaacttcaaccagaagt
tcaagggcagagtcaccatgaccacagacacatccacgagcacagcctacatggagctgaggagcct
gagatctgacgacacggccgtgtattactgtgcgagaagcgactactacggcccatacttcgactac
tggggccagggcaccaccctgaccgtgtccagcggcggtggcggatccggcggtggcggatccggcg
gtggcggatccgatattgtgatgacccagactccactctctctgtccgtcacccctggacagccggc
ctccatctcctgcaggtctagtcagaccattgtccatagtaatggaaacacctatttggagtggtac
ctgcagaagccaggccagtctccacagctcctgatctataaggtttccaaccggttctctggagtgc
cagataggttcagtggcagcgggtcagggacagatttcacactgaaaatcagccgggtggaggctga
ggatgttggggtttattactgcttccaaggtagccacgtgcctttcaccttcggcggagggaccaag
gtggagatcaaacgaactgagcccaaatcttgtgacaaaactcacacatgcccaccgtgcccagcac
ctgaactcctggggggaccgtcagtcttcctcttccccccaaaacccaaggacaccctcatgatctc
ccggacccctgaggtcacatgcgtggtggtggacgtgagccacgaagaccctgaggtcaagttcaac
tggtacgtggacggcgtggaggtgcataatgccaagacaaagccgcgggaggagcagtacaacagca
cgtaccgtgtggtcagcgtcctcaccgtcctgcaccaggactggctgaatggcaaggagtacaagtg
caaggtctccaacaaagccctcccagcccccatcgagaaaaccatctccaaagccaaagggcagccc
cgagaaccacaggtgtacaccctgcccccatcccgggaggagatgaccaagaaccaggtcagcctga
cctgcctggtcaaaggcttctatcccagcgacatcgccgtggagtgggagagcaatgggcagccgga
gaacaactacaagaccacgcctcccgtgctggactccgacggctccttcttcctctacagcaagctc
accgtggacaagagcaggtggcagcaggggaacgtcttctcatgctccgtgatgcatgaggctctgc
acaaccactacacgcagaagagcctctccctgtctccgggtaaatgataa
(amino acids)
(SEQ ID NO: 265)
METDTLLLWVLLLWVPGSTGDAAQPAQVQLVQSGAEVKKPGASVKVSCKASGYTFTDYAMNWVRQAPGQGLEW
MGVISTFSGNTNFNQKFKGRVTMTTDTSTSTAYMELRSLRSDDTAVYYCARSDYYGPYFDYWGQGTTLTVSSG
GGGSGGGGSGGGGSDIVMTQTPLSLSVTPGQPASISCRSSQTIVHSNGNTYLEWYLQKPGQSPQLLIYKVSNR
FSGVPDRFSGSGSGTDFTLKISRVEAEDVGVYYCFQGSHVPFTFGGGTKVEIKRTEPKSCDKTHTCPPCPAPE
LLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYTQKSLSLSPGK**
C3 GS scFV FC 1 gBLOCk sequence:
(DNA)
(SEQ ID NO: 266)
tgctctgggttccaggttccactggtgacgcggcccagccggcccaggttcagctggtgcagtctgg
agctgaggtgaagaagcctggggcctcagtgaaggtctcctgcaaggcttctggttacacctttacc
gactacgccatgaactgggtgcgacaggcccctggacaagggcttgagtggatgggagtgatcagca
ccttcagcggtaacacaaacttcaaccagaagttcaagggcagagtcaccatgaccacagacacatc
cacgagcacagcctacatggagctgaggagcctgagatctgacgacacggccgtgtattactgtgcg
agaagcgactactacggcccatacttcgactactggggccagggcaccaccctgaccgtgtccagcg
gcggtggcggatccggcggtggcggatccggcggtggcggatccgatattgtgatgacccagactcc
actctctctgt
C3 scFV FC2 gBLOCk sequence:
(DNA)
(SEQ ID NO: 267)
tattgtgatgacccagactccactctctctgtccgtcacccctggacagccggcctccatctcctgc
aggtctagtcagaccattgtccatagtaatggaaacacctatttggagtggtacctgcagaagccag
gccagtctccacagctcctgatctataaggtttccaaccggttctctggagtgccagataggttcag
tggcagcgggtcagggacagatttcacactgaaaatcagccgggtggaggctgaggatgttggggtt
tattactgcttccaaggtagccacgtgcctttcaccttcggcggagggaccaaggtggagatcaaac
gaactgagcccaaatcttgtgacaaaactcacacatgcccaccgtgcccagcacctgaactcctggg
gggaccgtcagtcttcctcttccccccaaaacccaaggacaccctcatgatctcccggacccctgag
gtcacatgcgtggtggtggacgtgagccacgaagaccctgaggtcaagttcaactggtacgtggacg
gcgtggaggtgcataatgccaagacaaagccgcgggaggagcagtacaacagcacgtaccgtgtggt
cagc
pSECTag2 C8 scFV-FC
(DNA)
(SEQ ID NO: 268)
atggagacagacacactcctgctatgggtactgctgctctgggttccaggttccactggtgacgcgg
cccagccggccgaggtgcagctggtggagtctgggggaggcctggtcaagcctggggggtccctgag
actctcctgtgcagcctctggattcaccttcagtggctatgccatgagctgggtccgccaggctcca
gggaaggggctggagtgggtctcaaccattagtagtggcggaacctacatatactaccctgactcag
tgaagggccgattcaccatctccagagacaacgccaagaactcactgtatctgcaaatgaacagcct
gagagccgaggacacggccgtgtattactgtgcgagactgggcggcgataactattatgaatattgg
ggcaaagggaccacggtcaccgtctcctccggcggtggcggatccggcggtggcggatccggcggtg
gcggatccgacatcgtgatgacccagtctccagactccctggctgtgtctctgggcgagagggccac
catcaactgcagggccagcaagagtgttagcaccagcggctacagctacatgcactggtaccagcag
aaaccaggacagcctcctaagctgctcatttacctggtgtctaacctggaatccggggtccctgacc
gattcagtggcagcgggtctgggacagatttcactctcaccatcagcagcctgcaggctgaagatgt
ggcagtttattactgtcaacacattcgggaactgaccaggagtgaattcggcggagggaccaaggtg
gagatcaaacgaactgagcccaaatcttgtgacaaaactcacacatgcccaccgtgcccagcacctg
aactcctggggggaccgtcagtcttcctcttccccccaaaacccaaggacaccctcatgatctcccg
gacccctgaggtcacatgcgtggtggtggacgtgagccacgaagaccctgaggtcaagttcaactgg
tacgtggacggcgtggaggtgcataatgccaagacaaagccgcgggaggagcagtacaacagcacgt
accgtgtggtcagcgtcctcaccgtcctgcaccaggactggctgaatggcaaggagtacaagtgcaa
ggtctccaacaaagccctcccagcccccatcgagaaaaccatctccaaagccaaagggcagccccga
gaaccacaggtgtacaccctgcccccatcccgggaggagatgaccaagaaccaggtcagcctgacct
gcctggtcaaaggcttctatcccagcgacatcgccgtggagtgggagagcaatgggcagccggagaa
caactacaagaccacgcctcccgtgctggactccgacggctccttcttcctctacagcaagctcacc
gtggacaagagcaggtggcagcaggggaacgtcttctcatgctccgtgatgcatgaggctctgcaca
accactacacgcagaagagcctctccctgtctccgggtaaatgataa
(amino acids)
(SEQ ID NO: 269)
METDTLLLWVLLLWVPGSTGDAAQPAEVQLVESGGGLVKPGGSLRLSCAASGFTFSGYAMSWVRQAPGKGLEW
VSTISSGGTYIYYPDSVKGRFTISRDNAKNSLYLQMNSLRAEDTAVYYCARLGGDNYYEYWGKGTTVTVSSGG
GGSGGGGSGGGGSDIVMTQSPDSLAVSLGERATINCRASKSVSTSGYSYMHWYQQKPGQPPKLLIYLVSNLES
GVPDRFSGSGSGTDFTLTISSLQAEDVAVYYCQHIRELTRSEFGGGTKVEIKRTEPKSCDKTHTCPPCPAPEL
LGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGK**
C8 scFV FC 1 gBLOCk sequence:
(DNA)
(SEQ ID NO: 270)
tgctctgggttccaggttccactggtgacgcggcccagccggccgaggtgcagctggtggagtctgg
gggaggcctggtcaagcctggggggtccctgagactctcctgtgcagcctctggattcaccttcagt
ggctatgccatgagctgggtccgccaggctccagggaaggggctggagtgggtctcaaccattagta
gtggcggaacctacatatactaccctgactcagtgaagggccgattcaccatctccagagacaacgc
caagaactcactgtatctgcaaatgaacagcctgagagccgaggacacggccgtgtattactgtgcg
agactgggcggcgataactattatgaatattggggcaaagggaccacggtcaccgtctcctccggcg
gtggcggatccggcggtggcggatccggcggtggcggatccgacatcgtgatgacccagtctccaga
ctccctgg
C8 scFV FC2 gBLOCk sequence:
(DNA)
(SEQ ID NO: 271)
catcgtgatgacccagtctccagactccctggctgtgtctctgggcgagagggccaccatcaactgc
agggccagcaagagtgttagcaccagcggctacagctacatgcactggtaccagcagaaaccaggac
agcctcctaagctgctcatttacctggtgtctaacctggaatccggggtccctgaccgattcagtgg
cagcgggtctgggacagatttcactctcaccatcagcagcctgcaggctgaagatgtggcagtttat
tactgtcaacacattcgggaactgaccaggagtgaattcggcggagggaccaaggtggagatcaaac
gaactgagcccaaatcttgtgacaaaactcacacatgcccaccgtgcccagcacctgaactcctggg
gggaccgtcagtcttcctcttccccccaaaacccaaggacaccctcatgatctcccggacccctgag
gtcacatgcgtggtggtggacgtgagccacgaagaccctgaggtcaagttcaactggtacgtggacg
gcgtggaggtgcataatgccaagacaaagccgcgggaggagcagtacaacagcacgtaccgtgtggt
cagc
Human IgG1 Fc sequence:
(DNA)
(SEQ ID NO: 272)
gagcccaaatcttgtgacaaaactcacacatgcccaccgtgcccagcacctgaactcctggggggaccgtcag
tcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctc
caaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggc
agccggagaacaactacaagaccacgcctcccgtgctggactccgacggctccttcttcctctacagcaagct
caccgtggacaagagcaggtggcagcaggggaacgtcttctcatgctccgtgatgcatgaggctctgcacaac
cactacacgcagaagagcctctccctgtctccgggtaaatgataa
(amino acids)
(SEQ ID NO: 273)
EPKSCDKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGNVFSCSVMHEALHN
HYTQKSLSLSPGK**
Human IgG1 CH2-CH3 domain sequence:
(DNA)
(SEQ ID NO: 274)
ccgtgcccagcacctgaactcctggggggaccgtcagtcttcctcttccccccaaaacccaaggacaccctca
tgatctcccggacccctgaggtcacatgcgtggtggtggacgtgagccacgaagaccctgaggtcaagttcaa
ctggtacgtggacggcgtggaggtgcataatgccaagacaaagccgcgggaggagcagtacaacagcacgtac
cgtgtggtcagcgtcctcaccgtcctgcaccaggactggctgaatggcaaggagtacaagtgcaaggtctcca
acaaagccctcccagcccccatcgagaaaaccatctccaaagccaaagggcagccccgagaaccacaggtgta
caccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgc
tggactccgacggctccttcttcctctacagcaagctcaccgtggacaagagcaggtggcagcaggggaacgt
cttctcatgctccgtgatgcatgaggctctgcacaaccactacacgcagaagagcctctccctgtctccgggt
aaatgataa
(amino acids)
(SEQ ID NO: 275)
PCPAPELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALHNHYTQKSLSLSPG
K**
Human IgG1 CH3 domain sequence:
(DNA)
(SEQ ID NO: 276)
gggcagccccgagaaccacaggtgtacaccctgcccccatcccgggaggagatgaccaagaaccaggtcagcc
tgacctgcctggtcaaaggcttctatcccagcgacatcgccgtggagtgggagagcaatgggcagccggagaa
caactacaagaccacgcctcccgtgctggactccgacggctccttcttcctctacagcaagctcaccgtggac
aagagcaggtggcagcaggggaacgtcttctcatgctccgtgatgcatgaggctctgcacaaccactacacgc
agaagagcctctccctgtctccgggtaaatgataa
(amino acids)
(SEQ ID NO: 277)
GQPREPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK**
Human IgG1 Fc Y407R sequence:
(DNA)
(SEQ ID NO: 278)
gagcccaaatcttgtgacaaaactcacacatgcccaccgtgcccagcacctgaactcctggggggaccgtcag
tcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctc
caaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggc
agccggagaacaactacaagaccacgcctcccgtgctggactccgacggctccttcttcctcaggagcaagct
caccgtggacaagagcaggtggcagcaggggaacgtcttctcatgctccgtgatgcatgaggctctgcacaac
cactacacgcagaagagcctctccctgtctccgggtaaatgataa
(amino acids)
(SEQ ID NO: 279)
EPKSCDKTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLRSKLTVDKSRWQQGNVFSCSVMHEALHN
HYTQKSLSLSPGK**
Human IgG1 Fc F405Q sequence:
(DNA)
(SEQ ID NO: 280)
gagcccaaatcttgtgacaaaactcacacatgcccaccgtgcccagcacctgaactcctggggggaccgtcag
tcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctc
caaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggc
agccggagaacaactacaagaccacgcctcccgtgctggactccgacggctccttccagctctacagcaagct
caccgtggacaagagcaggtggcagcaggggaacgtcttctcatgctccgtgatgcatgaggctctgcacaac
cactacacgcagaagagcctctccctgtctccgggtaaatgataa
(amino acids)
(SEQ ID NO: 281)
EPKSCDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFQLYSKLTVDKSR
WQQGNVFSCSVMHEALHNHYTQKSLSLSPGK**
Human IgG1 Fc T394D sequence:
(DNA)
(SEQ ID NO: 282)
gagcccaaatcttgtgacaaaactcacacatgcccaccgtgcccagcacctgaactcctggggggaccgtcag
tcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctc
caaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggc
agccggagaacaactacaagaccgaccctcccgtgctggactccgacggctccttcttcctctacagcaagct
caccgtggacaagagcaggtggcagcaggggaacgtcttctcatgctccgtgatgcatgaggctctgcacaac
cactacacgcagaagagcctctccctgtctccgggtaaatgataa
(amino acids)
(SEQ ID NO: 283)
EPKSCDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTDPPVLDSDGSFFLYSKLTVDKSR
WQQGNVFSCSVMHEALHNHYTQKSLSLSPGK**
Human IgG1 Fc T366W/L368W sequence:
(DNA)
(SEQ ID NO: 284)
gagcccaaatcttgtgacaaaactcacacatgcccaccgtgcccagcacctgaactcctggggggaccgtcag
tcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctc
caaagccaaagggcagccccgagaaccacaggtgtacaccctgcccccatcccgggaggagatgaccaagaac
caggtcagcctgtggtgctgggtcaaaggcttctatcccagcgacatcgccgtggagtgggagagcaatgggc
agccggagaacaactacaagaccacgcctcccgtgctggactccgacggctccttcttcctctacagcaagct
caccgtggacaagagcaggtggcagcaggggaacgtcttctcatgctccgtgatgcatgaggctctgcacaac
cactacacgcagaagagcctctccctgtctccgggtaaatgataa
(amino acids)
(SEQ ID NO: 285)
EPKSCDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSREEMTKNQVSLWCWVKGFYPSDIAVEWESNGQPENNYKTTPPVLDSDGSFFLYSKLTVDKSR
WQQGNVFSCSVMHEALHNHYTQKSLSLSPGK**
Human IgG1 Fc T364R/L368R sequence:
(DNA)
(SEQ ID NO: 286)
gagcccaaatcttgtgacaaaactcacacatgcccaccgtgcccagcacctgaactcctggggggaccgtcag
tcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaag
acaaagccgcgggaggagcagtacaacagcacgtaccgtgtggtcagcgtcctcaccgtcctgcaccaggact
ggctgaatggcaaggagtacaagtgcaaggtctccaacaaagccctcccagcccccatcgagaaaaccatctc
caaagccaaagggcagccccgagaaccacaggtgtacaccctgcccccatcccgggaggagatgaccaagaac
caggtcaggctgacctgcagggtcaaaggcttctatcccagcgacatcgccgtggagtgggagagcaatgggc
agccggagaacaactacaagaccacgcctcccgtgctggactccgacggctccttcttcctctacagcaagct
caccgtggacaagagcaggtggcagcaggggaacgtcttctcatgctccgtgatgcatgaggctctgcacaac
cactacacgcagaagagcctctccctgtctccgggtaaatgataa
(amino acids)
(SEQ ID NO: 287)
EPKSCDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSREEMTKNQVRLTCRVKGFYPSDIAVEWESNGQPENNYKTTPPVLDSDGSFFLYSKLTVDKSR
WQQGNVFSCSVMHEALHNHYTQKSLSLSPGK**
Human IgG1 Fc hingeless sequence:
(DNA)
(SEQ ID NO: 288)
gcacctgaactcctggggggaccgtcagtcttcctcttccccccaaaacccaaggacaccctcatgatctccc
ggacccctgaggtcacatgcgtggtggtggacgtgagccacgaagaccctgaggtcaagttcaactggtacgt
ggacggcgtggaggtgcataatgccaagacaaagccgcgggaggagcagtacaacagcacgtaccgtgtggtc
agcgtcctcaccgtcctgcaccaggactggctgaatggcaaggagtacaagtgcaaggtctccaacaaagccc
tcccagcccccatcgagaaaaccatctccaaagccaaagggcagccccgagaaccacaggtgtacaccctgcc
cccatcccgggaggagatgaccaagaaccaggtcagcctgacctgcctggtcaaaggcttctatcccagcgac
atcgccgtggagtgggagagcaatgggcagccggagaacaactacaagaccacgcctcccgtgctggactccg
acggctccttcttcctctacagcaagctcaccgtggacaagagcaggtggcagcaggggaacgtcttctcatg
ctccgtgatgcatgaggctctgcacaaccactacacgcagaagagcctctccctgtctccgggtaaatgataa
(amino acids)
(SEQ ID NO: 289)
APELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALHNHYTQKSLSLSPGK**
Human IgG1 G237A FC sequence:
(DNA)
(SEQ ID NO: 290)
gagcccaaatcttgtgacaaaactcacacatgcccaccgtgcccagcacctgaactcctgggggccc
cgtcagtcttcctcttccccccaaaacccaaggacaccctcatgatctcccggacccctgaggtcac
atgcgtggtggtggacgtgagccacgaagaccctgaggtcaagttcaactggtacgtggacggcgtg
gaggtgcataatgccaagacaaagccgcgggaggagcagtacaacagcacgtaccgtgtggtcagcg
tcctcaccgtcctgcaccaggactggctgaatggcaaggagtacaagtgcaaggtctccaacaaagc
cctcccagcccccatcgagaaaaccatctccaaagccaaagggcagccccgagaaccacaggtgtac
accctgcccccatcccgggaggagatgaccaagaaccaggtcagcctgacctgcctggtcaaaggct
tctatcccagcgacatcgccgtggagtgggagagcaatgggcagccggagaacaactacaagaccac
gcctcccgtgctggactccgacggctccttcttcctctacagcaagctcaccgtggacaagagcagg
tggcagcaggggaacgtcttctcatgctccgtgatgcatgaggctctgcacaaccactacacgcaga
agagcctctccctgtctccgggtaaa
(amino acids)
(SEQ ID NO: 291)
EPKSCDKTHTCPPCPAPELLGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
Human IgG1 L234A/L235A FC sequence:
(DNA)
(SEQ ID NO: 292)
gagcccaaatcttgtgacaaaactcacacatgcccaccgtgcccagcacctgaagccgccgggggac
cgtcagtcttcctcttccccccaaaacccaaggacaccctcatgatctcccggacccctgaggtcac
atgcgtggtggtggacgtgagccacgaagaccctgaggtcaagttcaactggtacgtggacggcgtg
gaggtgcataatgccaagacaaagccgcgggaggagcagtacaacagcacgtaccgtgtggtcagcg
tcctcaccgtcctgcaccaggactggctgaatggcaaggagtacaagtgcaaggtctccaacaaagc
cctcccagcccccatcgagaaaaccatctccaaagccaaagggcagccccgagaaccacaggtgtac
accctgcccccatcccgggaggagatgaccaagaaccaggtcagcctgacctgcctggtcaaaggct
tctatcccagcgacatcgccgtggagtgggagagcaatgggcagccggagaacaactacaagaccac
gcctcccgtgctggactccgacggctccttcttcctctacagcaagctcaccgtggacaagagcagg
tggcagcaggggaacgtcttctcatgctccgtgatgcatgaggctctgcacaaccactacacgcaga
agagcctctccctgtctccgggtaaa
(amino acids)
(SEQ ID NO: 293)
EPKSCDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
CAR-T E6 CD3z sequence:
(DNA)
(SEQ ID NO: 294)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggggggg
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaaacgacaaccccg
gcccccagaccaccaacgccagcccccaccatcgccagccaacccctgtctctgagaccagaagcct
gtaggcctgccgccggtggagctgtgcacacaagaggactggatttcgcctgtgatatctacatttg
ggccccgctcgcaggcacatgtggagtgctcctcctctccctggtgattaccctgtactgccgcgtt
aagttctcccgatcagccgacgcgcctgcttacaagcagggccagaaccaactgtacaacgagctga
atctcggtagacgggaagagtacgacgtgttggacaaacggagaggccgcgacccagaaatgggcgg
caagcctcgcaggaaaaacccccaggagggactgtacaatgagttgcagaaagataagatggcagaa
gcttatagcgagatcggaatgaagggggaaaggagacgagggaaaggacacgacggcctttatcagg
gcctgtccacagcaacaaaagatacgtatgacgccctccatatgcaggcacttccaccacggtgata
a
(amino acids)
(SEQ ID NO: 295)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKTTTP
APRPPTPAPTIASQPLSLRPEACRPAAGGAVHTRGLDFACDIYIWAPLAGTCGVLLLSLVITLYCRV
KFSRSADAPAYKQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR**
**
CAR-T E6 CD3z gBLOCK sequence:
(DNA)
(SEQ ID NO: 296)
tggagctgtgcacacaagaggactggatttcgcctgtgatatctacatttgggccccgctcgcaggcacatgt
ggagtgctcctcctctccctggtgattaccctgtactgccgcgttaagttctcccgatcagccgacgcgcctg
cttacaagcagggccagaaccaactgtacaacgagctgaatctcggtagacgggaagagtacgacgtgttgga
caaacggagaggccgcgacccagaaatgggcggcaagcctcgcaggaaaaacccccaggagggactgtacaat
gagttgcagaaagataagatggcagaagcttatagcgagatcggaatgaagggggaaaggagacgagggaaag
gacacgacggcctttatcagggcctgtccacagcaacaaaagatacgtatgacgccctccatatgcaggcact
tccaccacggtgataagtttaaacccgctgatcagcctcgactgtgc
CAR-T E6 CD28/CD3z sequence:
(DNA)
(SEQ ID NO: 297)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggggggg
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaaacgacaaccccg
gcccccagaccaccaacgccagcccccaccatcgccagccaacccctgtctctgagaccagaagcct
gtaggcctgccgccggtggagctgtgcacacaagaggactggatttcgcctgtgatatctacatttg
ggccccgctcgcaggcacatgtggagtgctcctcctctccctggtgattaccctgtactgcagaagc
aagcggtctcggctcctgcattctgattacatgaacatgaccccaagaagaccaggccccaccagga
aacattaccagccctacgctccgccacgcgacttcgctgcctaccggtcccgcgttaagttctcccg
atcagccgacgcgcctgcttacaagcagggccagaaccaactgtacaacgagctgaatctcggtaga
cgggaagagtacgacgtgttggacaaacggagaggccgcgacccagaaatgggcggcaagcctcgca
ggaaaaacccccaggagggactgtacaatgagttgcagaaagataagatggcagaagcttatagcga
gatcggaatgaagggggaaaggagacgagggaaaggacacgacggcctttatcagggcctgtccaca
gcaacaaaagatacgtatgacgccctccatatgcaggcacttccaccacggtgataa
(amino acids)
(SEQ ID NO: 298)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKTTTP
APRPPTPAPTIASQPLSLRPEACRPAAGGAVHTRGLDFACDIYIWAPLAGTCGVLLLSLVITLYCRS
KRSRLLHSDYMNMTPRRPGPTRKHYQPYAPPRDFAAYRSRVKFSRSADAPAYKQGQNQLYNELNLGR
REEYDVLDKRRGRDPEMGGKPRRKNPQEGLYNELQKDKMAEAYSEIGMKGERRRGKGHDGLYQGLST
ATKDTYDALHMQALPPR**
CAR-T E6 CD28/CD3z g BLOCK sequence:
(DNA)
(SEQ ID NO: 299)
tggagctgtgcacacaagaggactggatttcgcctgtgatatctacatttgggccccgctcgcaggcacatgt
ggagtgctcctcctctccctggtgattaccctgtactgcagaagcaagcggtctcggctcctgcattctgatt
acatgaacatgaccccaagaagaccaggccccaccaggaaacattaccagccctacgctccgccacgcgactt
cgctgcctaccggtcccgcgttaagttctcccgatcagccgacgcgcctgcttacaagcagggccagaaccaa
ctgtacaacgagctgaatctcggtagacgggaagagtacgacgtgttggacaaacggagaggccgcgacccag
aaatgggcggcaagcctcgcaggaaaaacccccaggagggactgtacaatgagttgcagaaagataagatggc
agaagcttatagcgagatcggaatgaagggggaaaggagacgagggaaaggacacgacggcctttatcagggc
ctgtccacagcaacaaaagatacgtatgacgccctccatatgcaggcacttccaccacggtgataagtttaaa
cccgctgatcagcctcgactgtgc
CAR-T E6 4-1BB/CD3z sequence:
(DNA)
(SEQ ID NO: 300)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggggggg
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaaacgacaaccccg
gcccccagaccaccaacgccagcccccaccatcgccagccaacccctgtctctgagaccagaagcct
gtaggcctgccgccggtggagctgtgcacacaagaggactggatttcgcctgtgatatctacatttg
ggccccgctcgcaggcacatgtggagtgctcctcctctccctggtgattaccctgtactgcaaaagg
ggccgcaaaaaactcctttacatttttaagcagccttttatgaggccagtacagacgactcaagagg
aagacgggtgctcatgccgctttcctgaggaggaggaaggagggtgcgaactgcgcgttaagttctc
ccgatcagccgacgcgcctgcttacaagcagggccagaaccaactgtacaacgagctgaatctcggt
agacgggaagagtacgacgtgttggacaaacggagaggccgcgacccagaaatgggcggcaagcctc
gcaggaaaaacccccaggagggactgtacaatgagttgcagaaagataagatggcagaagcttatag
cgagatcggaatgaagggggaaaggagacgagggaaaggacacgacggcctttatcagggcctgtcc
acagcaacaaaagatacgtatgacgccctccatatgcaggcacttccaccacggtgataa
(amino acids)
(SEQ ID NO: 301)
MALPVTALLLPLALLLHAARPEVQLVESGGGLVKPGGSLRLSCAASGFTFSRYGMSWVRQAPGKRLEWVSTIS
GGGTYIYYPDSVKGRFTISRDNAKNTLYLQMNSLRAEDTAVYYCTRDNYGRNYDYGMDYWGQGTLVTVSSGGG
GSGGGGSGGGGSEIVLTQSPATLSLSPGERATLTCSATSSVSYIHWYQQRPGQSPRLLIYSTSNLASGIPARF
SGSGSGSDYTLTISSLEPEDFAVYYCQQRSSSPFTFGSGTKVEIKTTTPAPRPPTPAPTIASQPLSLRPEACR
PAAGGAVHTRGLDFACDIYIWAPLAGTCGVLLLSLVITLYCKRGRKKLLYIFKQPFMRPVQTTQEEDGCSCRF
PEEEEGGCELRVKFSRSADAPAYKQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR**
CAR-T E6 4-1BB/CD3z gBLOCK sequence:
(DNA)
(SEQ ID NO: 302)
tggagctgtgcacacaagaggactggatttcgcctgtgatatctacatttgggccccgctcgcaggcacatgt
ggagtgctcctcctctccctggtgattaccctgtactgcaaaaggggccgcaaaaaactcctttacattttta
agcagccttttatgaggccagtacagacgactcaagaggaagacgggtgctcatgccgctttcctgaggagga
ggaaggagggtgcgaactgcgcgttaagttctcccgatcagccgacgcgcctgcttacaagcagggccagaac
caactgtacaacgagctgaatctcggtagacgggaagagtacgacgtgttggacaaacggagaggccgcgacc
cagaaatgggcggcaagcctcgcaggaaaaacccccaggagggactgtacaatgagttgcagaaagataagat
ggcagaagcttatagcgagatcggaatgaagggggaaaggagacgagggaaaggacacgacggcctttatcag
ggcctgtccacagcaacaaaagatacgtatgacgccctccatatgcaggcacttccaccacggtgataagttt
aaacccgctgatcagcctcgactgtgc
CAR-T E6 CD28/4-1BB/CD3z sequence:
(DNA)
(SEQ ID NO: 303)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggggggg
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaaacgacaaccccg
gcccccagaccaccaacgccagcccccaccatcgccagccaacccctgtctctgagaccagaagcct
gtaggcctgccgccggtggagctgtgcacacaagaggactggatttcgcctgtgatatctacatttg
ggccccgctcgcaggcacatgtggagtgctcctcctctccctggtgattaccctgtactgcagaagc
aagcggtctcggctcctgcattctgattacatgaacatgaccccaagaagaccaggccccaccagga
aacattaccagccctacgctccgccacgcgacttcgctgcctaccggtccaaaaggggccgcaaaaa
actcctttacatttttaagcagccttttatgaggccagtacagacgactcaagaggaagacgggtgc
tcatgccgctttcctgaggaggaggaaggagggtgcgaactgcgcgttaagttctcccgatcagccg
acgcgcctgcttacaagcagggccagaaccaactgtacaacgagctgaatctcggtagacgggaaga
gtacgacgtgttggacaaacggagaggccgcgacccagaaatgggcggcaagcctcgcaggaaaaac
ccccaggagggactgtacaatgagttgcagaaagataagatggcagaagcttatagcgagatcggaa
tgaagggggaaaggagacgagggaaaggacacgacggcctttatcagggcctgtccacagcaacaaa
agatacgtatgacgccctccatatgcaggcacttccaccacggtgataa
(amino acids)
(SEQ ID NO: 304)
MALPVTALLLPLALLLHAARPEVQLVESGGGLVKPGGSLRLSCAASGFTFSRYGMSWVRQAPGKRLEWVSTIS
GGGTYIYYPDSVKGRFTISRDNAKNTLYLQMNSLRAEDTAVYYCTRDNYGRNYDYGMDYWGQGTLVTVSSGGG
GSGGGGSGGGGSEIVLTQSPATLSLSPGERATLTCSATSSVSYIHWYQQRPGQSPRLLIYSTSNLASGIPARF
SGSGSGSDYTLTISSLEPEDFAVYYCQQRSSSPFTFGSGTKVEIKTTTPAPRPPTPAPTIASQPLSLRPEACR
PAAGGAVHTRGLDFACDIYIWAPLAGTCGVLLLSLVITLYCRSKRSRLLHSDYMNMTPRRPGPTRKHYQPYAP
PRDFAAYRSKRGRKKLLYIFKQPFMRPVQTTQEEDGCSCRFPEEEEGGCELRVKFSRSADAPAYKQGQNQLYN
ELNLGRREEYDVLDKRRGRDPEMGGKPRRKNPQEGLYNELQKDKMAEAYSEIGMKGERRRGKGHDGLYQGLST
ATKDTYDALHMQALPPR**
CAR-T E6 CD28/4-1BB/CD3z gBLOCK sequence:
(DNA)
(SEQ ID NO: 305)
atagggagacccaagctggctagttaagcttggtaccgagggccaccatggccctgcccgtgaccgc
tttgctgctccccctggcgctgctgctgcacgccgccaggccagaggtccagctggttgagagtggc
ggtgggctggttaagcctggcggctccctgcggctgagctgcgccgcgagtggatttactttcagcc
gatatgggatgagttgggtgcggcaagctcccgggaagaggctggaatgggtctcaacaatctccgg
ggggggcacttacatctattaccccgactcagtcaaggggagatttaccatttcacgagacaacgct
aagaataccctgtatttgcagatgaattctctgagagcagaggacacagctgtttactattgtaccc
gcgacaactatggcaggaactacgactacggtatggactattggggacaagggacattggttacagt
gagcagtggcggcgggggcagcggaggaggaggcagcggtggggggggcagcgagatagtgctcacg
cagtcacccgcgactctcagtctctcacctggggaacgagctaccctgacgtgctctgctacctcct
cagtgtcatatattcactggtatcagcaacggcccgggcagtcccctagattgctcatttatagtac
ctctaatctggcctcaggtatccctgcacgattttctggatctggttcaggttctgattacaccctc
actatctctagcctggagcctgaagactttgccgtttattactgccagcagaggtctagctccccat
tcacctttgggagtgggaccaaggttgaaattaaaacgacaaccccggcccccagaccaccaacgcc
agcccccaccatcgccagccaacccctgtctctgagaccagaagcctgtaggcctgccgccggtgga
gctgtgcacacaagaggactggatttcgcctgtgatatctacatttgggccccgctcgcaggcacat
gtggagtgctcctcctctccctggtgattaccctgtactgcagaagcaagcggtctcggctcctgca
ttctgattacatgaacatgaccccaagaagaccaggccccaccaggaaacattaccagccctacgct
ccgccacgcgacttcgctgcctaccggtccaaaaggggccgcaaaaaactcctttacatttttaagc
agccttttatgaggccagtacagacgactcaagaggaagacgggtgctcatgccgctttcctgagga
ggaggaaggagggtgcgaactgcgcgttaagttctcccgatcagccgacgcgcctgcttacaagcag
ggccagaaccaactgtacaacgagctgaatctcggtagacgggaagagtacgacgtgttggacaaac
ggagaggccgcgacccagaaatgggcggcaagcctcgcaggaaaaacccccaggagggactgtacaa
tgagttgcagaaagataagatggcagaagcttatagcgagatcggaatgaagggggaaaggagacga
gggaaaggacacgacggcctttatcagggcctgtccacagcaacaaaagatacgtatgacgccctcc
atatgcaggcacttccaccacggtgataagtttaaacccgctgatcagcctcgactgtgc
CAR-T C2 CD28/4-1BB/CD3z sequence:
(DNA)
(SEQ ID NO: 306)
atggccttgccagtgacggccctgctgctgccattggctcttctgttgcacgctgccaggcctgaagtgcagc
tcgtagagagtggcgggggactggtgaagcccggtggaagcctcagactcagttgcgccgcctcaggtttcac
tttttcaggttacgccatgtcctgggtaagacaggcaccggggaaaggactcgagtgggtgtctactatcagc
tcaggaggcacttatatatattatcctgactctgtaaaaggccgatttacgatttctcgcgacaatgcaaaga
actccctctacctccaaatgaacagtcttagggcagaagacactgctgtatactattgtgcacgcctoggcgg
cgacaactactacgagtactttgacgtgtgggggaaagggactaccgtgacagtttcaagoggaggaggtggc
tcaggtggaggcgggtcaggggggggaggaagtgatattgtgctcacacaatccccagcctccctggctgtgt
ctccoggccaacgcgctacaattacatgtcgggcctccaaaagcgtgagcaccagcggctacagctacatgca
ctggtatcaacagaaaccaggacaaccccccaaactgttgatttatctcgcttcaaacttggagtccggcgtg
cctgcgcgcttttcagggagtgggagcggcacagattttacgctgactatcaaccccgtagaagcaaacgata
cagcgaattattattgtcaacattcccgggaactcccctttacgttcggcgggggcacaaaggtcgaaattaa
gagaaccacgacaaccccggcccccagaccaccaacgccagcccccaccatcgccagccaacccctgtctctg
agaccagaagcctgtaggcctgccgccggtggagctgtgcacacaagaggactggatttcgcctgtgatatct
acatttgggccccgctcgcaggcacatgtggagtgctcctcctctccctggtgattaccctgtactgcagaag
caagcggtctcggctcctgcattctgattacatgaacatgaccccaagaagaccaggccccaccaggaaacat
taccagccctacgctccgccacgcgacttcgctgcctaccggtccaaaaggggccgcaaaaaactcctttaca
tttttaagcagccttttatgaggccagtacagacgactcaagaggaagacgggtgctcatgccgctttcctga
ggaggaggaaggagggtgcgaactgcgcgttaagttctcccgatcagccgacgcgcctgcttacaagcagggc
cagaaccaactgtacaacgagctgaatctcggtagacgggaagagtacgacgtgttggacaaacggagaggcc
gcgacccagaaatgggcggcaagcctcgcaggaaaaacccccaggagggactgtacaatgagttgcagaaaga
taagatggcagaagcttatagcgagatcggaatgaagggggaaaggagacgagggaaaggacacgacggcctt
tatcagggcctgtccacagcaacaaaagatacgtatgacgccctccatatgcaggcacttccaccacggtgat
aa
(amino acids)
(SEQ ID NO: 307)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSSGGGGSGGGGSGGGGSDIVLTQSPAS
LAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFA
CDIYIWAPLAGTCGVLLLSLVITLYCRSKRSRLLHSDYMNMTPRRPGPTRKHYQPYAPPRDFAAYRSKRGRKK
LLYIFKQPFMRPVQTTQEEDGCSCRFPEEEEGGCELRVKFSRSADAPAYKQGQNQLYNELNLGRREEYDVLDK
RRGRDPEMGGKPRRKNPQEGLYNELQKDKMAEAYSEIGMKGERRRGKGHDGLYQGLSTATKDTYDALHMQALP
PR**
CAR-T C2-1 gBLOCK sequence:
(DNA)
(SEQ ID NO: 308)
atagggagacccaagctggctagttaagcttggtaccgagggccaccatggccttgccagtgacggccctgct
gctgccattggctcttctgttgcacgctgccaggcctgaagtgcagctcgtagagagtggcgggggactggtg
aagcccggtggaagcctcagactcagttgcgccgcctcaggtttcactttttcaggttacgccatgtcctggg
taagacaggcaccggggaaaggactcgagtgggtgtctactatcagctcaggaggcacttatatatattatcc
tgactctgtaaaaggccgatttacgatttctcgcgacaatgcaaagaactccctctacctccaaatgaacagt
cttagggcagaagacactgctgtatactattgtgcacgcctcggcggcgacaactactacgagtactttgacg
tgtgggggaaagggactaccgtgacagtttcaagcggaggaggtggctcaggtggaggcgggtcagggggggg
aggaagtgatattgtgctcacacaatccccagcctccctggc
CAR-T C2-2 gBLOCK sequence:
(DNA)
(SEQ ID NO: 309)
aagtgatattgtgctcacacaatccccagcctccctggctgtgtctcccggccaacgcgctacaattacatgt
cgggcctccaaaagcgtgagcaccagcggctacagctacatgcactggtatcaacagaaaccaggacaacccc
ccaaactgttgatttatctcgcttcaaacttggagtccggcgtgcctgcgcgcttttcagggagtgggagcgg
cacagattttacgctgactatcaaccccgtagaagcaaacgatacagcgaattattattgtcaacattcccgg
gaactcccctttacgttcggcgggggcacaaaggtcgaaattaagagaaccacgacaaccccggcccccagac
caccaacgccagcccccaccatcgccagccaacccctgtctctgagaccagaagcctgtaggcctgccgccgg
tggagctgtgcacacaagaggactggatttcgcctgtgatatctacatttgggccccgctcgcaggcacatgt
ggagtgc
CAR E6 Fc/8/4-1BB/CD3z sequence:
(DNA)
(SEQ ID NO: 310)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaagagcccaaatct
tgtgacaaaactcacacatgcccaccgtgcccagcacctgaactcctggggggaccgtcagtcttcc
tcttccccccaaaacccaaggacaccctcatgatctcccggacccctgaggtcacatgcgtggtggt
ggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcc
tgcaccaggactggctgaatggcaaggagtacaagtgcaaggtctccaacaaagccctcccagcccc
catcgagaaaaccatctccaaagccaaagggcagccccgagaaccacaggtgtacaccctgccccca
tcccgggaggagatgaccaagaaccaggtcagcctgacctgcctggtcaaaggcttctatcccagcg
acatcgccgtggagtgggagagcaatgggcagccggagaacaactacaagaccacgcctcccgtgct
ggactccgacggctccttcttcctctacagcaagctcaccgtggacaagagcaggtggcagcagggg
aacgtcttctcatgctccgtgatgcatgaggctctgcacaaccactacacgcagaagagcctctccc
tgtctccgggtaaaatctacatttgggccccgctcgcaggcacatgtggagtgctcctcctctccct
ggtgattaccctgtactgcaaaaggggccgcaaaaaactcctttacatttttaagcagccttttatg
aggccagtacagacgactcaagaggaagacgggtgctcatgccgctttcctgaggaggaggaaggag
ggtgcgaactgcgcgttaagttctcccgatcagccgacgcgcctgcttacaagcagggccagaacca
actgtacaacgagctgaatctcggtagacgggaagagtacgacgtgttggacaaacggagaggccgc
gacccagaaatgggcggcaagcctcgcaggaaaaacccccaggagggactgtacaatgagttgcaga
aagataagatggcagaagcttatagcgagatcggaatgaagggggaaaggagacgagggaaaggaca
cgacggcctttatcagggcctgtccacagcaacaaaagatacgtatgacgccctccatatgcaggca
cttccaccacggtgataa
(amino acids)
(SEQ ID NO: 311)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKEPKS
CDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQG
NVFSCSVMHEALHNHYTQKSLSLSPGKIYIWAPLAGTCGVLLLSLVITLYCKRGRKKLLYIFKQPFM
RPVQTTQEEDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDTYDALHMQA
LPPR**
E6 CAR pCDH gBLOCK sequence:
(DNA)
(SEQ ID NO: 312)
acgctgttttgacctccatagaagattctagagctagctgtagagcttggtaccgagggccaccatg
gccctgcccgtgaccgctttgctgctccccctggcgctgctgctgcacgccgccaggccagaggtcc
agctggttgagagtggcggtgggctggttaagcctggcggctccctgcggctgagctgcgccgcgag
tggatttactttcagccgatatgggatgagttgggtgcggcaagctcccgggaagaggctggaatgg
gtctcaacaatctccggggggggcacttacatctattaccccgactcagtcaaggggagatttacca
tttcacgagacaacgctaagaataccctgtatttgcagatgaattctctgagagcagaggacacagc
tgtttactattgtacccgcgacaactatggcaggaactacgactacggtatggactattggggacaa
gggacattggttacagtgagcagtggcggcgggggcagcggaggaggaggcagcggtggcggaggca
gcgagatagtgctcacgcagtcacccgcgactctcagtctctcacctggggaacgagctaccctgac
gtgctctgctacctcctcagtgtcatatattcactggtatcagcaacggcccgggcagtcccctaga
ttgctcatttatagtacctctaatctggcctcaggtatccctgc
E6 CAR Fc pCDH gBLOCK sequence:
(DNA)
(SEQ ID NO: 313)
agtacctctaatctggcctcaggtatccctgcacgattttctggatctggttcaggttctgattaca
ccctcactatctctagcctggagcctgaagactttgccgtttattactgccagcagaggtctagctc
cccattcacctttgggagtgggaccaaggttgaaattaaagagcccaaatcttgtgacaaaactcac
acatgcccaccgtgcccagcacctgaactcctggggggaccgtcagtcttcctcttccccccaaaac
ccaaggacaccctcatgatctcccggacccctgaggtcacatgcgtggtggtggacgtgagccacga
agaccctgaggtcaagttcaactggtacgtggacggcgtggaggtgcataatgccaagacaaagccg
cgggaggagcagtacaacagcacgtaccgtgtggtcagcgtcctcaccgtcctgcaccaggactggc
tgaatggcaaggagtacaagtgcaaggtctccaacaaagccctcccagcccccatcgagaaaaccat
ctccaaagccaaagggcagccccgagaaccacaggtgtacaccctgcccccatcccgggaggagatg
accaagaaccaggtcagcctgacctgcctggtcaaaggcttctatcccagcgacatcgccgtggagt
gggagagcaatgggcagccggagaacaactacaagaccacgcctcccgtgctg
E6 CAR 8BB3 pCDH gBLOCK sequence:
(DNA)
(SEQ ID NO: 314)
agaacaactacaagaccacgcctcccgtgctggactccgacggctccttcttcctctacagcaagct
caccgtggacaagagcaggtggcagcaggggaacgtcttctcatgctccgtgatgcatgaggctctg
cacaaccactacacgcagaagagcctctccctgtctccgggtaaaatctacatttgggccccgctcg
caggcacatgtggagtgctcctcctctccctggtgattaccctgtactgcaaaaggggccgcaaaaa
actcctttacatttttaagcagccttttatgaggccagtacagacgactcaagaggaagacgggtgc
tcatgccgctttcctgaggaggaggaaggagggtgcgaactgcgcgttaagttctcccgatcagccg
acgcgcctgcttacaagcagggccagaaccaactgtacaacgagctgaatctcggtagacgggaaga
gtacgacgtgttggacaaacggagaggccgcgacccagaaatgggcggcaagcctcgcaggaaaaac
ccccaggagggactgtacaatgagttgcagaaagataagatggcagaagcttatagcgagatcggaa
tgaagggggaaaggagacgagggaaaggacacgacggcctttatcagggcctgtccacagcaacaaa
agatacgtatgacgccctccatatgcaggcacttccaccacggtgataagtttaaacccgctgatca
ggcggccgcgaaggatctgcgatcgctccggtgcccgtcag
CAR E6 FcH/8/4-1BB/CD3z sequence:
(DNA)
(SEQ ID NO: 315)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaagcacctgaactc
ctggggggaccgtcagtcttcctcttccccccaaaacccaaggacaccctcatgatctcccggaccc
ctgaggtcacatgcgtggtggtggacgtgagccacgaagaccctgaggtcaagttcaactggtacgt
ggacggcgtggaggtgcataatgccaagacaaagccgcgggaggagcagtacaacagcacgtaccgt
gtggtcagcgtcctcaccgtcctgcaccaggactggctgaatggcaaggagtacaagtgcaaggtct
ccaacaaagccctcccagcccccatcgagaaaaccatctccaaagccaaagggcagccccgagaacc
acaggtgtacaccctgcccccatcccgggaggagatgaccaagaaccaggtcagcctgacctgcctg
gtcaaaggcttctatcccagcgacatcgccgtggagtgggagagcaatgggcagccggagaacaact
acaagaccacgcctcccgtgctggactccgacggctccttcttcctctacagcaagctcaccgtgga
caagagcaggtggcagcaggggaacgtcttctcatgctccgtgatgcatgaggctctgcacaaccac
tacacgcagaagagcctctccctgtctccgggtaaaatctacatttgggccccgctcgcaggcacat
gtggagtgctcctcctctccctggtgattaccctgtactgcaaaaggggccgcaaaaaactccttta
catttttaagcagccttttatgaggccagtacagacgactcaagaggaagacgggtgctcatgccgc
tttcctgaggaggaggaaggagggtgcgaactgcgcgttaagttctcccgatcagccgacgcgcctg
cttacaagcagggccagaaccaactgtacaacgagctgaatctcggtagacgggaagagtacgacgt
gttggacaaacggagaggccgcgacccagaaatgggcggcaagcctcgcaggaaaaacccccaggag
ggactgtacaatgagttgcagaaagataagatggcagaagcttatagcgagatcggaatgaaggggg
aaaggagacgagggaaaggacacgacggcctttatcagggcctgtccacagcaacaaaagatacgta
tgacgccctccatatgcaggcacttccaccacggtgataa
(amino acids)
(SEQ ID NO: 316)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKAPEL
LGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEALHNH
YTQKSLSLSPGKIYIWAPLAGTCGVLLLSLVITLYCKRGRKKLLYIFKQPFMRPVQTTQEEDGCSCR
FPEEEEGGCELRVKFSRSADAPAYKQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR**
E6 CAR FcH pCDH gBLOCK sequence:
(DNA)
(SEQ ID NO: 317)
agtacctctaatctggcctcaggtatccctgcacgattttctggatctggttcaggttctgattaca
ccctcactatctctagcctggagcctgaagactttgccgtttattactgccagcagaggtctagctc
cccattcacctttgggagtgggaccaaggttgaaattaaagcacctgaactcctggggggaccgtca
gtcttcctcttccccccaaaacccaaggacaccctcatgatctcccggacccctgaggtcacatgcg
tggtggtggacgtgagccacgaagaccctgaggtcaagttcaactggtacgtggacggcgtggaggt
gcataatgccaagacaaagccgcgggaggagcagtacaacagcacgtaccgtgtggtcagcgtcctc
accgtcctgcaccaggactggctgaatggcaaggagtacaagtgcaaggtctccaacaaagccctcc
cagcccccatcgagaaaaccatctccaaagccaaagggcagccccgagaaccacaggtgtacaccct
gcccccatcccgggaggagatgaccaagaaccaggtcagcctgacctgcctggtcaaaggcttctat
cccagcgacatcgccgtggagtgggagagcaatgggcagccggagaacaactacaagaccacgcctc
ccgtgctg
CAR E6 Fc/4/4-1BB/CD3z sequence:
(DNA)
(SEQ ID NO: 318)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaagagcccaaatct
tgtgacaaaactcacacatgcccaccgtgcccagcacctgaactcctggggggaccgtcagtcttcc
tcttccccccaaaacccaaggacaccctcatgatctcccggacccctgaggtcacatgcgtggtggt
ggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcc
tgcaccaggactggctgaatggcaaggagtacaagtgcaaggtctccaacaaagccctcccagcccc
catcgagaaaaccatctccaaagccaaagggcagccccgagaaccacaggtgtacaccctgccccca
tcccgggaggagatgaccaagaaccaggtcagcctgacctgcctggtcaaaggcttctatcccagcg
acatcgccgtggagtgggagagcaatgggcagccggagaacaactacaagaccacgcctcccgtgct
ggactccgacggctccttcttcctctacagcaagctcaccgtggacaagagcaggtggcagcagggg
aacgtcttctcatgctccgtgatgcatgaggctctgcacaaccactacacgcagaagagcctctccc
tgtctccgggtaaaatggccctgattgtgctggggggcgtcgccggcctcctgcttttcattgggct
aggcatcttcttcaaaaggggccgcaaaaaactcctttacatttttaagcagccttttatgaggcca
gtacagacgactcaagaggaagacgggtgctcatgccgctttcctgaggaggaggaaggagggtgcg
aactgcgcgttaagttctcccgatcagccgacgcgcctgcttacaagcagggccagaaccaactgta
caacgagctgaatctcggtagacgggaagagtacgacgtgttggacaaacggagaggccgcgaccca
gaaatgggcggcaagcctcgcaggaaaaacccccaggagggactgtacaatgagttgcagaaagata
agatggcagaagcttatagcgagatcggaatgaagggggaaaggagacgagggaaaggacacgacgg
cctttatcagggcctgtccacagcaacaaaagatacgtatgacgccctccatatgcaggcacttcca
ccacggtgataa
(amino acids)
(SEQ ID NO: 319)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKEPKS
CDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTVDKSRWQQG
NVFSCSVMHEALHNHYTQKSLSLSPGKMALIVLGGVAGLLLFIGLGIFFKRGRKKLLYIFKQPFMRP
VQTTQEEDGCSCRFPEEEEGGCELRVKFSRSADAPAYKQGQNQLYNELNLGRREEYDVLDKRRGRDP
EMGGKPRRKNPQEGLYNELQKDKMAEAYSEIGMKGERRRGKGHDGLYQGLSTATKDTYDALHMQALP
PR**
E6 CAR 44BB3 pCDH gBLOCK sequence:
(DNA)
(SEQ ID NO: 320)
agaacaactacaagaccacgcctcccgtgctggactccgacggctccttcttcctctacagcaagct
caccgtggacaagagcaggtggcagcaggggaacgtcttctcatgctccgtgatgcatgaggctctg
cacaaccactacacgcagaagagcctctccctgtctccgggtaaaatggccctgattgtgctggggg
gcgtcgccggcctcctgcttttcattgggctaggcatcttcttcaaaaggggccgcaaaaaactcct
ttacatttttaagcagccttttatgaggccagtacagacgactcaagaggaagacgggtgctcatgc
cgctttcctgaggaggaggaaggagggtgcgaactgcgcgttaagttctcccgatcagccgacgcgc
ctgcttacaagcagggccagaaccaactgtacaacgagctgaatctcggtagacgggaagagtacga
cgtgttggacaaacggagaggccgcgacccagaaatgggcggcaagcctcgcaggaaaaacccccag
gagggactgtacaatgagttgcagaaagataagatggcagaagcttatagcgagatcggaatgaagg
gggaaaggagacgagggaaaggacacgacggcctttatcagggcctgtccacagcaacaaaagatac
gtatgacgccctccatatgcaggcacttccaccacggtgataagtttaaacccgctgatcaggcggc
cgcgaaggatctgcgatcgctccggtgcccgtcag
CAR E6 FcH/4/4-1BB/CD3z sequence:
(DNA)
(SEQ ID NO: 321)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaagcacctgaactc
ctggggggaccgtcagtcttcctcttccccccaaaacccaaggacaccctcatgatctcccggaccc
ctgaggtcacatgcgtggtggtggacgtgagccacgaagaccctgaggtcaagttcaactggtacgt
ggacggcgtggaggtgcataatgccaagacaaagccgcgggaggagcagtacaacagcacgtaccgt
gtggtcagcgtcctcaccgtcctgcaccaggactggctgaatggcaaggagtacaagtgcaaggtct
ccaacaaagccctcccagcccccatcgagaaaaccatctccaaagccaaagggcagccccgagaacc
acaggtgtacaccctgcccccatcccgggaggagatgaccaagaaccaggtcagcctgacctgcctg
gtcaaaggcttctatcccagcgacatcgccgtggagtgggagagcaatgggcagccggagaacaact
acaagaccacgcctcccgtgctggactccgacggctccttcttcctctacagcaagctcaccgtgga
caagagcaggtggcagcaggggaacgtcttctcatgctccgtgatgcatgaggctctgcacaaccac
tacacgcagaagagcctctccctgtctccgggtaaaatggccctgattgtgctggggggcgtcgccg
gcctcctgcttttcattgggctaggcatcttcttcaaaaggggccgcaaaaaactcctttacatttt
taagcagccttttatgaggccagtacagacgactcaagaggaagacgggtgctcatgccgctttcct
gaggaggaggaaggagggtgcgaactgcgcgttaagttctcccgatcagccgacgcgcctgcttaca
agcagggccagaaccaactgtacaacgagctgaatctcggtagacgggaagagtacgacgtgttgga
caaacggagaggccgcgacccagaaatgggcggcaagcctcgcaggaaaaacccccaggagggactg
tacaatgagttgcagaaagataagatggcagaagcttatagcgagatcggaatgaagggggaaagga
gacgagggaaaggacacgacggcctttatcagggcctgtccacagcaacaaaagatacgtatgacgc
cctccatatgcaggcacttccaccacggtgataa
(amino acids)
(SEQ ID NO: 322)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKAPEL
LGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEALHNH
YTQKSLSLSPGKMALIVLGGVAGLLLFIGLGIFFKRGRKKLLYIFKQPFMRPVQTTQEEDGCSCRFP
EEEEGGCELRVKFSRSADAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR**
CAR E6 IgD/8/4-1BB/CD3z sequence:
(DNA)
(SEQ ID NO: 323)
atggccctgcccgtgaccgctttgctgctccccctggcgctgctgctgcacgccgccaggccagaggtccagc
tggttgagagtggcggtgggctggttaagcctggcggctccctgcggctgagctgcgccgcgagtggatttac
tttcagccgatatgggatgagttgggtgcggcaagctcccgggaagaggctggaatgggtctcaacaatctcc
ggggggggcacttacatctattaccccgactcagtcaaggggagatttaccatttcacgagacaacgctaaga
ataccctgtatttgcagatgaattctctgagagcagaggacacagctgtttactattgtacccgcgacaacta
tggcaggaactacgactacggtatggactattggggacaagggacattggttacagtgagcagtggcggcggg
ggcagcggaggaggaggcagcggtggcggaggcagcgagatagtgctcacgcagtcacccgcgactctcagtc
tctcacctggggaacgagctaccctgacgtgctctgctacctcctcagtgtcatatattcactggtatcagca
acggcccgggcagtcccctagattgctcatttatagtacctctaatctggcctcaggtatccctgcacgattt
tctggatctggttcaggttctgattacaccctcactatctctagcctggagcctgaagactttgccgtttatt
actgccagcagaggtctagctccccattcacctttgggagtgggaccaaggttgaaattaaagagtctccaaa
ggcacaggcctcctcagtgcccactgcacaaccccaagcagagggcagcctcgccaaggcaaccacagcccca
gccaccacccgtaacacaggaagaggcggcgaagagaagaaaaaggagaaggagaaagaggaacaagaagaga
gagagacaaagacaccaatctacatttgggccccgctcgcaggcacatgtggagtgctcctcctctccctggt
gattaccctgtactgcaaaaggggccgcaaaaaactcctttacatttttaagcagccttttatgaggccagta
cagacgactcaagaggaagacgggtgctcatgccgctttcctgaggaggaggaaggagggtgcgaactgcgcg
ttaagttctcccgatcagccgacgcgcctgcttacaagcagggccagaaccaactgtacaacgagctgaatct
cggtagacgggaagagtacgacgtgttggacaaacggagaggccgcgacccagaaatgggcggcaagcctcgc
aggaaaaacccccaggagggactgtacaatgagttgcagaaagataagatggcagaagcttatagcgagatcg
gaatgaagggggaaaggagacgagggaaaggacacgacggcctttatcagggcctgtccacagcaacaaaaga
tacgtatgacgccctccatatgcaggcacttccaccacggtgataa
(amino acids)
(SEQ ID NO: 324)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKESPK
AQASSVPTAQPQAEGSLAKATTAPATTRNTGRGGEEKKKEKEKEEQEERETKTPIYIWAPLAGTCGV
LLLSLVITLYCKRGRKKLLYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQKDKMAEAYSEIGMKGERR
RGKGHDGLYQGLSTATKDTYDALHMQALPPR**
E6 CAR IgD8 pcDH gBLOCK sequence:
(DNA)
(SEQ ID NO: 325)
agtacctctaatctggcctcaggtatccctgcacgattttctggatctggttcaggttctgattaca
ccctcactatctctagcctggagcctgaagactttgccgtttattactgccagcagaggtctagctc
cccattcacctttgggagtgggaccaaggttgaaattaaagagtctccaaaggcacaggcctcctca
gtgcccactgcacaaccccaagcagagggcagcctcgccaaggcaaccacagccccagccaccaccc
gtaacacaggaagaggcggcgaagagaagaaaaaggagaaggagaaagaggaacaagaagagagaga
gacaaagacaccaatctacatttgggccccgctcgcaggcacatgtggagtgctcctcctctccctg
gtgattaccctgtactgcaaaaggggccgcaaaaaactcctttacatttttaagcagccttttatga
ggccag
E6 CAR BB 3 pCDH gBLOCK sequence:
(DNA)
(SEQ ID NO: 326)
acatttttaagcagccttttatgaggccagtacagacgactcaagaggaagacgggtgctcatgccg
ctttcctgaggaggaggaaggagggtgcgaactgcgcgttaagttctcccgatcagccgacgcgcct
gcttacaagcagggccagaaccaactgtacaacgagctgaatctcggtagacgggaagagtacgacg
tgttggacaaacggagaggccgcgacccagaaatgggcggcaagcctcgcaggaaaaacccccagga
gggactgtacaatgagttgcagaaagataagatggcagaagcttatagcgagatcggaatgaagggg
gaaaggagacgagggaaaggacacgacggcctttatcagggcctgtccacagcaacaaaagatacgt
atgacgccctccatatgcaggcacttccaccacggtgataagtttaaacccgctgatcaggcggccg
cgaaggatctgcgatcgctccggtgcccgtcag
CAR E6 IgD/4/4-1BB/CD3z sequence:
(DNA)
(SEQ ID NO: 327)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaagagtctccaaag
gcacaggcctcctcagtgcccactgcacaaccccaagcagagggcagcctcgccaaggcaaccacag
ccccagccaccacccgtaacacaggaagaggcggcgaagagaagaaaaaggagaaggagaaagagga
acaagaagagagagagacaaagacaccaatggccctgattgtgctggggggcgtcgccggcctcctg
cttttcattgggctaggcatcttcttcaaaaggggccgcaaaaaactcctttacatttttaagcagc
cttttatgaggccagtacagacgactcaagaggaagacgggtgctcatgccgctttcctgaggagga
ggaaggagggtgcgaactgcgcgttaagttctcccgatcagccgacgcgcctgcttacaagcagggc
cagaaccaactgtacaacgagctgaatctcggtagacgggaagagtacgacgtgttggacaaacgga
gaggccgcgacccagaaatgggcggcaagcctcgcaggaaaaacccccaggagggactgtacaatga
gttgcagaaagataagatggcagaagcttatagcgagatcggaatgaagggggaaaggagacgaggg
aaaggacacgacggcctttatcagggcctgtccacagcaacaaaagatacgtatgacgccctccata
tgcaggcacttccaccacggtgataa
(amino acids)
(SEQ ID NO: 328)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKESPK
AQASSVPTAQPQAEGSLAKATTAPATTRNTGRGGEEKKKEKEKEEQEERETKTPMALIVLGGVAGLL
LFIGLGIFFKRGRKKLLYIFKQPFMRPVQTTQEEDGCSCRFPEEEEGGCELRVKFSRSADAPAYKQG
QNQLYNELNLGRREEYDVLDKRRGRDPEMGGKPRRKNPQEGLYNELQKDKMAEAYSEIGMKGERRRG
KGHDGLYQGLSTATKDTYDALHMQALPPR**
E6 CAR IgD4 pcDH gBLOCK sequence:
(DNA)
(SEQ ID NO: 329)
agtacctctaatctggcctcaggtatccctgcacgattttctggatctggttcaggttctgattaca
ccctcactatctctagcctggagcctgaagactttgccgtttattactgccagcagaggtctagctc
cccattcacctttgggagtgggaccaaggttgaaattaaagagtctccaaaggcacaggcctcctca
gtgcccactgcacaaccccaagcagagggcagcctcgccaaggcaaccacagccccagccaccaccc
gtaacacaggaagaggcggcgaagagaagaaaaaggagaaggagaaagaggaacaagaagagagaga
gacaaagacaccaatggccctgattgtgctggggggcgtcgccggcctcctgcttttcattgggcta
ggcatcttcttcaaaaggggccgcaaaaaactcctttacatttttaagcagccttttatgaggccag
CAR E6 X4/8/4-1BB/CD3z sequence:
(DNA)
(SEQ ID NO: 330)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaagacaagacgcac
accaagccacctaaaccagctccagaactgctcggaggtcctggcaccggaaccggaggacctacca
tcaaaccacctaagccacctaagcctgctcctaacctgctcggaggacctatctacatttgggcccc
gctcgcaggcacatgtggagtgctcctcctctccctggtgattaccctgtactgcaaaaggggccgc
aaaaaactcctttacatttttaagcagccttttatgaggccagtacagacgactcaagaggaagacg
ggtgctcatgccgctttcctgaggaggaggaaggagggtgcgaactgcgcgttaagttctcccgatc
agccgacgcgcctgcttacaagcagggccagaaccaactgtacaacgagctgaatctcggtagacgg
gaagagtacgacgtgttggacaaacggagaggccgcgacccagaaatgggcggcaagcctcgcagga
aaaacccccaggagggactgtacaatgagttgcagaaagataagatggcagaagcttatagcgagat
cggaatgaagggggaaaggagacgagggaaaggacacgacggcctttatcagggcctgtccacagca
acaaaagatacgtatgacgccctccatatgcaggcacttccaccacggtgataa
(amino acids)
(SEQ ID NO: 331)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKDKTH
TKPPKPAPELLGGPGTGTGGPTIKPPKPPKPAPNLLGGPIYIWAPLAGTCGVLLLSLVITLYCKRGR
KKLLYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQGLSTA
TKDTYDALHMQALPPR**
E6 CAR X48 pCDH gBLOCK sequence:
(DNA)
(SEQ ID NO: 332)
agtacctctaatctggcctcaggtatccctgcacgattttctggatctggttcaggttctgattaca
ccctcactatctctagcctggagcctgaagactttgccgtttattactgccagcagaggtctagctc
cccattcacctttgggagtgggaccaaggttgaaattaaagacaagacgcacaccaagccacctaaa
ccagctccagaactgctcggaggtcctggcaccggaaccggaggacctaccatcaaaccacctaagc
cacctaagcctgctcctaacctgctcggaggacctatctacatttgggccccgctcgcaggcacatg
tggagtgctcctcctctccctggtgattaccctgtactgcaaaaggggccgcaaaaaactcctttac
atttttaagcagccttttatgaggccag
CAR E6 X4/4/4-1BB/CD3z sequence:
(DNA)
(SEQ ID NO: 333)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaagacaagacgcac
accaagccacctaaaccagctccagaactgctcggaggtcctggcaccggaaccggaggacctacca
tcaaaccacctaagccacctaagcctgctcctaacctgctcggaggacctatggccctgattgtgct
ggggggcgtcgccggcctcctgcttttcattgggctaggcatcttcttcaaaaggggccgcaaaaaa
ctcctttacatttttaagcagccttttatgaggccagtacagacgactcaagaggaagacgggtgct
catgccgctttcctgaggaggaggaaggagggtgcgaactgcgcgttaagttctcccgatcagccga
cgcgcctgcttacaagcagggccagaaccaactgtacaacgagctgaatctcggtagacgggaagag
tacgacgtgttggacaaacggagaggccgcgacccagaaatgggcggcaagcctcgcaggaaaaacc
cccaggagggactgtacaatgagttgcagaaagataagatggcagaagcttatagcgagatcggaat
gaagggggaaaggagacgagggaaaggacacgacggcctttatcagggcctgtccacagcaacaaaa
gatacgtatgacgccctccatatgcaggcacttccaccacggtgataa
(amino acids)
(SEQ ID NO: 334)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKDKTH
TKPPKPAPELLGGPGTGTGGPTIKPPKPPKPAPNLLGGPMALIVLGGVAGLLLFIGLGIFFKRGRKK
LLYIFKQPFMRPVQTTQEEDGCSCRFPEEEEGGCELRVKFSRSADAPAYKQGQNQLYNELNLGRREE
YDVLDKRRGRDPEMGGKPRRKNPQEGLYNELQKDKMAEAYSEIGMKGERRRGKGHDGLYQGLSTATK
DTYDALHMQALPPR**
E6 CAR X44 pCDH gBLOCK sequence:
(DNA)
(SEQ ID NO: 335)
agtacctctaatctggcctcaggtatccctgcacgattttctggatctggttcaggttctgattaca
ccctcactatctctagcctggagcctgaagactttgccgtttattactgccagcagaggtctagctc
cccattcacctttgggagtgggaccaaggttgaaattaaagacaagacgcacaccaagccacctaaa
ccagctccagaactgctcggaggtcctggcaccggaaccggaggacctaccatcaaaccacctaagc
cacctaagcctgctcctaacctgctcggaggacctatggccctgattgtgctggggggcgtcgccgg
cctcctgcttttcattgggctaggcatcttcttcaaaaggggccgcaaaaaactcctttacattttt
aagcagccttttatgaggccag
CAR E6 8 + 4/4/4-1BB/CD3z sequence:
(DNA)
(SEQ ID NO: 336)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggcggag
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaaacgacaaccccg
gcccccagaccaccaacgccagcccccaccatcgccagccaacccctgtctctgagaccagaagcct
gtaggcctgccgccggtggagctgtgcacacaagaggactggatttcgcctgtgatatggccctgat
tgtgctggggggcgtcgccggcctcctgcttttcattgggctaggcatcttcttcaaaaggggccgc
aaaaaactcctttacatttttaagcagccttttatgaggccagtacagacgactcaagaggaagacg
ggtgctcatgccgctttcctgaggaggaggaaggagggtgcgaactgcgcgttaagttctcccgatc
agccgacgcgcctgcttacaagcagggccagaaccaactgtacaacgagctgaatctcggtagacgg
gaagagtacgacgtgttggacaaacggagaggccgcgacccagaaatgggcggcaagcctcgcagga
aaaacccccaggagggactgtacaatgagttgcagaaagataagatggcagaagcttatagcgagat
cggaatgaagggggaaaggagacgagggaaaggacacgacggcctttatcagggcctgtccacagca
acaaaagatacgtatgacgccctccatatgcaggcacttccaccacggtgataa
(amino acids)
(SEQ ID NO: 337)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKTTTP
APRPPTPAPTIASQPLSLRPEACRPAAGGAVHTRGLDFACDMALIVLGGVAGLLLFIGLGIFFKRGR
KKLLYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQGLSTA
TKDTYDALHMQALPPR**
E6 CAR CD844 pCDH gBLOCK sequence:
(DNA)
(SEQ ID NO: 338)
agtacctctaatctggcctcaggtatccctgcacgattttctggatctggttcaggttctgattaca
ccctcactatctctagcctggagcctgaagactttgccgtttattactgccagcagaggtctagctc
cccattcacctttgggagtgggaccaaggttgaaattaaaacgacaaccccggcccccagaccacca
acgccagcccccaccatcgccagccaacccctgtctctgagaccagaagcctgtaggcctgccgccg
gtggagctgtgcacacaagaggactggatttcgcctgtgatatggccctgattgtgctggggggcgt
cgccggcctcctgcttttcattgggctaggcatcttcttcaaaaggggccgcaaaaaactcctttac
atttttaagcagccttttatgaggccag
Humanized C2 scFV sequence in CAR:
(DNA)
(SEQ ID NO: 339)
gagggccaccatggccttgccagtgacggccctgctgctgccattggctcttctgttgcacgctgccaggcct
gaagtgcagctcgtagagagtggcgggggactggtgaagcccggtggaagcctcagactcagttgcgccgcct
caggtttcactttttcaggttacgccatgtcctgggtaagacaggcaccggggaaaggactcgagtgggtgtc
tactatcagctcaggaggcacttatatatattatcctgactctgtaaaaggccgatttacgatttctcgcgac
aatgcaaagaactccctctacctccaaatgaacagtcttagggcagaagacactgctgtatactattgtgcac
gcctcggcggcgacaactactacgagtactttgacgtgtgggggaaagggactaccgtgacagtttcaagcgg
aggaggtggctcaggtggaggcgggtcaggggggggaggaagtgatattgtgctcacacaatccccagcctcc
ctggctgtgtctcccggccaacgcgctacaattacatgtcgggcctccaaaagcgtgagcaccagcggctaca
gctacatgcactggtatcaacagaaaccaggacaaccccccaaactgttgatttatctcgcttcaaacttgga
gtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaagg
tcgaaattaagagaacc
(amino acids)
(SEQ ID NO: 340)
EVQLVESGGGLVKPGGSLRLSCAASGFTFSGYAMSWVRQAPGKGLEWVSTISSGGTYIYYPDSVKGR
FTISRDNAKNSLYLQMNSLRAEDTAVYYCARLGGDNYYEYFDVWGKGTTVTVSSGGGGSGGGGSGGG
GSDIVLTQSPASLAVSPGQRATITCRASKSVSTSGYSYMHWYQQKPGQPPKLLIYLASNLESGVPAR
FSGSGSGTDFTLTINPVEANDTANYYCQHSRELPFTFGGGTKVEIKRT
Humanized E6 scFV sequence in CAR:
(DNA)
(SEQ ID NO: 341)
gaggtccagctggttgagagtggcggtgggctggttaagcctggcggctccctgcggctgagctgcg
ccgcgagtggatttactttcagccgatatgggatgagttgggtgcggcaagctcccgggaagaggct
ggaatgggtctcaacaatctccggggggggcacttacatctattaccccgactcagtcaaggggaga
tttaccatttcacgagacaacgctaagaataccctgtatttgcagatgaattctctgagagcagagg
acacagctgtttactattgtacccgcgacaactatggcaggaactacgactacggtatggactattg
gggacaagggacattggttacagtgagcagtggcggcgggggcagcggaggaggaggcagcggtggg
gggggcagcgagatagtgctcacgcagtcacccgcgactctcagtctctcacctggggaacgagcta
ccctgacgtgctctgctacctcctcagtgtcatatattcactggtatcagcaacggcccgggcagtc
ccctagattgctcatttatagtacctctaatctggcctcaggtatccctgcacgattttctggatct
ggttcaggttctgattacaccctcactatctctagcctggagcctgaagactttgccgtttattact
gccagcagaggtctagctccccattcacctttgggagtgggaccaaggttgaaattaaa
(amino acids)
(SEQ ID NO: 342)
EVQLVESGGGLVKPGGSLRLSCAASGFTFSRYGMSWVRQAPGKRLEWVSTISGGGTYIYYPDSVKGR
FTISRDNAKNTLYLQMNSLRAEDTAVYYCTRDNYGRNYDYGMDYWGQGTLVTVSSGGGGSGGGGSGG
GGSEIVLTQSPATLSLSPGERATLTCSATSSVSYIHWYQQRPGQSPRLLIYSTSNLASGIPARFSGS
GSGSDYTLTISSLEPEDFAVYYCQQRSSSPFTFGSGTKVEIK
CD8 leader sequence:
(DNA)
(SEQ ID NO: 343)
atggccctgcccgtgaccgctttgctgctccccctggcgctgctgctgcacgccgccaggcca
(amino acids)
(SEQ ID NO: 344)
MALPVTALLLPLALLLHAARP
CD8 hinge domain sequence:
(DNA)
(SEQ ID NO: 345)
acgacaaccccggcccccagaccaccaacgccagcccccaccatcgccagccaacccctgtctctga
gaccagaagcctgtaggcctgccgccggtggagctgtgcacacaagaggactggatttcgcctgtga
t
(amino acids)
(SEQ ID NO: 346)
TTTPAPRPPTPAPTIASQPLSLRPEACRPAAGGAVHTRGLDFACD
CD4 hinge domain sequence:
(DNA)
(SEQ ID NO: 347)
tcgggacaggtcctgctggaatccaacatcaaggttctgcccacatggtccaccccggtgcagcca
(amino acids)
(SEQ ID NO: 348)
SGQVLLESNIKVLPTWSTPVQP
CD28 hinge domain sequence:
(DNA)
(SEQ ID NO: 349)
aaacacctttgtccaagtcccctatttcccggaccttctaagccc
(amino acids)
(SEQ ID NO: 350)
KHLCPSPLFPGPSKP
CD8 + CD4 hinge domain sequence:
(DNA)
(SEQ ID NO: 351)
acgacaaccccggcccccagaccaccaacgccagcccccaccatcgccagccaacccctgtctctga
gaccagaagcctgtaggcctgccgccggtggagctgtgcacacaagaggactggatttcgcctgtga
ttcgggacaggtcctgctggaatccaacatcaaggttctgcccacatggtccaccccggtgcagcca
(amino acids)
(SEQ ID NO: 352)
TTTPAPRPPTPAPTIASQPLSLRPEACRPAAGGAVHTRGLDFACDSGQVLLESNIKVLPTWSTPVQP
CD8 + CD28 hinge domain sequence:
(DNA)
(SEQ ID NO: 353)
acgacaaccccggcccccagaccaccaacgccagcccccaccatcgccagccaacccctgtctctga
gaccagaagcctgtaggcctgccgccggtggagctgtgcacacaagaggactggatttcgcctgtga
taaacacctttgtccaagtcccctatttcccggaccttctaagccc
(amino acids)
(SEQ ID NO: 354)
TTTPAPRPPTPAPTIASQPLSLRPEACRPAAGGAVHTRGLDFACDKHLCPSPLFPGPSKP
CD28 + CD4 hinge domain sequence:
(DNA)
(SEQ ID NO: 355)
aaacacctttgtccaagtcccctatttcccggaccttctaagccctcgggacaggtcctgctggaat
ccaacatcaaggttctgcccacatggtccaccccggtgcagcca
(amino acids)
(SEQ ID NO: 356)
KHLCPSPLFPGPSKPSGQVLLESNIKVLPTWSTPVQP
Human IgD hinge domain sequence:
(DNA)
(SEQ ID NO: 357)
gagtctccaaaggcacaggcctcctcagtgcccactgcacaaccccaagcagagggcagcctcgcca
aggcaaccacagccccagccaccacccgtaacacaggaagaggcggcgaagagaagaaaaaggagaa
ggagaaagaggaacaagaagagagagagacaaagacacca
(amino acids)
(SEQ ID NO: 358)
ESPKAQASSVPTAQPQAEGSLAKATTAPATTRNTGRGGEEKKKEKEKEEQEERETKTP
X4 linker (IgG1 and IgG2 modified hinge region) sequence:
(DNA)
(SEQ ID NO: 359)
gacaagacgcacaccaagccacctaaaccagctccagaactgctcggaggtcctggcaccggaaccg
gaggacctaccatcaaaccacctaagccacctaagcctgctcctaacctgctcggaggacct
(amino acids)
(SEQ ID NO: 360)
DKTHTKPPKPAPELLGGPGTGTGGPTIKPPKPPKPAPNLLGGP
CD3 zeta transmembrane domain sequence:
(DNA)
(SEQ ID NO: 361)
ctctgctacctgctggatggaatcctcttcatctatggtgtcattctcactgccttgttcctg
(amino acids)
(SEQ ID NO: 362)
LCYLLDGILFIYGVILTALFL
CD8 transmembrane domain sequence:
(DNA)
(SEQ ID NO: 363)
atctacatttgggccccgctcgcaggcacatgtggagtgctcctcctctccctggtgattaccctgt
actgc
(amino acids)
(SEQ ID NO: 364)
IYIWAPLAGTCGVLLLSLVITLYC
CD4 transmembrane domain sequence:
(DNA)
(SEQ ID NO: 365)
atggccctgattgtgctggggggcgtcgccggcctcctgcttttcattgggctaggcatcttcttc
(amino acids)
(SEQ ID NO: 366)
MALIVLGGVAGLLLFIGLGIFF
CD28 transmembrane domain sequence:
(DNA)
(SEQ ID NO: 367)
ttttgggtgctggtggtggttggtggagtcctggcttgctatagcttgctagtaacagtggccttta
ttattttctgggtg
(amino acids)
(SEQ ID NO: 368)
FWVLVVVGGVLACYSLLVTVAFIIFWV
4-1BB transmembrane domain sequence:
(DNA)
(SEQ ID NO: 369)
atcatctccttctttcttgcgctgacgtcgactgcgttgctcttcctgctgttcttcctcacgctcc
gtttctctgttgtt
(amino acids)
(SEQ ID NO: 370)
IISFFLALTSTALLFLLFFLTLRFSVV
OX40 transmembrane domain sequence:
(DNA)
(SEQ ID NO: 371)
gttgccgccatcctgggcctgggcctggtgctggggctgctgggccccctggccatcctgctggccc
tgtacctgctc
(amino acids)
(SEQ ID NO: 372)
VAAILGLGLVLGLLGPLAILLALYLL
CD3 zeta domain sequence:
(DNA)
(SEQ ID NO: 373)
cgcgttaagttctcccgatcagccgacgcgcctgcttacaagcagggccagaaccaactgtacaacg
agctgaatctcggtagacgggaagagtacgacgtgttggacaaacggagaggccgcgacccagaaat
gggcggcaagcctcgcaggaaaaacccccaggagggactgtacaatgagttgcagaaagataagatg
gcagaagcttatagcgagatcggaatgaagggggaaaggagacgagggaaaggacacgacggccttt
atcagggcctgtccacagcaacaaaagatacgtatgacgccctccatatgcaggcacttccaccacg
g
(amino acids)
(SEQ ID NO: 374)
RVKFSRSADAPAYKQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
CD3 zeta domain variant sequence:
(DNA)
(SEQ ID NO: 375)
agagtgaagttcagcaggagcgcagacgcccccgcgtaccagcagggccagaaccagctctataacg
agctcaatctaggacgaagagaggagtacgatgttttggacaagagacgtggccgggaccctgagat
ggggggaaagccgagaaggaagaaccctcaggaaggcctgtacaatgaactgcagaaagataagatg
gcggaggcctacagtgagattgggatgaaaggcgagcgccggaggggcaaggggcacgatggccttt
accagggtctcagtacagccaccaaggacacctacgacgcccttcacatgcaggccctgccccctcg
c
(amino acids)
(SEQ ID NO: 376)
RVKFSRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
CD28 domain sequence:
(DNA)
(SEQ ID NO: 377)
agaagcaagcggtctcggctcctgcattctgattacatgaacatgaccccaagaagaccaggcccca
ccaggaaacattaccagccctacgctccgccacgcgacttcgctgcctaccggtcc
(amino acids)
(SEQ ID NO: 378)
RSKRSRLLHSDYMNMTPRRPGPTRKHYQPYAPPRDFAAYRS
4-1BB domain sequence:
(DNA)
(SEQ ID NO: 379)
aaaaggggccgcaaaaaactcctttacatttttaagcagccttttatgaggccagtacagacgactc
aagaggaagacgggtgctcatgccgctttcctgaggaggaggaaggagggtgcgaactg
(amino acids)
(SEQ ID NO: 380)
KRGRKKLLYIFKQPFMRPVQTTQEEDGCSCRFPEEEEGGCEL
OX40 domain sequence:
(DNA)
(SEQ ID NO: 381)
cggagggaccagaggctgccccccgatgcccacaagccccctgggggaggcagtttccggaccccca
tccaagaggagcaggccgacgcccactccaccctggccaagatc
(amino acids)
(SEQ ID NO: 382)
RRDQRLPPDAHKPPGGGSFRTPIQEEQADAHSTLAKI
Humanized anti CD3 scFV clone 12F6 (VH-VL) sequence:
(DNA)
(SEQ ID NO: 383)
caggtgcagctggtgcagagcggaggtggagtggtccaacctggaagatctctgagactgagctgta
aggctagcgggtacacgttcacatcttacacgatgcactgggtgaggcaagcccccggtaagggcct
ggaatggatcggatatataaaccccagctcagggtataccaaatataatcagaagttcaaagatcgg
ttcacgatttctgctgataaaagtaagtccaccgctttcctgcagatggactcactcaggccagaag
atactggtgtttatttctgtgcaaggtggcaggactacgacgtgtactttgactattgggggcaggg
gacgcctgtaacagtatcaagcggcggtggcggatccggcggtggcggatccggcggtggcggatcc
gatattcagatgacccagagcccgagcagcctgagcgcgagcgtgggcgatcgcgtgaccatgacct
gccgcgcgagcagcagcgtgagctatatgcattggtatcagcagaccccgggcaaagcgccgaaacc
gtggatttatgcgaccagcaacctggcgagcggcgtgccgagccgctttagcggcagcggcagcggc
accgattataccctgaccattagcagcctgcagccggaagatattgcgacctattattgccagcagt
ggagcagcaacccgccgacctttggccagggcaccaaactgcagattacccgc
(amino acids)
(SEQ ID NO: 384)
QVQLVQSGGGVVQPGRSLRLSCKASGYTFTSYTMHWVRQAPGKGLEWIGYINPSSGYTKYNQKFKDR
FTISADKSKSTAFLQMDSLRPEDTGVYFCARWQDYDVYFDYWGQGTPVTVSSGGGGSGGGGSGGGGS
DIQMTQSPSSLSASVGDRVTMTCRASSSVSYMHWYQQTPGKAPKPWIYATSNLASGVPSRFSGSGSG
TDYTLTISSLQPEDIATYYCQQWSSNPPTFGQGTKLQITR
Humanized anti CD3 scFV clone 12F6 (VL-VH) sequence:
(DNA)
(SEQ ID NO: 385)
gatattcagatgacccagagcccgagcagcctgagcgcgagcgtgggcgatcgcgtgaccatgacct
gccgcgcgagcagcagcgtgagctatatgcattggtatcagcagaccccgggcaaagcgccgaaacc
gtggatttatgcgaccagcaacctggcgagcggcgtgccgagccgctttagcggcagcggcagcggc
accgattataccctgaccattagcagcctgcagccggaagatattgcgacctattattgccagcagt
ggagcagcaacccgccgacctttggccagggcaccaaactgcagattacccgcggcggtggcggatc
cggcggtggcggatccggcggtggcggatcccaggtgcagctggtgcagagcggaggtggagtggtc
caacctggaagatctctgagactgagctgtaaggctagcgggtacacgttcacatcttacacgatgc
actgggtgaggcaagcccccggtaagggcctggaatggatcggatatataaaccccagctcagggta
taccaaatataatcagaagttcaaagatcggttcacgatttctgctgataaaagtaagtccaccgct
ttcctgcagatggactcactcaggccagaagatactggtgtttatttctgtgcaaggtggcaggact
acgacgtgtactttgactattgggggcaggggacgcctgtaacagtatcaagc
(amino acids)
(SEQ ID NO: 386)
DIQMTQSPSSLSASVGDRVTMTCRASSSVSYMHWYQQTPGKAPKPWIYATSNLASGVPSRFSGSGSG
TDYTLTISSLQPEDIATYYCQQWSSNPPTFGQGTKLQITRGGGGSGGGGSGGGGSQVQLVQSGGGVV
QPGRSLRLSCKASGYTFTSYTMHWVRQAPGKGLEWIGYINPSSGYTKYNQKFKDRFTISADKSKSTA
FLQMDSLRPEDTGVYFCARWQDYDVYFDYWGQGTPVTVSS
Humanized anti CD3 scFV clone OKT3 (VH-VL) sequence:
(DNA)
(SEQ ID NO: 387)
caggtgcagctggtgcagagcggaggcggagtggtgcagcctggaagaagcctgcgcctgagctgca
aagcgagcggctatacctttacccgctataccatgcattgggtgcgccaggcgccgggcaaaggcct
ggaatggattggctatattaacccgagccgcggctataccaactataaccagaaagtgaaagatcgc
tttaccattagcaccgataaaagcaaaagcaccgcgtttctgcagatggatagcctgcgcccggaag
ataccgcggtgtattattgcgcgcgctattatgatgatcattattgcctggattattggggccaggg
caccaccctgaccgtgagcagcggcggtggcggatccggcggtggcggatccggcggtggcggatcc
gatattcagatgacccagagcccgagcagcctgagcgcgagcgtgggcgatcgcgtgaccattacct
gcagcgcgagcagcagcgtgagctatatgaactggtatcagcagaccccgggcaaagcgccgaaacg
ctggatttatgataccagcaaactggcgagcggcgtgccgagccgctttagcggcagcggcagcggc
accgattatacctttaccattagcagcctgcagccggaagatattgcgacctattattgccagcagt
ggagcagcaacccgtttacctttggccagggcaccaaactgcagattacccgc
(amino acids)
(SEQ ID NO: 388)
QVQLVQSGGGVVQPGRSLRLSCKASGYTFTRYTMHWVRQAPGKGLEWIGYINPSRGYTNYNQKVKDR
FTISTDKSKSTAFLQMDSLRPEDTAVYYCARYYDDHYCLDYWGQGTTLTVSSGGGGSGGGGSGGGGS
DIQMTQSPSSLSASVGDRVTITCSASSSVSYMNWYQQTPGKAPKRWIYDTSKLASGVPSRFSGSGSG
TDYTFTISSLQPEDIATYYCQQWSSNPFTFGQGTKLQITR
Humanized anti CD3 scFV clone OKT3 (VH-VM) sequence:
(DNA)
(SEQ ID NO: 389)
gatattcagatgacccagagcccgagcagcctgagcgcgagcgtgggcgatcgcgtgaccattacct
gcagcgcgagcagcagcgtgagctatatgaactggtatcagcagaccccgggcaaagcgccgaaacg
ctggatttatgataccagcaaactggcgagcggcgtgccgagccgctttagcggcagcggcagcggc
accgattatacctttaccattagcagcctgcagccggaagatattgcgacctattattgccagcagt
ggagcagcaacccgtttacctttggccagggcaccaaactgcagattacccgcggcggtggcggatc
cggcggtggcggatccggcggtggcggatcccaggtgcagctggtgcagagcggaggcggagtggtg
cagcctggaagaagcctgcgcctgagctgcaaagcgagcggctatacctttacccgctataccatgc
attgggtgcgccaggcgccgggcaaaggcctggaatggattggctatattaacccgagccgcggcta
taccaactataaccagaaagtgaaagatcgctttaccattagcaccgataaaagcaaaagcaccgcg
tttctgcagatggatagcctgcgcccggaagataccgcggtgtattattgcgcgcgctattatgatg
atcattattgcctggattattggggccagggcaccaccctgaccgtgagcagc
(amino acids)
(SEQ ID NO: 390)
DIQMTQSPSSLSASVGDRVTITCSASSSVSYMNWYQQTPGKAPKRWIYDTSKLASGVPSRFSGSGSG
TDYTFTISSLQPEDIATYYCQQWSSNPFTFGQGTKLQITRGGGGSGGGGSGGGGSQVQLVQSGGGVV
QPGRSLRLSCKASGYTFTRYTMHWVRQAPGKGLEWIGYINPSRGYTNYNQKVKDRFTISTDKSKSTA
FLQMDSLRPEDTAVYYCARYYDDHYCLDYWGQGTTLTVSS
HumanizeE6 scFV (VH-VL) sequence:
(DNA)
(SEQ ID NO: 391)
gaggtgcagctggtggagtctgggggaggcctggtcaagcctggggggtccctgagactctcctgtg
cagcctctggattcaccttcagtaggtatggcatgagctgggtccgccaggctccagggaagaggct
ggagtgggtctcaaccattagtggcggaggcacctacatatactacccagactcagtgaagggccga
ttcaccatctccagagacaacgccaagaacaccctgtatctgcaaatgaacagcctgagagccgagg
acacggctgtgtattactgtaccagagataactatggccgcaactatgattatggcatggattattg
gggccagggcaccctggtgaccgtgagcagcggcggtggcggatccggcggtggcggatccggcggt
ggcggatccgaaattgtgttgacacagtctccagccaccctgtctttgtctccaggggaaagagcca
ccctcacctgcagcgccaccagcagtgttagctacatccactggtaccaacagaggcctggccagag
ccccaggctcctcatctatagcacctccaacctggccagcggcatcccagccaggttcagtggcagt
gggtctgggagcgactacactctcaccatcagcagcctagagcctgaagattttgcagtttattact
gtcagcagcgtagcagctcccctttcacctttggcagcggcaccaaagtggaaattaaa
(amino acids)
(SEQ ID NO: 392)
EVQLVESGGGLVKPGGSLRLSCAASGFTFSRYGMSWVRQAPGKRLEWVSTISGGGTYIYYPDSVKGR
FTISRDNAKNTLYLQMNSLRAEDTAVYYCTRDNYGRNYDYGMDYWGQGTLVTVSSGGGGSGGGGSGG
GGSEIVLTQSPATLSLSPGERATLTCSATSSVSYIHWYQQRPGQSPRLLIYSTSNLASGIPARFSGS
GSGSDYTLTISSLEPEDFAVYYCQQRSSSPFTFGSGTKVEIK
HumanizeE6 scFV (VL-VH) sequence:
(DNA)
(SEQ ID NO: 393)
gaaattgtgttgacacagtctccagccaccctgtctttgtctccaggggaaagagccaccctcacct
gcagcgccaccagcagtgttagctacatccactggtaccaacagaggcctggccagagccccaggct
cctcatctatagcacctccaacctggccagcggcatcccagccaggttcagtggcagtgggtctggg
agcgactacactctcaccatcagcagcctagagcctgaagattttgcagtttattactgtcagcagc
gtagcagctcccctttcacctttggcagcggcaccaaagtggaaattaaaggcggtggcggatccgg
cggtggcggatccggcggtggcggatccgaggtgcagctggtggagtctgggggaggcctggtcaag
cctggggggtccctgagactctcctgtgcagcctctggattcaccttcagtaggtatggcatgagct
gggtccgccaggctccagggaagaggctggagtgggtctcaaccattagtggcggaggcacctacat
atactacccagactcagtgaagggccgattcaccatctccagagacaacgccaagaacaccctgtat
ctgcaaatgaacagcctgagagccgaggacacggctgtgtattactgtaccagagataactatggcc
gcaactatgattatggcatggattattggggccagggcaccctggtgaccgtgagcagc
(amino acids)
(SEQ ID NO: 394)
EIVLTQSPATLSLSPGERATLTCSATSSVSYIHWYQQRPGQSPRLLIYSTSNLASGIPARFSGSGSG
SDYTLTISSLEPEDFAVYYCQQRSSSPFTFGSGTKVEIKGGGGSGGGGSGGGGSEVQLVESGGGLVK
PGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSS
HumanizeC2 scFV (VH-VL) sequence:
(DNA)
(SEQ ID NO: 395)
gaggtgcagctggtggagtctgggggaggcctggtcaagcctggggggtccctgagactctcctgtg
cagcctctggattcaccttcagtggctatgccatgagctgggtccgccaggctccagggaaggggct
ggagtgggtctcaaccattagtagtggcggaacctacatatactaccccgactcagtgaagggccga
ttcaccatctccagagacaacgccaagaactcactgtatctgcaaatgaacagcctgagagccgagg
acacggccgtgtattactgtgcgagacttgggggggataattactacgaatacttcgatgtctgggg
caaagggaccacggtcaccgtctcctccggcggtggcggatccggcggtggcggatccggcggtggc
ggatccgacattgtgctgacccagtctccagcctccttggccgtgtctccaggacagagggccacca
tcacctgcagagccagtaagagtgtcagtaccagcggatactcctacatgcactggtatcagcagaa
accaggacaacctcctaaactcctgatttacctggcatccaatctggagagcggggtcccagccagg
ttcagcggcagtgggtctgggaccgatttcaccctcacaattaatcctgtggaagctaatgatactg
caaattattactgtcagcacagtagggagctgcctttcacattcggcggagggaccaaggtggagat
caaacgaact
(amino acids)
(SEQ ID NO: 396)
EVQLVESGGGLVKPGGSLRLSCAASGFTFSGYAMSWVRQAPGKGLEWVSTISSGGTYIYYPDSVKGR
FTISRDNAKNSLYLQMNSLRAEDTAVYYCARLGGDNYYEYFDVWGKGTTVTVSSGGGGSGGGGSGGG
GSDIVLTQSPASLAVSPGQRATITCRASKSVSTSGYSYMHWYQQKPGQPPKLLIYLASNLESGVPAR
FSGSGSGTDFTLTINPVEANDTANYYCQHSRELPFTFGGGTKVEIKRT
HumanizeE6 scFV (VL-VH) sequence:
(DNA)
(SEQ ID NO: 397)
gacattgtgctgacccagtctccagcctccttggccgtgtctccaggacagagggccaccatcacct
gcagagccagtaagagtgtcagtaccagcggatactcctacatgcactggtatcagcagaaaccagg
acaacctcctaaactcctgatttacctggcatccaatctggagagcggggtcccagccaggttcagc
ggcagtgggtctgggaccgatttcaccctcacaattaatcctgtggaagctaatgatactgcaaatt
attactgtcagcacagtagggagctgcctttcacattcggcggagggaccaaggtggagatcaaacg
aactggcggtggcggatccggcggtggcggatccggcggtggcggatccgaggtgcagctggtggag
tctgggggaggcctggtcaagcctggggggtccctgagactctcctgtgcagcctctggattcacct
tcagtggctatgccatgagctgggtccgccaggctccagggaaggggctggagtgggtctcaaccat
tagtagtggcggaacctacatatactaccccgactcagtgaagggccgattcaccatctccagagac
aacgccaagaactcactgtatctgcaaatgaacagcctgagagccgaggacacggccgtgtattact
gtgcgagacttgggggggataattactacgaatacttcgatgtctggggcaaagggaccacggtcac
cgtctcctcc
(amino acids)
(SEQ ID NO: 398)
DIVLTQSPASLAVSPGQRATITCRASKSVSTSGYSYMHWYQQKPGQPPKLLIYLASNLESGVPARFS
GSGSGTDFTLTINPVEANDTANYYCQHSRELPFTFGGGTKVEIKRTGGGGSGGGGSGGGGSEVQLVE
SGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYFDVWGKGTTVTVSS
G₄S₁ linker sequence:
(DNA)
(SEQ ID NO: 399)
ggcggtggcggatcc
(amino acids)
(SEQ ID NO:400)
GGGGS
[G₄S₁]x3 linker sequence:
(DNA)
(SEQ ID NO: 401)
ggcggtggcggatccggcggtggcggatccggcggtggcggatcc
(amino acids)
(SEQ ID NO: 402)
GGGGSGGGGSGGGGS
8 aa GS linker sequence:
(DNA)
(SEQ ID NO: 403)
ggcggttccggcggtggatccgga
(amino acids)
(SEQ ID NO: 404)
GGSGGGSG
12 aa GS linker sequence:
(DNA)
(SEQ ID NO: 405)
ggcggttccggcggtggatccggcggtggcggatccgga
(amino acids)
(SEQ ID NO: 406)
GGSGGGSGGGSG
13 aa GS linker sequence:
(DNA)
(SEQ ID NO: 407)
ggcggtggatccggcggtggcggatccggcggtggatcc
(amino acids)
(SEQ ID NO: 408)
GGGSGGGGSGGGS
22 aa GS linker sequence:
(DNA)
(SEQ ID NO: 409)
ggcggtggaagcggcggtggcggatccggcagcggcggaagcggcggtggcggatccggcggtgga
(amino acids)
(SEQ ID NO: 4110)
GGGSGGGGSGSGGSGGGGSGGG
24 aa GS linker sequence:
(DNA)
(SEQ ID NO: 411)
ggcggttccggcggtggatccggcggtggcggatccggaggcggttccggcggtggatccggcggtg
gcggatccgga
(amino acids)
(SEQ ID NO: 412)
GGSGGGSGGGSGGGSGGGSGGGSG
Mouse C3 Heavy chain variable region sequence:
(DNA)
(SEQ ID NO: 413)
caggtccagctgcagcagtctgggcctgagctggtgaggcctggggtctcagtgaagatttcctgcaagggtt
ccggctacagattcactgattatgctatgaactgggtgaagcagagtcatgcaaagagtctagagtggattgg
agttattagtactttctctggtaatacaaacttcaaccagaagtttaagggcaaggccacaatgactgtagac
aaatcctccagcacagcctatatggaacttgccagattgacatctgaggattctgccatgtattactgtgcaa
gatcggattactacggcccatactttgactactggggccaaggcaccactctcacagtctcctca
(amino acids)
(SEQ ID NO: 414)
QVQLQQSGPELVRPGVSVKISCKGSGYRFTDYAMNWVKQSHAKSLEWIGVISTFSGNTNFNQKFKGKATMTVD
KSSSTAYMELARLTSEDSAMYYCARSDYYGPYFDYWGQGTTLTVSS
Mouse C3 heavy chain variable framework region 1 (FWR1) sequence:
(DNA)
(SEQ ID NO: 415)
caggtccagctgcagcagtctgggcctgagctggtgaggcctggggtctcagtgaagatttcctgcaagggtt
ccggctacagattcact
(amino acids)
(SEQ ID NO: 416)
QVQLQQSGPELVRPGVSVKISCKGSGYRFT
Mouse C3 heavy chain variable complementarity determining regions 1
(CDR1) sequence:
(DNA)
(SEQ ID NO: 417)
gattatgctatgaac
(amino acids)
(SEQ ID NO: 418)
DYAMN
Mouse C3 heavy chain variable framework region 2 (FWR2) sequence:
(DNA)
(SEQ ID NO: 419)
tgggtgaagcagagtcatgcaaagagtctagagtggattgga
(amino acids)
(SEQ ID NO: 420)
WVKQSHAKSLEWIG
Mouse C3 heavy chain variable complementarity determining regions 2
(CDR2) sequence:
(DNA)
(SEQ ID NO: 421)
gttattagtactttctctggtaatacaaacttcaaccagaagtttaagggc
(amino acids)
(SEQ ID NO: 422)
VISTFSGNINFNQKFKG
Mouse C3 heavy chain variable framework region 3 (FWR3) acid sequence:
(DNA)
(SEQ ID NO: 423)
aaggccacaatgactgtagacaaatcctccagcacagcctatatggaacttgccagattgacatctgaggatt
ctgccatgtattactgtgcaaga
(amino acids)
(SEQ ID NO: 424)
KATMTVDKSSSTAYMELARLTSEDSAMYYCAR
Mouse C3 heavy chain variable complementarity determining regions 3
(CDR3) sequence:
(DNA)
(SEQ ID NO: 425)
tcggattactacggcccatactttgactac
(amino acids)
(SEQ ID NO: 426)
SDYYGPYFDY
IGHV1-18*04 heavy chain variable region sequence:
(DNA)
(SEQ ID NO: 427)
caggttcagctggtgcagtctggagctgaggtgaagaagcctggggcctcagtgaaggtctcctgca
aggcttctggttacacctttaccagctacggtatcagctgggtgcgacaggcccctggacaagggct
tgagtggatgggatggatcagcgcttacaatggtaacacaaactatgcacagaagctccagggcaga
gtcaccatgaccacagacacatccacgagcacagcctacatggagctgaggagcctgagatctgacg
acacggccgtgtattactgtgcgagaga
(amino acids)
(SEQ ID NO: 428)
QVQLVQSGAEVKKPGASVKVSCKASGYTFTSYGISWVRQAPGQGLEWMGWISAYNGNINYAQKLQGRVIMITD
TSTSTAYMELRSLRSDDTAVYYCAR
IGHV1-18*04 heavy chain variable framework region 1 (FWR1) sequence:
(DNA)
(SEQ ID NO: 429)
caggttcagctggtgcagtctggagctgaggtgaagaagcctggggcctcagtgaaggtctcctgcaaggctt
ctggttacacctttacc
(amino acids)
(SEQ ID NO: 430)
QVQLVQSGAEVKKPGASVKVSCKASGYTFT
IGHV1-18*04 heavy chain variable complementarity determining regions 1
(CDR1) sequence:
(DNA)
(SEQ ID NO: 431)
agctacggtatcagc
(amino acids)
(SEQ ID NO: 432)
SYGIS
IGHV1-18*04 heavy chain variable framework region 2 (FWR2) sequence:
(DNA)
(SEQ ID NO: 433)
tgggtgcgacaggcccctggacaagggcttgagtggatggga
(amino acids)
(SEQ ID NO: 434)
WVRQAPGQGLEWMG
IGHV1-18*04 heavy chain variable complementarity determining regions 2
(CDR2) sequence:
(DNA)
(SEQ ID NO: 435)
tggatcagcgcttacaatggtaacacaaactatgcacagaagctccagggc
(amino acids)
(SEQ ID NO: 436)
WISAYNGNTNYAQKLQG
IGHV1-18*04 heavy chain variable framework region 3 (FWR3) sequence:
(DNA)
(SEQ ID NO: 437)
agagtcaccatgaccacagacacatccacgagcacagcctacatggagctgaggagcctgagatctgacgaca
cggccgtgtattactgtgcgaga
(amino acids)
(SEQ ID NO: 438)
RVTMTTDTSTSTAYMELRSLRSDDTAVYYCAR
Humanized C3 heavy chain variable region sequence:
(DNA)
(SEQ ID NO: 439)
caggttcagctggtgcagtctggagctgaggtgaagaagcctggggcctcagtgaaggtctcctgcaaggctt
ctggttacacctttaccgactacgccatgaactgggtgcgacaggcccctggacaagggcttgagtggatggg
agtgatcagcaccttcagcggtaacacaaacttcaaccagaagttcaagggcagagtcaccatgaccacagac
acatccacgagcacagcctacatggagctgaggagcctgagatctgacgacacggccgtgtattactgtgcga
gaagcgactactacggcccatacttcgactactggggccagggcaccaccctgaccgtgtccagc
(amino acids)
(SEQ ID NO: 440)
QVQLVQSGAEVKKPGASVKVSCKASGYTFTDYAMNWVRQAPGQGLEWMGVISTFSGNTNFNQKFKGRVTMTTD
TSTSTAYMELRSLRSDDTAVYYCARSDYYGPYFDYWGQGTTLTVSS
Humanized C3 heavy chain variable framework region 1 (FWR1) acid
sequence:
(DNA)
(SEQ ID NO: 441)
caggttcagctggtgcagtctggagctgaggtgaagaagcctggggcctcagtgaaggtctcctgcaaggctt
ctggttacacctttacc
(amino acids)
(SEQ ID NO: 442)
QVQLVQSGAEVKKPGASVKVSCKASGYTFT
Humanized C3 heavy chain variable complementarity determining regions 1
(CDR1) sequence:
(DNA)
(SEQ ID NO: 443)
gactacgccatgaac
(amino acids)
(SEQ ID NO: 444)
DYAMN
Humanized C3 heavy chain variable framework region 2 (FWR2) acid
sequence:
(DNA)
(SEQ ID NO: 445)
tgggtgcgacaggcccctggacaagggcttgagtggatggga
(amino acids)
(SEQ ID NO: 446)
WVRQAPGQGLEWMG
Humanized C3 heavy chain variable complementarity determining regions 2
(CDR2) sequence:
(DNA)
(SEQ ID NO: 447)
gtgatcagcaccttcagcggtaacacaaacttcaaccagaagttcaagggc
(amino acids)
(SEQ ID NO: 448)
VISTFSGNTNFNQKFKG
Humanized C3 heavy chain variable framework region 3 (FWR3) acid
sequence:
(DNA)
(SEQ ID NO: 449)
agagtcaccatgaccacagacacatccacgagcacagcctacatggagctgaggagcctgagatctgacgaca
cggccgtgtattactgtgcgaga
(amino acids)
(SEQ ID NO: 450)
RVTMTTDTSTSTAYMELRSLRSDDTAVYYCAR
Humanized C3 heavy chain variable complementarity determining regions 3
(CDR3) sequence:
(DNA)
(SEQ ID NO: 451)
agcgactactacggcccatacttcgactac
(amino acids)
(SEQ ID NO: 452)
SDYYGPYFDY
Humanized C3 IgG1 heavy chain sequence
(DNA)
(SEQ ID NO: 453)
caggttcagctggtgcagtctggagctgaggtgaagaagcctggggcctcagtgaaggtctcctgcaaggctt
ctggttacacctttaccgactacgccatgaactgggtgcgacaggcccctggacaagggcttgagtggatggg
agtgatcagcaccttcagcggtaacacaaacttcaaccagaagttcaagggcagagtcaccatgaccacagac
acatccacgagcacagcctacatggagctgaggagcctgagatctgacgacacggccgtgtattactgtgcga
gaagcgactactacggcccatacttcgactactggggccagggcaccaccctgaccgtgtccagcgctagcac
caagggcccatcggtcttccccctggcaccctcctccaagagcacctctgggggcacagcggccctgggctgc
ctggtcaaggactacttccccgaaccggtgacggtgtcgtggaactcaggcgccctgaccagcggcgtgcaca
ccttcccggctgtcctacagtcctcaggactctactccctcagcagcgtggtgacagtgccctccagcagctt
gggcacccagacctacatctgcaacgtgaatcacaagcccagcaacaccaaggtggacaagaaagttgagccc
aaatcttgtgacaaaactcacacatgcccaccgtgcccagcacctgaactcctggggggaccgtcagtcttcc
tcttccccccaaaacccaaggacaccctcatgatctcccggacccctgaggtcacatgcgtggtggtggacgt
gagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctga
atggcaaggagtacaagtgcaaggtctccaacaaagccctcccagcccccatcgagaaaaccatctccaaagc
caaagggcagccccgagaaccacaggtgtacaccctgcccccatcccgggaggagatgaccaagaaccaggtc
agcctgacctgcctggtcaaaggcttctatcccagcgacatcgccgtggagtgggagagcaatgggcagccgg
agaacaactacaagaccacgcctcccgtgctggactccgacggctccttcttcctctacagcaagctcaccgt
ggacaagagcaggtggcagcaggggaacgtcttctcatgctccgtgatgcatgaggctctgcacaaccactac
acgcagaagagcctctccctgtctccgggtaaatgataa
(amino acids)
(SEQ ID NO: 454)
QVQLVQSGAEVKKPGASVKVSCKASGYTFTDYAMNWVRQAPGQGLEWMGVISTFSGNTNFNQKFKGRVTMTTD
TSTSTAYMELRSLRSDDTAVYYCARSDYYGPYFDYWGQGTTLTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFSCSVMHEALHNHY
TQKSLSLSPGK**
Humanized C3 IgG2 heavy chain sequence
(DNA)
(SEQ ID NO: 455)
caggttcagctggtgcagtctggagctgaggtgaagaagcctggggcctcagtgaaggtctcctgcaaggctt
ctggttacacctttaccgactacgccatgaactgggtgcgacaggcccctggacaagggcttgagtggatggg
agtgatcagcaccttcagcggtaacacaaacttcaaccagaagttcaagggcagagtcaccatgaccacagac
acatccacgagcacagcctacatggagctgaggagcctgagatctgacgacacggccgtgtattactgtgcga
gaagcgactactacggcccatacttcgactactggggccagggcaccaccctgaccgtgtccagcgcctccac
caagggcccatcggtcttccccctggcgccctgctccaggagcacctccgagagcacagccgccctgggctgc
ctggtcaaggactacttccccgaaccggtgacggtgtcgtggaactcaggcgctctgaccagcggcgtgcaca
ccttcccagctgtcctacagtcctcaggactctactccctcagcagcgtggtgaccgtgccctccagcaactt
cggcacccagacctacacctgcaacgtagatcacaagcccagcaacaccaaggtggacaagacagttgagcgc
aaatgttgtgtcgagtgcccaccgtgcccagcaccacctgtggcaggaccgtcagtcttcctcttccccccaa
aacccaaggacaccctcatgatctcccggacccctgaggtcacgtgcgtggtggtggacgtgagccacgaaga
ccccgaggtccagttcaactggtacgtggacggcgtggaggtgcataatgccaagacaaagccacgggaggag
cagttcaacagcacgttccgtgtggtcagcgtcctcaccgttgtgcaccaggactggctgaacggcaaggagt
acaagtgcaaggtctccaacaaaggcctcccagcccccatcgagaaaaccatctccaaaaccaaagggcagcc
ccgagaaccacaggtgtacaccctgcccccatcccgggaggagatgaccaagaaccaggtcagcctgacctgc
ctggtcaaaggcttctaccccagcgacatcgccgtggagtgggagagcaatgggcagccggagaacaactaca
agaccacacctcccatgctggactccgacggctccttcttcctctacagcaagctcaccgtggacaagagcag
gtggcagcaggggaacgtcttctcatgctccgtgatgcatgaggctctgcacaaccactacacgcagaagagc
ctctccctgtctccgggtaaatagtaa
(amino acids)
(SEQ ID NO: 456)
QVQLVQSGAEVKKPGASVKVSCKASGYTFTDYAMNWVRQAPGQGLEWMGVISTFSGNTNFNQKFKGRVTMTTD
TSTSTAYMELRSLRSDDTAVYYCARSDYYGPYFDYWGQGTTLTVSSASTKGPSVFPLAPCSRSTSESTAALGC
LVKDYFPEPVTVSWNSGALTSGVHTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSRWQQGNVFSCSVMHEALHNHYTQKS
LSLSPGK**
Humanized C3 heavy chain IgG1 gBLOCK sequence:
(DNA)
(SEQ ID NO: 457)
tgctctgggttccaggttccactggtgacgcggcccagccggcccaggttcagctggtgcagtctggagctga
ggtgaagaagcctggggcctcagtgaaggtctcctgcaaggcttctggttacacctttaccgactacgccatg
aactgggtgcgacaggcccctggacaagggcttgagtggatgggagtgatcagcaccttcagcggtaacacaa
acttcaaccagaagttcaagggcagagtcaccatgaccacagacacatccacgagcacagcctacatggagct
gaggagcctgagatctgacgacacggccgtgtattactgtgcgagaagcgactactacggcccatacttcgac
tactggggccagggcaccaccctgaccgtgtccagcgctagcaccaagggcccatcggtcttccccctggcac
cctcctccaagagcacctctgggggcacagcggccctgggctgcctggtcaaggactacttccccgaaccggt
gacggtgtcgtggaactcaggcgccctgaccagc
Mouse C3 Light Chain variable region sequence:
(DNA)
(SEQ ID NO: 458)
gatgttttgatgacccaaactccactctccctgcctgtcagtcttggagatcaagcctccatctcttgcagat
ctagtcagaccattgtacatagtaatggaaacacctatttagaatggtacctgcagaaaccaggccagtctcc
aaagctcctgatctacaaagtttccaaccgattttctggggtcccagacaggttcagtggcagtggatcaggg
acagatttcacactcaagatcaacagagtggaggctgaggatctgggagtttattactgctttcaaggttcac
atgttccattcacgttcggctcggggacaaagttggaaataaaa
(amino acids)
(SEQ ID NO: 459)
DVLMTQTPLSLPVSLGDQASISCRSSQTIVHSNGNTYLEWYLQKPGQSPKLLIYKVSNRFSGVPDRFSGSGSG
TDFTLKINRVEAEDLGVYYCFQGSHVPFTFGSGTKLEIK
Mouse C3 light chain variable framework region 1 (FWR1) sequence:
(DNA)
(SEQ ID NO: 460)
gatgttttgatgacccaaactccactctccctgcctgtcagtcttggagatcaagcctccatctcttgc
(amino acids)
(SEQ ID NO: 461)
DVLMTQTPLSLPVSLGDQASISC
Mouse C3 light chain variable complementarity determining regions 1
(CDR1) sequence:
(DNA)
(SEQ ID NO: 462)
agatctagtcagaccattgtacatagtaatggaaacacctatttagaa
(amino acids)
(SEQ ID NO: 463)
RSSQTIVHSNGNTYLE
Mouse C3 light chain variable framework region 2 (FWR2) sequence:
(DNA)
(SEQ ID NO: 464)
tggtacctgcagaaaccaggccagtctccaaagctcctgatctac
(amino acids)
(SEQ ID NO: 465)
WYLQKPGQSPKLLIY
Mouse C3 light chain variable complementarity determining regions 2
(CDR2) sequence:
(DNA)
(SEQ ID NO: 466)
aaagtttccaaccgattttct
(amino acids)
(SEQ ID NO: 467)
KVSNRFS
Mouse C3 light chain variable framework region 3 (FWR3) sequence:
(DNA)
(SEQ ID NO: 468)
ggggtcccagacaggttcagtggcagtggatcagggacagatttcacactcaagatcaacagagtggaggctg
aggatctgggagtttattactgc
(amino acids)
(SEQ ID NO: 469)
GVPDRFSGSGSGTDFTLKINRVEAEDLGVYYC
Mouse C3 light chain variable complementarity determining regions 3
(CDR3) sequence:
(DNA)
(SEQ ID NO: 470)
tttcaaggttcacatgttccattcacg
(amino acids)
(SEQ ID NO: 471)
FQGSHVPFT
IGKV2-29*03 light chain variable region sequence:
(DNA)
(SEQ ID NO: 472)
gatattgtgatgacccagactccactctctctgtccgtcacccctggacagccggcctccatctcctgcaagt
ctagtcagagcctcctgcatagtgatggaaagacctatttgtattggtacctgcagaagccaggccagtctcc
acagctcctgatctatgaagtttccagccggttctctggagtgccagataggttcagtggcagcgggtcaggg
acagatttcacactgaaaatcagccgggtggaggctgaggatgttggggtttattactgcatgcaaggtatac
accttcct
(amino acids)
(SEQ ID NO: 473)
DIVMTQTPLSLSVTPGQPASISCKSSQSLLHSDGKTYLYWYLQKPGQSPQLLIYEVSSRFSGVPDRFSGSGSG
TDFTLKISRVEAEDVGVYYCMQGIHLP
IGKV2-29*03 light chain variable framework region 1 (FWR1) acid sequence:
(DNA)
(SEQ ID NO: 474)
gatattgtgatgacccagactccactctctctgtccgtcacccctggacagccggcctccatctcctgc
(amino acids)
(SEQ ID NO: 475)
DIVMTQTPLSLSVTPGQPASISC
IGKV2-29*03 light chain variable complementarity determining regions 1
(CDR1) sequence:
(DNA)
(SEQ ID NO: 476)
aagtctagtcagagcctcctgcatagtgatggaaagacctatttgtat
(amino acids)
(SEQ ID NO: 477)
KSSQSLLHSDGKTYLY
IGKV2-29*03 light chain variable framework region 2 (FWR2) sequence:
(DNA)
(SEQ ID NO: 478)
tggtacctgcagaagccaggccagtctccacagctcctgatctat
(amino acids)
(SEQ ID NO: 479)
WYLQKPGQSPQLLIY
IGKV2-29*03 light chain variable complementarity determining regions 2
(CDR2) sequence:
(DNA)
(SEQ ID NO: 480)
gaagtttccagccggttc
(amino acids)
(SEQ ID NO: 481)
EVSSRFS
IGKV2-29*03 light chain variable framework region 3 (FWR3) sequence:
(DNA)
(SEQ ID NO: 482)
ggagtgccagataggttcagtggcagcgggtcagggacagatttcacactgaaaatcagccgggtggaggctg
aggatgttggggtttattactgc
(amino acids)
(SEQ ID NO: 483)
GVPDRFSGSGSGTDFTLKISRVEAEDVGVYYC
IGKV2-29*03 light chain variable complementarity determining regions3
(CDR3) sequence:
(DNA)
(SEQ ID NO: 484)
atgcaaggtatacaccttcct
(amino acids)
(SEQ ID NO: 485)
MQGIHLP
Humanized C3 light chain variable region sequence:
(DNA)
(SEQ ID NO: 486)
gatattgtgatgacccagactccactctctctgtccgtcacccctggacagccggcctccatctcct
gcaggtctagtcagaccattgtccatagtaatggaaacacctatttggagtggtacctgcagaagcc
aggccagtctccacagctcctgatctataaggtttccaaccggttctctggagtgccagataggttc
agtggcagcgggtcagggacagatttcacactgaaaatcagccgggtggaggctgaggatgttgggg
tttattactgcttccaaggtagccacgtgcctttcaccttcggcggagggaccaaggtggagatcaa
acgaact
(amino acids)
(SEQ ID NO: 487)
DIVMTQTPLSLSVTPGQPASISCRSSQTIVHSNGNTYLEWYLQKPGQSPQLLIYKVSNRFSGVPDRF
SGSGSGTDFTLKISRVEAEDVGVYYCFQGSHVPFTFGGGTKVEIKRT
Humanized C3 light chain variable framework region 1 (FWR1) acid
sequence:
(DNA)
(SEQ ID NO: 488)
gatattgtgatgacccagactccactctctctgtccgtcacccctggacagccggcctccatctcct
gc
(amino acids)
(SEQ ID NO: 489)
DIVMTQTPLSLSVTPGQPASISC
Humanized C3 light chain variable complementarity determining regions 1
(CDR1) sequence:
(DNA)
(SEQ ID NO: 490)
ggtctagtcagaccattgtccatagtaatggaaacacctatttggag
(amino acids)
(SEQ ID NO: 491)
RSSQTIVHSNGNTYLE
Humanized C3 light chain variable framework region 2 (FWR2) acid
sequence:
(DNA)
(SEQ ID NO: 492)
tggtacctgcagaagccaggccagtctccacagctcctgatctat
(amino acids)
(SEQ ID NO: 493)
WYLQKPGQSPQLLIY
Humanized C3 light chain variable complementarity determining regions 2
(CDR2) sequence:
(DNA)
(SEQ ID NO: 494)
aaggtttccaaccggttctct
(amino acids)
(SEQ ID NO: 495)
KVSNRFS
Humanized C3 light chain variable framework region 3 (FWR3) acid
sequence:
(DNA)
(SEQ ID NO: 496)
ggagtgccagataggttcagtggcagcgggtcagggacagatttcacactgaaaatcagccgggtggaggctg
aggatgttggggtttattactgc
(amino acids)
(SEQ ID NO: 497)
GVPDRFSGSGSGTDFTLKISRVEAEDVGVYYC
Humanized C3 light chain variable complementarity determining regions 3
(CDR3) sequence:
(DNA)
(SEQ ID NO: 498)
ttccaaggtagccacgtgcctttcacc
(amino acids)
(SEQ ID NO: 499)
FQGSHVPFT
Humanized C3 lambda light chain sequence
(DNA)
(SEQ ID NO: 500)
gatattgtgatgacccagactccactctctctgtccgtcacccctggacagccggcctccatctcctgcaggt
ctagtcagaccattgtccatagtaatggaaacacctatttggagtggtacctgcagaagccaggccagtctcc
acagctcctgatctataaggtttccaaccggttctctggagtgccagataggttcagtggcagcgggtcaggg
acagatttcacactgaaaatcagccgggtggaggctgaggatgttggggtttattactgcttccaaggtagcc
acgtgcctttcaccttcggcggagggaccaaggtggagatcaaacgaactggtcagcccaaggctgccccctc
ggtcactctgttcccgccctcctctgaggagcttcaagccaacaaggccacactggtgtgtctcataagtgac
ttctacccgggagccgtgacagtggcctggaaggcagatagcagccccgtcaaggcgggagtggagaccacca
caccctccaaacaaagcaacaacaagtacgcggccagcagctatctgagcctgacgcctgagcagtggaagtc
ccacagaagctacagctgccaggtcacgcatgaagggagcaccgtggagaagacagtggcccctacagaatgt
tcatagtaa
(amino acids)
(SEQ ID NO: 501)
DIVMTQTPLSLSVTPGQPASISCRSSQTIVHSNGNTYLEWYLQKPGQSPQLLIYKVSNRFSGVPDRFSGSGSG
TDFTLKISRVEAEDVGVYYCFQGSHVPFTFGGGTKVEIKRTGQPKAAPSVTLFPPSSEELQANKATLVCLISD
FYPGAVTVAWKADSSPVKAGVETTTPSKQSNNKYAASSYLSLTPEQWKSHRSYSCQVTHEGSTVEKTVAPTEC
S**
Humanized C3 Kappa light chain
(DNA)
(SEQ ID NO: 502)
gatattgtgatgacccagactccactctctctgtccgtcacccctggacagccggcctccatctcctgcaggt
ctagtcagaccattgtccatagtaatggaaacacctatttggagtggtacctgcagaagccaggccagtctcc
acagctcctgatctataaggtttccaaccggttctctggagtgccagataggttcagtggcagcgggtcaggg
acagatttcacactgaaaatcagccgggtggaggctgaggatgttggggtttattactgcttccaaggtagcc
acgtgcctttcaccttcggcggagggaccaaggtggagatcaaacgaactacggtggctgcaccatctgtctt
catcttcccgccatctgatgagcagttgaaatctggaactgcctctgttgtgtgcctgctgaataacttctat
cccagagaggccaaagtacagtggaaggtggataacgccctccaatcgggtaactcccaggagagtgtcacag
agcaggacagcaaggacagcacctacagcctcagcagcaccctgacgctgagcaaagcagactacgagaaaca
caaagtctacgcctgcgaagtcacccatcagggcctgagctcgcccgtcacaaagagcttcaacaggggagag
tgttagtaa
(amino acids)
(SEQ ID NO: 503)
DIVMTQTPLSLSVTPGQPASISCRSSQTIVHSNGNTYLEWYLQKPGQSPQLLIYKVSNRFSGVPDRFSGSGSG
TDFTLKISRVEAEDVGVYYCFQGSHVPFTFGGGTKVEIKRTTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFNRGE
C**
Humanized C3 Kappa light gBLOCK sequence:
(DNA)
(SEQ ID NO: 504)
agctggctaggtaagcttggtaccgagctcggatccacgccaccatggagacagacacactcctgct
atgggtactgctgctctgggttccaggttccactggtgacgatattgtgatgacccagactccactc
tctctgtccgtcacccctggacagccggcctccatctcctgcaggtctagtcagaccattgtccata
gtaatggaaacacctatttggagtggtacctgcagaagccaggccagtctccacagctcctgatcta
taaggtttccaaccggttctctggagtgccagataggttcagtggcagcgggtcagggacagatttc
acactgaaaatcagccgggtggaggctgaggatgttggggtttattactgcttccaaggtagccacg
tgcctttcaccttcggcggagggaccaaggtggagatcaaacgaactacggtggctgcaccatctgt
cttcatcttcccgccatctgatgagcagttgaaatctggaactgcctctgttgtgtgcctgctgaat
aacttctatcccagagaggccaaagtacagtggaaggtggataacgccctccaatcgggtaactccc
aggagagtgtcacagagcaggacagcaaggacagcacctacagcctcagcagcaccctgacgctgag
caaagcagactacgagaaacacaaagtctacgcctgcgaagtcacccatcagggcctgagctcgccc
gtcacaaagagcttcaacaggggagagtgttagtaagtttaaacccgctgatcagcctcgactgtgc
cttctagttgc
Mouse C8 heavy chain variable region sequence
(DNA)
(SEQ ID NO: 505)
gaagtgatggtcgtggaaagcggcggtggtctggtaaagccggggggatcccttaagctttcttgcg
ccgcatccgggttcacgttctccggctatgccatgtcctgggtccgacagactcccgaaaagcgctt
ggaatgggtggccactatctcctccggggggacgtacatctactaccccgacagtgtgaaaggaaga
tttacaatatctcgcgacaacgcaaaaaataccttgtatcttcaaatgagctccctgcggtcagagg
acactgccatgtactattgcgcccgcctgggcggcgacaattactatgagtat
(amino acids)
(SEQ ID NO: 506)
EVMVVESGGGLVKPGGSLKLSCAASGFTFSGYAMSWVRQTPEKRLEWVATISSGGTYIYYPDSVKGRF
TISRDNAKNTLYLQMSSLRSEDTAMYYCARLGGDNYYEY
Mouse C8 heavy chain variable complementarity determining region 1 (CDR1)
sequence:
(DNA)
(SEQ ID NO: 507)
ggctatgccatgtcc
(amino acids)
(SEQ ID NO: 508)
GYAMS
Mouse C8 heavy chain variable complementarity determining region 2 (CDR2)
sequence:
(DNA)
(SEQ ID NO: 509)
actatctoctccggggggacgtacatctactaccccgacagtgtgaaagga
(amino acids)
(SEQ ID NO: 510)
TISSGGTYIYYPDSVKG
Mouse C8 heavy chain variable complementarity determining region 3 (CDR3)
sequence:
(DNA)
(SEQ ID NO: 511)
ctgggcggcgacaattactatgagtat
(amino acids)
(SEQ ID NO: 512)
LGGDNYYEY
IGHV3-21*04 heavy chain variable region sequence:
(DNA)
(SEQ ID NO: 513)
gaggtgcagctggtggagtctgggggaggcctggtcaagcctggggggtccctgagactctcctgtg
cagcctctggattcaccttcagtagctatagcatgaactgggtccgccaggctccagggaaggggct
ggagtgggtctcatccattagtagtagtagtagttacatatactacgcagactcagtgaagggccga
ttcaccatctccagagacaacgccaagaactcactgtatctgcaaatgaacagcctgagagccgagg
acacggccgtgtattactgtgcga
(amino acids)
(SEQ ID NO: 514)
EVQLVESGGGLVKPGGSLRLSCAASGFTFSSYSMNWVRQAPGKGLEWVSSISSSSSYIYYADSVKGRFTISRD
NAKNSLYLQMNSLRAEDTAVYYCAR
IGHV3-21*04 heavy chain variable framework region 1 (FWR1) sequence:
(DNA)
(SEQ ID NO: 515)
gaggtgcagctggtggagtctgggggaggcctggtcaagcctggggggtccctgagactctcctgtgcagcct
ctggattcaccttcagt
(amino acids)
(SEQ ID NO: 516)
EVQLVESGGGLVKPGGSLRLSCAASGFTFS
IGHV3-21*04 heavy chain variable complementarity determining regions 1
(CDR1) sequence:
(DNA)
(SEQ ID NO: 517)
agctatagcatgaac
(amino acids)
(SEQ ID NO: 518)
SYSMN
IGHV3-21*04 heavy chain variable framework region 2 (FWR2) sequence:
(DNA)
(SEQ ID NO: 519)
tgggtccgccaggctccagggaaggggctggagtgggtc
(amino acids)
(SEQ ID NO: 520)
WVRQAPGKGLEWV
IGHV3-21*04 heavy chain variable complementarity determining regions 2
(CDR2) sequence:
(DNA)
(SEQ ID NO: 521)
tcatccattagtagtagtagtagttacatatactacgcagactcagtgaagggc
(amino acids)
(SEQ ID NO: 522)
SSISSSSSYIYYADSVKG
IGHV3-21*04 heavy chain variable framework region 3 (FWR3) sequence:
(DNA)
(SEQ ID NO: 523)
cgattcaccatctccagagacaacgccaagaactcactgtatctgcaaatgaacagcctgagagccgaggaca
cggccgtgtattactgtgcga
(amino acids)
(SEQ ID NO: 524)
RFTISRDNAKNSLYLQMNSLRAEDTAVYYCAR
Humanized C8 heavy chain variable region sequence:
(DNA)
(SEQ ID NO: 525)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccctgactcagtgaagggccgattcaccatctccagagac
aacgccaagaactcactgtatctgcaaatgaacagcctgagagccgaggacacggccgtgtattactgtgcga
gactgggcggcgataactattatgaatattggggcaaagggaccacggtcaccgtctcctcc
(amino acids)
(SEQ ID NO: 526)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYWGKGTTVTVSS
Humanized C8 heavy chain variable framework region 1 (FWR1) sequence:
(DNA)
(SEQ ID NO: 527)
gaggtgcagctggtggagtctgggggaggcctggtcaagcctggggggtccctgagactctcctgtgcagcct
ctggattcaccttcagt
(amino acids)
(SEQ ID NO: 528)
EVQLVESGGGLVKPGGSLRLSCAASGFTFS
Humanized C8 heavy chain variable complementarity determining region 1
(CDR1) sequence:
(DNA)
(SEQ ID NO: 529)
ggctatgccatgagc
(amino acids)
(SEQ ID NO: 530)
GYAMS
Humanized C8 heavy chain variable framework region 2 (FWR2) sequence:
(DNA)
(SEQ ID NO: 531)
tgggtccgccaggctccagggaaggggctggagtgggtctca
(amino acids)
(SEQ ID NO: 532)
WVRQAPGKGLEWVS
Humanized C8 heavy chain variable complementarity determining region 2
(CDR2) sequence:
(DNA)
(SEQ ID NO: 533)
accattagtagtggcggaacctacatatactaccctgactcagtgaagggc
(amino acids)
(SEQ ID NO: 534)
TISSGGTYIYYPDSVKG
Humanized C8 heavy chain variable framework region 3 (FWR3) sequence:
(DNA)
(SEQ ID NO: 535)
cgattcaccatctccagagacaacgccaagaactcactgtatctgcaaatgaacagcctgagagccgaggaca
cggccgtgtattactgtgcgaga
(amino acids)
(SEQ ID NO: 536)
RFTISRDNAKNSLYLQMNSLRAEDTAVYYCAR
Humanized C8 heavy chain variable complementarity determining region 3
(CDR3) sequence:
(DNA)
(SEQ ID NO: 537)
ctgggcggcgataactattatgaatat
(amino acids)
(SEQ ID NO: 538)
LGGDNYYEY
Humanized C8 IgG1 heavy chain sequence
(DNA)
(SEQ ID NO: 539)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccctgactcagtgaagggccgattcaccatctccagagac
aacgccaagaactcactgtatctgcaaatgaacagcctgagagccgaggacacggccgtgtattactgtgcga
gactgggcggcgataactattatgaatattggggcaaagggaccacggtcaccgtctcctccgctagcaccaa
gggcccatcggtcttccccctggcaccctcctccaagagcacctctgggggcacagcggccctgggctgcctg
gtcaaggactacttccccgaaccggtgacggtgtcgtggaactcaggcgccctgaccagcggcgtgcacacct
tcccggctgtcctacagtcctcaggactctactccctcagcagcgtggtgacagtgccctccagcagcttggg
cacccagacctacatctgcaacgtgaatcacaagcccagcaacaccaaggtggacaagaaagttgagcccaaa
tcttgtgacaaaactcacacatgcccaccgtgcccagcacctgaactcctggggggaccgtcagtcttcctct
tccccccaaaacccaaggacaccctcatgatctcccggacccctgaggtcacatgcgtggtggtggacgtgag
ccacgaagaccctgaggtcaagttcaactggtacgtggacggcgtggaggtgcataatgccaagacaaagccg
cgggaggagcagtacaacagcacgtaccgtgtggtcagcgtcctcaccgtcctgcaccaggactggctgaatg
gcaaggagtacaagtgcaaggtctccaacaaagccctcccagcccccatcgagaaaaccatctccaaagccaa
agggcagccccgagaaccacaggtgtacaccctgcccccatcccgggaggagatgaccaagaaccaggtcagc
ctgacctgcctggtcaaaggcttctatcccagcgacatcgccgtggagtgggagagcaatgggcagccggaga
acaactacaagaccacgcctcccgtgctggactccgacggctccttcttcctctacagcaagctcaccgtgga
caagagcaggtggcagcaggggaacgtcttctcatgctccgtgatgcatgaggctctgcacaaccactacacg
cagaagagcctctccctgtctccgggtaaatgataa
(amino acids)
(SEQ ID NO: 540)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYWGKGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYT
QKSLSLSPGK**
Humanized C8 IgG2 heavy chain sequence
(DNA)
(SEQ ID NO: 541)
gaggtgcagctggtggagtctgggggaggcctggtcaagcctggggggtccctgagactctcctgtgcagcct
ctggattcaccttcagtggctatgccatgagctgggtccgccaggctccagggaaggggctggagtgggtctc
aaccattagtagtggcggaacctacatatactaccctgactcagtgaagggccgattcaccatctccagagac
aacgccaagaactcactgtatctgcaaatgaacagcctgagagccgaggacacggccgtgtattactgtgcga
gactgggcggcgataactattatgaatattggggcaaagggaccacggtcaccgtctcctccgcctccaccaa
gggcccatcggtcttccccctggcgccctgctccaggagcacctccgagagcacagccgccctgggctgcctg
gtcaaggactacttccccgaaccggtgacggtgtcgtggaactcaggcgctctgaccagcggcgtgcacacct
tcccagctgtcctacagtcctcaggactctactccctcagcagcgtggtgaccgtgccctccagcaacttcgg
cacccagacctacacctgcaacgtagatcacaagcccagcaacaccaaggtggacaagacagttgagcgcaaa
tgttgtgtcgagtgcccaccgtgcccagcaccacctgtggcaggaccgtcagtcttcctcttccccccaaaac
ccaaggacaccctcatgatctcccggacccctgaggtcacgtgcgtggtggtggacgtgagccacgaagaccc
cgaggtccagttcaactggtacgtggacggcgtggaggtgcataatgccaagacaaagccacgggaggagcag
ttcaacagcacgttccgtgtggtcagcgtcctcaccgttgtgcaccaggactggctgaacggcaaggagtaca
agtgcaaggtctccaacaaaggcctcccagcccccatcgagaaaaccatctccaaaaccaaagggcagccccg
agaaccacaggtgtacaccctgcccccatcccgggaggagatgaccaagaaccaggtcagcctgacctgcctg
gtcaaaggcttctaccccagcgacatcgccgtggagtgggagagcaatgggcagccggagaacaactacaaga
ccacacctcccatgctggactccgacggctccttcttcctctacagcaagctcaccgtggacaagagcaggtg
gcagcaggggaacgtcttctcatgctccgtgatgcatgaggctctgcacaaccactacacgcagaagagcctc
tccctgtctccgggtaaatagtaa
(amino acids)
(SEQ ID NO: 542)
EVQLVESGGGLVKPGGSLRLSCAASGFTFSGYAMSWVRQAPGKGLEWVSTISSGGTYIYYPDSVKGRFTISRD
NAKNSLYLQMNSLRAEDTAVYYCARLGGDNYYEYWGKGTTVTVSSASTKGPSVFPLAPCSRSTSESTAALGCL
VKDYFPEPVTVSWNSGALTSGVHTFPAVLQSSGLYSLSSVVTVPSSNFGTQTYTCNVDHKPSNTKVDKTVERK
CCVECPPCPAPPVAGPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVMHEALHNHYTQKSL
SLSPGK**
Mouse C8 light chain variable region sequence
(DNA)
(SEQ ID NO: 543)
gacatcgtcattacgcagacccctgccagtcttgccgtttctctgggccagagggccactatcagttacaggg
cgagtaagtctgtgagtaccagcggctatagttacatgcattggaaccagcagaaaccgggacagccaccacg
cctgcttatttatctggtgtctaatcttgagtccggggtgcccgccaggttcagcggcagcggctctgggacc
gacttcacactcaacattcatccagtggaagaagaggacgctgctacatactactgtcaacacattcgggaac
tgaccaggagtgaa
(amino acids)
(SEQ ID NO: 544)
DIVITQTPASLAVSLGQRATISYRASKSVSTSGYSYMHWNQQKPGQPPRLLIYLVSNLESGVPARFS
GSGSGTDFTLNIHPVEEEDAATYYCQHIRELTRSE
Mouse C8 light chain variable complementarity determining region 1 (CDR1)
sequence:
(DNA)
(SEQ ID NO: 545)
agggcgagtaagtctgtgagtaccagcggctatagttacatgcat
(amino acids)
(SEQ ID NO: 546)
RASKSVSTSGYSYMH
Mouse C8 light chain variable complementarity determining region 2 (CDR2)
sequence:
(DNA)
(SEQ ID NO: 547)
ctggtgtctaatcttgagtcc
(amino acids)
(SEQ ID NO: 548)
LVSNLES
Mouse C8 light chain variable complementarity determining region 3 (CDR3)
sequence:
(DNA)
(SEQ ID NO: 549)
caacacattcgggaactgaccaggagtgaa
(amino acids)
(SEQ ID NO: 550)
QHIRELTRSE
NCBI germline z00023 light chain variable region sequence:
(DNA)
(SEQ ID NO: 551)
gacatcgtgatgacccagtctccagactccctggctgtgtctctgggcgagagggccaccatcaactgcaagt
ccagccagagtgttttatacagctccaacaataagaactacttagcttggtaccagcagaaaccaggacagcc
tcctaagctgctcatttactgggcatctacccgggaatccggggtccctgaccgattcagtggcagcgggtct
gggacagatttcactctcaccatcagcagcctgcaggctgaagatgtggcagtttattactgtcagcaatatt
atagtactcct
(amino acids)
(SEQ ID NO: 552)
DIVMTQSPDSLAVSLGERATINCKSSQSVLYSSNNKNYLAWYQQKPGQPPKLLIYWASTRESGVPDRFSGSGS
GTDFTLTISSLQAEDVAVYYCQQYYSTP
NCBI germline z00023 light chain variable framework region 1 (FWR1) acid
sequence:
(DNA)
(SEQ ID NO: 553)
gacatcgtgatgacccagtctccagactccctggctgtgtctctgggcgagagggccaccatcaactgc
(amino acids)
(SEQ ID NO: 554)
DIVMTQSPDSLAVSLGERATINC
NCBI germline z00023 light chain variable complementarity determining
regions 1 (CDR1) sequence:
(DNA)
(SEQ ID NO: 555)
aagtccagccagagtgttttatacagctccaacaataagaactacttagct
(amino acids)
(SEQ ID NO: 556)
KSSQSVLYSSNNKNYLA
NCBI germline z00023 light chain variable framework region 2 (FWR2)
sequence:
(DNA)
(SEQ ID NO: 557)
tggtaccagcagaaaccaggacagcctcctaagctgctcatttac
(amino acids)
(SEQ ID NO: 558)
WYQQKPGQPPKLLIY
NCBI germline z00023 light chain variable complementarity determining
regions 2 (CDR2) sequence:
(DNA)
(SEQ ID NO: 559)
tgggcatctacccgggaatcc
(amino acids)
(SEQ ID NO: 560)
WASTRES
NCBI germline z00023 light chain variable framework region 3 (FWR3)
sequence:
(DNA)
(SEQ ID NO: 561)
ggggtccctgaccgattcagtggcagcgggtctgggacagatttcactctcaccatcagcagcctgcaggctg
aagatgtggcagtttattactgt
(amino acids)
(SEQ ID NO: 562)
GVPDRFSGSGSGTDFTLTISSLQAEDVAVYYC
NCBI germline z00023 light chain variable complementarity determining
regions3 (CDR3) sequence:
(DNA)
(SEQ ID NO: 563)
cagcaatattatagtactcct
(amino acids)
(SEQ ID NO: 564)
QQYYSTP
Humanized C8 light chain variable region sequence
(DNA)
(SEQ ID NO: 565)
gacatcgtgatgacccagtctccagactccctggctgtgtctctgggcgagagggccaccatcaact
gcagggccagcaagagtgttagcaccagcggctacagctacatgcactggtaccagcagaaaccagg
acagcctcctaagctgctcatttacctggtgtctaacctggaatccggggtccctgaccgattcagt
ggcagcgggtctgggacagatttcactctcaccatcagcagcctgcaggctgaagatgtggcagttt
attactgtcaacacattcgggaactgaccaggagtgaattcggcggagggaccaaggtggagatcaa
acgaact
(amino acids)
(SEQ ID NO: 566)
DIVMTQSPDSLAVSLGERATINCRASKSVSTSGYSYMHWYQQKPGQPPKLLIYLVSNLESGVPDRFS
GSGSGTDFTLTISSLQAEDVAVYYCQHIRELTRSEFGGGTKVEIKRT
Humanized C8 light chain variable framework region 1 (FWR1)
sequence:
(DNA)
(SEQ ID NO: 567)
gacatcgtgatgacccagtctccagactccctggctgtgtctctgggcgagagggccaccatcaact
gc
(amino acids)
(SEQ ID NO: 568)
DIVMTQSPDSLAVSLGERATINC
Humanized C8 light chain variable complementarity determining region 1
(CDR1) sequence:
(DNA)
(SEQ ID NO: 569)
agggccagcaagagtgttagcaccagcggctacagctacatg
(amino acids)
(SEQ ID NO: 570)
RASKSVSTSGYSYM
Humanized C8 light chain variable framework region 2 (FWR2) sequence:
(DNA)
(SEQ ID NO: 571)
cactggtaccagcagaaaccaggacagcctcctaagctgctcatttac
(amino acids)
(SEQ ID NO: 572)
HWYQQKPGQPPKLLIY
Humanized C8 light chain variable complementarity determining region 2
(CDR2) sequence:
(DNA)
(SEQ ID NO: 573)
ctggtgtctaacctggaatcc
(amino acids)
(SEQ ID NO: 574)
LVSNLES
Humanized C8 light chain variable framework region 3 (FWR3) sequence:
(DNA)
(SEQ ID NO: 575)
ggggtccctgaccgattcagtggcagcgggtctgggacagatttcactctcaccatcagcagcctgcaggctg
aagatgtggcagtttattactgt
(amino acids)
(SEQ ID NO: 576)
GVPDRFSGSGSGTDFTLTISSLQAEDVAVYYC
Humanized C8 light chain variable complementarity determining region 3
(CDR3) sequence:
(DNA)
(SEQ ID NO: 577)
caacacattcgggaactgaccaggagtgaa
(amino acids)
(SEQ ID NO: 578)
QHIRELTRSE
Humanized C8 Lambda light chain sequence
(DNA)
(SEQ ID NO: 579)
gacatcgtgatgacccagtctccagactccctggctgtgtctctgggcgagagggccaccatcaactgcaggg
ccagcaagagtgttagcaccagcggctacagctacatgcactggtaccagcagaaaccaggacagcctcctaa
gctgctcatttacctggtgtctaacctggaatccggggtccctgaccgattcagtggcagcgggtctgggaca
gatttcactctcaccatcagcagcctgcaggctgaagatgtggcagtttattactgtcaacacattcgggaac
tgaccaggagtgaattcggcggagggaccaaggtggagatcaaacgaactggtcagcccaaggctgccccctc
ggtcactctgttcccgccctcctctgaggagcttcaagccaacaaggccacactggtgtgtctcataagtgac
ttctacccgggagccgtgacagtggcctggaaggcagatagcagccccgtcaaggcgggagtggagaccacca
caccctccaaacaaagcaacaacaagtacgcggccagcagctatctgagcctgacgcctgagcagtggaagtc
ccacagaagctacagctgccaggtcacgcatgaagggagcaccgtggagaagacagtggcccctacagaatgt
tcatagtaa
(amino acids)
(SEQ ID NO: 580)
DIVMTQSPDSLAVSLGERATINCRASKSVSTSGYSYMHWYQQKPGQPPKLLIYLVSNLESGVPDRFSGSGSGT
DFTLTISSLQAEDVAVYYCQHIRELTRSEFGGGTKVEIKRTGQPKAAPSVTLFPPSSEELQANKATLVCLISD
FYPGAVTVAWKADSSPVKAGVETTTPSKQSNNKYAASSYLSLTPEQWKSHRSYSCQVTHEGSTVEKTVAPTEC
S**
Humanized C8 Kappa light chain sequence
(DNA)
(SEQ ID NO: 581)
gacatcgtgatgacccagtctccagactccctggctgtgtctctgggcgagagggccaccatcaactgcaggg
ccagcaagagtgttagcaccagcggctacagctacatgcactggtaccagcagaaaccaggacagcctcctaa
gctgctcatttacctggtgtctaacctggaatccggggtccctgaccgattcagtggcagcgggtctgggaca
gatttcactctcaccatcagcagcctgcaggctgaagatgtggcagtttattactgtcaacacattcgggaac
tgaccaggagtgaattcggcggagggaccaaggtggagatcaaacgaactacggtggctgcaccatctgtctt
catcttcccgccatctgatgagcagttgaaatctggaactgcctctgttgtgtgcctgctgaataacttctat
cccagagaggccaaagtacagtggaaggtggataacgccctccaatcgggtaactcccaggagagtgtcacag
agcaggacagcaaggacagcacctacagcctcagcagcaccctgacgctgagcaaagcagactacgagaaaca
caaagtctacgcctgcgaagtcacccatcagggcctgagctcgcccgtcacaaagagcttcaacaggggagag
tgttagtaa
(amino acids)
(SEQ ID NO: 582)
DIVMTQSPDSLAVSLGERATINCRASKSVSTSGYSYMHWYQQKPGQPPKLLIYLVSNLESGVPDRFSGSGSGT
DFTLTISSLQAEDVAVYYCQHIRELTRSEFGGGTKVEIKRTTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFNRGE
C**
Humanized C8 Kappa light chain gBLOCk sequence:
(DNA)
(SEQ ID NO: 583)
agctggctaggtaagcttggtaccgagctcggatccacgccaccatggagacagacacactcctgctatgggt
actgctgctctgggttccaggttccactggtgacgacatcgtgatgacccagtctccagactccctggctgtg
tctctgggcgagagggccaccatcaactgcagggccagcaagagtgttagcaccagcggctacagctacatgc
actggtaccagcagaaaccaggacagcctcctaagctgctcatttacctggtgtctaacctggaatccggggt
ccctgaccgattcagtggcagcgggtctgggacagatttcactctcaccatcagcagcctgcaggctgaagat
gtggcagtttattactgtcaacacattcgggaactgaccaggagtgaattcggcggagggaccaaggtggaga
tcaaacgaactacggtggctgcaccatctgtcttcatcttcccgccatctgatgagcagttgaaatctggaac
tgcctctgttgtgtgcctgctgaataacttctatcccagagaggccaaagtacagtggaaggtggataacgcc
ctccaatcgggtaactcccaggagagtgtcacagagcaggacagcaaggacagcacctacagcctcagcagca
ccctgacgctgagcaaagcagactacgagaaacacaaagtctacgcctgcgaagtcacccatcagggcctgag
ctcgcccgtcacaaagagcttcaacaggggagagtgttagtaagtttaaacccgctgatcagcctcgactgtg
ccttctagttgc
CAR-T E6 CD8 sequence:
(DNA)
(SEQ ID NO: 584)
gaggtccagctggttgagagtggcggtgggctggttaagcctggcggctccctgcggctgagctgcgccgcga
gtggatttactttcagccgatatgggatgagttgggtgcggcaagctcccgggaagaggctggaatgggtctc
aacaatctccggggggggcacttacatctattaccccgactcagtcaaggggagatttaccatttcacgagac
aacgctaagaataccctgtatttgcagatgaattctctgagagcagaggacacagctgtttactattgtaccc
gcgacaactatggcaggaactacgactacggtatggactattggggacaagggacattggttacagtgagcag
tggcggcgggggcagcggaggaggaggcagcggtggggggggcagcgagatagtgctcacgcagtcacccgcg
actctcagtctctcacctggggaacgagctaccctgacgtgctctgctacctcctcagtgtcatatattcact
ggtatcagcaacggcccgggcagtcccctagattgctcatttatagtacctctaatctggcctcaggtatccc
tgcacgattttctggatctggttcaggttctgattacaccctcactatctctagcctggagcctgaagacttt
gccgtttattactgccagcagaggtctagctccccattcacctttgggagtgggaccaaggttgaaattaaaa
cgacaaccccggcccccagaccaccaacgccagcccccaccatcgccagccaacccctgtctctgagaccaga
agcctgtaggcctgccgccggtggagctgtgcacacaagaggactggatttcgcctgtgatatctacatttgg
gccccgctcgcaggcacatgtggagtgctcctcctctccctggtgattaccctgtactgctgataa
(amino acids)
(SEQ ID NO: 585)
EVQLVESGGGLVKPGGSLRLSCAASGFTFSRYGMSWVRQAPGKRLEWVSTISGGGTYIYYPDSVKGR
FTISRDNAKNTLYLQMNSLRAEDTAVYYCTRDNYGRNYDYGMDYWGQGTLVTVSSGGGGSGGGGSGG
GGSEIVLTQSPATLSLSPGERATLTCSATSSVSYIHWYQQRPGQSPRLLIYSTSNLASGIPARFSGS
GSGSDYTLTISSLEPEDFAVYYCQQRSSSPFTFGSGTKVEIKTTTPAPRPPTPAPTIASQPLSLRPE
ACRPAAGGAVHTRGLDFACDIYIWAPLAGTCGVLLLSLVITLYC**
CAR-T C2 CD8 sequence:
(DNA)
(SEQ ID NO: 586)
gaagtgcagctcgtagagagtggcgggggactggtgaagcccggtggaagcctcagactcagttgcg
ccgcctcaggtttcactttttcaggttacgccatgtcctgggtaagacaggcaccggggaaaggact
cgagtgggtgtctactatcagctcaggaggcacttatatatattatcctgactctgtaaaaggccga
tttacgatttctcgcgacaatgcaaagaactccctctacctccaaatgaacagtcttagggcagaag
acactgctgtatactattgtgcacgcctcggcggcgacaactactacgagtactttgacgtgtgggg
gaaagggactaccgtgacagtttcaagcggaggaggtggctcaggtggaggcgggtcagggggggga
ggaagtgatattgtgctcacacaatccccagcctccctggctgtgtctcccggccaacgcgctacaa
ttacatgtcgggcctccaaaagcgtgagcaccagcggctacagctacatgcactggtatcaacagaa
accaggacaaccccccaaactgttgatttatctcgcttcaaacttggagtccggcgtgcctgcgcgc
ttttcagggagtgggagcggcacagattttacgctgactatcaaccccgtagaagcaaacgatacag
cgaattattattgtcaacattcccgggaactcccctttacgttcggcgggggcacaaaggtcgaaat
taagagaaccacgacaaccccggcccccagaccaccaacgccagcccccaccatcgccagccaaccc
ctgtctctgagaccagaagcctgtaggcctgccgccggtggagctgtgcacacaagaggactggatt
tcgcctgtgatatctacatttgggccccgctcgcaggcacatgtggagtgctcctcctctccctggt
gattaccctgtactgctgataa
(amino acids)
(SEQ ID NO: 587)
EVQLVESGGGLVKPGGSLRLSCAASGFTFSGYAMSWVRQAPGKGLEWVSTISSGGTYIYYPDSVKGR
FTISRDNAKNSLYLQMNSLRAEDTAVYYCARLGGDNYYEYFDVWGKGTTVTVSSGGGGSGGGGSGGG
GSDIVLTQSPASLAVSPGQRATITCRASKSVSTSGYSYMHWYQQKPGQPPKLLIYLASNLESGVPAR
FSGSGSGTDFTLTINPVEANDTANYYCQHSRELPFTFGGGTKVEIKRTTTTPAPRPPTPAPTIASQP
LSLRPEACRPAAGGAVHTRGLDFACDIYIWAPLAGTCGVLLLSLVITLYC**
CD8/4-1BB sequence
(DNA)
(SEQ ID NO: 588)
acgacaaccccggcccccagaccaccaacgccagcccccaccatcgccagccaacccctgtctctga
gaccagaagcctgtaggcctgccgccggtggagctgtgcacacaagaggactggatttcgcctgtga
tatctacatttgggccccgctcgcaggcacatgtggagtgctcctcctctccctggtgattaccctg
tactgcaaaaggggccgcaaaaaactcctttacatttttaagcagccttttatgaggccagtacaga
cgactcaagaggaagacgggtgctcatgccgctttcctgaggaggaggaaggagggtgcgaactgtg
ataa
(amino acids)
(SEQ ID NO: 589)
TTTPAPRPPTPAPTIASQPLSLRPEACRPAAGGAVHTRGLDFACDIYIWAPLAGTCGVLLLSLVITL
YCKRGRKKLLYIFKQPFMRPVQTTQEEDGCSCRFPEEEEGGCEL**
CD8/CD28 sequence
(DNA)
(SEQ ID NO: 590)
acgacaaccccggcccccagaccaccaacgccagcccccaccatcgccagccaacccctgtctctga
gaccagaagcctgtaggcctgccgccggtggagctgtgcacacaagaggactggatttcgcctgtga
tatctacatttgggccccgctcgcaggcacatgtggagtgctcctcctctccctggtgattaccctg
tactgcagaagcaagcggtctcggctcctgcattctgattacatgaacatgaccccaagaagaccag
gccccaccaggaaacattaccagccctacgctccgccacgcgacttcgctgcctaccggtcctgata
a
(amino acids)
(SEQ ID NO: 591)
TTTPAPRPPTPAPTIASQPLSLRPEACRPAAGGAVHTRGLDFACDIYIWAPLAGTCGVLLLSLVITL
YCRSKRSRLLHSDYMNMTPRRPGPTRKHYQPYAPPRDFAAYRS**
CD8/CD3z sequence:
(DNA)
(SEQ ID NO: 592)
acgacaaccccggcccccagaccaccaacgccagcccccaccatcgccagccaacccctgtctctga
gaccagaagcctgtaggcctgccgccggtggagctgtgcacacaagaggactggatttcgcctgtga
tatctacatttgggccccgctcgcaggcacatgtggagtgctcctcctctccctggtgattaccctg
tactgccgcgttaagttctcccgatcagccgacgcgcctgcttacaagcagggccagaaccaactgt
acaacgagctgaatctcggtagacgggaagagtacgacgtgttggacaaacggagaggccgcgaccc
agaaatgggcggcaagcctcgcaggaaaaacccccaggagggactgtacaatgagttgcagaaagat
aagatggcagaagcttatagcgagatcggaatgaagggggaaaggagacgagggaaaggacacgacg
gcctttatcagggcctgtccacagcaacaaaagatacgtatgacgccctccatatgcaggcacttcc
accacggtgataa
(amino acids)
(SEQ ID NO: 593)
TTTPAPRPPTPAPTIASQPLSLRPEACRPAAGGAVHTRGLDFACDIYIWAPLAGTCGVLLLSLVITL
YCRVKFSRSADAPAYKQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR**
CD8/CD28/CD3z sequence:
(DNA)
(SEQ ID NO: 594)
acgacaaccccggcccccagaccaccaacgccagcccccaccatcgccagccaacccctgtctctga
gaccagaagcctgtaggcctgccgccggtggagctgtgcacacaagaggactggatttcgcctgtga
tatctacatttgggccccgctcgcaggcacatgtggagtgctcctcctctccctggtgattaccctg
tactgcagaagcaagcggtctcggctcctgcattctgattacatgaacatgaccccaagaagaccag
gccccaccaggaaacattaccagccctacgctccgccacgcgacttcgctgcctaccggtcccgcgt
taagttctcccgatcagccgacgcgcctgcttacaagcagggccagaaccaactgtacaacgagctg
aatctcggtagacgggaagagtacgacgtgttggacaaacggagaggccgcgacccagaaatgggcg
gcaagcctcgcaggaaaaacccccaggagggactgtacaatgagttgcagaaagataagatggcaga
agcttatagcgagatcggaatgaagggggaaaggagacgagggaaaggacacgacggcctttatcag
ggcctgtccacagcaacaaaagatacgtatgacgccctccatatgcaggcacttccaccacggtgat
aa
(amino acids)
(SEQ ID NO: 595)
TTTPAPRPPTPAPTIASQPLSLRPEACRPAAGGAVHTRGLDFACDIYIWAPLAGTCGVLLLSLVITL
YCRSKRSRLLHSDYMNMTPRRPGPTRKHYQPYAPPRDFAAYRSRVKFSRSADAPAYKQGQNQLYNEL
NLGRREEYDVLDKRRGRDPEMGGKPRRKNPQEGLYNELQKDKMAEAYSEIGMKGERRRGKGHDGLYQ
GLSTATKDTYDALHMQALPPR**
CD8/4-1BB/CD3z sequence:
(DNA)
(SEQ ID NO: 596)
acgacaaccccggcccccagaccaccaacgccagcccccaccatcgccagccaacccctgtctctga
gaccagaagcctgtaggcctgccgccggtggagctgtgcacacaagaggactggatttcgcctgtga
tatctacatttgggccccgctcgcaggcacatgtggagtgctcctcctctccctggtgattaccctg
tactgcaaaaggggccgcaaaaaactcctttacatttttaagcagccttttatgaggccagtacaga
cgactcaagaggaagacgggtgctcatgccgctttcctgaggaggaggaaggagggtgcgaactgcg
cgttaagttctcccgatcagccgacgcgcctgcttacaagcagggccagaaccaactgtacaacgag
ctgaatctcggtagacgggaagagtacgacgtgttggacaaacggagaggccgcgacccagaaatgg
gcggcaagcctcgcaggaaaaacccccaggagggactgtacaatgagttgcagaaagataagatggc
agaagcttatagcgagatcggaatgaagggggaaaggagacgagggaaaggacacgacggcctttat
cagggcctgtccacagcaacaaaagatacgtatgacgccctccatatgcaggcacttccaccacggt
gataa
(amino acids)
(SEQ ID NO: 597)
TTTPAPRPPTPAPTIASQPLSLRPEACRPAAGGAVHTRGLDFACDIYIWAPLAGTCGVLLLSLVITL
YCKRGRKKLLYIFKQPFMRPVQTTQEEDGCSCRFPEEEEGGCELRVKFSRSADAPAYKQGQNQLYNE
LNLGRREEYDVLDKRRGRDPEMGGKPRRKNPQEGLYNELQKDKMAEAYSEIGMKGERRRGKGHDGLY
QGLSTATKDTYDALHMQALPPR**
CD8/CD28/4-1BB/CD3z sequence:
(DNA)
(SEQ ID NO: 598)
acgacaaccccggcccccagaccaccaacgccagcccccaccatcgccagccaacccctgtctctga
gaccagaagcctgtaggcctgccgccggtggagctgtgcacacaagaggactggatttcgcctgtga
tatctacatttgggccccgctcgcaggcacatgtggagtgctcctcctctccctggtgattaccctg
tactgcagaagcaagcggtctcggctcctgcattctgattacatgaacatgaccccaagaagaccag
gccccaccaggaaacattaccagccctacgctccgccacgcgacttcgctgcctaccggtccaaaag
gggccgcaaaaaactcctttacatttttaagcagccttttatgaggccagtacagacgactcaagag
gaagacgggtgctcatgccgctttcctgaggaggaggaaggagggtgcgaactgcgcgttaagttct
cccgatcagccgacgcgcctgcttacaagcagggccagaaccaactgtacaacgagctgaatctcgg
tagacgggaagagtacgacgtgttggacaaacggagaggccgcgacccagaaatgggcggcaagcct
cgcaggaaaaacccccaggagggactgtacaatgagttgcagaaagataagatggcagaagcttata
gcgagatcggaatgaagggggaaaggagacgagggaaaggacacgacggcctttatcagggcctgtc
cacagcaacaaaagatacgtatgacgccctccatatgcaggcacttccaccacggtgataa
(amino acids)
(SEQ ID NO: 599)
TTTPAPRPPTPAPTIASQPLSLRPEACRPAAGGAVHTRGLDFACDIYIWAPLAGTCGVLLLSLVITL
YCRSKRSRLLHSDYMNMTPRRPGPTRKHYQPYAPPRDFAAYRSKRGRKKLLYIFKQPFMRPVQTTQE
EDGCSCRFPEEEEGGCELRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR**
CAR-T C3 4-1BB/CD3z sequence:
(DNA)
(SEQ ID NO: 600)
atggccctgcccgtgaccgctttgctgctccccctggcgctgctgctgcacgccgccaggccacagg
ttcagctggtgcagtctggagctgaggtgaagaagcctggggcctcagtgaaggtctcctgcaaggc
ttctggttacacctttaccgactacgccatgaactgggtgcgacaggcccctggacaagggcttgag
tggatgggagtgatcagcaccttcagcggtaacacaaacttcaaccagaagttcaagggcagagtca
ccatgaccacagacacatccacgagcacagcctacatggagctgaggagcctgagatctgacgacac
ggccgtgtattactgtgcgagaagcgactactacggcccatacttcgactactggggccagggcacc
accctgaccgtgtccagcggcggtggcggatccggcggtggcggatccggcggtggcggatccgata
ttgtgatgacccagactccactctctctgtccgtcacccctggacagccggcctccatctcctgcag
gtctagtcagaccattgtccatagtaatggaaacacctatttggagtggtacctgcagaagccaggc
cagtctccacagctcctgatctataaggtttccaaccggttctctggagtgccagataggttcagtg
gcagcgggtcagggacagatttcacactgaaaatcagccgggtggaggctgaggatgttggggttta
ttactgcttccaaggtagccacgtgcctttcaccttcggcggagggaccaaggtggagatcaaacga
actacgacaaccccggcccccagaccaccaacgccagcccccaccatcgccagccaacccctgtctc
tgagaccagaagcctgtaggcctgccgccggtggagctgtgcacacaagaggactggatttcgcctg
tgatatctacatttgggccccgctcgcaggcacatgtggagtgctcctcctctccctggtgattacc
ctgtactgcaaaaggggccgcaaaaaactcctttacatttttaagcagccttttatgaggccagtac
agacgactcaagaggaagacgggtgctcatgccgctttcctgaggaggaggaaggagggtgcgaact
gcgcgttaagttctcccgatcagccgacgcgcctgcttacaagcagggccagaaccaactgtacaac
gagctgaatctcggtagacgggaagagtacgacgtgttggacaaacggagaggccgcgacccagaaa
tgggcggcaagcctcgcaggaaaaacccccaggagggactgtacaatgagttgcagaaagataagat
ggcagaagcttatagcgagatcggaatgaagggggaaaggagacgagggaaaggacacgacggcctt
tatcagggcctgtccacagcaacaaaagatacgtatgacgccctccatatgcaggcacttccaccac
ggtgataa
(amino acids)
(SEQ ID NO: 601)
QVQLVQSGAEVKKPGASVKVSCKASGYTFTDYAMNWVRQAPGQGLEWMGVISTFSGNTNFNQKFKGR
VTMTTDTSTSTAYMELRSLRSDDTAVYYCARSDYYGPYFDYWGQGTTLTVSSGGGGSGGGGSGGGGS
DIVMTQTPLSLSVTPGQPASISCRSSQTIVHSNGNTYLEWYLQKPGQSPQLLIYKVSNRFSGVPDRF
SGSGSGTDFTLKISRVEAEDVGVYYCFQGSHVPFTFGGGTKVEIKRTTTTPAPRPPTPAPTIASQPL
SLRPEACRPAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYKQGQNQLYNELNLGRREEYDVLDKRRGRDP
EMGGKPRRKNPQEGLYNELQKDKMAEAYSEIGMKGERRRGKGHDGLYQGLSTATKDTYDALHMQALP
PR**
C3 CAR gBLOCK 1 sequence:
(DNA)
(SEQ ID NO: 602)
atccacgctgttttgacctccatagaagattctagagctagctgtagagcttggtaccgagggccac
catggccctgcccgtgaccgctttgctgctccccctggcgctgctgctgcacgccgccaggccacag
gttcagctggtgcagtctggagctgaggtgaagaagcctggggcctcagtgaaggtctcctgcaagg
cttctggttacacctttaccgactacgccatgaactgggtgcgacaggcccctggacaagggcttga
gtggatgggagtgatcagcaccttcagcggtaacacaaacttcaaccagaagttcaagggcagagtc
accatgaccacagacacatccacgagcacagcctacatggagctgaggagcctgagatctgacgaca
cggccgtgtattactgtgcgagaagcgactactacggcccatacttcgactactggggccagggcac
caccctgaccgtgtccagcggcggtggcggatccggcggtggcggatccggcggtggcggatccgat
attgtgatgacccagactccactctctctgt
C3 CAR gBLOCK 2 sequence:
(DNA)
(SEQ ID NO: 603)
tattgtgatgacccagactccactctctctgtccgtcacccctggacagccggcctccatctcctgc
aggtctagtcagaccattgtccatagtaatggaaacacctatttggagtggtacctgcagaagccag
gccagtctccacagctcctgatctataaggtttccaaccggttctctggagtgccagataggttcag
tggcagcgggtcagggacagatttcacactgaaaatcagccgggtggaggctgaggatgttggggtt
tattactgcttccaaggtagccacgtgcctttcaccttcggcggagggaccaaggtggagatcaaac
gaactacgacaaccccggcccccagaccaccaacgccagcccccaccatcgccagccaacccctgtc
tctgagaccagaagcctgtaggcctgccgccggtggagctgtgcacacaagaggactggatttcgcc
tgtgatatctacatttgggccccgctcgcaggcacatgtg
E6 scFV gBLOCK 1 sequence:
(DNA)
(SEQ ID NO: 604)
tgctctgggttccaggttccactggtgacgcggcccagccggccgaggtgcagctggtggagtctgg
gggaggcctggtcaagcctggggggtccctgagactctcctgtgcagcctctggattcaccttcagt
aggtatggcatgagctgggtccgccaggctccagggaagaggctggagtgggtctcaaccattagtg
gcggaggcacctacatatactacccagactcagtgaagggccgattcaccatctccagagacaacgc
caagaacaccctgtatctgcaaatgaacagcctgagagccgaggacacggctgtgtattactgtacc
agagataactatggccgcaactatgattatggcatggattattggggccagggcaccctggtgaccg
tgagcagcggcggtggcggatccggcggtggcggatccggcggtggcggatcc
E6 scFV gBLOCK 2 sequence:
(DNA)
(SEQ ID NO: 605)
ggcggtggcggatccggcggtggcggatccggcggtggcggatccgaaattgtgttgacacagtctc
cagccaccctgtctttgtctccaggggaaagagccaccctcacctgcagcgccaccagcagtgttag
ctacatccactggtaccaacagaggcctggccagagccccaggctcctcatctatagcacctccaac
ctggccagcggcatcccagccaggttcagtggcagtgggtctgggagcgactacactctcaccatca
gcagcctagagcctgaagattttgcagtttattactgtcagcagcgtagcagctcccctttcacctt
tggcagcggcaccaaagtggaaattaaaaccggtcatcatcaccatcaccactgataagtttaaacc
cgctgatcagcctcgactgtgccttctagt
CAR-T C2 CD3z sequence:
(DNA)
(SEQ ID NO: 606)
atggccttgccagtgacggccctgctgctgccattggctcttctgttgcacgctgccaggcctgaagtgcagc
tcgtagagagtggcgggggactggtgaagcccggtggaagcctcagactcagttgcgccgcctcaggtttcac
tttttcaggttacgccatgtcctgggtaagacaggcaccggggaaaggactcgagtgggtgtctactatcagc
tcaggaggcacttatatatattatcctgactctgtaaaaggccgatttacgatttctcgcgacaatgcaaaga
actccctctacctccaaatgaacagtcttagggcagaagacactgctgtatactattgtgcacgcctcggcgg
cgacaactactacgagtactttgacgtgtgggggaaagggactaccgtgacagtttcaagcggaggaggtggc
tcaggtggaggcgggtcaggggggggaggaagtgatattgtgctcacacaatccccagcctccctggctgtgt
ctcccggccaacgcgctacaattacatgtcgggcctccaaaagcgtgagcaccagcggctacagctacatgca
ctggtatcaacagaaaccaggacaaccccccaaactgttgatttatctcgcttcaaacttggagtccggcgtg
cctgcgcgcttttcagggagtgggagcggcacagattttacgctgactatcaaccccgtagaagcaaacgata
cagcgaattattattgtcaacattcccgggaactcccctttacgttcggcgggggcacaaaggtcgaaattaa
gagaaccacgacaaccccggcccccagaccaccaacgccagcccccaccatcgccagccaacccctgtctctg
agaccagaagcctgtaggcctgccgccggtggagctgtgcacacaagaggactggatttcgcctgtgatatct
acatttgggccccgctcgcaggcacatgtggagtgctcctcctctccctggtgattaccctgtactgccgcgt
taagttctcccgatcagccgacgcgcctgcttacaagcagggccagaaccaactgtacaacgagctgaatctc
ggtagacgggaagagtacgacgtgttggacaaacggagaggccgcgacccagaaatgggcggcaagcctcgca
ggaaaaacccccaggagggactgtacaatgagttgcagaaagataagatggcagaagcttatagcgagatcgg
aatgaagggggaaaggagacgagggaaaggacacgacggcctttatcagggcctgtccacagcaacaaaagat
acgtatgacgccctccatatgcaggcacttccaccacggtgataa
(amino acids)
(SEQ ID NO: 607)
MALPVTALLLPLALLLHAARPEVQLVESGGGLVKPGGSLRLSCAASGFTFSGYAMSWVRQAPGKGLE
WVSTISSGGTYIYYPDSVKGRFTISRDNAKNSLYLQMNSLRAEDTAVYYCARLGGDNYYEYFDVWGK
GTTVTVSSGGGGSGGGGSGGGGSDIVLTQSPASLAVSPGQRATITCRASKSVSTSGYSYMHWYQQKP
GQPPKLLIYLASNLESGVPARFSGSGSGTDFTLTINPVEANDTANYYCQHSRELPFTFGGGTKVEIK
RTTTTPAPRPPTPAPTIASQPLSLRPEACRPAAGGAVHTRGLDFACDIYIWAPLAGTCGVLLLSLVI
TLYCRVKFSRSADAPAYKQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR**
CAR-T C2 CD28/CD3z sequence:
(DNA)
(SEQ ID NO: 608)
atggccttgccagtgacggccctgctgctgccattggctcttctgttgcacgctgccaggcctgaagtgcagc
tcgtagagagtggcgggggactggtgaagcccggtggaagcctcagactcagttgcgccgcctcaggtttcac
tttttcaggttacgccatgtcctgggtaagacaggcaccggggaaaggactcgagtgggtgtctactatcagc
tcaggaggcacttatatatattatcctgactctgtaaaaggccgatttacgatttctcgcgacaatgcaaaga
actccctctacctccaaatgaacagtcttagggcagaagacactgctgtatactattgtgcacgcctcggcgg
cgacaactactacgagtactttgacgtgtgggggaaagggactaccgtgacagtttcaagcggaggaggtggc
tcaggtggaggcgggtcaggggggggaggaagtgatattgtgctcacacaatccccagcctccctggctgtgt
ctcccggccaacgcgctacaattacatgtcgggcctccaaaagcgtgagcaccagcggctacagctacatgca
ctggtatcaacagaaaccaggacaaccccccaaactgttgatttatctcgcttcaaacttggagtccggcgtg
cctgcgcgcttttcagggagtgggagcggcacagattttacgctgactatcaaccccgtagaagcaaacgata
cagcgaattattattgtcaacattcccgggaactcccctttacgttcggcgggggcacaaaggtcgaaattaa
gagaaccacgacaaccccggcccccagaccaccaacgccagcccccaccatcgccagccaacccctgtctctg
agaccagaagcctgtaggcctgccgccggtggagctgtgcacacaagaggactggatttcgcctgtgatatct
acatttgggccccgctcgcaggcacatgtggagtgctcctcctctccctggtgattaccctgtactgcagaag
caagcggtctcggctcctgcattctgattacatgaacatgaccccaagaagaccaggccccaccaggaaacat
taccagccctacgctccgccacgcgacttcgctgcctaccggtcccgcgttaagttctcccgatcagccgacg
cgcctgcttacaagcagggccagaaccaactgtacaacgagctgaatctcggtagacgggaagagtacgacgt
gttggacaaacggagaggccgcgacccagaaatgggcggcaagcctcgcaggaaaaacccccaggagggactg
tacaatgagttgcagaaagataagatggcagaagcttatagcgagatcggaatgaagggggaaaggagacgag
ggaaaggacacgacggcctttatcagggcctgtccacagcaacaaaagatacgtatgacgccctccatatgca
ggcacttccaccacggtgataa
(amino acids)
(SEQ ID NO: 609)
MALPVTALLLPLALLLHAARPEVQLVESGGGLVKPGGSLRLSCAASGFTFSGYAMSWVRQAPGKGLE
WVSTISSGGTYIYYPDSVKGRFTISRDNAKNSLYLQMNSLRAEDTAVYYCARLGGDNYYEYFDVWGK
GTTVTVSSGGGGSGGGGSGGGGSDIVLTQSPASLAVSPGQRATITCRASKSVSTSGYSYMHWYQQKP
GQPPKLLIYLASNLESGVPARFSGSGSGTDFTLTINPVEANDTANYYCQHSRELPFTFGGGTKVEIK
RTTTTPAPRPPTPAPTIASQPLSLRPEACRPAAGGAVHTRGLDFACDIYIWAPLAGTCGVLLLSLVI
TLYCRSKRSRLLHSDYMNMTPRRPGPTRKHYQPYAPPRDFAAYRSRVKFSRSADAPAYKQGQNQLYN
ELNLGRREEYDVLDKRRGRDPEMGGKPRRKNPQEGLYNELQKDKMAEAYSEIGMKGERRRGKGHDGL
YQGLSTATKDTYDALHMQALPPR**
CAR-T C2 4-1BB/CD3z sequence:
(DNA)
(SEQ ID NO: 610)
atggccttgccagtgacggccctgctgctgccattggctcttctgttgcacgctgccaggcctgaagtgcagc
tcgtagagagtggcgggggactggtgaagcccggtggaagcctcagactcagttgcgccgcctcaggtttcac
tttttcaggttacgccatgtcctgggtaagacaggcaccggggaaaggactcgagtgggtgtctactatcagc
tcaggaggcacttatatatattatcctgactctgtaaaaggccgatttacgatttctcgcgacaatgcaaaga
actccctctacctccaaatgaacagtcttagggcagaagacactgctgtatactattgtgcacgcctcggcgg
cgacaactactacgagtactttgacgtgtgggggaaagggactaccgtgacagtttcaagcggaggaggtggc
tcaggtggaggcgggtcaggggggggaggaagtgatattgtgctcacacaatccccagcctccctggctgtgt
ctcccggccaacgcgctacaattacatgtcgggcctccaaaagcgtgagcaccagcggctacagctacatgca
ctggtatcaacagaaaccaggacaaccccccaaactgttgatttatctcgcttcaaacttggagtccggcgtg
cctgcgcgcttttcagggagtgggagcggcacagattttacgctgactatcaaccccgtagaagcaaacgata
cagcgaattattattgtcaacattcccgggaactcccctttacgttcggcgggggcacaaaggtcgaaattaa
gagaaccacgacaaccccggcccccagaccaccaacgccagcccccaccatcgccagccaacccctgtctctg
agaccagaagcctgtaggcctgccgccggtggagctgtgcacacaagaggactggatttcgcctgtgatatct
acatttgggccccgctcgcaggcacatgtggagtgctcctcctctccctggtgattaccctgtactgcaaaag
gggccgcaaaaaactcctttacatttttaagcagccttttatgaggccagtacagacgactcaagaggaagac
gggtgctcatgccgctttcctgaggaggaggaaggagggtgcgaactgcgcgttaagttctcccgatcagccg
acgcgcctgcttacaagcagggccagaaccaactgtacaacgagctgaatctcggtagacgggaagagtacga
cgtgttggacaaacggagaggccgcgacccagaaatgggcggcaagcctcgcaggaaaaacccccaggaggga
ctgtacaatgagttgcagaaagataagatggcagaagcttatagcgagatcggaatgaagggggaaaggagac
gagggaaaggacacgacggcctttatcagggcctgtccacagcaacaaaagatacgtatgacgccctccatat
gcaggcacttccaccacggtgataa
(amino acids)
(SEQ ID NO: 611)
MALPVTALLLPLALLLHAARPEVQLVESGGGLVKPGGSLRLSCAASGFTFSGYAMSWVRQAPGKGLE
WVSTISSGGTYIYYPDSVKGRFTISRDNAKNSLYLQMNSLRAEDTAVYYCARLGGDNYYEYFDVWGK
GTTVTVSSGGGGSGGGGSGGGGSDIVLTQSPASLAVSPGQRATITCRASKSVSTSGYSYMHWYQQKP
GQPPKLLIYLASNLESGVPARFSGSGSGTDFTLTINPVEANDTANYYCQHSRELPFTFGGGTKVEIK
RTTTTPAPRPPTPAPTIASQPLSLRPEACRPAAGGAVHTRGLDFACDIYIWAPLAGTCGVLLLSLVI
TLYCKRGRKKLLYIFKQPFMRPVQTTQEEDGCSCRFPEEEEGGCELRVKFSRSADAPAYKQGQNQLY
NELNLGRREEYDVLDKRRGRDPEMGGKPRRKNPQEGLYNELQKDKMAEAYSEIGMKGERRRGKGHDG
LYQGLSTATKDTYDALHMQALPPR**
CAR-T C2 OX40/CD3z sequence:
(DNA)
(SEQ ID NO: 612)
atggccttgccagtgacggccctgctgctgccattggctcttctgttgcacgctgccaggcctgaagtgcagc
tcgtagagagtggcgggggactggtgaagcccggtggaagcctcagactcagttgcgccgcctcaggtttcac
tttttcaggttacgccatgtcctgggtaagacaggcaccggggaaaggactcgagtgggtgtctactatcagc
tcaggaggcacttatatatattatcctgactctgtaaaaggccgatttacgatttctcgcgacaatgcaaaga
actccctctacctccaaatgaacagtcttagggcagaagacactgctgtatactattgtgcacgcctcggcgg
cgacaactactacgagtactttgacgtgtgggggaaagggactaccgtgacagtttcaagcggaggaggtggc
tcaggtggaggcgggtcaggggggggaggaagtgatattgtgctcacacaatccccagcctccctggctgtgt
ctcccggccaacgcgctacaattacatgtcgggcctccaaaagcgtgagcaccagcggctacagctacatgca
ctggtatcaacagaaaccaggacaaccccccaaactgttgatttatctcgcttcaaacttggagtccggcgtg
cctgcgcgcttttcagggagtgggagcggcacagattttacgctgactatcaaccccgtagaagcaaacgata
cagcgaattattattgtcaacattcccgggaactcccctttacgttcggcgggggcacaaaggtcgaaattaa
gagaaccacgacaaccccggcccccagaccaccaacgccagcccccaccatcgccagccaacccctgtctctg
agaccagaagcctgtaggcctgccgccggtggagctgtgcacacaagaggactggatttcgcctgtgatatct
acatttgggccccgctcgcaggcacatgtggagtgctcctcctctccctggtgattaccctgtactgccggag
ggaccagaggctgccccccgatgcccacaagccccctgggggaggcagtttccggacccccatccaagaggag
caggccgacgcccactccaccctggccaagatccgcgttaagttctcccgatcagccgacgcgcctgcttaca
agcagggccagaaccaactgtacaacgagctgaatctcggtagacgggaagagtacgacgtgttggacaaacg
gagaggccgcgacccagaaatgggcggcaagcctcgcaggaaaaacccccaggagggactgtacaatgagttg
cagaaagataagatggcagaagcttatagcgagatcggaatgaagggggaaaggagacgagggaaaggacacg
acggcctttatcagggcctgtccacagcaacaaaagatacgtatgacgccctccatatgcaggcacttccacc
acggtgataa
(amino acids)
(SEQ ID NO: 613)
MALPVTALLLPLALLLHAARPEVQLVESGGGLVKPGGSLRLSCAASGFTFSGYAMSWVRQAPGKGLE
WVSTISSGGTYIYYPDSVKGRFTISRDNAKNSLYLQMNSLRAEDTAVYYCARLGGDNYYEYFDVWGK
GTTVTVSSGGGGSGGGGSGGGGSDIVLTQSPASLAVSPGQRATITCRASKSVSTSGYSYMHWYQQKP
GQPPKLLIYLASNLESGVPARFSGSGSGTDFTLTINPVEANDTANYYCQHSRELPFTFGGGTKVEIK
RTTTTPAPRPPTPAPTIASQPLSLRPEACRPAAGGAVHTRGLDFACDIYIWAPLAGTCGVLLLSLVI
TLYCRRDQRLPPDAHKPPGGGSFRTPIQEEQADAHSTLAKIRVKFSRSADAPAYKQGQNQLYNELNL
GRREEYDVLDKRRGRDPEMGGKPRRKNPQEGLYNELQKDKMAEAYSEIGMKGERRRGKGHDGLYQGL
STATKDTYDALHMQALPPR**
CAR-T C2 CD28/OX40/CD3z sequence:
(DNA)
(SEQ ID NO: 614)
atggccttgccagtgacggccctgctgctgccattggctcttctgttgcacgctgccaggcctgaagtgcagc
tcgtagagagtggcgggggactggtgaagcccggtggaagcctcagactcagttgcgccgcctcaggtttcac
tttttcaggttacgccatgtcctgggtaagacaggcaccggggaaaggactcgagtgggtgtctactatcagc
tcaggaggcacttatatatattatcctgactctgtaaaaggccgatttacgatttctcgcgacaatgcaaaga
actccctctacctccaaatgaacagtcttagggcagaagacactgctgtatactattgtgcacgcctcggcgg
cgacaactactacgagtactttgacgtgtgggggaaagggactaccgtgacagtttcaagcggaggaggtggc
tcaggtggaggcgggtcaggggggggaggaagtgatattgtgctcacacaatccccagcctccctggctgtgt
ctcccggccaacgcgctacaattacatgtcgggcctccaaaagcgtgagcaccagcggctacagctacatgca
ctggtatcaacagaaaccaggacaaccccccaaactgttgatttatctcgcttcaaacttggagtccggcgtg
cctgcgcgcttttcagggagtgggagcggcacagattttacgctgactatcaaccccgtagaagcaaacgata
cagcgaattattattgtcaacattcccgggaactcccctttacgttcggcgggggcacaaaggtcgaaattaa
gagaaccacgacaaccccggcccccagaccaccaacgccagcccccaccatcgccagccaacccctgtctctg
agaccagaagcctgtaggcctgccgccggtggagctgtgcacacaagaggactggatttcgcctgtgatatct
acatttgggccccgctcgcaggcacatgtggagtgctcctcctctccctggtgattaccctgtactgcagaag
caagcggtctcggctcctgcattctgattacatgaacatgaccccaagaagaccaggccccaccaggaaacat
taccagccctacgctccgccacgcgacttcgctgcctaccggtcccggagggaccagaggctgccccccgatg
cccacaagccccctgggggaggcagtttccggacccccatccaagaggagcaggccgacgcccactccaccct
ggccaagatccgcgttaagttctcccgatcagccgacgcgcctgcttacaagcagggccagaaccaactgtac
aacgagctgaatctcggtagacgggaagagtacgacgtgttggacaaacggagaggccgcgacccagaaatgg
gcggcaagcctcgcaggaaaaacccccaggagggactgtacaatgagttgcagaaagataagatggcagaagc
ttatagcgagatcggaatgaagggggaaaggagacgagggaaaggacacgacggcctttatcagggcctgtcc
acagcaacaaaagatacgtatgacgccctccatatgcaggcacttccaccacggtgataa
(amino acids)
(SEQ ID NO: 615)
MALPVTALLLPLALLLHAARPEVQLVESGGGLVKPGGSLRLSCAASGFTFSGYAMSWVRQAPGKGLE
WVSTISSGGTYIYYPDSVKGRFTISRDNAKNSLYLQMNSLRAEDTAVYYCARLGGDNYYEYFDVWGK
GTTVTVSSGGGGSGGGGSGGGGSDIVLTQSPASLAVSPGQRATITCRASKSVSTSGYSYMHWYQQKP
GQPPKLLIYLASNLESGVPARFSGSGSGTDFTLTINPVEANDTANYYCQHSRELPFTFGGGTKVEIK
RTTTTPAPRPPTPAPTIASQPLSLRPEACRPAAGGAVHTRGLDFACDIYIWAPLAGTCGVLLLSLVI
TLYCRSKRSRLLHSDYMNMTPRRPGPTRKHYQPYAPPRDFAAYRSRRDQRLPPDAHKPPGGGSFRTP
IQEEQADAHSTLAKIRVKFSRSADAPAYKQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPPR**
CAR-T E6 OX40/CD3z sequence:
(DNA)
(SEQ ID NO: 616)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggggggg
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaaacgacaaccccg
gcccccagaccaccaacgccagcccccaccatcgccagccaacccctgtctctgagaccagaagcct
gtaggcctgccgccggtggagctgtgcacacaagaggactggatttcgcctgtgatatctacatttg
ggccccgctcgcaggcacatgtggagtgctcctcctctccctggtgattaccctgtactgccggagg
gaccagaggctgccccccgatgcccacaagccccctgggggaggcagtttccggacccccatccaag
aggagcaggccgacgcccactccaccctggccaagatccgcgttaagttctcccgatcagccgacgc
gcctgcttacaagcagggccagaaccaactgtacaacgagctgaatctcggtagacgggaagagtac
gacgtgttggacaaacggagaggccgcgacccagaaatgggcggcaagcctcgcaggaaaaaccccc
aggagggactgtacaatgagttgcagaaagataagatggcagaagcttatagcgagatcggaatgaa
gggggaaaggagacgagggaaaggacacgacggcctttatcagggcctgtccacagcaacaaaagat
acgtatgacgccctccatatgcaggcacttccaccacggtgataa
(amino acids)
(SEQ ID NO: 617)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKTTTP
APRPPTPAPTIASQPLSLRPEACRPAAGGAVHTRGLDFACDIYIWAPLAGTCGVLLLSLVITLYCRR
DQRLPPDAHKPPGGGSFRTPIQEEQADAHSTLAKIRVKFSRSADAPAYKQGQNQLYNELNLGRREEY
DVLDKRRGRDPEMGGKPRRKNPQEGLYNELQKDKMAEAYSEIGMKGERRRGKGHDGLYQGLSTATKD
TYDALHMQALPPR**
CAR-T E6 CD28/OX40/CD3z sequence:
(DNA)
(SEQ ID NO: 618)
atggccctgcccgtgaccgctttgctgctccccctggcgctgctgctgcacgccgccaggccagagg
tccagctggttgagagtggcggtgggctggttaagcctggcggctccctgcggctgagctgcgccgc
gagtggatttactttcagccgatatgggatgagttgggtgcggcaagctcccgggaagaggctggaa
tgggtctcaacaatctccggggggggcacttacatctattaccccgactcagtcaaggggagattta
ccatttcacgagacaacgctaagaataccctgtatttgcagatgaattctctgagagcagaggacac
agctgtttactattgtacccgcgacaactatggcaggaactacgactacggtatggactattgggga
caagggacattggttacagtgagcagtggcggcgggggcagcggaggaggaggcagcggtggggggg
gcagcgagatagtgctcacgcagtcacccgcgactctcagtctctcacctggggaacgagctaccct
gacgtgctctgctacctcctcagtgtcatatattcactggtatcagcaacggcccgggcagtcccct
agattgctcatttatagtacctctaatctggcctcaggtatccctgcacgattttctggatctggtt
caggttctgattacaccctcactatctctagcctggagcctgaagactttgccgtttattactgcca
gcagaggtctagctccccattcacctttgggagtgggaccaaggttgaaattaaaacgacaaccccg
gcccccagaccaccaacgccagcccccaccatcgccagccaacccctgtctctgagaccagaagcct
gtaggcctgccgccggtggagctgtgcacacaagaggactggatttcgcctgtgatatctacatttg
ggccccgctcgcaggcacatgtggagtgctcctcctctccctggtgattaccctgtactgcagaagc
aagcggtctcggctcctgcattctgattacatgaacatgaccccaagaagaccaggccccaccagga
aacattaccagccctacgctccgccacgcgacttcgctgcctaccggtcccggagggaccagaggct
gccccccgatgcccacaagccccctgggggaggcagtttccggacccccatccaagaggagcaggcc
gacgcccactccaccctggccaagatccgcgttaagttctcccgatcagccgacgcgcctgcttaca
agcagggccagaaccaactgtacaacgagctgaatctcggtagacgggaagagtacgacgtgttgga
caaacggagaggccgcgacccagaaatgggcggcaagcctcgcaggaaaaacccccaggagggactg
tacaatgagttgcagaaagataagatggcagaagcttatagcgagatcggaatgaagggggaaagga
gacgagggaaaggacacgacggcctttatcagggcctgtccacagcaacaaaagatacgtatgacgc
cctccatatgcaggcacttccaccacggtgataa
(amino acids)
(SEQ ID NO: 619)
MALPVTALLLPLALLLHAARPEVQLVESGGGLVKPGGSLRLSCAASGFTFSRYGMSWVRQAPGKRLE
WVSTISGGGTYIYYPDSVKGRFTISRDNAKNTLYLQMNSLRAEDTAVYYCTRDNYGRNYDYGMDYWG
QGTLVTVSSGGGGSGGGGSGGGGSEIVLTQSPATLSLSPGERATLTCSATSSVSYIHWYQQRPGQSP
RLLIYSTSNLASGIPARFSGSGSGSDYTLTISSLEPEDFAVYYCQQRSSSPFTFGSGTKVEIKTTTP
APRPPTPAPTIASQPLSLRPEACRPAAGGAVHTRGLDFACDIYIWAPLAGTCGVLLLSLVITLYCRS
KRSRLLHSDYMNMTPRRPGPTRKHYQPYAPPRDFAAYRSRRDQRLPPDAHKPPGGGSFRTPIQEEQA
DAHSTLAKIRVKFSRSADAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR**
MUC1 truncated cytoplasmic sequence
(amino acids)
(SEQ ID NO: 620)
SNIKFRPGSVVVQLTLAFREGTINVHDVETQFNQYKTEAASRY
MUC1 truncated cytoplasmic sequence
(amino acids)
(SEQ ID NO: 621)
SVVVQLTLAFREGTINVHDVETQFNQYKTEAASRY
MUC1 truncated cytoplasmic sequence
(amino acids)
(SEQ ID NO: 622)
VQLTLAFREGTINVHDVETQFNQY
MUC1 truncated cytoplasmic sequence
(amino acids)
(SEQ ID NO: 623)
SNIKFRPGSVVVQLTLAFREGTIN
Primers
(SEQ ID NO: 624)
attctaagcttgggccaccatggaactg
(SEQ ID NO: 625)
tctagagtttaaacttactatttacccggagacagggagag
(SEQ ID NO: 626)
agtatggcccagccggccgaggtgcagctggtggagtctgg
(SEQ ID NO: 627)
tagaaggcacagtcgaggctgatcag
(SEQ ID NO: 628)
attctaagcttgggccaccatggaagc
(SEQ ID NO: 629)
tctagagtttaaacttactaacactctcccctgttgaagc
(SEQ ID NO: 630)
agtatggcccagccggccgaaattgtgttgacacagtctccag
(SEQ ID NO: 631)
tagaaggcacagtcgaggctgatcag
(SEQ ID NO: 632)
actgtcatatggaggtgcagctggtggagtctg
(SEQ ID NO: 633)
actgtctcgagtttaatttccactttggtgccgctgc
(SEQ ID NO: 634)
actgtcatatggaggtgcagctggtggagtctg
(SEQ ID NO: 635)
actgtaccggttttaatttccactttggtgccgctgc
(SEQ ID NO: 636)
cttcttcctcaggagcaagctcaccgtgg
(SEQ ID NO: 637)
gagccgtcggagtccagc
(SEQ ID NO: 638)
gcacctgaactcctgggg
(SEQ ID NO: 639)
tttaatttccactttggtgccg
(SEQ ID NO: 640)
cgcggctagcttaagcttggtaccgagggcca
(SEQ ID NO: 641)
cgcggcggccgcctgatcagcgggtttaaacttatc

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1—ELISA Competition with NME1 and NME7

PSMGFR peptide was covalently coupled to BSA using Imject Maleimide activated BSA kit (Thermo Fisher). PSMGFR peptide coupled BSA was diluted to 7.5 ug/mL in 0.1M carbonate/bicarbonate buffer pH 9.6 and 50 uL was added to each well of a 96 well plate. After overnight incubation at 4° C., the plate was wash twice with PBS-T and a 3% BSA solution was added to block remaining binding site on the well. After 1h at RT the plate was washed twice with PBS-T and NMElor NME7, diluted in PBS-T+1% BSA, was added at saturating concentration. After 1h at RT the plate was washed 3× with PBS-T and anti-MUC1* antibody (or antibody fragments), diluted in PBS-T+1% BSA, was added (5× molar excess comapred to NME1/NME7). After 1h at RT the plate was washed 3× with PBS-T and goat anti HisTag-HRP, diluted in PBS-T+1% BSA, was added at 1/10000 dilution. After 1 h at RT the plate was washed 3× with PBS-T and remaining NME1 or NME7 bound to the PSMGFR peptide was measured at 415 nm using a ABTS solution (Thermo Fisher).

### Example 2—Humanization of Anti-MUC1* Extracellular Domain Monoclonal Antibodies

We generated humanized antibodies that bind to the extracellular domain of MUC1* by a process called complementarity determining region. CDR′, grafting. First, homology searches were performed to independently align the heavy chain variable region and light chain variable region nucleotides sequences of mouse monoclonal anti-MUC1* antibody (E6 HC SEQ ID NOS: 12-13: LC SEQ ID NOS: 65-66 and MN-C2 HC SEQ ID NO: 118-119; LC SEQ ID NO: 168-169) against a repertoire of human antibody sequences (IMGT, the international ImMunoGeneTics information system). The sequences with the highest homology were selected. IGHV3-21*01 is a human IgG heavy chain variable region sequence with 82.9% (DNA) and 74.5% (amino acids) identity to MouseMN-E6 heavy chain variable region. IGKV3-11*02 is a human IgG light chain variable region sequence with 68.8% (DNA) and 61.1% (amino acids) identity to MouseMN-E6 light chain variable region. IGHV3-21*04 is a human IgG heavy chain variable region sequence with 85% (DNA) and 81.6% (amino acids) identity to Mouse MN-C2 heavy chain variable region. IGKV7-3*01 is a human IgG light chain variable region sequence with 76.9% (DNA) and 71.3% (amino acids) identity to Mouse MN-C2 light chain variable region. Second, a model of the mouse scFv was generated to select and keep the mouse residues important for the stability of the CDR and framework. Finally, CDRs from the human germlines were replaced by the corresponding mouse CDRs.

### Humanized MN-E6 19G2 Heavy Chain Cloning

The Kozak consensus sequence followed by the IGHV3-21*03 leader sequence, the humanizedMN-E6 heavy chain variable region and the constant region of human IgG2 was synthesized by our request by GenScript, NJ (SEQ ID NOS: 52-53. The cDNA was amplified by polymerase chain reaction (PCR) using the following primer: 5′-ATTCTAAGCTTGGGCCACCATGGAACTG-3′ (SEQ ID NO:624) and 5′-TCTAGAGTTTAAACTTACTATTTACCCGGAGACAGGGAGAG-3′ (SEQ ID NO:625). After digestion with HindIII and Pmel restriction enzymes (New England Biolabs), the purified fragment was cloned into the pCDNA 3.1 V5 vector (Life Technologies) digested with the same restriction enzymes.

Humanized MN-E6 heavy chain cDNA was amplified by polymerase chain reaction (PCR) using the following primer: 5′-AGTATGGCCCAGCCGGCCGAGGTGCAGCTGGTGGAGTCTGG-3′ (SEQ ID NO:626) and 5′-TAGAAGGCACAGTCGAGGCTGATCAG-3′ (SEQ ID NO:627). After digestion with SfiI and Pmel restriction enzymes (New England Biolabs), the purified fragment was cloned into the pSECTag2 vector (Life Technologies) digested with the same restriction enzymes.

### Humanized MN-E6 Kappa Light Chain Cloning

The Kozak consensus sequence followed by the IGHV3-11*02 leader sequence, the humanizedMN-E6 light chain variable region and the constant region of human Kappa light chain was synthesized by our request by GenScript, NJ (SEQ ID NOS: 107-108). The cDNA was amplified by polymerase chain reaction (PCR) using the following primer: 5′-ATTCTAAGCTTGGGCCACCATGGAAGC-3′ (SEQ ID NO:628) and 5′-TCTAGAGTTTAAACTTACTAACACTCTCCCCTGTTGAAGC-3′ (SEQ ID NO:629). After digestion with HindIII and Pmel restriction enzymes (New England Biolabs), the purified fragment was cloned into the pCDNA 3.1 V5 vector (Life Technologies) digested with the same restriction enzymes.

HumanizedMN-E6 light chain cDNA was amplified by polymerase chain reaction (PCR) using the following primer: 5′-AGTATGGCCCAGCCGGCCGAAATTGTGTTGACACAGTCTCCAG-3′ (SEQ ID NO: 630) and 5′-TAGAAGGCACAGTCGAGGCTGATCAG-3′ (SEQ ID NO:631). After digestion with SfiI and Pmel restriction enzymes (New England Biolabs), the purified fragment was cloned into the pSECTag2 vector (Life Technologies) digested with the same restriction enzymes.

Humanized MN-E6 IgG1 heavy chain cloning

HumanizedMN-E6 IgG2 constructs (pCDNA 3.1 V5 and pSECTag2) were digested with BstEII and Pmel (New England Biolabs) to remove the IgG2 heavy chain constant region. The vector with humanizedMN-E6 heavy chain variable region was purified. Human IgG1 heavy chain constant region was synthesized by our request by IDT. IA (SEq ID NOS: 60-61). Both gBLOCKS and the purified vector with humanizedMN-E6 variable region were ligated using the Gibson assembly cloning kit (New England Biolabs).

HumanizedMN-E6 lambda light chain cloning

HumanizedMN-E6 kappa light chain constructs (pCDNA 3.1 V5 vector and pSECTag2 vector) were digested with KpnI and Pmel (New England Biolabs) to remove the kappa light chain constant region. The vector with humanizedMN-E6 light chain variable region was purified. Human lambda light chain constant region was synthesized by our request by IDT. IA (SEQ ID NO: 115). Both, gBLOCK and the purified vector with humanizedMN-E6 light chain variable region were ligated using the Gibson assembly cloning kit (New England Biolabs).

Humanized MN-C2 IgG1 and IgG2 heavy chain cloning

HumanizedMN-E6 IgG1 and IgG2 heavy chain in pSECTag2 were digested with SfiI and Agel to remove theMN-E6 variable region. HumanizedMN-E6 IgG1 and IgG2 heavy chain in pCDNA 3.1 V5 were digested with HindIII and Agel to remove theMN-E6 variable region The vectors with human IgG1 or IgG2 constant region were purified. Humanized MN-C2 heavy chains were synthesized by our request by IDT. IA (SEQ ID NOS: 160 and 165). Sequence to be cloned into pCDNA 3.1 V5 contains in 5′ the murine Ig kappa chain leader sequence (SEQ ID NO 160). Both, gBLOCK and purified vector with human IgG1 or IgG2 constant region were ligated using the Gibson assembly cloning kit (New England Biolabs).

### Humanized MN-C2 Kappa/Lambda Light Chain Cloning

Two humanized MN-C2 variable region fused to the kappa light chain constant region and two humanized MN-C2 variable region fused to the lambda light chain constant region were synthesized by our request by IDT. IA (SEQ ID NOS: 210 and 213 and SEQ ID NOS: 216 and 219, respectively), pCDNA 3.1 V5 was digested with HindIII and Pmel restriction enzymes (New England Biolabs) and pSEC Tag2 was digested with SfiI and Pmel restriction enzymes (New England Biolabs). Both plasmids were then purified. SEQ ID NOS: 210 and 216 were ligated into digested pCDNA 3.1 V5 and SEQ ID NOS: 213 and 219 were ligated into digested pSEC Tag2 using the Gibson assembly cloning kit (New England Biolabs).

### Humanized C3 IgG1 Heavy Chain Cloning

Humanized E6 IgG1 construct (pSECTag2) was digested with SfiI and Agel (New England Biolabs) to remove the E6 heavy chain variable region. The vector without humanized E6 heavy chain variable region was purified. Humanized C3 heavy chain variable region was synthesized by our request by IDT. IA (SEQ ID NO:457), gBLOCK and the purified vector were ligated using the Gibson assembly cloning kit (New England Biolabs).

### Humanized C3 Kappa Light Chain Cloning

pEF V5-His was digested with BamHI and Pmel (New England Biolabs) and purified. Humanized C3 kappa light chain was synthesized by our request by IDT. IA (SEq ID NO: 504). Both, gBLOCK and the purified vector were ligated using the Gibson assembly cloning kit (New England Biolabs).

### Humanized C8 Kappa Light Chain Cloning

pEF V5-His was digested with BamHi and Pmel (New England Biolabs) and purified. Humanized C8 kappa light chain was synthesized by our request by IDT. IA (SEq ID NO: 583). Both, gBLOCK and the purified vector were ligated using the Gibson assembly cloning kit (New England Biolabs).

### Example 3—Cloning of Humanized scFV of Anti-MUC1* Extracellular Domain Antibodies

Humanized E6 scFV Cloning:

pSEC Tag2 was digested with SfiI and Pmel (New England Biolabs) and purified. Humanized E6 scFV gBLOCKS were synthesized by our request by IDT. IA (SEQ ID NOS: 604-605). Both, gBLOCKs and the purified vector were ligated using the Gibson assembly cloning kit (New England Biolabs).

Humanized E6 scFV cDNA was amplified by polymerase chain reaction (PCR) using the following primers: 5-ACTGTCATATGGAGGTGCAGCTGGTGGAGTCTG-3′ (SEQ ID NO: 632) and 5′-ACTGTCTCGAGTTTAATTTCCACTTTGGTGCCGCTGC-3′ (SEQ ID NO: 633). After digestion with Ndel and Xhol restriction enzymes (New England Biolabs), the purified fragment was cloned into the pET21b vector (Novagen) digested with the same restriction enzymes. Humanized E6 scFV cDNA was cloned 5′ of the Histidine Tag for protein purification.

Humanized E6 scFV cDNA was amplified by polymerase chain reaction (PCR) using the following primers: 5-ACTGTCATATGGAGGTGCAGCTGGTGGAGTCTG-3′ (SEQ ID NO: 634) and 5′-ACTGTACCGGTTTTAATTTCCACTTTGGTGCCGCTGC-3′ (SEQ ID NO: 635). After digestion with Ndel and Agel restriction enzymes (New England Biolabs), the purified fragment was cloned into a modified pET21b vector (Novagen) digested with the same restriction enzymes. The vector was modified to include the StrepTag2 sequence followed by 2 stop codons 5′ of the Histidine Tag. Humanized E6 scFV cDNA was cloned 5′ of the StrepTag2 for protein purification.

Humanized E6, C2, C3 and C8 scFV-Fc Cloning

Humanized E6 IgG1 construct (pSECTag2) was digested with SfiI and SacII (New England Biolabs) to remove the E6 heavy chain variable region and part of the IgG1 heavy chain constant region. The vector without humanized E6 heavy chain variable region was purified. Humanized E6. C2, C3 and C8 scFV gBLOCKS were synthesized by our request by IDT. IA (SEQ ID NO:258-259. 262-263. 266-267 and 270-271). E6. C2. C3 and C8 gBLOCKS and the purified vector were ligated using the Gibson assembly cloning kit (New England Biolabs) to assemble the corresponding scFV in frame of the human IgG1 Fc region.

Humanized E6 scFV-Fc Y407R Cloning

Humanized E6 scFV-Fc tyrosine 407 was mutated to an arginine (Y407R) by site directed mutagenesis. The Q5 site directed mutagenesis kit (NEB) was used with the following primers: 5′-CTTCTTCCTCAGGAGCAAGCTCACCGTGG-3′ (SEQ ID NO:636) and 5′-GAGCCGTCGGAGTCCAGC-3′ (SEQ ID NO:637)

Humanized E6 scFV-Fc Hingeless Cloning

Hinge region of humanized E6 scFV-Fc was removed by site directed mutagenesis. The Q5 site directed mutagenesis kit (NEB) was used with the following primers: 5′-GCACCTGAACTCCTGGGG-3″ (SEQ ID NO:638) and 5′-TTTAATTTCCACTTTGGTGCCG-3′ (SEQ ID NO:639)

### Example 4—Cloning of CAR-T of Anti-MUC1* Extracellular Domain Antibodies

### Car E6 Cd28/4-1Bb/Cd3z Cloning:

pCDNA 3.1 V5 was digested with KpnI and Pmel (New England Biolabs) and purified. Full CAR-T E6 (CD8/CD28/4-1BB/CD3z) gBLOCK was synthesized by our request by IDT. IA (SEq ID NO:305). Both, gBLOCK and the purified vector were ligated using the Gibson assembly cloning kit (New England Biolabs).

### Car E6 Cd3z Cloning:

pCDNA 3.1 V5 CAR-T E6 CD8/CD28/4-1BB/CD3% was digested with EcoRV and Pmel (New England Biolabs) to remove cytoplasmic domains. The vector without cytoplasmic domains was purified. CAR-T E6 CD8/CD3% gBLOCK was synthesized by our request by IDT. IA (SEq ID NO:296). Both, gBLOCK and the purified vector were ligated using the Gibson assembly cloning kit (New England Biolabs).

### Car E6 Cd28/Cd3z Cloning:

pCDNA 3.1 V5 CAR-T E6 CD8/CD28/4-1BB/CD3z was digested with EcoRV and Pmel (New England Biolabs) to remove cytoplasmic domains. The vector without cytoplasmic domains was purified. CAR-T E6 CD8/CD28/CD3z gBLOCK was synthesized by our request by IDT. IA (SEq ID NO:299). Both, gBLOCK and the purified vector were ligated using the Gibson assembly cloning kit (New England Biolabs).

### Car E6 4-1Bb/Cd3z Cloning:

pCDNA 3.1 V5 CAR-T E6 CD8/CD28/4-1BB/CD3z was digested with EcoRV and Pmel (New England Biolabs) to remove cytoplasmic domains. The vector without cytoplasmic domains was purified. CAR-T E6 CD8/4-1BB/CD3% gBLOCK was synthesized by our request by IDT. IA (SEq ID NO:302). Both, gBLOCK and the purified vector were ligated using the Gibson assembly cloning kit (New England Biolabs).

### Car C2 Cd28/4-1Bb/Cd3z Cloning:

pCDNA 3.1 V5 CAR-T E6 CD8/CD28/4-1BB/CD3% was digested with KpnI and EcoRV (New England Biolabs) E6 scFV. The vector without E6 scFV was purified. CAR-T C2 gBLOCKs were synthesized by our request by IDT. IA (SEq ID NOS: 308-309). Both, gBLOCKs and the purified vector were ligated using the Gibson assembly cloning kit (New England Biolabs).

CAR Sub-Cloning into Lentiviral Vectors:

All pcDNA 3.1 V5 CAR cDNAs were amplified by polymerase chain reaction (PCR) using the following primers: 5-CGCGGCTAGCTTAAGCTTGGTACCGAGGGCCA-3′ (SEQ ID NO:640) and 5′-CGCGGCGGCCGCCTGATCAGCGGGTTTAAACTTATC-3′ (SEQ ID NO: 641). After digestion with Nhel and NotI restriction enzymes (New England Biolabs), the purified fragments were cloned into lentiviral vectors (pCDH-EF1-MCS-IRES GFP and pCDH-CMV-MCS-EF1-copGFP+puro. SBI) digested with the same restriction enzymes.

### Car-E6-Fc/8/41Bb/Cd3z Cloning:

pCDH-CMV-MCS-EF1-copGFP+puro (SBI) was digested with Nhel and NotI (New England Biolabs) and the vector was purified, gBLOCKs were synthesized by our request by IDT. IA (SEq ID NOS: 312. 313 and 314). The gBLOCKs and the purified vector were ligated using the Gibson assembly cloning kit (New England Biolabs).

### Car-E6-Fch/8/41Bb/Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 317 and 314). The gBLOCKs and the purified vector were ligated using the Gibson assembly cloning kit (New England Biolabs).

### Car-E6-Fc-4-41Bb-Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 313 and 320). The gBLOCKs and the purified vector were ligated using the Gibson assembly cloning kit (New England Biolabs).

### Car-E6 Fch/4/41Bb/Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 317 and 320). The gBLOCKs and the purified vector were ligated using the Gibson assembly cloning kit (New England Biolabs).

### Car-E6 Igd/8/41Bb/Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 325 and 326). The gBLOCKs and the purified vector were ligated using the Gibson assembly cloning kit (New England Biolabs).

### Car-E6 Igd/4/41Bb/Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 329 and 326). The gBLOCKs and the purified vector were ligated using the Gibson assembly cloning kit (New England Biolabs).

### Car-E6 X4/8/41Bb/Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 332 and 326). The gBLOCKs and the purified vector were ligated using the Gibson assembly cloning kit (New England Biolabs).

### Car-E6 X4/4/41Bb/Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 335 and 326). The gBLOCKs and the purified vector were ligated using the Gibson assembly cloning kit (New England Biolabs).

### Car E6-8+4-4-41Bb-Cd3z Cloning:

pCDH-CMV-MCS-EF1-copGFP+puro (SBI) was digested with Nhel and NotI (New England Biolabs) and the vector was purified. gBLOCKs were synthesized by our request by IDT. IA (SEq ID NOS: 312. 338 and 326). The gBLOCKs and the purified vector were ligated using the Gibson assembly cloning kit (New England Biolabs).

### Example 5-Lentivirus production

HEK 293T cells (ATCC) were used to produce lentivirus. The day prior transfection plates (6well plate) were coated with poly-D-lysine and cells seeded so that cell density reaches 90-95% at the time of transfection and cultures in a 5% CO2 atmosphere. The next daycells were transfected with Lipofectamine 3000 (life technologies) and Opti-MEMR I Reduced Serum Medium according to the manufacturer instructions (0.75 ug of lentiviral expression vecotr and 2.25 ug of pPACKH1 packaging mix was used). After 6h incubation, the media was changed and media containing lentivirus was harvested after 24 and 48 hours. Lentivirus was concentrated with Lenti-X concentrator (Clontech) and titer was calculated using the Lenti-X p@4 Rapid Titer Kit (Clontech). Lentivirus was store at −80C in single-use aliquots.

### Example 6—Lipofectamine Transient Expression

HEK 293T cells (ATCC) were used to test expression of humanized IgG. The night before transfection, cells were passed at ⅓ dilution (6well plate) and cultures in a 5% CO2 atmosphere. The next day. 1 hour before transfection, the media was change to complete media without antibiotics (DMEM high glucose from ATCC containing 10% fetal calf serum). For transfection, we used Lipofectamine 3000 (life technologies) and Opti-MEMR: I Reduced Serum Medium according to the manufacturer instructions. 1.25 ug of the heavy chain construct and 1.25 ug of the light chain construct or 2.5 ug of Fc-fusion constructs was used. After 48h incubation, the media was collected, cleared by centrifugation and used in an ELISA assay to quantify the level of humanized IgG expression and binding to PSMGFR peptide.

### Example 7—Polyethylenimine (PEI) Large Scale Transient Expression

HEK 293T cells (ATCC) were used for large scale expression of Fc-fusion protein. The night before transfection, cells were passed (6.5×10⁶ cells in 150 mm dish) and cultures in a 5% CO2 atmosphere. The next day. 1 hour before transfection.cell were washed once with PBS pH 7.4 and the media was change to complete media without antibiotics (DMEM high glucose from ATCC containing 10% ultra low IgG fetal calf serum). For transfection, we used Polyethylenimine “Max” (PEI “Max”. Polysciences) and Opti-MEMR I Reduced Serum Medium (25 ug of Fc-fusion constructs+250 ug of PEI). After 72h incubation, the media was collected and stored at −20° C., or cleared by centrifugation/filtration for purification.

### Example 8—Jurkat Cells Lentivirus Transduction

Protocol #1: A 50/50 solution (2 mL) of lentivirus was prepared in fresh media, supplemented with 8 ug/mL of polybrene and added to a well of a 6 well plate. Jurkat E6-1 cells (ATCC. TIB-152) were pelleted at 1200 rpm for 5 min at RT and resuspended in fresh media (RPMI containing 10% fetal calf serum and 1% penicillin/streptomycin/amphotericin b). Cells were counted and add 2×10⁵ cells to the well containing the virus+Polybrene solution. Incubate for 24-48h and add fresh media and/or split the cells. After 72h, start growing cells with antibiotic selection (puromycin).

Protocol #2: Jurkat E6-1 cells (ATCC. TIB-152) were pelleted at 1200 rpm for 5 min at RT and resuspended in fresh media (RPMI containing 10% fetal calf serum and 1% penicillin/streptomycin/amphotericin b) at 2.5×10⁵ cells/mL. Add 2 mL of cells to a 15 mL sterile conical tube, add 1× of Transdux infection reagent (1X. SBI) and lentivirus. Mix gently and incubate at RT for 20 min. Centrifuge cells at 1900 rpm for 30 min at 32° C. remove supernatant, resuspend cells in 2 mL of fresh media and transfer cells to a well of a 6 well plate. Inspect cells for GFP expression after 48h.

### Example 9—T Cells Lentivirus Transduction

Highly purified T cells (AllCells) were pelleted at 200xg for 5 min at RT and resuspended at 1×10⁶ cells/mL in fresh media (RPMI1640) containing 10% fetal calf serum and 1% penicillin/streptomycin). Add CD3/CD28 activator Dynabeads (Thermo Fisher. 25 uL for 1×10⁶ cells) and seed 24 well plate with ImL of cells and add IL2 (Thermo Fisher). Monitor cells daily and split cells if needed. The day before the transduction coat a plate with Retronectin (Takara) and store it overnight at 4° C. The next, remove the Retronectin solution add add a blocking solution (2% BSA in PBS) and incubate 30 min at RT. Remove BSA solution add add PBS until cells are ready. Collect activated T cells and resuspend them at 0.5×10⁶ cells/mL in fresh media. Add ImL of cells to the retronectin treated plate. ImL of lentivirus solution and IL2. Cells were spinoculated by centrifugation of the pate at 1000xg for 90 min at RT. The plate was return to the incubator overnight. Next, remove ImL of media, add ImL of virus and repeat spinoculation. Monitor cells and split them if necessarry at a density of 0.5-1×10⁶ cells/mL. T cells can be used for cytokine release assay or cytotoxicity assay 48h post transduction.

### Example 10—IL2 Detection

IL-2 secretion in media was measured using a human IL-2 ELISA kit (Thermo Fisher). Plates were coated with and anti-IL-2 antibody (coating antibody. 1/100 in PBS). After overnight incubation at 4° C., the plate was wash 3 times with PBS-T and a 4% BSA solution was added to block remaining binding site on the well. After 1h at RT the plate was washed once with PBS-T and conditioned media (CM) and IL-2 standard diluted in PBS+4% BSA, was added. After 2 h at RT the plate was washed 3× with PBS-T and anti-human IL-2 (detection antibody) diluted in PBS+4% BSA (1/100), was added. After 2 h at RT the plate was washed 5× with PBS-T and Streptavidin-HRP (1/400) was added. After 30 min at RT, the plate was washed 7× with PBS-T (soak Imin each wash) and substrate solution was added. The reaction was stopped after 20 min by adding the stop solution and absorbance was read at 450 nm (minus absorbance at 550 nm) within 30 min of stopping.

### Example 11—IFN-γ Detection

IFN-γ secretion in media was measured using a human IFN-γ ELISA kit (Biolegend). Plates were coated with and anti-IFN-γ antibody (capture antibody. 1× in coating buffer). After overnight incubation at 4° C., the plate was washed 4 times with PBS-T and blocking solution was added to block remaining binding site on the well. After 1h at RT (shaking at 500 rpm) the plate was washed 4 times with PBS-T and conditioned media (CM) and IFN-γ standard, was added. After 2 h at RT with shaking, the plate was washed 4 times with PBS-T and detection antibody (1×), was added. After 1h at RT with shaking, the plate was washed 4 times with PBS-T and Avidin-HRP (1×) was added. After 30 min at RT with shaking, the plate was washed 5 times with PBS-T (soak Imin each wash) and TMB substrate solution was added. The reaction was stopped after 20 min by adding the stop solution and absorbance was read at 450 nm (minus absorbance at 570 nm) within 15 min of stopping.

### Example 12—CAR T Cytotoxicity Assay

Human T cells were isolated from whole blood according to standard protocols. The T cells were then separately transduced twice with lenti virus bearing the CAR constructs, wherein the CAR constructs bear a GFP tag. Following 2-3 days of culture in RPMI 10% FBS and IL-2, the cells were stained with F (ab) 2 to label surface expression of MN-E6, MN-C2, MN-C3 and MN-C8. Cells were then sorted by flow cytometry for Fab-positive. GFP-positive cells. That means that the double positive population had a CAR inserted and that the CAR exposed the correct antibody fragment. The CAR T cells were then ready to be mixed with the MUC1* negative control cells or the target MUC1* positive cancer cells.

The target cells were prepared as follows: Harvest target cells and resuspend cells in serum-free medium containing 15 uM of CMTMr dye (Cell Tracker Orange. 5-and-6-4-chloromethyl benzoyl amino tetramethylrhodamine. Thermo Fisher) at 1-1.5×10⁶ cells/mL. Incubate 30 min under growth conditions appropriate to particular cell type. Wash in culture media and transfer stained cells to a new tube and incubate the cells 60 min in media. Wash 2 more times in culture media to get rid of all excess dye. Set up the assay in 24 well plates with 0.5 ml media total volume. Resuspend the target cells (and control target cells) so that there are always 20.000 cells per well (20.000 cells/250 ul). Plate 250 ul in each well. Add 250 ul of the T cells so that the ratio of T cell: target cells=20:1. 10:1. 5:1 or 1:1 . . . . Analyse cells after 24 h and 72h. For suspension target cells, take off the 0).5 ml media from the well and place in tube, wash the well with 0.5 ml media or PBS. For adherent target cells, take off the 0).5 ml media from the well and place in tube, wash the well with 0.5 ml PBS. Add the PBS to the same tube and add 120 ul trypsin to the well. Incubate for 4 min then add 0.5 ml media to neutralize trypsin and place that in the tube as well. Spin cells and resuspend pellet in 100 ul FACS buffer. Spin cells again. Resuspend cells in 100 ul buffer+5 ul anti-CD3 antibody, for 30 min on ice (to stain T cells). After 30 min, wash stained cells 2× with FACS buffer and resuspend in 250 ul buffer. Run the cells through the filter cap of the FACS tube. 10 min prior to analysis, add 10 ul 7AAD dye to each tube and analyze with Fortessa under the Cytotoxicity template.

### Example 13—ELISA Expression Level of Humanized IgG

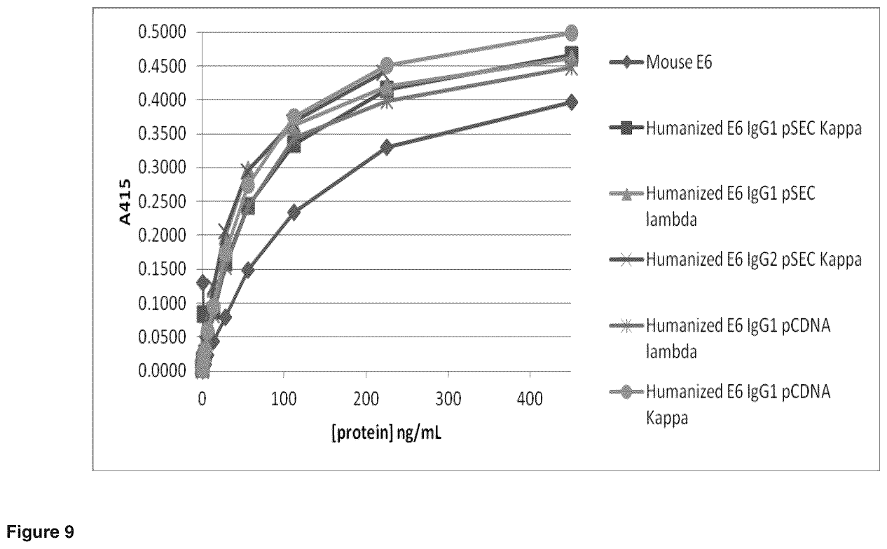

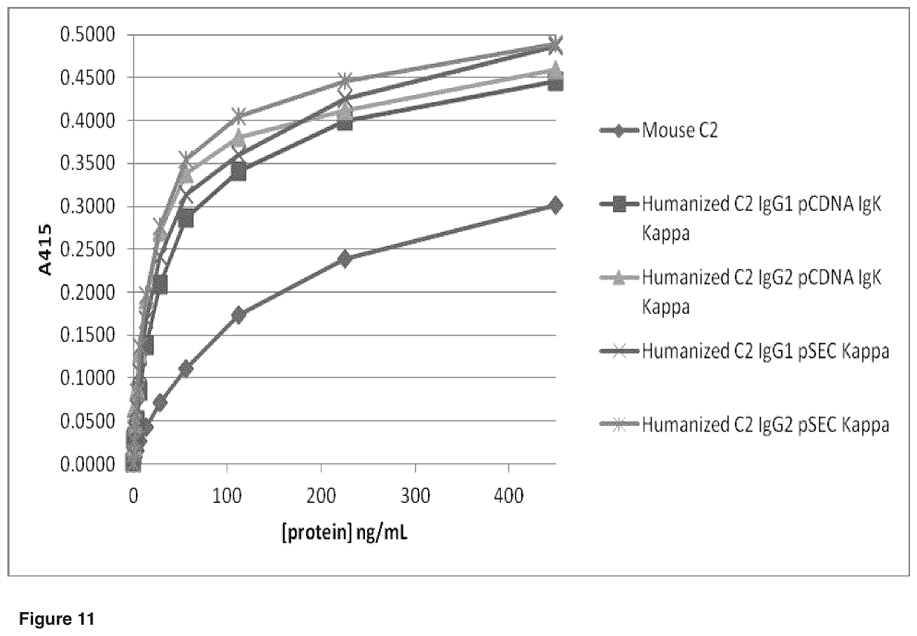

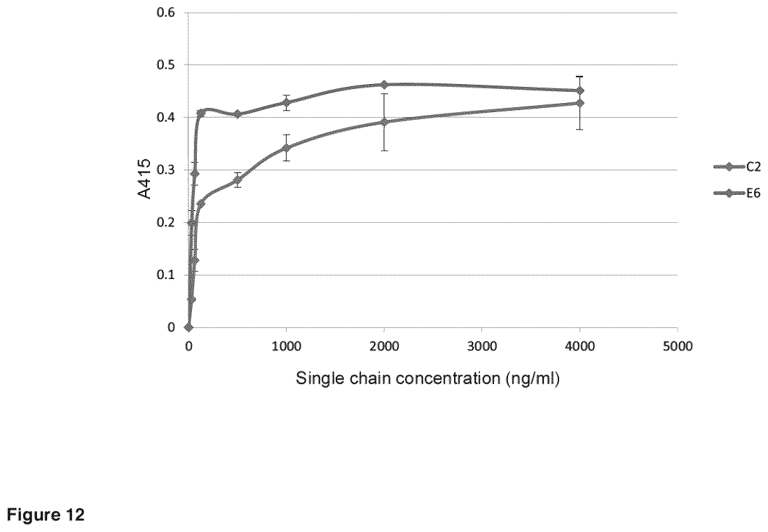

Goat Anti-human Fc specific antibody was diluted to 5 ug/mL in 0.1M carbonate/bicarbonate buffer pH 9.6 and 50 uL was added to each well of a 96 well plate. After overnight incubation at 4° C., the plate was wash twice with PBS-T and a 3% BSA solution was added to block remaining binding site on the well. After 1h at RT the plate was washed twice with PBS-T and conditioned media (CM), diluted in PBS-T+1% BSA, was added at different concentrations. Also, purified human IgG (life technologies), diluted in PBS-T+1% BSA, was added at different concentrations to make a standard curve for determination of the expression level of the humanized IgG or Fc-fusion protein. After 1h at RT the plate was washed 3× with PBS-T and anti-human (H+L) HRP (life technologies) diluted in PBS-T+1% BSA, was added at 1/2500. After 1h at RT the plate was washed 3× with PBS-T and binding of human IgG and humanized IgG was measured at 415 nm using a ABTS solution (ThermoFisher) (FIG. 9) (MN-E6) and FIG. 11 (MN-C2)).

### Example 14—ELISA Humanized IgG Binding to PSMGFR Peptide of the MUC1* Extracellular Domain

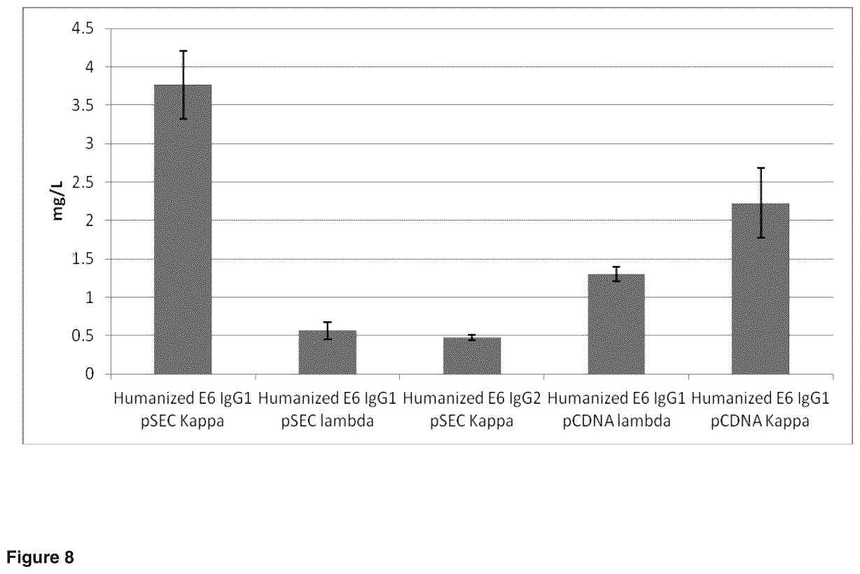

A synthetic peptide of sequence PSMGFR was covalently coupled to BSA using Imject Maleimide activated BSA kit (Thermo Fisher). PSMGFR coupled BSA was diluted to 7.5 ug/mL in 0.1M carbonate/bicarbonate buffer pH 9.6 and 50 uL was added to each well of a 96 well plate. After overnight incubation at 4° C., the plate was wash twice with PBS-T and a 3% BSA solution was added to block remaining binding site on the well. After 1h at RT the plate was washed twice with PBS-T and conditioned media (CM), diluted in PBS-T+1% BSA, was added at different concentrations. At the same time corresponding mouse IgG was diluted in PBS-T+1% BSA and added at different concentrations as binding control. After 1 h at RT the plate was washed 3× with PBS-T and anti-human (H+L) HRP (life technologies) diluted in PBS-T+1% BSA, was added at 1/5000 to detect binding of humanized IgG. Anti-Mouse HRP (life technologies) diluted in PBS-T+1% BSA, was added at 1/2500 to detect binding of mouse IgG. After 1h at RT the plate was washed 3× with PBS-T and binding was measured at 415 nm using a ABTS solution (ThermoFisher) (FIG. 8 (MN-E6) and FIG. 10 (MN-C2)).

### Example 15—Stable Cell Lines Generation

CHO-K1 cells (ATCC) were used to create stable cell lines expressing high level of humanized IgG. HEK293 cells (ATCC) were used to create stable cell lines expressing high level of Fc-fusion proteins. The night before transfection, cells were passed at ⅓ dilution (6well plate) and cultures in a 5% CO2 atmosphere. The next day. 1 hour before transfection, the media was change to complete media without antibiotics (F12K or DMEM containing 10% fetal calf serum). For transfection, we used Lipofectamine 3000 (life technologies) and Opti-MEMR I Reduced Serum Medium according to the manufacturer instructions. 1.25 ug of the heavy chain construct and 1.25 ug of the light chain construct or 2.5 ug of Fc-fusion constructs was used. After 24h, cells were trypsinized and plated into a T75 flask (in F12K or DMEM containing 10% fetal calf serum). After 24h, cells were trypsinized, diltuted to 100 cells/mL and 1000cells/mL in F12K or DMEM containing 10% FCS and selection agent (Zeocin for pSECTag2 or G418 for pCDNA 3.1 V5), plated in 96 well plate (100 uL per well) and cultures in a 5% CO2 atmosphere. After 2-3 weeks, the culture media from single clones were collected, cleared by centrifugation and used in an ELISA assay to quantify the level of humanized IgG expression and binding to PSMGFR peptide. The clones with the highest expression and PSMGFR binding were expanded for large scale expression.

### Example 16—scFV Expression

pET21b E6 scFV plasmid (with HisTag or StrepTagII) was transformed into Shuffle T7 express competent cells (NEB). TB broth (Terrific broth) was inoculated with 1/100 of an overnight culture (LB broth−30° C.-200 rpm) and cultured at 30° C./200 rpm. When OD600 reached ˜1, temperature was reduced to 20° C., and growth was continued. After 2h. recombinant protein expression was induced with 0.2 mM Isopropyl-β-D-thio-galactoside (IPTG. Gold Biotechnology) and culture was stopped after 22 h. After harvesting the cells by centrifugation (6000 rpm for 10 min at 4° C.), cell pellet was resupended with running buffer. For Histag protein buffer was: 50 mM Tris pH8.0. 300 mM NaCl and 5 mM imidazole. For StrepTagII protein buffer was 100 mM Tris pH 8.0 and 150 mM NaCl.

### Example 17—His Tag EscFV Purification

MgCl2 (0.5 mM). DNAse (0.5 ug/mL. Sigma). PMSF (ImM. Gold Bitotechnology) and BugBuster (1X. Novagen) was added. Cell suspension was incubated on a rotating platformfor 20 min at RT. Insoluble cell debris was removed by centrifugation (20000 rpm for 30 min at 4° C.). The cleared lysate was then applied to a Ni-NTA column (Qiagen) equilibrated with the running buffer. The column was washed before eluting the protein off the column with the running buffer supplemented with 495 mM imidazole. The protein was further purified by size exclusion chromatography (Superdex 200). The fractions containing the protein were pooled, aliquoted and stored at −80° C.

### Example 18—StrepTagII EscFV Purification

MgCl2 (0.5 mM), DNAse (0.5 ug/mL. Sigma), PMSF (1 mM, Gold Bitotechnology) and BugBuster (1X. Novagen) was added. Cell suspension was incubated on a rotating platformfor 20 min at RT. Insoluble cell debris was removed by centrifugation (20000 rpm for 30 min at 4° C.). The cleared lysate was then applied to a Strep-Tactin column (IBA) equilibrated with the running buffer. The column was washed before eluting the protein off the column with the running buffer supplemented with 5 mM d-Desthiobiotin. The protein was further purified by size exclusion chromatography (Superdex 200). The fractions containing the protein were pooled, aliquoted and stored at −80° C.

### Example 19—Humanize IgG/Fc-Fusion Purification

Condition media (from transient transfection or stable cell line) was collected, cleared by centrifugation and filtered (0).2 um). The media was then loaded on a protein A (Genscript) or CaptureSelect FcXL (Thermo Fisher) and the protein purified according to manufacturer instructions using acid condition for elution. The eluted protein was then dialyzed against PBS pH 7.4 and further purified by size exclusion chromatography (Superdex 200). The fractions containing the protein were pooled, aliquoted and stored at −80° C.

### Example 20Immunohistochemistry

Human tissue specimens were purchased from Biomax. The tissues were either normal or cancerous as determined by a board certified pathologist. Tissues were anonymized but were labeled with a number, tissue type, stage of cancer and if available, a TNM tumor grading designation. TNM grading is as follows: T is primary tumor. Tx is primary tumor cannot be assessed. TO is no evidence of a tumor. This is carcinoma in situ, intraepithelial or invasion of lamina propia. T1 is tumor invades submucosa. T2 is tumor invades muscularis propia. T3 is tumor invades through muscularis propia into subserosa or into non-peritonealized pericolic or perirectal tissues. T4 is tumor directly invades other organs or structures and/or perforate visceral peritoneum. N is regional lymph nodes. NO is no regional lymph node metastasis. N1 is metastasis in 1 to 3 regional lymph nodes. N2 is metastatic in 4 or more regional lymph nodes. M is for distant metastasis. M0 means no distant metastasis. M1 is distant metastasis.

Tissues were stained with a primary anti-MUC1* antibody mouse monoclonal MN-C2, MN-E6, humanized MN-E6 scFv-Fc, or humanized MN-E6 scFv-Fc-biotin. If the primary were a mouse monoclonal antibody, then the secondary antibody used was a rabbit anti-mouse HRP-conjugated antibody. If the primary were a humanized antibody, then the secondary was a goat-anti-human HRP conjugated antibody antibody. If the primary were a biotinylated antibody, then the secondary was a streptavidin HRP conjugated antibody.

Tissue specimens were de-paraffinized using xylene and ethanol according to standard protocols. An antigen retrieval procedure was used for some tissues which involved 10 mM Sodium Citrate-0.05% Tween pH 6 buffer (pre boil buffer, keep warm) boil 10′, cool down 20′ in rice cooker, then rinse cold tap water 5 minutes then two 5 min, washes in TBS. Tissues were blocked for 1 hr at RT in 10% NGS plus 5% BSA in TBS. If the primary antibody used was humanized MN-E6scFv, which was conjugated to biotin so that it could be visualized by a secondary antibody, the tissues were pre-blocked with an avidin solution then a biotin solution. Primary antibodies were incubated with tissues overnight at 4 degrees C., in 1% BSA-TBS with gentle orbital rotation. Tissues were rinsed with TBS-T for 5 minutes with gentle rocking. For HRP-conjugate detection only, mounted tissues were incubated in 3% H₂O₂ in TBS for 15 minutes at RT. For tissues incubated with biotinylated primary antibodies, they were then bathed in StreptAvidin for 10 min with Streptavidin-HRP label (Biocare Cat #: HP604 G, H, L), then washed 3 times for 5 minutes at RT in TBS-T with gentle rocking. They were then developed with chromogen (DAB-ImL diluent: 1 drop DAB substrate) for 5 minutes at RT, then rinsed with running tap water for 5 minutes. They were then counterstained for 1 second hematoxylin then brief dip in 0.08% NH₄OH ‘bluing reagent’ followed by 5 minutes in running water. Tissues were then dehydrated and mounted with Cytoseal XYL (1 drop/section) and coverslipped.

All of the references cited herein are incorporated by reference in their entirety.

Those skilled in the art will recognize, or be able to ascertain using no more than routine experimentation, many equivalents to the specific embodiments of the invention specifically described herein.

1. A method of treating a MUC1* positive cancer in a subject comprising administering immune cells that express a MUC1* targeting chimeric antigen receptor (CAR) to the subject.

2. The method of claim 1, wherein the MUC1* targeting CAR comprises an anti-MUC1* antibody fragment that specifically binds to a cancer-specific epitope of MUC1* comprising SEQ ID NO: 642 and does not bind to a peptide comprising SEQ ID NO: 643.

3. The method of claim 1, wherein the MUC1* targeting CAR comprises an anti-MUC1* antibody fragment comprising:
a heavy chain variable (HCV) region comprising complementarity determining regions (CDRs) comprising SEQ ID NO: 17 (CDR1), SEQ ID NO: 21 (CDR2), and SEQ ID NO: 25 (CDR3), and
a light chain variable (LCV) region comprising CDRs comprising SEQ ID NO: 70 (CDR1), SEQ ID NO: 74 (CDR2), and SEQ ID NO: 78 (CDR3); or
an HCV region comprising CDRs comprising SEQ ID NO: 123 (CDR1), SEQ ID NO: 127 (CDR2), and SEQ ID NO: 131 (CDR3), and
an LCV region comprising CDRs comprising SEQ ID NO: 173 (CDR1), SEQ ID NO: 177 (CDR2), and SEQ ID NO: 181 (CDR3).

4. The method of claim 3, wherein the anti-MUC1* antibody fragment comprises an HCV sequence having at least 90% identity to SEQ ID NO: 39 and an LCV sequence having at least 90% identity to SEQ ID NO: 94.

5. The method of claim 3, wherein the anti-MUC1* antibody fragment comprises an HCV sequence having at least 90% identity to SEQ ID NO: 145 and an LCV sequence having at least 90% identity to SEQ ID NO: 195.

6. The method of claim 3, wherein the anti-MUC1* antibody fragment comprises an scFv sequence comprising SEQ ID NO: 392 or SEQ ID NO: 394.

7. The method of claim 3, wherein the anti-MUC1* antibody fragment comprises an scFv sequence comprising SEQ ID NO: 396.

8. The method of claim 3, wherein the anti-MUC1* antibody fragment comprises an scFv sequence comprising SEQ ID NO: 398.

9. The method of claim 3, wherein the MUC1* targeting CAR further comprises a T cell receptor co-stimulatory domain.

10. The method of claim 3, wherein the MUC1* targeting CAR further comprises a CD3-zeta T cell receptor co-stimulatory domain, a CD28 T cell receptor co-stimulatory domain, or a 4-1BB T cell receptor co-stimulatory domain.

11. The method of claim 3, wherein the MUC1* targeting CAR further comprises a CD28 T cell receptor co-stimulatory domain and a CD3-zeta T cell receptor co-stimulatory domain.

12. The method of claim 3, wherein the MUC1* targeting CAR further comprises a 4-1BB T cell receptor co-stimulatory domain and a CD3-zeta T cell receptor co-stimulatory domain.

13. The method of claim 3, wherein the MUC1* targeting CAR comprises SEQ ID NO: 611.

14. The method of claim 3, wherein the immune cells comprise T cells.

15. The method of claim 3, wherein the immune cells are isolated from the subject.

16. The method of claim 3, wherein the MUC1* positive cancer comprises a breast cancer.

17. The method of claim 3, wherein the MUC1* positive cancer comprises a prostate cancer or a pancreatic cancer.

18. The method of claim 3, wherein the MUC1* positive cancer comprises a lung cancer or a colon cancer.


SEQUENCE LISTING (643 sequences)

SEQ ID NO: 1 - protein, 1255 aa, synthetic construct
  REGION 1..1255: note = MUC1 Receptor
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 2 - protein, 45 aa, synthetic construct
  REGION 1..45: note = PSMGFR
GTINVHDVETQFNQYKTEAASRYNLTISDVSVSDVPFPFSAQSGA

SEQ ID NO: 3 - DNA, 459 bp, synthetic construct
  misc_feature 1..459: note = Human NME1
  source 1..459: mol_type = other DNA
atggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatga

SEQ ID NO: 4 - protein, 152 aa, synthetic construct
  REGION 1..152: note = Human NME1
MANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 5 - DNA, 1131 bp, synthetic construct
  misc_feature 1..1131: note = Human NME7
  source 1..1131: mol_type = other DNA
atgaatcatagtgaaagattcgttttcattgcagagtggtatgatccaaatgcttcacttcttcgacgttatgagcttttattttacccaggggatggatctgttgaaatgcatgatgtaaagaatcatcgcacctttttaaagcggaccaaatatgataacctgcacttggaagatttatttataggcaacaaagtgaatgtcttttctcgacaactggtattaattgactatggggatcaatatacagctcgccagctgggcagtaggaaagaaaaaacgctagccctaattaaaccagatgcaatatcaaaggctggagaaataattgaaataataaacaaagctggatttactataaccaaactcaaaatgatgatgctttcaaggaaagaagcattggattttcatgtagatcaccagtcaagaccctttttcaatgagctgatccagtttattacaactggtcctattattgccatggagattttaagagatgatgctatatgtgaatggaaaagactgctgggacctgcaaactctggagtggcacgcacagatgcttctgaaagcattagagccctctttggaacagatggcataagaaatgcagcgcatggccctgattcttttgcttctgcggccagagaaatggagttgttttttccttcaagtggaggttgtgggccggcaaacactgctaaatttactaattgtacctgttgcattgttaaaccccatgctgtcagtgaaggactgttgggaaagatcctgatggctatccgagatgcaggttttgaaatctcagctatgcagatgttcaatatggatcgggttaatgttgaggaattctatgaagtttataaaggagtagtgaccgaatatcatgacatggtgacagaaatgtattctggcccttgtgtagcaatggagattcaacagaataatgctacaaagacatttcgagaattttgtggacctgctgatcctgaaattgcccggcatttacgccctggaactctcagagcaatctttggtaaaactaagatccagaatgctgttcactgtactgatctgccagaggatggcctattagaggttcaatacttcttcaagatcttggataattag

SEQ ID NO: 6 - protein, 376 aa, synthetic construct
  REGION 1..376: note = Human NME7
MNHSERFVFIAEWYDPNASLLRRYELLFYPGDGSVEMHDVKNHRTFLKRTKYDNLHLEDLFIGNKVNVFSRQLVLIDYGDQYTARQLGSRKEKTLALIKPDAISKAGEIIEIINKAGFTITKLKMMMLSRKEALDFHVDHQSRPFFNELIQFITTGPIIAMEILRDDAICEWKRLLGPANSGVARTDASESIRALFGTDGIRNAAHGPDSFASAAREMELFFPSSGGCGPANTAKFTNCTCCIVKPHAVSEGLLGKILMAIRDAGFEISAMQMFNMDRVNVEEFYEVYKGVVTEYHDMVTEMYSGPCVAMEIQQNNATKTFREFCGPADPEIARHLRPGTLRAIFGKTKIQNAVHCTDLPEDGLLEVQYFFKILDN

SEQ ID NO: 7 - protein, 16 aa, synthetic construct
  REGION 1..16: note = NME7 peptide 1 (A domain)
MLSRKEALDFHVDHQS

SEQ ID NO: 8 - protein, 11 aa, synthetic construct
  REGION 1..11: note = NME7A peptide 2 (A domain)
SGVARTDASES

SEQ ID NO: 9 - protein, 20 aa, synthetic construct
  REGION 1..20: note = NME7B peptide 1 (B domain)
DAGFEISAMQMFNMDRVNVE

SEQ ID NO: 10 - protein, 16 aa, synthetic construct
  REGION 1..16: note = NME7B peptide 2 (B domain)
EVYKGVVTEYHDMVTE

SEQ ID NO: 11 - protein, 29 aa, synthetic construct
  REGION 1..29: note = NME7B peptide 3 (B domain)
AIFGKTKIQNAVHCTDLPEDGLLEVQYFF

SEQ ID NO: 12 - DNA, 366 bp, synthetic construct
  misc_feature 1..366: note = Mouse E6 Heavy chain variable region sequence
  source 1..366: mol_type = other DNA
gaggtgaaggtggtggagtctgggggagacttagtgaagcctggagggtccctgaaactctcctgtgtagtctctggattcactttcagtagatatggcatgtcttgggttcgccagactccaggcaagaggctggagtgggtcgcaaccattagtggtggcggtacttacatctactatccagacagtgtgaaggggcgattcaccatctccagagacaatgccaagaacaccctgtacctgcaaatgagcagtctgaagtctgaggacacagccatgtatcactgtacaagggataactacggtaggaactacgactacggtatggactactggggtcaaggaacctcagtcaccgtctcctca

SEQ ID NO: 13 - protein, 122 aa, synthetic construct
  REGION 1..122: note = Mouse E6 Heavy chain variable region sequence
EVKVVESGGDLVKPGGSLKLSCVVSGFTFSRYGMSWVRQTPGKRLEWVATISGGGTYIYYPDSVKGRFTISRDNAKNTLYLQMSSLKSEDTAMYHCTRDNYGRNYDYGMDYWGQGTSVTVSS

SEQ ID NO: 14 - DNA, 75 bp, synthetic construct
  misc_feature 1..75: note = Mouse E6 heavy chain variable framework region 1 (FWR1) sequence
  source 1..75: mol_type = other DNA
gaggtgaaggtggtggagtctgggggagacttagtgaagcctggagggtccctgaaactctcctgtgtagtctct

SEQ ID NO: 15 - protein, 30 aa, synthetic construct
  REGION 1..30: note = Mouse E6 heavy chain variable framework region 1 (FWR1) sequence
EVKVVESGGDLVKPGGSLKLSCVVSGFTFS

SEQ ID NO: 16 - DNA, 30 bp, synthetic construct
  misc_feature 1..30: note = Mouse E6 heavy chain variable complementarity determining regions1 (CDR1) sequence
  source 1..30: mol_type = other DNA
ggattcactttcagtagatatggcatgtct

SEQ ID NO: 17 - protein, 5 aa, synthetic construct
  REGION 1..5: note = Mouse E6 heavy chain variable complementarity determining regions1 (CDR1) sequence
RYGMS

SEQ ID NO: 18 - DNA, 42 bp, synthetic construct
  misc_feature 1..42: note = Mouse E6 heavy chain variable framework region 2 (FWR2) sequence
  source 1..42: mol_type = other DNA
tgggttcgccagactccaggcaagaggctggagtgggtcgca

SEQ ID NO: 19 - protein, 14 aa, synthetic construct
  REGION 1..14: note = Mouse E6 heavy chain variable framework region 2 (FWR2) sequence
WVRQTPGKRLEWVA

SEQ ID NO: 20 - DNA, 51 bp, synthetic construct
  misc_feature 1..51: note = Mouse E6 heavy chain variable complementarity determining regions2 (CDR2) sequence
  source 1..51: mol_type = other DNA
accattagtggtggcggtacttacatctactatccagacagtgtgaagggg

SEQ ID NO: 21 - protein, 17 aa, synthetic construct
  REGION 1..17: note = Mouse E6 heavy chain variable complementarity determining regions2 (CDR2) sequence
TISGGGTYIYYPDSVKG

SEQ ID NO: 22 - DNA, 96 bp, synthetic construct
  misc_feature 1..96: note = Mouse E6 heavy chain variable framework region 3 (FWR3) acidsequence
  source 1..96: mol_type = other DNA
cgattcaccatctccagagacaatgccaagaacaccctgtacctgcaaatgagcagtctgaagtctgaggacacagccatgtatcactgtacaagg

SEQ ID NO: 23 - protein, 32 aa, synthetic construct
  REGION 1..32: note = Mouse E6 heavy chain variable framework region 3 (FWR3) acidsequence
RFTISRDNAKNTLYLQMSSLKSEDTAMYHCTR

SEQ ID NO: 24 - DNA, 39 bp, synthetic construct
  misc_feature 1..39: note = Mouse E6 heavy chain variable complementarity determining regions3 (CDR3) sequence
  source 1..39: mol_type = other DNA
gataactacggtaggaactacgactacggtatggactac

SEQ ID NO: 25 - protein, 13 aa, synthetic construct
  REGION 1..13: note = Mouse E6 heavy chain variable complementarity determining regions3 (CDR3) sequence
DNYGRNYDYGMDY

SEQ ID NO: 26 - DNA, 294 bp, synthetic construct
  misc_feature 1..294: note = IGHV3-21*03 heavy chain variable region sequence
  source 1..294: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgaga

SEQ ID NO: 27 - protein, 98 aa, synthetic construct
  REGION 1..98: note = IGHV3-21*03 heavy chain variable region sequence
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 28 - DNA, 90 bp, synthetic construct
  misc_feature 1..90: note = IGHV3-21*01 heavy chain variable framework region 1 (FWR1)sequence
  source 1..90: mol_type = other DNA
gaggtgcagctggtggagtctgggggaggcctggtcaagcctggggggtccctgagactctcctgtgcagcctctggattcaccttcagt

SEQ ID NO: 29 - protein, 30 aa, synthetic construct
  REGION 1..30: note = IGHV3-21*01 heavy chain variable framework region 1 (FWR1)sequence
EVQLVESGGGLVKPGGSLRLSCAASGFTFS

SEQ ID NO: 30 - DNA, 15 bp, synthetic construct
  misc_feature 1..15: note = IGHV3-21*01 heavy chain variable complementarity determiningregions 1 (CDR1) sequence
  source 1..15: mol_type = other DNA
agctatagcatgaac

SEQ ID NO: 31 - protein, 5 aa, synthetic construct
  REGION 1..5: note = IGHV3-21*01 heavy chain variable complementarity determiningregions 1 (CDR1) sequence
SYSMN

SEQ ID NO: 32 - DNA, 42 bp, synthetic construct
  misc_feature 1..42: note = IGHV3-21*01 heavy chain variable framework region 2 (FWR2)sequence
  source 1..42: mol_type = other DNA
tgggtccgccaggctccagggaaggggctggagtgggtctca

SEQ ID NO: 33 - protein, 14 aa, synthetic construct
  REGION 1..14: note = IGHV3-21*01 heavy chain variable framework region 2 (FWR2)sequence
WVRQAPGKGLEWVS

SEQ ID NO: 34 - DNA, 51 bp, synthetic construct
  misc_feature 1..51: note = IGHV3-21*01 heavy chain variable complementarity determiningregions 2 (CDR2) sequence
  source 1..51: mol_type = other DNA
tccattagtagtagtagtagttacatatactacgcagactcagtgaagggc

SEQ ID NO: 35 - protein, 17 aa, synthetic construct
  REGION 1..17: note = IGHV3-21*01 heavy chain variable complementarity determiningregions 2 (CDR2) sequence
SISSSSSYIYYADSVKG

SEQ ID NO: 36 - DNA, 96 bp, synthetic construct
  misc_feature 1..96: note = IGHV3-21*01 heavy chain variable framework region 3 (FWR3)sequence
  source 1..96: mol_type = other DNA
cgattcaccatctccagagacaacgccaagaactcactgtatctgcaaatgaacagcctgagagccgaggacacggctgtgtattactgtgcgaga

SEQ ID NO: 37 - protein, 32 aa, synthetic construct
  REGION 1..32: note = IGHV3-21*01 heavy chain variable framework region 3 (FWR3)sequence
RFTISRDNAKNSLYLQMNSLRAEDTAVYYCAR

SEQ ID NO: 38 - DNA, 366 bp, synthetic construct
  misc_feature 1..366: note = Humanized E6 heavy chain variable region sequence
  source 1..366: mol_type = other DNA
gaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagc

SEQ ID NO: 39 - protein, 122 aa, synthetic construct
  REGION 1..122: note = Humanized E6 heavy chain variable region sequence
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 40 - DNA, 90 bp, synthetic construct; identical to SEQ ID NO: 28
  misc_feature 1..90: note = Humanized E6 heavy chain variable framework region 1 (FWR1) acidsequence
  source 1..90: mol_type = other DNA

SEQ ID NO: 41 - protein, 30 aa, synthetic construct; identical to SEQ ID NO: 29
  REGION 1..30: note = Humanized E6 heavy chain variable framework region 1 (FWR1) acidsequence

SEQ ID NO: 42 - DNA, 15 bp, synthetic construct
  misc_feature 1..15: note = Humanized E6 heavy chain variable complementarity determiningregions 1 (CDR1) sequence
  source 1..15: mol_type = other DNA
aggtatggcatgagc

SEQ ID NO: 43 - protein, 5 aa, synthetic construct
  REGION 1..5: note = Humanized E6 heavy chain variable complementarity determiningregions 1 (CDR1) sequence
RYGMS

SEQ ID NO: 44 - DNA, 42 bp, synthetic construct
  misc_feature 1..42: note = Humanized E6 heavy chain variable framework region 2 (FWR2) acidsequence
  source 1..42: mol_type = other DNA
tgggtccgccaggctccagggaagaggctggagtgggtctca

SEQ ID NO: 45 - protein, 14 aa, synthetic construct
  REGION 1..14: note = Humanized E6 heavy chain variable framework region 2 (FWR2) acidsequence
WVRQAPGKRLEWVS

SEQ ID NO: 46 - DNA, 51 bp, synthetic construct
  misc_feature 1..51: note = Humanized E6 heavy chain variable complementarity determiningregions 2 (CDR2) sequence
  source 1..51: mol_type = other DNA
accattagtggcggaggcacctacatatactacccagactcagtgaagggc

SEQ ID NO: 47 - protein, 17 aa, synthetic construct
  REGION 1..17: note = Humanized E6 heavy chain variable complementarity determiningregions 2 (CDR2) sequence
TISGGGTYIYYPDSVKG

SEQ ID NO: 48 - DNA, 96 bp, synthetic construct
  misc_feature 1..96: note = Humanized E6 heavy chain variable framework region 3 (FWR3) acidsequence
  source 1..96: mol_type = other DNA
cgattcaccatctccagagacaacgccaagaacaccctgtatctgcaaatgaacagcctgagagccgaggacacggctgtgtattactgtaccaga

SEQ ID NO: 49 - protein, 32 aa, synthetic construct
  REGION 1..32: note = Humanized E6 heavy chain variable framework region 3 (FWR3) acidsequence
RFTISRDNAKNTLYLQMNSLRAEDTAVYYCTR

SEQ ID NO: 50 - DNA, 39 bp, synthetic construct
  misc_feature 1..39: note = Humanized E6 heavy chain variable complementarity determiningregions 3 (CDR3) sequence
  source 1..39: mol_type = other DNA
gataactatggccgcaactatgattatggcatggattat

SEQ ID NO: 51 - protein, 13 aa, synthetic construct
  REGION 1..13: note = Humanized E6 heavy chain variable complementarity determiningregions 3 (CDR3) sequence
DNYGRNYDYGMDY

SEQ ID NO: 52 - DNA, 1442 bp, synthetic construct
  misc_feature 1..1442: note = Humanized E6 IgG2 heavy chain synthesized by Genescript
  source 1..1442: mol_type = other DNA
gaattctaagcttgggccaccatggaactggggctccgctgggttttccttgttgctattttagaaggtgtccagtgtgaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagtaagtttaaactctaga

SEQ ID NO: 53 - protein, 477 aa, synthetic construct
  REGION 1..477: note = Humanized E6 IgG2 heavy chain synthesized by Genescript
  SITE 477: note = misc_feature - Xaa can be any naturally occurring amino acid
EFAWATMELGLRWVFLVAILEGVQCEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKVTLX

SEQ ID NO: 54 - DNA, 981 bp, synthetic construct
  misc_feature 1..981: note = Human IgG2 heavy chain constant region sequence
  source 1..981: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 55 - protein, 326 aa, synthetic construct
  REGION 1..326: note = Human IgG2 heavy chain constant region sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - DNA, 1362 bp, synthetic construct
  misc_feature 1..1362: note = Humanized E6 IgG1 heavy chain sequence
  source 1..1362: mol_type = other DNA
gaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacccactgtatctgcaaatgaacagcctgagagccgaggacacggctgtgtattactgtcccagagataactatggccgcaactatgattatggcatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 57 - protein, 452 aa, synthetic construct
  REGION 1..452: note = Humanized E6 IgG1 heavy chain sequence
EVQLVESGGGLVKPGGSLRLSCAASGFTFSRYGMSWVRQAPGKRLEWVSTISGGGTYIYYPDSVKGRFTISRDNAKNPLYLQMNSLRAEDTAVYYCPRDNYGRNYDYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 996 bp, synthetic construct
  misc_feature 1..996: note = Human IgG1 heavy chain constant region sequence
  source 1..996: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 59 - protein, 330 aa, synthetic construct
  REGION 1..330: note = Human IgG1 heavy chain constant region sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 560 bp, synthetic construct
  misc_feature 1..560: note = Human IgG1 heavy chain constant region gBLOCK#1 sequence
  source 1..560: mol_type = other DNA
atggcatggattattggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaag

SEQ ID NO: 61 - DNA, 557 bp, synthetic construct
  misc_feature 1..557: note = Human IgG1 heavy chain constant region gBLOCK#2 sequence
  source 1..557: mol_type = other DNA
tacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataagtttaaacccgctgatcagcctcgactgtgccttctagttg

SEQ ID NO: 62 - DNA, 31 bp, synthetic construct
  misc_feature 1..31: note = E6 heavy chain variable region overlapping sequence
  source 1..31: mol_type = other DNA
atggcatggattattggggccagggcaccct

SEQ ID NO: 63 - DNA, 33 bp, synthetic construct
  misc_feature 1..33: note = IgG1 heavy chain constant region overlapping region sequence
  source 1..33: mol_type = other DNA
tacgtggacggcgtggaggtgcataatgccaag

SEQ ID NO: 64 - DNA, 33 bp, synthetic construct
  misc_feature 1..33: note = pCDNA3.1 V5 and pSECTag2 overlapping sequence
  source 1..33: mol_type = other DNA
ccgctgatcagcctcgactgtgccttctagttg

SEQ ID NO: 65 - DNA, 318 bp, synthetic construct
  misc_feature 1..318: note = Mouse E6 Light Chain variable region sequence
  source 1..318: mol_type = other DNA
caaattgttctcacccagtctccagcaatcatgtctgcatctccaggggaggaggtcaccctaacctgcagtgccacctcaagtgtaagttacatacactggttccagcagaggccaggcacttctcccaaactctggatttatagcacatccaacctggcttctggagtccctgttcgcttcagtggcagtggatatgggacctcttactctctcacaatcagccgaatggaggctgaagatgctgccacttattactgccagcaaaggagtagttccccattcacgttcggctcggggacaaagttggaaataaaa

SEQ ID NO: 66 - protein, 106 aa, synthetic construct
  REGION 1..106: note = Mouse E6 Light Chain variable region sequence
QIVLTQSPAIMSASPGEEVTLTCSATSSVSYIHWFQQRPGTSPKLWIYSTSNLASGVPVRFSGSGYGTSYSLTISRMEAEDAATYYCQQRSSSPFTFGSGTKLEIK

SEQ ID NO: 67 - DNA, 69 bp, synthetic construct
  misc_feature 1..69: note = Mouse E6 light chain variable framework region 1 (FWR1) sequence
  source 1..69: mol_type = other DNA
caaattgttctcacccagtctccagcaatcatgtctgcatctccaggggaggaggtcaccctaacctgc

SEQ ID NO: 68 - protein, 23 aa, synthetic construct
  REGION 1..23: note = Mouse E6 light chain variable framework region 1 (FWR1) sequence
QIVLTQSPAIMSASPGEEVTLTC

SEQ ID NO: 69 - DNA, 30 bp, synthetic construct
  misc_feature 1..30: note = Mouse E6 light chain variable complementarity determining regions1 (CDR1) sequence
  source 1..30: mol_type = other DNA
agtgccacctcaagtgtaagttacatacac

SEQ ID NO: 70 - protein, 10 aa, synthetic construct
  REGION 1..10: note = Mouse E6 light chain variable complementarity determining regions1 (CDR1) sequence
SATSSVSYIH

SEQ ID NO: 71 - DNA, 45 bp, synthetic construct
  misc_feature 1..45: note = Mouse E6 light chain variable framework region 2 (FWR2) sequence
  source 1..45: mol_type = other DNA
tggttccagcagaggccaggcacttctcccaaactctggatttat

SEQ ID NO: 72 - protein, 15 aa, synthetic construct
  REGION 1..15: note = Mouse E6 light chain variable framework region 2 (FWR2) sequence
WFQQRPGTSPKLWIY

SEQ ID NO: 73 - DNA, 21 bp, synthetic construct
  misc_feature 1..21: note = Mouse E6 light chain variable complementarity determining regions2 (CDR2) sequence
  source 1..21: mol_type = other DNA
agcacatccaacctggcttct

SEQ ID NO: 74 - protein, 7 aa, synthetic construct
  REGION 1..7: note = Mouse E6 light chain variable complementarity determining regions2 (CDR2) sequence
STSNLAS

SEQ ID NO: 75 - DNA, 96 bp, synthetic construct
  misc_feature 1..96: note = Mouse E6 light chain variable framework region 3 (FWR3) sequence
  source 1..96: mol_type = other DNA
ggagtccctgttcgcttcagtggcagtggatatgggacctcttactctctcacaatcagccgaatggaggctgaagatgctgccacttattactgc

SEQ ID NO: 76 - protein, 32 aa, synthetic construct
  REGION 1..32: note = Mouse E6 light chain variable framework region 3 (FWR3) sequence
GVPVRFSGSGYGTSYSLTISRMEAEDAATYYC

SEQ ID NO: 77 - DNA, 27 bp, synthetic construct
  misc_feature 1..27: note = Mouse E6 light chain variable complementarity determining regions3 (CDR3) sequence
  source 1..27: mol_type = other DNA
cagcaaaggagtagttccccattcacg

SEQ ID NO: 78 - protein, 9 aa, synthetic construct
  REGION 1..9: note = Mouse E6 light chain variable complementarity determining regions3 (CDR3) sequence
QQRSSSPFT

SEQ ID NO: 79 - DNA, 287 bp, synthetic construct
  misc_feature 1..287: note = IGKV3-11*02 light chain variable region sequence
  source 1..287: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggagagacttcactctcaccatcagcagcctagagcctgaagattttgcagtttattactgtcagcagcgtagcaactggcctcc

SEQ ID NO: 80 - protein, 96 aa, synthetic construct
  REGION 1..96: note = IGKV3-11*02 light chain variable region sequence
EIVLTQSPATLSLSPGERATLSCRASQSVSSYLAWYQQKPGQAPRLLIYDASNRATGIPARFSGSGSGRDFTLTISSLEPEDFAVYYCQQRSNWPP

SEQ ID NO: 81 - DNA, 69 bp, synthetic construct
  misc_feature 1..69: note = IGKV3-11*02 light chain variable framework region 1 (FWR1) acidsequence
  source 1..69: mol_type = other DNA
gaaattgtgttgacacagtctccagccaccctgtctttgtctccaggggaaagagccaccctctcctgc

SEQ ID NO: 82 - protein, 23 aa, synthetic construct
  REGION 1..23: note = IGKV3-11*02 light chain variable framework region 1 (FWR1) acidsequence
EIVLTQSPATLSLSPGERATLSC

SEQ ID NO: 83 - DNA, 33 bp, synthetic construct
  misc_feature 1..33: note = IGKV3-11*02 light chain variable complementarity determiningregions 1 (CDR1) sequence
  source 1..33: mol_type = other DNA
agggccagtcagagtgttagcagctacttagcc

SEQ ID NO: 84 - protein, 11 aa, synthetic construct
  REGION 1..11: note = IGKV3-11*02 light chain variable complementarity determiningregions 1 (CDR1) sequence
RASQSVSSYLA

SEQ ID NO: 85 - DNA, 45 bp, synthetic construct
  misc_feature 1..45: note = IGKV3-11*02 light chain variable framework region 2 (FWR2)sequence
  source 1..45: mol_type = other DNA
tggtaccaacagaaacctggccaggctcccaggctcctcatctat

SEQ ID NO: 86 - protein, 15 aa, synthetic construct
  REGION 1..15: note = IGKV3-11*02 light chain variable framework region 2 (FWR2)sequence
WYQQKPGQAPRLLIY

SEQ ID NO: 87 - DNA, 21 bp, synthetic construct
  misc_feature 1..21: note = IGKV3-11*02 light chain variable complementarity determiningregions 2 (CDR2) sequence
  source 1..21: mol_type = other DNA
gatgcatccaacagggccact

SEQ ID NO: 88 - protein, 7 aa, synthetic construct
  REGION 1..7: note = IGKV3-11*02 light chain variable complementarity determiningregions 2 (CDR2) sequence
DASNRAT

SEQ ID NO: 89 - DNA, 96 bp, synthetic construct
  misc_feature 1..96: note = IGKV3-11*02 light chain variable framework region 3 (FWR3)sequence
  source 1..96: mol_type = other DNA
ggcatcccagccaggttcagtggcagtgggtctgggagagacttcactctcaccatcagcagcctagagcctgaagattttgcagtttattactgt

SEQ ID NO: 90 - protein, 32 aa, synthetic construct
  REGION 1..32: note = IGKV3-11*02 light chain variable framework region 3 (FWR3)sequence
GIPARFSGSGSGTDFTLTISSLEPEDFAVYYC

SEQ ID NO: 91 - DNA, 23 bp, synthetic construct
  misc_feature 1..23: note = IGKV3-11*02 light chain variable complementarity determiningregions3 (CDR3) sequence
  source 1..23: mol_type = other DNA
cagcagcgtagcaactggcctcc

SEQ ID NO: 92 - protein, 8 aa, synthetic construct
  REGION 1..8: note = IGKV3-11*02 light chain variable complementarity determiningregions3 (CDR3) sequence
QQRSNWPP

SEQ ID NO: 93 - DNA, 318 bp, synthetic construct
  misc_feature 1..318: note = Humanized E6 light chain variable region sequence
  source 1..318: mol_type = other DNA
gaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaa

SEQ ID NO: 94 - protein, 106 aa, synthetic construct
  REGION 1..106: note = Humanized E6 light chain variable region sequence
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 95 - DNA, 69 bp, synthetic construct
  misc_feature 1..69: note = Humanized E6 light chain variable framework region 1 (FWR1) acidsequence
  source 1..69: mol_type = other DNA
gaaattgtgttgacacagtctccagccaccctgtctttgtctccaggggaaagagccaccctcacctgc

SEQ ID NO: 96 - protein, 23 aa, synthetic construct
  REGION 1..23: note = Humanized E6 light chain variable framework region 1 (FWR1) acidsequence
EIVLTQSPATLSLSPGERATLTC

SEQ ID NO: 97 - DNA, 30 bp, synthetic construct
  misc_feature 1..30: note = Humanized E6 light chain variable complementarity determiningregions 1 (CDR1) sequence
  source 1..30: mol_type = other DNA
agcgccaccagcagtgttagctacatccac

SEQ ID NO: 98 - protein, 10 aa, synthetic construct
  REGION 1..10: note = Humanized E6 light chain variable complementarity determiningregions 1 (CDR1) sequence
SATSSVSYIH

SEQ ID NO: 99 - DNA, 45 bp, synthetic construct
  misc_feature 1..45: note = Humanized E6 heavy light variable framework region 2 (FWR2) acidsequence
  source 1..45: mol_type = other DNA
tggtaccaacagaggcctggccagagccccaggctcctcatctat

SEQ ID NO: 100 - protein, 15 aa, synthetic construct
  REGION 1..15: note = Humanized E6 heavy light variable framework region 2 (FWR2) acidsequence
WYQQRPGQSPRLLIY

SEQ ID NO: 101 - DNA, 21 bp, synthetic construct
  misc_feature 1..21: note = Humanized E6 light chain variable complementarity determiningregions 2 (CDR2) sequence
  source 1..21: mol_type = other DNA
agcacctccaacctggccagc

SEQ ID NO: 102 - protein, 7 aa, synthetic construct
  REGION 1..7: note = Humanized E6 light chain variable complementarity determiningregions 2 (CDR2) sequence
STSNLAS

SEQ ID NO: 103 - DNA, 96 bp, synthetic construct
  misc_feature 1..96: note = Humanized E6 light chain variable framework region 3 (FWR3) acidsequence
  source 1..96: mol_type = other DNA
ggcatcccagccaggttcagtggcagtgggtctgggagcgactacactctcaccatcagcagcctagagcctgaagattttgcagtttattactgt

SEQ ID NO: 104 - protein, 32 aa, synthetic construct
  REGION 1..32: note = Humanized E6 light chain variable framework region 3 (FWR3) acidsequence
GIPARFSGSGSGSDYTLTISSLEPEDFAVYYC

SEQ ID NO: 105 - DNA, 27 bp, synthetic construct
  misc_feature 1..27: note = Humanized E6 light chain variable complementarity determiningregions 3 (CDR3) sequence
  source 1..27: mol_type = other DNA
cagcagcgtagcagctcccctttcacc

SEQ ID NO: 106 - protein, 9 aa, synthetic construct
  REGION 1..9: note = Humanized E6 light chain variable complementarity determiningregions 3 (CDR3) sequence
QQRSSSPFT

SEQ ID NO: 107 - DNA, 740 bp, synthetic construct
  misc_feature 1..740: note = Humanized E6 Kappa light chain synthesized by Genescript
  source 1..740: mol_type = other DNA
gaattctaagcttgggccaccatggaagccccagcgcagcttctcttcctcctgctactctggctcccagataccactggagaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaagtttaaactctaga

SEQ ID NO: 108 - protein, 243 aa, synthetic construct
  REGION 1..243: note = Humanized E6 Kappa light chain synthesized by Genescript
  SITE 243: note = misc_feature - Xaa can be any naturally occurring amino acid
EFAWATMEAPAQLLFLLLLWLPDTTGEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECVTLX

SEQ ID NO: 109 - DNA, 324 bp, synthetic construct
  misc_feature 1..324: note = Human Kappa light chain constant region sequence
  source 1..324: mol_type = other DNA
aggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 110 - protein, 107 aa, synthetic construct [sequence omitted]

SEQ ID NO: 111 - DNA, 642 bp, synthetic construct
  misc_feature 1..642: note = Humanized E6 lambda light chain sequence
  source 1..642: mol_type = other DNA
gaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagtaa

SEQ ID NO: 112 - protein, 212 aa, synthetic construct
  REGION 1..212: note = Humanized E6 lambda light chain sequence
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 113 - DNA, 324 bp, synthetic construct
  misc_feature 1..324: note = Humanized lambda light chain constant region sequence
  source 1..324: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagtaa

SEQ ID NO: 114 - protein, 106 aa, synthetic construct
  REGION 1..106: note = Humanized lambda light chain constant region sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 115 - DNA, 614 bp, synthetic construct
  misc_feature 1..614: note = Human lambda light chain constant region gBLOCK#3 sequence
  source 1..614: mol_type = other DNA
agcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagtaagtttaaacccgctgatcagcctcgactgtgccttctagttg

SEQ ID NO: 116 - DNA, 31 bp, synthetic construct
  misc_feature 1..31: note = E6 light chain variable region overlapping sequence
  source 1..31: mol_type = other DNA
agcgccaccagcagtgttagctacatccact

SEQ ID NO: 117 - DNA, 33 bp, synthetic construct; identical to SEQ ID NO: 64
  misc_feature 1..33: note = pCDNA3.1 V5 and pSECTag2 overlapping sequence
  source 1..33: mol_type = other DNA

SEQ ID NO: 118 - DNA, 390 bp, synthetic construct
  misc_feature 1..390: note = Mouse C2 heavy chain variable region sequence
  source 1..390: mol_type = other DNA
gaggtccagctggaggagtcagggggaggcttagtgaagcctggagggtccctgaaactctcctgtgcagcctctggattcactttcagtggctatgccatgtcttgggttcgccagactccggagaagaggctggagtgggtcgcaaccattagtagtggtggtacttatatctactatccagacagtgtgaaggggcgattcaccatctccagagacaatgccaagaacaccctgtacctgcaaatgagcagtctgaggtctgaggacacggccatgtattactgtgcaagacttgggggggataattactacgaatacttcgatgtctggggcgcagggaccacggtcaccgtctcctccgccaaaacgacacccccatctgtctat

SEQ ID NO: 119 - protein, 130 aa, synthetic construct
  REGION 1..130: note = Mouse C2 heavy chain variable region sequence
EVQLEESGGGLVKPGGSLKLSCAASGFTFSGYAMSWVRQTPEKRLEWVATISSGGTYIYYPDSVKGRFTISRDNAKNTLYLQMSSLRSEDTAMYYCARLGGDNYYEYFDVWGAGTTVTVSSAKTTPPSVY

SEQ ID NO: 120 - DNA, 90 bp, synthetic construct
  misc_feature 1..90: note = Mouse C2 heavy chain variable framework region 1 (FWR1) sequence
  source 1..90: mol_type = other DNA
gaggtccagctggaggagtcagggggaggcttagtgaagcctggagggtccctgaaactctcctgtgcagcctctggattcactttcagt

SEQ ID NO: 121 - protein, 30 aa, synthetic construct
  REGION 1..30: note = Mouse C2 heavy chain variable framework region 1 (FWR1) sequence
EVQLEESGGGLVKPGGSLKLSCAASGFTFS

SEQ ID NO: 122 - DNA, 15 bp, synthetic construct
  misc_feature 1..15: note = Mouse C2 heavy chain variable complementarity determining regions1 (CDR1) sequence
  source 1..15: mol_type = other DNA
ggctatgccatgtct

SEQ ID NO: 123 - protein, 5 aa, synthetic construct
  REGION 1..5: note = Mouse C2 heavy chain variable complementarity determining regions1 (CDR1) sequence
GYAMS

SEQ ID NO: 124 - DNA, 42 bp, synthetic construct
  misc_feature 1..42: note = Mouse C2 heavy chain variable framework region 2 (FWR2) sequence
  source 1..42: mol_type = other DNA
tgggttcgccagactccggagaagaggctggagtgggtcgca

SEQ ID NO: 125 - protein, 14 aa, synthetic construct
  REGION 1..14: note = Mouse C2 heavy chain variable framework region 2 (FWR2) sequence
WVRQTPEKRLEWVA

SEQ ID NO: 126 - DNA, 51 bp, synthetic construct
  misc_feature 1..51: note = Mouse C2 heavy chain variable complementarity determining regions2 (CDR2) sequence
  source 1..51: mol_type = other DNA
accattagtagtggtggtacttatatctactatccagacagtgtgaagggg

SEQ ID NO: 127 - protein, 17 aa, synthetic construct
  REGION 1..17: note = Mouse C2 heavy chain variable complementarity determining regions2 (CDR2) sequence
TISSGGTYIYYPDSVKG

SEQ ID NO: 128 - DNA, 96 bp, synthetic construct
  misc_feature 1..96: note = Mouse C2 heavy chain variable framework region 3 (FWR3) sequence
  source 1..96: mol_type = other DNA
cgattcaccatctccagagacaatgccaagaacaccctgtacctgcaaatgagcagtctgaggtctgaggacacggccatgtattactgtgcaaga

SEQ ID NO: 129 - protein, 32 aa, synthetic construct
  REGION 1..32: note = Mouse C2 heavy chain variable framework region 3 (FWR3) sequence
RFTISRDNAKNTLYLQMSSLRSEDTAMYYCAR

SEQ ID NO: 130 - DNA, 36 bp, synthetic construct
  misc_feature 1..36: note = Mouse C2 heavy chain variable complementarity determining regions3 (CDR3) sequence
  source 1..36: mol_type = other DNA
cttgggggggataattactacgaatacttcgatgtc

SEQ ID NO: 131 - protein, 12 aa, synthetic construct
  REGION 1..12: note = Mouse C2 heavy chain variable complementarity determining regions3 (CDR3) sequence
LGGDNYYEYFDV

SEQ ID NO: 132 - DNA, 294 bp, synthetic construct
  misc_feature 1..294: note = IGHV3-21*04 heavy chain variable region sequence
  source 1..294: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggccgtgtattactgtgcgaga

SEQ ID NO: 133 - protein, 98 aa, synthetic construct; identical to SEQ ID NO: 27
  REGION 1..98: note = IGHV3-21*04 heavy chain variable region sequence

SEQ ID NO: 134 - DNA, 90 bp, synthetic construct; identical to SEQ ID NO: 28
  misc_feature 1..90: note = IGHV3-21*04 heavy chain variable framework region 1 (FWR1)sequence
  source 1..90: mol_type = other DNA

SEQ ID NO: 135 - protein, 30 aa, synthetic construct; identical to SEQ ID NO: 29
  REGION 1..30: note = IGHV3-21*04 heavy chain variable framework region 1 (FWR1)sequence

SEQ ID NO: 136 - DNA, 15 bp, synthetic construct
  misc_feature 1..15: note = IGHV3-21*04 heavy chain variable complementarity determiningregions 1 (CDR1) sequence
  source 1..15: mol_type = other DNA
agctatagcatgaac

SEQ ID NO: 137 - protein, 5 aa, synthetic construct
  REGION 1..5: note = IGHV3-21*04 heavy chain variable complementarity determiningregions 1 (CDR1) sequence
SYSMN

SEQ ID NO: 138 - DNA, 41 bp, synthetic construct
  misc_feature 1..41: note = IGHV3-21*04 heavy chain variable framework region 2 (FWR2)sequence
  source 1..41: mol_type = other DNA
gggtccgccaggctccagggaaggggctggagtgggtctca

SEQ ID NO: 139 - protein, 14 aa, synthetic construct
  REGION 1..14: note = IGHV3-21*04 heavy chain variable framework region 2 (FWR2)sequence
WVRQAPGKGLEWVS

SEQ ID NO: 140 - DNA, 51 bp, synthetic construct; identical to SEQ ID NO: 34
  misc_feature 1..51: note = IGHV3-21*04 heavy chain variable complementarity determiningregions 2 (CDR2) sequence
  source 1..51: mol_type = other DNA

SEQ ID NO: 141 - protein, 17 aa, synthetic construct
  REGION 1..17: note = IGHV3-21*04 heavy chain variable complementarity determiningregions 2 (CDR2) sequence
SISSSSSYIYYADSVKG

SEQ ID NO: 142 - DNA, 96 bp, synthetic construct
  misc_feature 1..96: note = IGHV3-21*04 heavy chain variable framework region 3 (FWR3)sequence
  source 1..96: mol_type = other DNA
cgattcaccatctccagagacaacgccaagaactcactgtatctgcaaatgaacagcctgagagccgaggacacggccgtgtattactgtgcgaga

SEQ ID NO: 143 - protein, 32 aa, synthetic construct; identical to SEQ ID NO: 37
  REGION 1..32: note = IGHV3-21*04 heavy chain variable framework region 3 (FWR3)sequence

SEQ ID NO: 144 - DNA, 363 bp, synthetic construct
  misc_feature 1..363: note = Humanized C2 heavy chain variable region sequence
  source 1..363: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctcc

SEQ ID NO: 145 - protein, 121 aa, synthetic construct
  REGION 1..121: note = Humanized C2 heavy chain variable region sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSS

SEQ ID NO: 146 - DNA, 90 bp, synthetic construct; identical to SEQ ID NO: 28
  misc_feature 1..90: note = Humanized C2 heavy chain variable framework region 1 (FWR1)sequence
  source 1..90: mol_type = other DNA

SEQ ID NO: 147 - protein, 30 aa, synthetic construct; identical to SEQ ID NO: 29
  REGION 1..30: note = Humanized C2 heavy chain variable framework region 1 (FWR1)sequence

SEQ ID NO: 148 - DNA, 15 bp, synthetic construct
  misc_feature 1..15: note = Humanized C2 heavy chain variable complementarity determiningregions 1 (CDR1) sequence
  source 1..15: mol_type = other DNA
ggctatgccatgagc

SEQ ID NO: 149 - protein, 5 aa, synthetic construct
  REGION 1..5: note = Humanized C2 heavy chain variable complementarity determiningregions 1 (CDR1) sequence
GYAMS

SEQ ID NO: 150 - DNA, 43 bp, synthetic construct
  misc_feature 1..43: note = Humanized C2 heavy chain variable framework region 2 (FWR2)sequence
  source 1..43: mol_type = other DNA
tgggtccgccaggctccagggaaggggctggagtgggtctcaa

SEQ ID NO: 151 - protein, 14 aa, synthetic construct
  REGION 1..14: note = Humanized C2 heavy chain variable framework region 2 (FWR2)sequence
WVRQAPGKGLEWVS

SEQ ID NO: 152 - DNA, 51 bp, synthetic construct
  misc_feature 1..51: note = Humanized C2 heavy chain variable complementarity determiningregions 2 (CDR2) sequence
  source 1..51: mol_type = other DNA
accattagtagtggcggaacctacatatactaccccgactcagtgaagggc

SEQ ID NO: 153 - protein, 17 aa, synthetic construct
  REGION 1..17: note = Humanized C2 heavy chain variable complementarity determiningregions 2 (CDR2) sequence
TISSGGTYIYYPDSVKG

SEQ ID NO: 154 - DNA, 96 bp, synthetic construct; identical to SEQ ID NO: 142
  misc_feature 1..96: note = Humanized C2 heavy chain variable framework region 3 (FWR3)sequence
  source 1..96: mol_type = other DNA

SEQ ID NO: 155 - protein, 32 aa, synthetic construct; identical to SEQ ID NO: 37
  REGION 1..32: note = Humanized C2 heavy chain variable framework region 3 (FWR3)sequence

SEQ ID NO: 156 - DNA, 36 bp, synthetic construct; identical to SEQ ID NO: 130
  misc_feature 1..36: note = Humanized C2 heavy chain variable complementarity determiningregions 3 (CDR3) sequence
  source 1..36: mol_type = other DNA

SEQ ID NO: 157 - protein, 12 aa, synthetic construct
  REGION 1..12: note = Humanized C2 heavy chain variable complementarity determiningregions 3 (CDR3) sequence
LGGDNYYEYFDV

SEQ ID NO: 158 - DNA, 1359 bp, synthetic construct
  misc_feature 1..1359: note = Humanized C2 IgG1 heavy chain sequence
  source 1..1359: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 159 - protein, 451 aa, synthetic construct
  REGION 1..451: note = Humanized C2 IgG1 heavy chain sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - DNA, 616 bp, synthetic construct
  misc_feature 1..616: note = Humanized C2 gBLOCK#4 sequence
  source 1..616: mol_type = other DNA
actcactatagggagacccaagctggctagttaagcttgggccaccatggagacagacacactcctgctatgggtactgctgctctgggttccaggttccactggtgacgaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagc

SEQ ID NO: 161 - DNA, 32 bp, synthetic construct
  misc_feature 1..32: note = pCDNA3.1 V5 overlapping sequence
  source 1..32: mol_type = other DNA
actcactatagggagacccaagctggctagtt

SEQ ID NO: 162 - DNA, 34 bp, synthetic construct
  misc_feature 1..34: note = Human IgG1 constant region overlapping sequence
  source 1..34: mol_type = other DNA
gacggtgtcgtggaactcaggcgccctgaccagc

SEQ ID NO: 163 - DNA, 1347 bp, synthetic construct
  misc_feature 1..1347: note = Humanized C2 IgG2 heavy chain sequence
  source 1..1347: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagtaa

SEQ ID NO: 164 - protein, 447 aa, synthetic construct
  REGION 1..447: note = Humanized C2 IgG2 heavy chain sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 165 - DNA, 549 bp, synthetic construct
  misc_feature 1..549: note = Humanized C2 gBLOCK#5 sequence
  source 1..549: mol_type = other DNA
tgctctgggttccaggttccactggtgacgcggcccagccggccgaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccgcctccaccaagggcccatcggtcttccccctggcgccctgctccaggagcacctccgagagcacagccgccctgggctgcctggtcaaggactacttccccgaaccggtgacggtgtcgtggaactcaggcgctctgacca

SEQ ID NO: 166 - DNA, 31 bp, synthetic construct
  misc_feature 1..31: note = pSEC Tag2 overlapping sequence
  source 1..31: mol_type = other DNA
tgctctgggttccaggttccactggtgacgc

SEQ ID NO: 167 - DNA, 32 bp, synthetic construct
  misc_feature 1..32: note = Human IgG2 constant region overlapping sequence
  source 1..32: mol_type = other DNA
gacggtgtcgtggaactcaggcgctctgacca

SEQ ID NO: 168 - DNA, 360 bp, synthetic construct
  misc_feature 1..360: note = Mouse C2 light chain variable region sequence
  source 1..360: mol_type = other DNA
gacattgtgatcacacagtctacagcttccttaggtgtatctctggggcagagggccaccatctcatgcagggccagcaaaagtgtcagtacatctggctatagttatatgcactggtaccaacagagaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgttcacgttcggaggggggaccaagctggagataaaacgggctgatgctgcaccaactgtatcc

SEQ ID NO: 169 - protein, 120 aa, synthetic construct
  REGION 1..120: note = Mouse C2 light chain variable region sequence
DIVITQSTASLGVSLGQRATISCRASKSVSTSGYSYMHWYQQRPGQPPKLLIYLASNLESGVPARFSGSGSGTDFTLNIHPVEEEDAATYYCQHSRELPFTFGGGTKLEIKRADAAPTVS

SEQ ID NO: 170 - DNA, 69 bp, synthetic construct
  misc_feature 1..69: note = Mouse C2 light chain variable framework region 1 (FWR1) sequence
  source 1..69: mol_type = other DNA
gacattgtgatcacacagtctacagcttccttaggtgtatctctggggcagagggccaccatctcatgc

SEQ ID NO: 171 - protein, 23 aa, synthetic construct
  REGION 1..23: note = Mouse C2 light chain variable framework region 1 (FWR1) sequence
DIVITQSTASLGVSLGQRATISC

SEQ ID NO: 172 - DNA, 45 bp, synthetic construct
  misc_feature 1..45: note = Mouse C2 light chain variable complementarity determining regions1 (CDR1) sequence
  source 1..45: mol_type = other DNA
agggccagcaaaagtgtcagtacatctggctatagttatatgcac

SEQ ID NO: 173 - protein, 15 aa, synthetic construct
  REGION 1..15: note = Mouse C2 light chain variable complementarity determining regions1 (CDR1) sequence
RASKSVSTSGYSYMH

SEQ ID NO: 174 - DNA, 45 bp, synthetic construct
  misc_feature 1..45: note = Mouse C2 light chain variable framework region 2 (FWR2) sequence
  source 1..45: mol_type = other DNA
tggtaccaacagagaccaggacagccacccaaactcctcatctat

SEQ ID NO: 175 - protein, 15 aa, synthetic construct
  REGION 1..15: note = Mouse C2 light chain variable framework region 2 (FWR2) sequence
WYQQRPGQPPKLLIY

SEQ ID NO: 176 - DNA, 20 bp, synthetic construct
  misc_feature 1..20: note = Mouse C2 light chain variable complementarity determining regions2 (CDR2) sequence
  source 1..20: mol_type = other DNA
cttgcatccaacctagaatc

SEQ ID NO: 177 - protein, 7 aa, synthetic construct
  REGION 1..7: note = Mouse C2 light chain variable complementarity determining regions2 (CDR2) sequence
LASNLES

SEQ ID NO: 178 - DNA, 97 bp, synthetic construct
  misc_feature 1..97: note = Mouse C2 light chain variable framework region 3 (FWR3) sequence
  source 1..97: mol_type = other DNA
tggggtccctgccaggttcagtggcagtgggtctgggacagacttcaccctcaacatccatcctgtggaggaggaggatgctgcaacctattactgt

SEQ ID NO: 179 - protein, 32 aa, synthetic construct
  REGION 1..32: note = Mouse C2 light chain variable framework region 3 (FWR3) sequence
GVPARFSGSGSGTDFTLNIHPVEEEDAATYYC

SEQ ID NO: 180 - DNA, 27 bp, synthetic construct
  misc_feature 1..27: note = Mouse C2 light chain variable complementarity determining regions3 (CDR3) sequence
  source 1..27: mol_type = other DNA
cagcacagtagggagcttccgttcacg

SEQ ID NO: 181 - protein, 9 aa, synthetic construct
  REGION 1..9: note = Mouse C2 light chain variable complementarity determining regions3 (CDR3) sequence
QHSRELPFT

SEQ ID NO: 182 - DNA, 303 bp, synthetic construct
  misc_feature 1..303: note = IGKV7-3*01 light chain variable region sequence
  source 1..303: mol_type = other DNA
gacattgtgctgacccagtctccagcctccttggccgtgtctccaggacagagggccaccatcacctgcagagccagtgagagtgtcagtttcttgggaataaacttaattcactggtatcagcagaaaccaggacaacctcctaaactcctgatttaccaagcatccaataaagacactggggtcccagccaggttcagcggcagtgggtctgggaccgatttcaccctcacaattaatcctgtggaagctaatgatactgcaaattattactgtctgcagagtaagaattttcctcccaca

SEQ ID NO: 183 - protein, 101 aa, synthetic construct
  REGION 1..101: note = IGKV7-3*01 light chain variable region sequence
DIVLTQSPASLAVSPGQRATITCRASESVSFLGINLIHWYQQKPGQPPKLLIYQASNKDTGVPARFSGSGSGTDFTLTINPVEANDTANYYCLQSKNFPPT

SEQ ID NO: 184 - DNA, 69 bp, synthetic construct
  misc_feature 1..69: note = IGKV7-3*01 light chain variable framework region 1 (FWR1)sequence
  source 1..69: mol_type = other DNA
gacattgtgctgacccagtctccagcctccttggccgtgtctccaggacagagggccaccatcacctgc

SEQ ID NO: 185 - protein, 23 aa, synthetic construct
  REGION 1..23: note = IGKV7-3*01 light chain variable framework region 1 (FWR1)sequence
DIVLTQSPASLAVSPGQRATITC

SEQ ID NO: 186 - DNA, 45 bp, synthetic construct
  misc_feature 1..45: note = IGKV7-3*01 light chain variable complementarity determiningregions 1 (CDR1) sequence
  source 1..45: mol_type = other DNA
agagccagtgagagtgtcagtttcttgggaataaacttaattcac

SEQ ID NO: 187 - protein, 15 aa, synthetic construct
  REGION 1..15: note = IGKV7-3*01 light chain variable complementarity determiningregions 1 (CDR1) sequence
RASESVSFLGINLIH

SEQ ID NO: 188 - DNA, 45 bp, synthetic construct
  misc_feature 1..45: note = IGKV7-3*01 light chain variable framework region 2 (FWR2)sequence
  source 1..45: mol_type = other DNA
tggtatcagcagaaaccaggacaacctcctaaactcctgatttac

SEQ ID NO: 189 - protein, 15 aa, synthetic construct
  REGION 1..15: note = IGKV7-3*01 light chain variable framework region 2 (FWR2)sequence
WYQQKPGQPPKLLIY

SEQ ID NO: 190 - DNA, 21 bp, synthetic construct
  misc_feature 1..21: note = IGKV7-3*01 light chain variable complementarity determiningregions 2 (CDR2) sequence
  source 1..21: mol_type = other DNA
caagcatccaataaagacact

SEQ ID NO: 191 - protein, 7 aa, synthetic construct
  REGION 1..7: note = IGKV7-3*01 light chain variable complementarity determiningregions 2 (CDR2) sequence
QASNKDT

SEQ ID NO: 192 - DNA, 96 bp, synthetic construct
  misc_feature 1..96: note = IGKV7-3*01 light chain variable framework region 3 (FWR3)sequence
  source 1..96: mol_type = other DNA
ggggtcccagccaggttcagcggcagtgggtctgggaccgatttcaccctcacaattaatcctgtggaagctaatgatactgcaaattattactgt

SEQ ID NO: 193 - protein, 32 aa, synthetic construct
  REGION 1..32: note = IGKV7-3*01 light chain variable framework region 3 (FWR3)sequence
GVPARFSGSGSGTDFTLTINPVEANDTANYYC

SEQ ID NO: 194 - DNA, 339 bp, synthetic construct
  misc_feature 1..339: note = Humanized C2 light chain variable region sequence
  source 1..339: mol_type = other DNA
gacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaact

SEQ ID NO: 195 - protein, 113 aa, synthetic construct
  REGION 1..113: note = Humanized C2 light chain variable region sequence
DIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 196 - DNA, 69 bp, synthetic construct; identical to SEQ ID NO: 184
  misc_feature 1..69: note = Humanized C2 light chain variable framework region 1 (FWR1) acidsequence
  source 1..69: mol_type = other DNA

SEQ ID NO: 197 - protein, 23 aa, synthetic construct
  REGION 1..23: note = Humanized C2 light chain variable framework region 1 (FWR1) acidsequence
DIVLTQSPASLAVSPGQRATITC

SEQ ID NO: 198 - DNA, 45 bp, synthetic construct
  misc_feature 1..45: note = Humanized C2 light chain variable complementarity determiningregions 1 (CDR1) sequence
  source 1..45: mol_type = other DNA
agagccagtaagagtgtcagtaccagcggatactcctacatgcac

SEQ ID NO: 199 - protein, 15 aa, synthetic construct
  REGION 1..15: note = Humanized C2 light chain variable complementarity determiningregions 1 (CDR1) sequence
RASKSVSTSGYSYMH

SEQ ID NO: 200 - DNA, 45 bp, synthetic construct; identical to SEQ ID NO: 188
  misc_feature 1..45: note = Humanized C2 heavy light variable framework region 2 (FWR2) acidsequence
  source 1..45: mol_type = other DNA

SEQ ID NO: 201 - protein, 15 aa, synthetic construct
  REGION 1..15: note = Humanized C2 heavy light variable framework region 2 (FWR2) acidsequence
WYQQKPGQPPKLLIY

SEQ ID NO: 202 - DNA, 21 bp, synthetic construct
  misc_feature 1..21: note = Humanized C2 light chain variable complementarity determiningregions 2 (CDR2) sequence
  source 1..21: mol_type = other DNA
ctggcatccaatctggagagc

SEQ ID NO: 203 - protein, 7 aa, synthetic construct
  REGION 1..7: note = Humanized C2 light chain variable complementarity determiningregions 2 (CDR2) sequence
LASNLES

SEQ ID NO: 204 - DNA, 96 bp, synthetic construct; identical to SEQ ID NO: 192
  misc_feature 1..96: note = Humanized C2 light chain variable framework region 3 (FWR3) acidsequence
  source 1..96: mol_type = other DNA

SEQ ID NO: 205 - protein, 32 aa, synthetic construct; identical to SEQ ID NO: 193
  REGION 1..32: note = Humanized C2 light chain variable framework region 3 (FWR3) acidsequence

SEQ ID NO: 206 - DNA, 27 bp, synthetic construct
  misc_feature 1..27: note = Humanized C2 light chain variable complementarity determiningregions 3 (CDR3) sequence
  source 1..27: mol_type = other DNA
cagcacagtagggagctgcctttcaca

SEQ ID NO: 207 - protein, 9 aa, synthetic construct
  REGION 1..9: note = Humanized C2 light chain variable complementarity determiningregions 3 (CDR3) sequence
QHSRELPFT

SEQ ID NO: 208 - DNA, 27 bp, synthetic construct
  misc_feature 1..27: note = Humanized C2 light chain variable complementarity determiningregions 3 (CDR3) sequence
  source 1..27: mol_type = other DNA
ctgcagagtaagaattttcctcccaca

SEQ ID NO: 209 - protein, 9 aa, synthetic construct
  REGION 1..9: note = Humanized C2 light chain variable complementarity determiningregions 3 (CDR3) sequence
LQSKNFPPT

SEQ ID NO: 210 - DNA, 813 bp, synthetic construct
  misc_feature 1..813: note = Humanized C2 gBLOCK#6 sequence (Kappa light chain in pCDNA3.1 V5)
  source 1..813: mol_type = other DNA
actcactatagggagacccaagctggctagttaagcttgggccaccatggagacagacacactcctgctatgggtactgctgctctgggttccaggttccactggtgacgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaagtttaaacccgctgatcagcctcgactgtgccttctagttg

SEQ ID NO: 211 - DNA, 32 bp, synthetic construct; identical to SEQ ID NO: 161
  misc_feature 1..32: note = pCDNA3.1 V5 5' overlapping sequence
  source 1..32: mol_type = other DNA

SEQ ID NO: 212 - DNA, 33 bp, synthetic construct; identical to SEQ ID NO: 64
  misc_feature 1..33: note = pCDNA3.1 V5 3' overlapping sequence
  source 1..33: mol_type = other DNA

SEQ ID NO: 213 - DNA, 748 bp, synthetic construct
  misc_feature 1..748: note = Humanized C2 gBLOCK#7 sequence (Kappa light chain in pSEC Tag2)
  source 1..748: mol_type = other DNA
tgctctgggttccaggttccactggtgacgcggcccagccggccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaagtttaaacccgctgatcagcctcgactgtgccttctagttg

SEQ ID NO: 214 - DNA, 31 bp, synthetic construct; identical to SEQ ID NO: 166
  misc_feature 1..31: note = pSEC Tag2 5' overlapping sequence
  source 1..31: mol_type = other DNA

SEQ ID NO: 215 - DNA, 33 bp, synthetic construct; identical to SEQ ID NO: 64
  misc_feature 1..33: note = pSEC Tag2 3' overlapping sequence
  source 1..33: mol_type = other DNA

SEQ ID NO: 216 - DNA, 813 bp, synthetic construct
  misc_feature 1..813: note = Humanized C2 gBLOCK#8 sequence (lambda light chain in pCDNA3.1V5)
  source 1..813: mol_type = other DNA
actcactatagggagacccaagctggctagttaagcttgggccaccatggagacagacacactcctgctatgggtactgctgctctgggttccaggttccactggtgacgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagtaagtttaaacccgctgatcagcctcgactgtgccttctagttg

SEQ ID NO: 217 - DNA, 32 bp, synthetic construct; identical to SEQ ID NO: 161
  misc_feature 1..32: note = pCDNA3.1 V5 5' overlapping sequence
  source 1..32: mol_type = other DNA

SEQ ID NO: 218 - DNA, 33 bp, synthetic construct; identical to SEQ ID NO: 64
  misc_feature 1..33: note = pCDNA3.1 V5 3' overlapping sequence
  source 1..33: mol_type = other DNA

SEQ ID NO: 219 - DNA, 748 bp, synthetic construct
  misc_feature 1..748: note = Humanized C2 gBLOCK#9 sequence (lambda light chain in pSEC Tag2)
  source 1..748: mol_type = other DNA
tgctctgggttccaggttccactggtgacgcggcccagccggccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagtaagtttaaacccgctgatcagcctcgactgtgccttctagttg

SEQ ID NO: 220 - DNA, 31 bp, synthetic construct; identical to SEQ ID NO: 166
  misc_feature 1..31: note = pSEC Tag2 5' overlapping sequence
  source 1..31: mol_type = other DNA

SEQ ID NO: 221 - DNA, 33 bp, synthetic construct; identical to SEQ ID NO: 64
  misc_feature 1..33: note = pSEC Tag2 3' overlapping sequence
  source 1..33: mol_type = other DNA

SEQ ID NO: 222 - DNA, 63 bp, synthetic construct
  misc_feature 1..63: note = Murine Ig kappa chain leader sequence
  source 1..63: mol_type = other DNA
atggagacagacacactcctgctatgggtactgctgctctgggttccaggttccactggtgac

SEQ ID NO: 223 - protein, 21 aa, synthetic construct
  REGION 1..21: note = Murine Ig kappa chain leader sequence
METDTLLLWVLLLWVPGSTGD

SEQ ID NO: 224 - DNA, 60 bp, synthetic construct
  misc_feature 1..60: note = Interleukin-2 (IL-2) leader sequence
  source 1..60: mol_type = other DNA
atgtacaggatgcaactcctgtcttgcattgcactaagtcttgcacttgtcacaaacagt

SEQ ID NO: 225 - protein, 20 aa, synthetic construct
  REGION 1..20: note = Interleukin-2 (IL-2) leader sequence
MYRMQLLSCIALSLALVTNS

SEQ ID NO: 226 - DNA, 48 bp, synthetic construct
  misc_feature 1..48: note = CD33 leader sequence
  source 1..48: mol_type = other DNA
atgcctcttctgcttctgcttcctctgctttgggctggagctcttgct

SEQ ID NO: 227 - protein, 16 aa, synthetic construct
  REGION 1..16: note = CD33 leader sequence
MPLLLLLPLLWAGALA

SEQ ID NO: 228 - DNA, 57 bp, synthetic construct
  misc_feature 1..57: note = IGHV3-21*03 leader sequence
  source 1..57: mol_type = other DNA
atggaactggggctccgctgggttttccttgttgctattttagaaggtgtccagtgt

SEQ ID NO: 229 - protein, 19 aa, synthetic construct
  REGION 1..19: note = IGHV3-21*03 leader sequence
MELGLRWVFLVAILEGVQC

SEQ ID NO: 230 - DNA, 60 bp, synthetic construct
  misc_feature 1..60: note = IGHV3-11*02 leader sequence
  source 1..60: mol_type = other DNA
atggaagccccagcgcagcttctcttcctcctgctactctggctcccagataccactgga

SEQ ID NO: 231 - protein, 20 aa, synthetic construct
  REGION 1..20: note = IGHV3-11*02 leader sequence
MEAPAQLLFLLLLWLPDTTG

SEQ ID NO: 232 - DNA, 729 bp, synthetic construct
  misc_feature 1..729: note = Humanized E6 single chain GS3
  source 1..729: mol_type = other DNA
gaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggcggtggcggatccggcggtggcggatccggcggtggcggatccgaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaa

SEQ ID NO: 233 - protein, 243 aa, synthetic construct
  REGION 1..243: note = Humanized E6 single chain GS3
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 234 - DNA, 747 bp, synthetic construct
  misc_feature 1..747: note = Humanized E6 single chain IgG1noC
  source 1..747: mol_type = other DNA
gaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcgataaaacccatactaaaccgccaaaaccggcgccggaactgctgggtggtcctggtaccggtgaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaa

SEQ ID NO: 235 - protein, 249 aa, synthetic construct
  REGION 1..249: note = Humanized E6 single chain IgG1noC
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSDKTHTKPPKPAPELLGGPGTGEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 236 - DNA, 813 bp, synthetic construct
  misc_feature 1..813: note = Humanized E6 single chain X4 (linker is IgG1 and IgG2 modifiedhinge region)
  source 1..813: mol_type = other DNA
gaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcgataaaacccatactaaaccgccaaaaccggcgccggaactgctgggtggtcctggtaccggtactggtggtccgactattaaacctccgaaacctccgaaacctgctccgaacctgctgggtggtccggaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaa

SEQ ID NO: 237 - protein, 271 aa, synthetic construct
  REGION 1..271: note = Humanized E6 single chain X4 (linker is IgG1 and IgG2 modifiedhinge region)
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSDKTHTKPPKPAPELLGGPGTGTGGPTIKPPKPPKPAPNLLGGPEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 238 - DNA, 747 bp, synthetic construct
  misc_feature 1..747: note = Humanized C2 single chain GS3
  source 1..747: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaact

SEQ ID NO: 239 - protein, 249 aa, synthetic construct
  REGION 1..249: note = Humanized C2 single chain GS3
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 240 - DNA, 765 bp, synthetic construct
  misc_feature 1..765: note = Humanized C2 single chain IgG (no Cysteine)
  source 1..765: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccgataaaacccatactaaaccgccaaaaccggcgccggaactgctgggtggtcctggtaccggtgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaact

SEQ ID NO: 241 - protein, 255 aa, synthetic construct
  REGION 1..255: note = Humanized C2 single chain IgG (no Cysteine)
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSDKTHTKPPKPAPELLGGPGTGDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 242 - DNA, 831 bp, synthetic construct
  misc_feature 1..831: note = Humanized C2 single chain X4 (linker is IgG1 and IgG2 modifiedhinge region)
  source 1..831: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccgataaaacccatactaaaccgccaaaaccggcgccggaactgctgggtggtcctggtaccggtactggtggtccgactattaaacctccgaaacctccgaaacctgctccgaacctgctgggtggtccggacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaact

SEQ ID NO: 243 - protein, 277 aa, synthetic construct
  REGION 1..277: note = Humanized C2 single chain X4 (linker is IgG1 and IgG2 modifiedhinge region)
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSDKTHTKPPKPAPELLGGPGTGTGGPTIKPPKPPKPAPNLLGGPDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 244 - DNA, 744 bp, synthetic construct
  misc_feature 1..744: note = Humanized C3 single chain GS3
  source 1..744: mol_type = other DNA
caggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcggcggtggcggatccggcggtggcggatccggcggtggcggatccgatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaact

SEQ ID NO: 245 - protein, 248 aa, synthetic construct
  REGION 1..248: note = Humanized C3 single chain GS3
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSGGGGSGGGGSGGGGSDIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRT

SEQ ID NO: 246 - DNA, 762 bp, synthetic construct
  misc_feature 1..762: note = Humanized C3 single chain IgG1 (no Cysteine)
  source 1..762: mol_type = other DNA
caggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcgataaaacccatactaaaccgccaaaaccggcgccggaactgctgggtggtcctggtaccggtgatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaact

SEQ ID NO: 247 - protein, 254 aa, synthetic construct
  REGION 1..254: note = Humanized C3 single chain IgG1 (no Cysteine)
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSDKTHTKPPKPAPELLGGPGTGDIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRT

SEQ ID NO: 248 - DNA, 828 bp, synthetic construct
  misc_feature 1..828: note = Humanized C3 single chain X4 (linker is IgG1 and IgG2 modifiedhinge region)
  source 1..828: mol_type = other DNA
caggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcgataaaacccatactaaaccgccaaaaccggcgccggaactgctgggtggtcctggtaccggtactggtggtccgactattaaacctccgaaacctccgaaacctgctccgaacctgctgggtggtccggatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaact

SEQ ID NO: 249 - protein, 276 aa, synthetic construct
  REGION 1..276: note = Humanized C3 single chain X4 (linker is IgG1 and IgG2 modifiedhinge region)
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSDKTHTKPPKPAPELLGGPGTGTGGPTIKPPKPPKPAPNLLGGPDIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRT

SEQ ID NO: 250 - DNA, 741 bp, synthetic construct
  misc_feature 1..741: note = Humanized C8 single chain GS3 (linker is [Gly4Ser1]3)
  source 1..741: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccctgactcagtgaagggccgattcaccatctccagagacaacgccaagaactcactgtatctgcaaatgaacagcctgagagccgaggacacggccgtgtattactgtgcgagactgggcggcgataactattatgaatattggggcaaagggaccacggtcaccgtctcctccggcggtggcggatccggcggtggcggatccggcggtggcggatccgacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaact

SEQ ID NO: 251 - protein, 247 aa, synthetic construct
  REGION 1..247: note = Humanized C8 single chain GS3 (linker is [Gly4Ser1]3)
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSSGGGGSGGGGSGGGGSDIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRT

SEQ ID NO: 252 - DNA, 759 bp, synthetic construct
  misc_feature 1..759: note = Humanized C8 single chain IgG1 (no Cysteine)
  source 1..759: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccctgactcagtgaagggccgattcaccatctccagagacaacgccaagaactcactgtatctgcaaatgaacagcctgagagccgaggacacggccgtgtattactgtgcgagactgggcggcgataactattatgaatattggggcaaagggaccacggtcaccgtctcctccgataaaacccatactaaaccgccaaaaccggcgccggaactgctgggtggtcctggtaccggtgacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaact

SEQ ID NO: 253 - protein, 253 aa, synthetic construct
  REGION 1..253: note = Humanized C8 single chain IgG1 (no Cysteine)
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSSDKTHTKPPKPAPELLGGPGTGDIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRT

SEQ ID NO: 254 - DNA, 825 bp, synthetic construct
  misc_feature 1..825: note = Humanized C8 single chain X4 (linker is IgG1 and IgG2 modifiedhinge region)
  source 1..825: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactacccagactcagtgaagggccgattcaccatctccagagacaacgccaagaactcactgtatctgcaaatgaacagcctgagagccgaggacacggccgtgtattactgtgcgagactgggcggcgacaattactatgagtattggggcaaagggaccacggtcaccgtctcctccgataaaacccatactaaaccgccaaaaccggcgccggaactgctgggtggtcctggtaccggtactggtggtccgactattaaacctccgaaacctccgaaacctgctccgaacctgctgggtggtccggacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaact

SEQ ID NO: 255 - protein, 275 aa, synthetic construct
  REGION 1..275: note = Humanized C8 single chain X4 (linker is IgG1 and IgG2 modifiedhinge region)
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSSDKTHTKPPKPAPELLGGPGTGTGGPTIKPPKPPKPAPNLLGGPDIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRT

SEQ ID NO: 256 - DNA, 1509 bp, synthetic construct
  misc_feature 1..1509: note = pSECTag2 E6 scFV-FC
  source 1..1509: mol_type = other DNA
atggagacagacacactcctgctatgggtactgctgctctgggttccaggttccactggtgacgcggcccagccggccgaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggcggtggcggatccggcggtggcggatccggcggtggcggatccgaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 257 - protein, 501 aa, synthetic construct
  REGION 1..501: note = pSECTag2 E6 scFV-FC
METDTLLLWVLLLWVPGSTGDAAQPAEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - DNA, 496 bp, synthetic construct
  misc_feature 1..496: note = E6 scFC-FC 1 gBLOCk sequence
  source 1..496: mol_type = other DNA
tgctctgggttccaggttccactggtgacgcggcccagccggccgaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggcggtggcggatccggcggtggcggatccggcggtggcggatccgaaattgtgttgacacagtctccagccaccctgtctttgtc

SEQ ID NO: 259 - DNA, 583 bp, synthetic construct
  misc_feature 1..583: note = E6 scFC-FC 2 gBLOCk sequence
  source 1..583: mol_type = other DNA
aattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagc

SEQ ID NO: 260 - DNA, 1527 bp, synthetic construct
  misc_feature 1..1527: note = pSECTag2 C2 scFV-FC
  source 1..1527: mol_type = other DNA
atggagacagacacactcctgctatgggtactgctgctctgggttccaggttccactggtgacgcggcccagccggccgaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 261 - protein, 507 aa, synthetic construct
  REGION 1..507: note = pSECTag2 C2 scFV-FC
METDTLLLWVLLLWVPGSTGDAAQPAEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - DNA, 487 bp, synthetic construct
  misc_feature 1..487: note = C2 scFV-FC 1 gBLOCk sequence
  source 1..487: mol_type = other DNA
tgctctgggttccaggttccactggtgacgcggcccagccggccgaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctccggcggtggcggatccggcggtggcggatccggcggtggcggatccgacattgtgctgacccagtctccagcctccttggc

SEQ ID NO: 263 - DNA, 604 bp, synthetic construct
  misc_feature 1..604: note = C2 scFV-FC 2 gBLOCk sequence
  source 1..604: mol_type = other DNA
cattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagc

SEQ ID NO: 264 - DNA, 1524 bp, synthetic construct
  misc_feature 1..1524: note = pSECTag2 C3 scFV-FC
  source 1..1524: mol_type = other DNA
atggagacagacacactcctgctatgggtactgctgctctgggttccaggttccactggtgacgcggcccagccggcccaggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcggcggtggcggatccggcggtggcggatccggcggtggcggatccgatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaactgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 265 - protein, 506 aa, synthetic construct
  REGION 1..506: note = pSECTag2 C3 scFV-FC
METDTLLLWVLLLWVPGSTGDAAQPAQVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSGGGGSGGGGSGGGGSDIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 266 - DNA, 480 bp, synthetic construct
  misc_feature 1..480: note = C3 GS scFV FC 1 gBLOCk sequence
  source 1..480: mol_type = other DNA
tgctctgggttccaggttccactggtgacgcggcccagccggcccaggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcggcggtggcggatccggcggtggcggatccggcggtggcggatccgatattgtgatgacccagactccactctctctgt

SEQ ID NO: 267 - DNA, 607 bp, synthetic construct
  misc_feature 1..607: note = C3 scFV FC2 gBLOCk sequence
  source 1..607: mol_type = other DNA
tattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaactgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagc

SEQ ID NO: 268 - DNA, 1521 bp, synthetic construct
  misc_feature 1..1521: note = pSECTag2 C8 scFV-FC
  source 1..1521: mol_type = other DNA
atggagacagacacactcctgctatgggtactgctgctctgggttccaggttccactggtgacgcggcccagccggccgaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccctgactcagtgaagggccgattcaccatctccagagacaacgccaagaactcactgtatctgcaaatgaacagcctgagagccgaggacacggccgtgtattactgtgcgagactgggcggcgataactattatgaatattggggcaaagggaccacggtcaccgtctcctccggcggtggcggatccggcggtggcggatccggcggtggcggatccgacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaactgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 269 - protein, 505 aa, synthetic construct
  REGION 1..505: note = pSECTag2 C8 scFV-FC
METDTLLLWVLLLWVPGSTGDAAQPAEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSSGGGGSGGGGSGGGGSDIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - DNA, 477 bp, synthetic construct
  misc_feature 1..477: note = C8 scFV FC 1 gBLOCk sequence
  source 1..477: mol_type = other DNA
tgctctgggttccaggttccactggtgacgcggcccagccggccgaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccctgactcagtgaagggccgattcaccatctccagagacaacgccaagaactcactgtatctgcaaatgaacagcctgagagccgaggacacggccgtgtattactgtgcgagactgggcggcgataactattatgaatattggggcaaagggaccacggtcaccgtctcctccggcggtggcggatccggcggtggcggatccggcggtggcggatccgacatcgtgatgacccagtctccagactccctgg

SEQ ID NO: 271 - DNA, 607 bp, synthetic construct
  misc_feature 1..607: note = C8 scFV FC2 gBLOCk sequence
  source 1..607: mol_type = other DNA
catcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaactgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagc

SEQ ID NO: 272 - DNA, 702 bp, synthetic construct
  misc_feature 1..702: note = Human IgG1 Fc sequence
  source 1..702: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 273 - protein, 232 aa, synthetic construct
  REGION 1..232: note = Human IgG1 Fc sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - DNA, 666 bp, synthetic construct
  misc_feature 1..666: note = Human IgG1 CH2-CH3 domain sequence
  source 1..666: mol_type = other DNA
ccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 275 - protein, 220 aa, synthetic construct
  REGION 1..220: note = Human IgG1 CH2-CH3 domain sequence
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 276 - DNA, 327 bp, synthetic construct
  misc_feature 1..327: note = Human IgG1 CH3 domain sequence
  source 1..327: mol_type = other DNA
gggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 277 - protein, 107 aa, synthetic construct
  REGION 1..107: note = Human IgG1 CH3 domain sequence
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - DNA, 702 bp, synthetic construct
  misc_feature 1..702: note = Human IgG1 Fc Y407R sequence
  source 1..702: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaggagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 279 - protein, 232 aa, synthetic construct
  REGION 1..232: note = Human IgG1 Fc Y407R sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLRSKLTVDKSRWQQGNVFSCSVMHEALHNHYTQKSLSLSPGK

SEQ ID NO: 280 - DNA, 702 bp, synthetic construct
  misc_feature 1..702: note = Human IgG1 Fc F405Q sequence
  source 1..702: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttccagctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 281 - protein, 232 aa, synthetic construct
  REGION 1..232: note = Human IgG1 Fc F405Q sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFQLYSKLTVDKSRWQQGNVFSCSVMHEALHNHYTQKSLSLSPGK

SEQ ID NO: 282 - DNA, 702 bp, synthetic construct
  misc_feature 1..702: note = Human IgG1 Fc T394D sequence
  source 1..702: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccgaccctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 283 - protein, 232 aa, synthetic construct
  REGION 1..232: note = Human IgG1 Fc T394D sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTDPPVLDSDGSFFLYSKLTVDKSRWQQGNVFSCSVMHEALHNHYTQKSLSLSPGK

SEQ ID NO: 284 - DNA, 702 bp, synthetic construct
  misc_feature 1..702: note = Human IgG1 Fc T366W/L368W sequence
  source 1..702: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgctgggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 285 - protein, 232 aa, synthetic construct
  REGION 1..232: note = Human IgG1 Fc T366W/L368W sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCWVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - DNA, 702 bp, synthetic construct
  misc_feature 1..702: note = Human IgG1 Fc T364R/L368R sequence
  source 1..702: mol_type = other DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcaggctgacctgcagggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 287 - protein, 232 aa, synthetic construct
  REGION 1..232: note = Human IgG1 Fc T364R/L368R sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVRLTCRVKGFYPSDIAVEWESNGQPENNYKTTPPVLDSDGSFFLYSKLTVDKSRWQQGNVFSCSVMHEALHNHYTQKSLSLSPGK

SEQ ID NO: 288 - DNA, 657 bp, synthetic construct
  misc_feature 1..657: note = Human IgG1 Fc hingeless sequence
  source 1..657: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 289 - protein, 217 aa, synthetic construct
  REGION 1..217: note = Human IgG1 Fc hingeless sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - DNA, 696 bp, synthetic construct
  misc_feature 1..696: note = Human IgG1 G237A FC sequence
  source 1..696: mol_type = other DNA
gagcccaaatcttgtgacaaaactcacacatgcccaccgtgcccagcacctgaactcctgggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 291 - protein, 232 aa, synthetic construct
  REGION 1..232: note = Human IgG1 G237A FC sequence
EPKSCDKTHTCPPCPAPELLGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 292 - DNA, 696 bp, synthetic construct
  misc_feature 1..696: note = Human IgG1 L234A/L235A FC sequence
  source 1..696: mol_type = other DNA
gagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 293 - protein, 232 aa, synthetic construct
  REGION 1..232: note = Human IgG1 L234A/L235A FC sequence
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 294 - DNA, 1341 bp, synthetic construct
  misc_feature 1..1341: note = CAR-T E6 CD3z sequence
  source 1..1341: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 295 - protein, 445 aa, synthetic construct
  REGION 1..445: note = CAR-T E6 CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 296 - DNA, 485 bp, synthetic construct
  misc_feature 1..485: note = CAR-T E6 CD3z gBLOCK sequence
  source 1..485: mol_type = other DNA
tggagctgtgcacacaagaggactggatttcgcctgtgatatctacatttgggccccgctcgcaggcacatgtggagtgctcctcctctccctggtgattaccctgtactgccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataagtttaaacccgctgatcagcctcgactgtgc

SEQ ID NO: 297 - DNA, 1464 bp, synthetic construct
  misc_feature 1..1464: note = CAR-T E6 CD28/CD3z sequence
  source 1..1464: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtcccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 298 - protein, 486 aa, synthetic construct
  REGION 1..486: note = CAR-T E6 CD28/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 299 - DNA, 608 bp, synthetic construct
  misc_feature 1..608: note = CAR-T E6 CD28/CD3z g BLOCK sequence
  source 1..608: mol_type = other DNA
tggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtcccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataagtttaaacccgctgatcagcctcgactgtgc

SEQ ID NO: 300 - DNA, 1467 bp, synthetic construct
  misc_feature 1..1467: note = CAR-T E6 4-1BB/CD3z sequence
  source 1..1467: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 301 - protein, 487 aa, synthetic construct
  REGION 1..487: note = CAR-T E6 4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - DNA, 611 bp, synthetic construct
  misc_feature 1..611: note = CAR-T E6 4-1BB/CD3z gBLOCK sequence
  source 1..611: mol_type = other DNA
tggagctgtgcacacaagaggactggatttcgcctgtgatatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataagtttaaacccgctgatcagcctcgactgtgc

SEQ ID NO: 303 - DNA, 1590 bp, synthetic construct
  misc_feature 1..1590: note = CAR-T E6 CD28/4-1BB/CD3z sequence
  source 1..1590: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtccaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 304 - protein, 528 aa, synthetic construct
  REGION 1..528: note = CAR-T E6 CD28/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 305 - DNA, 1668 bp, synthetic construct
  misc_feature 1..1668: note = CAR-T E6 CD28/4-1BB/CD3z gBLOCK sequence
  source 1..1668: mol_type = other DNA
atagggagacccaagctggctagttaagcttggtaccgagggccaccatggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtccaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataagtttaaacccgctgatcagcctcgactgtgc

SEQ ID NO: 306 - DNA, 1608 bp, synthetic construct
  misc_feature 1..1608: note = CAR-T C2 CD28/4-1BB/CD3z sequence
  source 1..1608: mol_type = other DNA
atggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtccaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 307 - protein, 513 aa, synthetic construct
  REGION 1..513: note = CAR-T C2 CD28/4-1BB/CD3z sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 308 - DNA, 553 bp, synthetic construct
  misc_feature 1..553: note = CAR-T C2-1 gBLOCK sequence
  source 1..553: mol_type = other DNA
atagggagacccaagctggctagttaagcttggtaccgagggccaccatggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggc

SEQ ID NO: 309 - DNA, 518 bp, synthetic construct
  misc_feature 1..518: note = CAR-T C2-2 gBLOCK sequence
  source 1..518: mol_type = other DNA
aagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgc

SEQ ID NO: 310 - DNA, 2028 bp, synthetic construct
  misc_feature 1..2028: note = CAR E6 Fc/8/4-1BB/CD3z sequence
  source 1..2028: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 311 - protein, 674 aa, synthetic construct
  REGION 1..674: note = CAR E6 Fc/8/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 312 - DNA, 714 bp, synthetic construct
  misc_feature 1..714: note = E6 CAR pCDH gBLOCK sequence
  source 1..714: mol_type = other DNA
acgctgttttgacctccatagaagattctagagctagctgtagagcttggtaccgagggccaccatggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgc

SEQ ID NO: 313 - DNA, 723 bp, synthetic construct
  misc_feature 1..723: note = E6 CAR Fc pCDH gBLOCK sequence
  source 1..723: mol_type = other DNA
agtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctg

SEQ ID NO: 314 - DNA, 778 bp, synthetic construct
  misc_feature 1..778: note = E6 CAR 8BB3 pCDH gBLOCK sequence
  source 1..778: mol_type = other DNA
agaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataagtttaaacccgctgatcaggcggccgcgaaggatctgcgatcgctccggtgcccgtcag

SEQ ID NO: 315 - DNA, 1983 bp, synthetic construct
  misc_feature 1..1983: note = CAR E6 FcH/8/4-1BB/CD3z sequence
  source 1..1983: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 316 - protein, 659 aa, synthetic construct
  REGION 1..659: note = CAR E6 FcH/8/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 317 - DNA, 678 bp, synthetic construct
  misc_feature 1..678: note = E6 CAR FcH pCDH gBLOCK sequence
  source 1..678: mol_type = other DNA
agtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctg

SEQ ID NO: 318 - DNA, 2022 bp, synthetic construct
  misc_feature 1..2022: note = CAR E6 Fc/4/4-1BB/CD3z sequence
  source 1..2022: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaatggccctgattgtgctggggggcgtcgccggcctcctgcttttcattgggctaggcatcttcttcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 319 - protein, 672 aa, synthetic construct
  REGION 1..672: note = CAR E6 Fc/4/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKMALIVLGGVAGLLLFIGLGIFFKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 320 - DNA, 772 bp, synthetic construct
  misc_feature 1..772: note = E6 CAR 44BB3 pCDH gBLOCK sequence
  source 1..772: mol_type = other DNA
agaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaatggccctgattgtgctggggggcgtcgccggcctcctgcttttcattgggctaggcatcttcttcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataagtttaaacccgctgatcaggcggccgcgaaggatctgcgatcgctccggtgcccgtcag

SEQ ID NO: 321 - DNA, 1977 bp, synthetic construct
  misc_feature 1..1977: note = CAR E6 FcH/4/4-1BB/CD3z sequence
  source 1..1977: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaatggccctgattgtgctggggggcgtcgccggcctcctgcttttcattgggctaggcatcttcttcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 322 - protein, 657 aa, synthetic construct
  REGION 1..657: note = CAR E6 FcH/4/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKMALIVLGGVAGLLLFIGLGIFFKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 323 - DNA, 1506 bp, synthetic construct
  misc_feature 1..1506: note = CAR E6 IgD/8/4-1BB/CD3z sequence
  source 1..1506: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagagtctccaaaggcacaggcctcctcagtgcccactgcacaaccccaagcagagggcagcctcgccaaggcaaccacagccccagccaccacccgtaacacaggaagaggcggcgaagagaagaaaaaggagaaggagaaagaggaacaagaagagagagagacaaagacaccaatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 324 - protein, 500 aa, synthetic construct
  REGION 1..500: note = CAR E6 IgD/8/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKESPKAQASSVPTAQPQAEGSLAKATTAPATTRNTGRGGEEKKKEKEKEEQEERETKTPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 325 - DNA, 475 bp, synthetic construct
  misc_feature 1..475: note = E6 CAR IgD8 pcDH gBLOCK sequence
  source 1..475: mol_type = other DNA
agtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagagtctccaaaggcacaggcctcctcagtgcccactgcacaaccccaagcagagggcagcctcgccaaggcaaccacagccccagccaccacccgtaacacaggaagaggcggcgaagagaagaaaaaggagaaggagaaagaggaacaagaagagagagagacaaagacaccaatctacatttgggccccgctcgcaggcacatgtggagtgctcctcctctccctggtgattaccctgtactgcaaaaggggccgcaaaaaactcctttacatttttaagcagccttttatgaggccag

SEQ ID NO: 326 - DNA, 502 bp, synthetic construct
  misc_feature 1..502: note = E6 CAR BB 3 pCDH gBLOCK sequence
  source 1..502: mol_type = other DNA
acatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataagtttaaacccgctgatcaggcggccgcgaaggatctgcgatcgctccggtgcccgtcag

SEQ ID NO: 327 - DNA, 1500 bp, synthetic construct
  misc_feature 1..1500: note = CAR E6 IgD/4/4-1BB/CD3z sequence
  source 1..1500: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagagtctccaaaggcacaggcctcctcagtgcccactgcacaaccccaagcagagggcagcctcgccaaggcaaccacagccccagccaccacccgtaacacaggaagaggcggcgaagagaagaaaaaggagaaggagaaagaggaacaagaagagagagagacaaagacaccaatggccctgattgtgctggggggcgtcgccggcctcctgcttttcattgggctaggcatcttcttcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 328 - protein, 498 aa, synthetic construct
  REGION 1..498: note = CAR E6 IgD/4/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKESPKAQASSVPTAQPQAEGSLAKATTAPATTRNTGRGGEEKKKEKEKEEQEERETKTPMALIVLGGVAGLLLFIGLGIFFKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 329 - DNA, 469 bp, synthetic construct
  misc_feature 1..469: note = E6 CAR IgD4 pcDH gBLOCK sequence
  source 1..469: mol_type = other DNA
agtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagagtctccaaaggcacaggcctcctcagtgcccactgcacaaccccaagcagagggcagcctcgccaaggcaaccacagccccagccaccacccgtaacacaggaagaggcggcgaagagaagaaaaaggagaaggagaaagaggaacaagaagagagagagacaaagacaccaatggccctgattgtgctggggggcgtcgccggcctcctgcttttcattgggctaggcatcttcttcaaaaggggccgcaaaaaactcctttacatttttaagcagccttttatgaggccag

SEQ ID NO: 330 - DNA, 1461 bp, synthetic construct
  misc_feature 1..1461: note = CAR E6 X4/8/4-1BB/CD3z sequence
  source 1..1461: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagacaagacgcacaccaagccacctaaaccagctccagaactgctcggaggtcctggcaccggaaccggaggacctaccatcaaaccacctaagccacctaagcctgctcctaacctgctcggaggacctatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 331 - protein, 485 aa, synthetic construct
  REGION 1..485: note = CAR E6 X4/8/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKDKTHTKPPKPAPELLGGPGTGTGGPTIKPPKPPKPAPNLLGGPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 332 - DNA, 430 bp, synthetic construct
  misc_feature 1..430: note = E6 CAR X48 pCDH gBLOCK sequence
  source 1..430: mol_type = other DNA
agtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagacaagacgcacaccaagccacctaaaccagctccagaactgctcggaggtcctggcaccggaaccggaggacctaccatcaaaccacctaagccacctaagcctgctcctaacctgctcggaggacctatctacatttgggccccgctcgcaggcacatgtggagtgctcctcctctccctggtgattaccctgtactgcaaaaggggccgcaaaaaactcctttacatttttaagcagccttttatgaggccag

SEQ ID NO: 333 - DNA, 1455 bp, synthetic construct
  misc_feature 1..1455: note = CAR E6 X4/4/4-1BB/CD3z sequence
  source 1..1455: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagacaagacgcacaccaagccacctaaaccagctccagaactgctcggaggtcctggcaccggaaccggaggacctaccatcaaaccacctaagccacctaagcctgctcctaacctgctcggaggacctatggccctgattgtgctggggggcgtcgccggcctcctgcttttcattgggctaggcatcttcttcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 334 - protein, 483 aa, synthetic construct
  REGION 1..483: note = CAR E6 X4/4/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKDKTHTKPPKPAPELLGGPGTGTGGPTIKPPKPPKPAPNLLGGPMALIVLGGVAGLLLFIGLGIFFKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 335 - DNA, 424 bp, synthetic construct
  misc_feature 1..424: note = E6 CAR X44 pCDH gBLOCK sequence
  source 1..424: mol_type = other DNA
agtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaagacaagacgcacaccaagccacctaaaccagctccagaactgctcggaggtcctggcaccggaaccggaggacctaccatcaaaccacctaagccacctaagcctgctcctaacctgctcggaggacctatggccctgattgtgctggggggcgtcgccggcctcctgcttttcattgggctaggcatcttcttcaaaaggggccgcaaaaaactcctttacatttttaagcagccttttatgaggccag

SEQ ID NO: 336 - DNA, 1461 bp, synthetic construct
  misc_feature 1..1461: note = CAR E6 8+4/4/4-1BB/CD3z sequence
  source 1..1461: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggcggaggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatggccctgattgtgctggggggcgtcgccggcctcctgcttttcattgggctaggcatcttcttcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 337 - protein, 485 aa, synthetic construct
  REGION 1..485: note = CAR E6 8+4/4/4-1BB/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDMALIVLGGVAGLLLFIGLGIFFKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 338 - DNA, 430 bp, synthetic construct
  misc_feature 1..430: note = E6 CAR CD844 pCDH gBLOCK sequence
  source 1..430: mol_type = other DNA
agtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatggccctgattgtgctggggggcgtcgccggcctcctgcttttcattgggctaggcatcttcttcaaaaggggccgcaaaaaactcctttacatttttaagcagccttttatgaggccag

SEQ ID NO: 339 - DNA, 820 bp, synthetic construct
  misc_feature 1..820: note = Humanized C2 scFV sequence in CAR
  source 1..820: mol_type = other DNA
gagggccaccatggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaacc

SEQ ID NO: 340 - protein, 249 aa, synthetic construct; identical to SEQ ID NO: 239
  REGION 1..249: note = Humanized C2 scFV sequence in CAR

SEQ ID NO: 341 - DNA, 729 bp, synthetic construct
  misc_feature 1..729: note = Humanized E6 scFV sequence in CAR
  source 1..729: mol_type = other DNA
gaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaa

SEQ ID NO: 342 - protein, 243 aa, synthetic construct; identical to SEQ ID NO: 233
  REGION 1..243: note = Humanized E6 scFV sequence in CAR

SEQ ID NO: 343 - DNA, 63 bp, synthetic construct
  misc_feature 1..63: note = CD8 leader sequence
  source 1..63: mol_type = other DNA
atggccctgcccgtgaccgctttgctgctccccctggcgctgctgctgcacgccgccaggcca

SEQ ID NO: 344 - protein, 21 aa, synthetic construct
  REGION 1..21: note = CD8 leader sequence
MALPVTALLLPLALLLHAARP

SEQ ID NO: 345 - DNA, 135 bp, synthetic construct
  misc_feature 1..135: note = CD8 hinge domain sequence
  source 1..135: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgat

SEQ ID NO: 346 - protein, 45 aa, synthetic construct
  REGION 1..45: note = CD8 hinge domain sequence
TTTPAPRPPTPAPTIASQPLSLRPEACRPAAGGAVHTRGLDFACD

SEQ ID NO: 347 - DNA, 66 bp, synthetic construct
  misc_feature 1..66: note = CD4 hinge domain sequence
  source 1..66: mol_type = other DNA
tcgggacaggtcctgctggaatccaacatcaaggttctgcccacatggtccaccccggtgcagcca

SEQ ID NO: 348 - protein, 22 aa, synthetic construct
  REGION 1..22: note = CD4 hinge domain sequence
SGQVLLESNIKVLPTWSTPVQP

SEQ ID NO: 349 - DNA, 45 bp, synthetic construct
  misc_feature 1..45: note = CD28 hinge domain sequence
  source 1..45: mol_type = other DNA
aaacacctttgtccaagtcccctatttcccggaccttctaagccc

SEQ ID NO: 350 - protein, 15 aa, synthetic construct
  REGION 1..15: note = CD28 hinge domain sequence
KHLCPSPLFPGPSKP

SEQ ID NO: 351 - DNA, 201 bp, synthetic construct
  misc_feature 1..201: note = CD8+CD4 hinge domain sequence
  source 1..201: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgattcgggacaggtcctgctggaatccaacatcaaggttctgcccacatggtccaccccggtgcagcca

SEQ ID NO: 352 - protein, 67 aa, synthetic construct
  REGION 1..67: note = CD8+CD4 hinge domain sequence
TTTPAPRPPTPAPTIASQPLSLRPEACRPAAGGAVHTRGLDFACDSGQVLLESNIKVLPTWSTPVQP

SEQ ID NO: 353 - DNA, 180 bp, synthetic construct
  misc_feature 1..180: note = CD8+CD28 hinge domain sequence
  source 1..180: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgataaacacctttgtccaagtcccctatttcccggaccttctaagccc

SEQ ID NO: 354 - protein, 60 aa, synthetic construct
  REGION 1..60: note = CD8+CD28 hinge domain sequence
TTTPAPRPPTPAPTIASQPLSLRPEACRPAAGGAVHTRGLDFACDKHLCPSPLFPGPSKP

SEQ ID NO: 355 - DNA, 111 bp, synthetic construct
  misc_feature 1..111: note = CD28+CD4 hinge domain sequence
  source 1..111: mol_type = other DNA
aaacacctttgtccaagtcccctatttcccggaccttctaagccctcgggacaggtcctgctggaatccaacatcaaggttctgcccacatggtccaccccggtgcagcca

SEQ ID NO: 356 - protein, 37 aa, synthetic construct
  REGION 1..37: note = CD28+CD4 hinge domain sequence
KHLCPSPLFPGPSKPSGQVLLESNIKVLPTWSTPVQP

SEQ ID NO: 357 - DNA, 174 bp, synthetic construct
  misc_feature 1..174: note = Human IgD hinge domain sequence
  source 1..174: mol_type = other DNA
gagtctccaaaggcacaggcctcctcagtgcccactgcacaaccccaagcagagggcagcctcgccaaggcaaccacagccccagccaccacccgtaacacaggaagaggcggcgaagagaagaaaaaggagaaggagaaagaggaacaagaagagagagagacaaagacacca

SEQ ID NO: 358 - protein, 58 aa, synthetic construct
  REGION 1..58: note = Human IgD hinge domain sequence
ESPKAQASSVPTAQPQAEGSLAKATTAPATTRNTGRGGEEKKKEKEKEEQEERETKTP

SEQ ID NO: 359 - DNA, 129 bp, synthetic construct
  misc_feature 1..129: note = X4 linker (IgG1 and IgG2 modified hinge region) sequence
  source 1..129: mol_type = other DNA
gacaagacgcacaccaagccacctaaaccagctccagaactgctcggaggtcctggcaccggaaccggaggacctaccatcaaaccacctaagccacctaagcctgctcctaacctgctcggaggacct

SEQ ID NO: 360 - protein, 43 aa, synthetic construct
  REGION 1..43: note = X4 linker (IgG1 and IgG2 modified hinge region) sequence
DKTHTKPPKPAPELLGGPGTGTGGPTIKPPKPPKPAPNLLGGP

SEQ ID NO: 361 - DNA, 63 bp, synthetic construct
  misc_feature 1..63: note = CD3 zeta transmembrane domain sequence
  source 1..63: mol_type = other DNA
ctctgctacctgctggatggaatcctcttcatctatggtgtcattctcactgccttgttcctg

SEQ ID NO: 362 - protein, 21 aa, synthetic construct
  REGION 1..21: note = CD3 zeta transmembrane domain sequence
LCYLLDGILFIYGVILTALFL

SEQ ID NO: 363 - DNA, 72 bp, synthetic construct
  misc_feature 1..72: note = CD8 transmembrane domain sequence
  source 1..72: mol_type = other DNA
atctacatttgggccccgctcgcaggcacatgtggagtgctcctcctctccctggtgattaccctgtactgc

SEQ ID NO: 364 - protein, 24 aa, synthetic construct
  REGION 1..24: note = CD8 transmembrane domain sequence
IYIWAPLAGTCGVLLLSLVITLYC

SEQ ID NO: 365 - DNA, 66 bp, synthetic construct
  misc_feature 1..66: note = CD4 transmembrane domain sequence
  source 1..66: mol_type = other DNA
atggccctgattgtgctggggggcgtcgccggcctcctgcttttcattgggctaggcatcttcttc

SEQ ID NO: 366 - protein, 22 aa, synthetic construct
  REGION 1..22: note = CD4 transmembrane domain sequence
MALIVLGGVAGLLLFIGLGIFF

SEQ ID NO: 367 - DNA, 81 bp, synthetic construct
  misc_feature 1..81: note = CD28 transmembrane domain sequence
  source 1..81: mol_type = other DNA
ttttgggtgctggtggtggttggtggagtcctggcttgctatagcttgctagtaacagtggcctttattattttctgggtg

SEQ ID NO: 368 - protein, 27 aa, synthetic construct
  REGION 1..27: note = CD28 transmembrane domain sequence
FWVLVVVGGVLACYSLLVTVAFIIFWV

SEQ ID NO: 369 - DNA, 81 bp, synthetic construct
  misc_feature 1..81: note = 4-1BB transmembrane domain sequence
  source 1..81: mol_type = other DNA
atcatctccttctttcttgcgctgacgtcgactgcgttgctcttcctgctgttcttcctcacgctccgtttctctgttgtt

SEQ ID NO: 370 - protein, 27 aa, synthetic construct
  REGION 1..27: note = 4-1BB transmembrane domain sequence
IISFFLALTSTALLFLLFFLTLRFSVV

SEQ ID NO: 371 - DNA, 78 bp, synthetic construct
  misc_feature 1..78: note = OX40 transmembrane domain sequence
  source 1..78: mol_type = other DNA
gttgccgccatcctgggcctgggcctggtgctggggctgctgggccccctggccatcctgctggccctgtacctgctc

SEQ ID NO: 372 - protein, 26 aa, synthetic construct
  REGION 1..26: note = OX40 transmembrane domain sequence
VAAILGLGLVLGLLGPLAILLALYLL

SEQ ID NO: 373 - DNA, 336 bp, synthetic construct
  misc_feature 1..336: note = CD3 zeta domain sequence
  source 1..336: mol_type = other DNA
cgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacgg

SEQ ID NO: 374 - protein, 112 aa, synthetic construct
  REGION 1..112: note = CD3 zeta domain sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 375 - DNA, 336 bp, synthetic construct
  misc_feature 1..336: note = CD3 zeta domain variant sequence
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 376 - protein, 112 aa, synthetic construct
  REGION 1..112: note = CD3 zeta domain variant sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 377 - DNA, 123 bp, synthetic construct
  misc_feature 1..123: note = CD28 domain sequence
  source 1..123: mol_type = other DNA
agaagcaagcggtctcggctcctgcattctgattacatgaacatgaccccaagaagaccaggccccaccaggaaacattaccagccctacgctccgccacgcgacttcgctgcctaccggtcc

SEQ ID NO: 378 - protein, 41 aa, synthetic construct
  REGION 1..41: note = CD28 domain sequence
RSKRSRLLHSDYMNMTPRRPGPTRKHYQPYAPPRDFAAYRS

SEQ ID NO: 379 - DNA, 126 bp, synthetic construct
  misc_feature 1..126: note = 4-1BB domain sequence
  source 1..126: mol_type = other DNA
aaaaggggccgcaaaaaactcctttacatttttaagcagccttttatgaggccagtacagacgactcaagaggaagacgggtgctcatgccgctttcctgaggaggaggaaggagggtgcgaactg

SEQ ID NO: 380 - protein, 42 aa, synthetic construct
  REGION 1..42: note = 4-1BB domain sequence
KRGRKKLLYIFKQPFMRPVQTTQEEDGCSCRFPEEEEGGCEL

SEQ ID NO: 381 - DNA, 111 bp, synthetic construct
  misc_feature 1..111: note = OX40 domain sequence
  source 1..111: mol_type = other DNA
cggagggaccagaggctgccccccgatgcccacaagccccctgggggaggcagtttccggacccccatccaagaggagcaggccgacgcccactccaccctggccaagatc

SEQ ID NO: 382 - protein, 37 aa, synthetic construct
  REGION 1..37: note = OX40 domain sequence
RRDQRLPPDAHKPPGGGSFRTPIQEEQADAHSTLAKI

SEQ ID NO: 383 - DNA, 723 bp, synthetic construct
  misc_feature 1..723: note = Humanized anti CD3 scFV clone 12F6 (VH-VL) sequence
  source 1..723: mol_type = other DNA
caggtgcagctggtgcagagcggaggtggagtggtccaacctggaagatctctgagactgagctgtaaggctagcgggtacacgttcacatcttacacgatgcactgggtgaggcaagcccccggtaagggcctggaatggatcggatatataaaccccagctcagggtataccaaatataatcagaagttcaaagatcggttcacgatttctgctgataaaagtaagtccaccgctttcctgcagatggactcactcaggccagaagatactggtgtttatttctgtgcaaggtggcaggactacgacgtgtactttgactattgggggcaggggacgcctgtaacagtatcaagcggcggtggcggatccggcggtggcggatccggcggtggcggatccgatattcagatgacccagagcccgagcagcctgagcgcgagcgtgggcgatcgcgtgaccatgacctgccgcgcgagcagcagcgtgagctatatgcattggtatcagcagaccccgggcaaagcgccgaaaccgtggatttatgcgaccagcaacctggcgagcggcgtgccgagccgctttagcggcagcggcagcggcaccgattataccctgaccattagcagcctgcagccggaagatattgcgacctattattgccagcagtggagcagcaacccgccgacctttggccagggcaccaaactgcagattacccgc

SEQ ID NO: 384 - protein, 241 aa, synthetic construct
  REGION 1..241: note = Humanized anti CD3 scFV clone 12F6 (VH-VL) sequence
QVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSSGGGGSGGGGSGGGGSDIQMTQSPSSLSASVGDRVTMTCRASSSVSYMHWYQQTPGKAPKPWIYATSNLASGVPSRFSGSGSGTDYTLTISSLQPEDIATYYCQQWSSNPPTFGQGTKLQITR

SEQ ID NO: 385 - DNA, 723 bp, synthetic construct
  misc_feature 1..723: note = Humanized anti CD3 scFV clone 12F6 (VL-VH) sequence
  source 1..723: mol_type = other DNA
gatattcagatgacccagagcccgagcagcctgagcgcgagcgtgggcgatcgcgtgaccatgacctgccgcgcgagcagcagcgtgagctatatgcattggtatcagcagaccccgggcaaagcgccgaaaccgtggatttatgcgaccagcaacctggcgagcggcgtgccgagccgctttagcggcagcggcagcggcaccgattataccctgaccattagcagcctgcagccggaagatattgcgacctattattgccagcagtggagcagcaacccgccgacctttggccagggcaccaaactgcagattacccgcggcggtggcggatccggcggtggcggatccggcggtggcggatcccaggtgcagctggtgcagagcggaggtggagtggtccaacctggaagatctctgagactgagctgtaaggctagcgggtacacgttcacatcttacacgatgcactgggtgaggcaagcccccggtaagggcctggaatggatcggatatataaaccccagctcagggtataccaaatataatcagaagttcaaagatcggttcacgatttctgctgataaaagtaagtccaccgctttcctgcagatggactcactcaggccagaagatactggtgtttatttctgtgcaaggtggcaggactacgacgtgtactttgactattgggggcaggggacgcctgtaacagtatcaagc

SEQ ID NO: 386 - protein, 241 aa, synthetic construct
  REGION 1..241: note = Humanized anti CD3 scFV clone 12F6 (VL-VH) sequence
DIQMTQSPSSLSASVGDRVTMTCRASSSVSYMHWYQQTPGKAPKPWIYATSNLASGVPSRFSGSGSGTDYTLTISSLQPEDIATYYCQQWSSNPPTFGQGTKLQITRGGGGSGGGGSGGGGSQVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSS

SEQ ID NO: 387 - DNA, 723 bp, synthetic construct
  misc_feature 1..723: note = Humanized anti CD3 scFV clone OKT3 (VH-VL) sequence
  source 1..723: mol_type = other DNA
caggtgcagctggtgcagagcggaggcggagtggtgcagcctggaagaagcctgcgcctgagctgcaaagcgagcggctatacctttacccgctataccatgcattgggtgcgccaggcgccgggcaaaggcctggaatggattggctatattaacccgagccgcggctataccaactataaccagaaagtgaaagatcgctttaccattagcaccgataaaagcaaaagcaccgcgtttctgcagatggatagcctgcgcccggaagataccgcggtgtattattgcgcgcgctattatgatgatcattattgcctggattattggggccagggcaccaccctgaccgtgagcagcggcggtggcggatccggcggtggcggatccggcggtggcggatccgatattcagatgacccagagcccgagcagcctgagcgcgagcgtgggcgatcgcgtgaccattacctgcagcgcgagcagcagcgtgagctatatgaactggtatcagcagaccccgggcaaagcgccgaaacgctggatttatgataccagcaaactggcgagcggcgtgccgagccgctttagcggcagcggcagcggcaccgattatacctttaccattagcagcctgcagccggaagatattgcgacctattattgccagcagtggagcagcaacccgtttacctttggccagggcaccaaactgcagattacccgc

SEQ ID NO: 388 - protein, 241 aa, synthetic construct
  REGION 1..241: note = Humanized anti CD3 scFV clone OKT3 (VH-VL) sequence
QVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 389 - DNA, 723 bp, synthetic construct
  misc_feature 1..723: note = Humanized anti CD3 scFV clone OKT3 (VH-VL) sequence
  source 1..723: mol_type = other DNA
gatattcagatgacccagagcccgagcagcctgagcgcgagcgtgggcgatcgcgtgaccattacctgcagcgcgagcagcagcgtgagctatatgaactggtatcagcagaccccgggcaaagcgccgaaacgctggatttatgataccagcaaactggcgagcggcgtgccgagccgctttagcggcagcggcagcggcaccgattatacctttaccattagcagcctgcagccggaagatattgcgacctattattgccagcagtggagcagcaacccgtttacctttggccagggcaccaaactgcagattacccgcggcggtggcggatccggcggtggcggatccggcggtggcggatcccaggtgcagctggtgcagagcggaggcggagtggtgcagcctggaagaagcctgcgcctgagctgcaaagcgagcggctatacctttacccgctataccatgcattgggtgcgccaggcgccgggcaaaggcctggaatggattggctatattaacccgagccgcggctataccaactataaccagaaagtgaaagatcgctttaccattagcaccgataaaagcaaaagcaccgcgtttctgcagatggatagcctgcgcccggaagataccgcggtgtattattgcgcgcgctattatgatgatcattattgcctggattattggggccagggcaccaccctgaccgtgagcagc

SEQ ID NO: 390 - protein, 241 aa, synthetic construct
  REGION 1..241: note = Humanized anti CD3 scFV clone OKT3 (VH-VL) sequence
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGGGGSGGGGSGGGGSQVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSS

SEQ ID NO: 391 - DNA, 729 bp, synthetic construct; identical to SEQ ID NO: 232
  misc_feature 1..729: note = HumanizeE6 scFV (VH-VL) sequence
  source 1..729: mol_type = other DNA

SEQ ID NO: 392 - protein, 243 aa, synthetic construct; identical to SEQ ID NO: 233
  REGION 1..243: note = HumanizeE6 scFV (VH-VL) sequence

SEQ ID NO: 393 - DNA, 729 bp, synthetic construct
  misc_feature 1..729: note = HumanizeE6 scFV (VL-VH) sequence
  source 1..729: mol_type = other DNA
gaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaggcggtggcggatccggcggtggcggatccggcggtggcggatccgaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagc

SEQ ID NO: 394 - protein, 243 aa, synthetic construct
  REGION 1..243: note = HumanizeE6 scFV (VL-VH) sequence
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKGGGGSGGGGSGGGGSEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 395 - DNA, 747 bp, synthetic construct; identical to SEQ ID NO: 238
  misc_feature 1..747: note = HumanizeC2 scFV (VH-VL) sequence
  source 1..747: mol_type = other DNA

SEQ ID NO: 396 - protein, 249 aa, synthetic construct; identical to SEQ ID NO: 239
  REGION 1..249: note = HumanizeC2 scFV (VH-VL) sequence

SEQ ID NO: 397 - DNA, 747 bp, synthetic construct
  misc_feature 1..747: note = HumanizeE6 scFV (VL-VH) sequence
  source 1..747: mol_type = other DNA
gacattgtgctgacccagtctccagcctccttggccgtgtctccaggacagagggccaccatcacctgcagagccagtaagagtgtcagtaccagcggatactcctacatgcactggtatcagcagaaaccaggacaacctcctaaactcctgatttacctggcatccaatctggagagcggggtcccagccaggttcagcggcagtgggtctgggaccgatttcaccctcacaattaatcctgtggaagctaatgatactgcaaattattactgtcagcacagtagggagctgcctttcacattcggcggagggaccaaggtggagatcaaacgaactggcggtggcggatccggcggtggcggatccggcggtggcggatccgaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccccgactcagtgaagggccgattcaccatctccagagacaacgccaagaactcactgtatctgcaaatgaacagcctgagagccgaggacacggccgtgtattactgtgcgagacttgggggggataattactacgaatacttcgatgtctggggcaaagggaccacggtcaccgtctcctcc

SEQ ID NO: 398 - protein, 249 aa, synthetic construct
  REGION 1..249: note = HumanizeE6 scFV (VL-VH) sequence
DIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTGGGGSGGGGSGGGGSEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSS

SEQ ID NO: 399 - DNA, 15 bp, synthetic construct
  misc_feature 1..15: note = G4S1 linker sequence
  source 1..15: mol_type = other DNA
ggcggtggcggatcc

SEQ ID NO: 400 - protein, 5 aa, synthetic construct
  REGION 1..5: note = G4S1 linker sequence
GGGGS

SEQ ID NO: 401 - DNA, 45 bp, synthetic construct
  misc_feature 1..45: note = [G4S1]x3 linker sequence
  source 1..45: mol_type = other DNA
ggcggtggcggatccggcggtggcggatccggcggtggcggatcc

SEQ ID NO: 402 - protein, 15 aa, synthetic construct
  REGION 1..15: note = [G4S1]x3 linker sequence
GGGGSGGGGSGGGGS

SEQ ID NO: 403 - DNA, 24 bp, synthetic construct
  misc_feature 1..24: note = 8 aa GS linker sequence
  source 1..24: mol_type = other DNA
ggcggttccggcggtggatccgga

SEQ ID NO: 404 - protein, 8 aa, synthetic construct
  REGION 1..8: note = 8 aa GS linker sequence
GGSGGGSG

SEQ ID NO: 405 - DNA, 39 bp, synthetic construct
  misc_feature 1..39: note = 12 aa GS linker sequence
  source 1..39: mol_type = other DNA
ggcggttccggcggtggatccggcggtggcggatccgga

SEQ ID NO: 406 - protein, 12 aa, synthetic construct
  REGION 1..12: note = 12 aa GS linker sequence
GGSGGGSGGGSG

SEQ ID NO: 407 - DNA, 39 bp, synthetic construct
  misc_feature 1..39: note = 13 aa GS linker sequence
  source 1..39: mol_type = other DNA
ggcggtggatccggcggtggcggatccggcggtggatcc

SEQ ID NO: 408 - protein, 13 aa, synthetic construct
  REGION 1..13: note = 13 aa GS linker sequence
GGGSGGGGSGGGS

SEQ ID NO: 409 - DNA, 66 bp, synthetic construct
  misc_feature 1..66: note = 22 aa GS linker sequence
  source 1..66: mol_type = other DNA
ggcggtggaagcggcggtggcggatccggcagcggcggaagcggcggtggcggatccggcggtgga

SEQ ID NO: 410 - protein, 22 aa, synthetic construct
  REGION 1..22: note = 22 aa GS linker sequence
GGGSGGGGSGSGGSGGGGSGGG

SEQ ID NO: 411 - DNA, 78 bp, synthetic construct
  misc_feature 1..78: note = 24 aa GS linker sequence
  source 1..78: mol_type = other DNA
ggcggttccggcggtggatccggcggtggcggatccggaggcggttccggcggtggatccggcggtggcggatccgga

SEQ ID NO: 412 - protein, 24 aa, synthetic construct
  REGION 1..24: note = 24 aa GS linker sequence
GGSGGGSGGGSGGGSGGGSGGGSG

SEQ ID NO: 413 - DNA, 357 bp, synthetic construct
  misc_feature 1..357: note = Mouse C3 Heavy chain variable region sequence
  source 1..357: mol_type = other DNA
caggtccagctgcagcagtctgggcctgagctggtgaggcctggggtctcagtgaagatttcctgcaagggttccggctacagattcactgattatgctatgaactgggtgaagcagagtcatgcaaagagtctagagtggattggagttattagtactttctctggtaatacaaacttcaaccagaagtttaagggcaaggccacaatgactgtagacaaatcctccagcacagcctatatggaacttgccagattgacatctgaggattctgccatgtattactgtgcaagatcggattactacggcccatactttgactactggggccaaggcaccactctcacagtctcctca

SEQ ID NO: 414 - protein, 119 aa, synthetic construct
  REGION 1..119: note = Mouse C3 Heavy chain variable region sequence
QVQLQQSGPELVRPGVSVKISCKGSGYRFTDYAMNWVKQSHAKSLEWIGVISTFSGNTNFNQKFKGKATMTVDKSSSTAYMELARLTSEDSAMYYCARSDYYGPYFDYWGQGTTLTVSS

SEQ ID NO: 415 - DNA, 90 bp, synthetic construct
  misc_feature 1..90: note = Mouse C3 heavy chain variable framework region 1 (FWR1) sequence
  source 1..90: mol_type = other DNA
caggtccagctgcagcagtctgggcctgagctggtgaggcctggggtctcagtgaagatttcctgcaagggttccggctacagattcact

SEQ ID NO: 416 - protein, 30 aa, synthetic construct
  REGION 1..30: note = Mouse C3 heavy chain variable framework region 1 (FWR1) sequence
QVQLQQSGPELVRPGVSVKISCKGSGYRFT

SEQ ID NO: 417 - DNA, 15 bp, synthetic construct
  misc_feature 1..15: note = Mouse C3 heavy chain variable complementarity determining regions1 (CDR1) sequence
  source 1..15: mol_type = other DNA
gattatgctatgaac

SEQ ID NO: 418 - protein, 5 aa, synthetic construct
  REGION 1..5: note = Mouse C3 heavy chain variable complementarity determining regions1 (CDR1) sequence
DYAMN

SEQ ID NO: 419 - DNA, 42 bp, synthetic construct
  misc_feature 1..42: note = Mouse C3 heavy chain variable framework region 2 (FWR2) sequence
  source 1..42: mol_type = other DNA
tgggtgaagcagagtcatgcaaagagtctagagtggattgga

SEQ ID NO: 420 - protein, 14 aa, synthetic construct
  REGION 1..14: note = Mouse C3 heavy chain variable framework region 2 (FWR2) sequence
WVKQSHAKSLEWIG

SEQ ID NO: 421 - DNA, 51 bp, synthetic construct
  misc_feature 1..51: note = Mouse C3 heavy chain variable complementarity determining regions2 (CDR2) sequence
  source 1..51: mol_type = other DNA
gttattagtactttctctggtaatacaaacttcaaccagaagtttaagggc

SEQ ID NO: 422 - protein, 17 aa, synthetic construct
  REGION 1..17: note = Mouse C3 heavy chain variable complementarity determining regions2 (CDR2) sequence
VISTFSGNTNFNQKFKG

SEQ ID NO: 423 - DNA, 96 bp, synthetic construct
  misc_feature 1..96: note = Mouse C3 heavy chain variable framework region 3 (FWR3) acidsequence
  source 1..96: mol_type = other DNA
aaggccacaatgactgtagacaaatcctccagcacagcctatatggaacttgccagattgacatctgaggattctgccatgtattactgtgcaaga

SEQ ID NO: 424 - protein, 32 aa, synthetic construct
  REGION 1..32: note = Mouse C3 heavy chain variable framework region 3 (FWR3) acidsequence
KATMTVDKSSSTAYMELARLTSEDSAMYYCAR

SEQ ID NO: 425 - DNA, 30 bp, synthetic construct
  misc_feature 1..30: note = Mouse C3 heavy chain variable complementarity determining regions3 (CDR3) sequence
  source 1..30: mol_type = other DNA
tcggattactacggcccatactttgactac

SEQ ID NO: 426 - protein, 10 aa, synthetic construct
  REGION 1..10: note = Mouse C3 heavy chain variable complementarity determining regions3 (CDR3) sequence
SDYYGPYFDY

SEQ ID NO: 427 - DNA, 296 bp, synthetic construct
  misc_feature 1..296: note = IGHV1-18*04 heavy chain variable region sequence
  source 1..296: mol_type = other DNA
caggttcagctggtgcagtctggagctgaggtgaagaagcctggggcctcagtgaaggtctcctgcaaggcttctggttacacctttaccagctacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaga

SEQ ID NO: 428 - protein, 98 aa, synthetic construct
  REGION 1..98: note = IGHV1-18*04 heavy chain variable region sequence
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 429 - DNA, 90 bp, synthetic construct
  misc_feature 1..90: note = IGHV1-18*04 heavy chain variable framework region 1 (FWR1)sequence
  source 1..90: mol_type = other DNA
caggttcagctggtgcagtctggagctgaggtgaagaagcctggggcctcagtgaaggtctcctgcaaggcttctggttacacctttacc

SEQ ID NO: 430 - protein, 30 aa, synthetic construct
  REGION 1..30: note = IGHV1-18*04 heavy chain variable framework region 1 (FWR1)sequence
QVQLVQSGAEVKKPGASVKVSCKASGYTFT

SEQ ID NO: 431 - DNA, 15 bp, synthetic construct
  misc_feature 1..15: note = IGHV1-18*04 heavy chain variable complementarity determiningregions 1 (CDR1) sequence
  source 1..15: mol_type = other DNA
agctacggtatcagc

SEQ ID NO: 432 - protein, 5 aa, synthetic construct
  REGION 1..5: note = IGHV1-18*04 heavy chain variable complementarity determiningregions 1 (CDR1) sequence
SYGIS

SEQ ID NO: 433 - DNA, 42 bp, synthetic construct
  misc_feature 1..42: note = IGHV1-18*04 heavy chain variable framework region 2 (FWR2)sequence
  source 1..42: mol_type = other DNA
tgggtgcgacaggcccctggacaagggcttgagtggatggga

SEQ ID NO: 434 - protein, 14 aa, synthetic construct
  REGION 1..14: note = IGHV1-18*04 heavy chain variable framework region 2 (FWR2)sequence
WVRQAPGQGLEWMG

SEQ ID NO: 435 - DNA, 51 bp, synthetic construct
  misc_feature 1..51: note = IGHV1-18*04 heavy chain variable complementarity determiningregions 2 (CDR2) sequence
  source 1..51: mol_type = other DNA
tggatcagcgcttacaatggtaacacaaactatgcacagaagctccagggc

SEQ ID NO: 436 - protein, 17 aa, synthetic construct
  REGION 1..17: note = IGHV1-18*04 heavy chain variable complementarity determiningregions 2 (CDR2) sequence
WISAYNGNTNYAQKLQG

SEQ ID NO: 437 - DNA, 96 bp, synthetic construct
  misc_feature 1..96: note = IGHV1-18*04 heavy chain variable framework region 3 (FWR3)sequence
  source 1..96: mol_type = other DNA
agagtcaccatgaccacagacacatccacgagcacagcctacatggagctgaggagcctgagatctgacgacacggccgtgtattactgtgcgaga

SEQ ID NO: 438 - protein, 32 aa, synthetic construct
  REGION 1..32: note = IGHV1-18*04 heavy chain variable framework region 3 (FWR3)sequence
RVTMTTDTSTSTAYMELRSLRSDDTAVYYCAR

SEQ ID NO: 439 - DNA, 357 bp, synthetic construct
  misc_feature 1..357: note = Humanized C3 heavy chain variable region sequence
  source 1..357: mol_type = other DNA
caggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagc

SEQ ID NO: 440 - protein, 119 aa, synthetic construct
  REGION 1..119: note = Humanized C3 heavy chain variable region sequence
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSS

SEQ ID NO: 441 - DNA, 90 bp, synthetic construct; identical to SEQ ID NO: 429
  misc_feature 1..90: note = Humanized C3 heavy chain variable framework region 1 (FWR1) acidsequence
  source 1..90: mol_type = other DNA

SEQ ID NO: 442 - protein, 30 aa, synthetic construct; identical to SEQ ID NO: 430
  REGION 1..30: note = Humanized C3 heavy chain variable framework region 1 (FWR1) acidsequence

SEQ ID NO: 443 - DNA, 15 bp, synthetic construct
  misc_feature 1..15: note = Humanized C3 heavy chain variable complementarity determiningregions 1 (CDR1) sequence
  source 1..15: mol_type = other DNA
gactacgccatgaac

SEQ ID NO: 444 - protein, 5 aa, synthetic construct
  REGION 1..5: note = Humanized C3 heavy chain variable complementarity determiningregions 1 (CDR1) sequence
DYAMN

SEQ ID NO: 445 - DNA, 42 bp, synthetic construct; identical to SEQ ID NO: 433
  misc_feature 1..42: note = Humanized C3 heavy chain variable framework region 2 (FWR2) acidsequence
  source 1..42: mol_type = other DNA

SEQ ID NO: 446 - protein, 14 aa, synthetic construct
  REGION 1..14: note = Humanized C3 heavy chain variable framework region 2 (FWR2) acidsequence
WVRQAPGQGLEWMG

SEQ ID NO: 447 - DNA, 51 bp, synthetic construct
  misc_feature 1..51: note = Humanized C3 heavy chain variable complementarity determiningregions 2 (CDR2) sequence
  source 1..51: mol_type = other DNA
gtgatcagcaccttcagcggtaacacaaacttcaaccagaagttcaagggc

SEQ ID NO: 448 - protein, 17 aa, synthetic construct
  REGION 1..17: note = Humanized C3 heavy chain variable complementarity determiningregions 2 (CDR2) sequence
VISTFSGNTNFNQKFKG

SEQ ID NO: 449 - DNA, 96 bp, synthetic construct; identical to SEQ ID NO: 437
  misc_feature 1..96: note = Humanized C3 heavy chain variable framework region 3 (FWR3) acidsequence
  source 1..96: mol_type = other DNA

SEQ ID NO: 450 - protein, 32 aa, synthetic construct; identical to SEQ ID NO: 438
  REGION 1..32: note = Humanized C3 heavy chain variable framework region 3 (FWR3) acidsequence

SEQ ID NO: 451 - DNA, 30 bp, synthetic construct
  misc_feature 1..30: note = Humanized C3 heavy chain variable complementarity determiningregions 3 (CDR3) sequence
  source 1..30: mol_type = other DNA
agcgactactacggcccatacttcgactac

SEQ ID NO: 452 - protein, 10 aa, synthetic construct
  REGION 1..10: note = Humanized C3 heavy chain variable complementarity determiningregions 3 (CDR3) sequence
SDYYGPYFDY

SEQ ID NO: 453 - DNA, 1353 bp, synthetic construct
  misc_feature 1..1353: note = Humanized C3 IgG1 heavy chain sequence
  source 1..1353: mol_type = other DNA
caggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 454 - protein, 449 aa, synthetic construct
  REGION 1..449: note = Humanized C3 IgG1 heavy chain sequence
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 455 - DNA, 1341 bp, synthetic construct
  misc_feature 1..1341: note = Humanized C3 IgG2 heavy chain sequence
  source 1..1341: mol_type = other DNA
caggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagtaa

SEQ ID NO: 456 - protein, 445 aa, synthetic construct
  REGION 1..445: note = Humanized C3 IgG2 heavy chain sequence
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 457 - DNA, 545 bp, synthetic construct
  misc_feature 1..545: note = Humanized C3 heavy chain IgG1 gBLOCK sequence
  source 1..545: mol_type = other DNA
tgctctgggttccaggttccactggtgacgcggcccagccggcccaggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagc

SEQ ID NO: 458 - DNA, 336 bp, synthetic construct
  misc_feature 1..336: note = Mouse C3 Light Chain variable region sequence
  source 1..336: mol_type = other DNA
gatgttttgatgacccaaactccactctccctgcctgtcagtcttggagatcaagcctccatctcttgcagatctagtcagaccattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccattcacgttcggctcggggacaaagttggaaataaaa

SEQ ID NO: 459 - protein, 112 aa, synthetic construct
  REGION 1..112: note = Mouse C3 Light Chain variable region sequence
DVLMTQTPLSLPVSLGDQASISCRSSQTIVHSNGNTYLEWYLQKPGQSPKLLIYKVSNRFSGVPDRFSGSGSGTDFTLKINRVEAEDLGVYYCFQGSHVPFTFGSGTKLEIK

SEQ ID NO: 460 - DNA, 69 bp, synthetic construct
  misc_feature 1..69: note = Mouse C3 light chain variable framework region 1 (FWR1) sequence
  source 1..69: mol_type = other DNA
gatgttttgatgacccaaactccactctccctgcctgtcagtcttggagatcaagcctccatctcttgc

SEQ ID NO: 461 - protein, 23 aa, synthetic construct
  REGION 1..23: note = Mouse C3 light chain variable framework region 1 (FWR1) sequence
DVLMTQTPLSLPVSLGDQASISC

SEQ ID NO: 462 - DNA, 48 bp, synthetic construct
  misc_feature 1..48: note = Mouse C3 light chain variable complementarity determining regions1 (CDR1) sequence
  source 1..48: mol_type = other DNA
agatctagtcagaccattgtacatagtaatggaaacacctatttagaa

SEQ ID NO: 463 - protein, 16 aa, synthetic construct
  REGION 1..16: note = Mouse C3 light chain variable complementarity determining regions1 (CDR1) sequence
RSSQTIVHSNGNTYLE

SEQ ID NO: 464 - DNA, 45 bp, synthetic construct
  misc_feature 1..45: note = Mouse C3 light chain variable framework region 2 (FWR2) sequence
  source 1..45: mol_type = other DNA
tggtacctgcagaaaccaggccagtctccaaagctcctgatctac

SEQ ID NO: 465 - protein, 15 aa, synthetic construct
  REGION 1..15: note = Mouse C3 light chain variable framework region 2 (FWR2) sequence
WYLQKPGQSPKLLIY

SEQ ID NO: 466 - DNA, 21 bp, synthetic construct
  misc_feature 1..21: note = Mouse C3 light chain variable complementarity determining regions2 (CDR2) sequence
  source 1..21: mol_type = other DNA
aaagtttccaaccgattttct

SEQ ID NO: 467 - protein, 7 aa, synthetic construct
  REGION 1..7: note = Mouse C3 light chain variable complementarity determining regions2 (CDR2) sequence
KVSNRFS

SEQ ID NO: 468 - DNA, 96 bp, synthetic construct
  misc_feature 1..96: note = Mouse C3 light chain variable framework region 3 (FWR3) sequence
  source 1..96: mol_type = other DNA
ggggtcccagacaggttcagtggcagtggatcagggacagatttcacactcaagatcaacagagtggaggctgaggatctgggagtttattactgc

SEQ ID NO: 469 - protein, 32 aa, synthetic construct
  REGION 1..32: note = Mouse C3 light chain variable framework region 3 (FWR3) sequence
GVPDRFSGSGSGTDFTLKINRVEAEDLGVYYC

SEQ ID NO: 470 - DNA, 27 bp, synthetic construct
  misc_feature 1..27: note = Mouse C3 light chain variable complementarity determining regions3 (CDR3) sequence
  source 1..27: mol_type = other DNA
tttcaaggttcacatgttccattcacg

SEQ ID NO: 471 - protein, 9 aa, synthetic construct
  REGION 1..9: note = Mouse C3 light chain variable complementarity determining regions3 (CDR3) sequence
FQGSHVPFT

SEQ ID NO: 472 - DNA, 300 bp, synthetic construct
  misc_feature 1..300: note = IGKV2-29*03 light chain variable region sequence
  source 1..300: mol_type = other DNA
gatattgtgatgacccagactccactctctctgtccgtcacccctggacagccggcctccatctcctgcaagtctagtcagagcctcctgcatagtgatggaaagacctatttgtattggtacctgcagaagccaggccagtctccacagctcctgatctatgaagtttccagccggttctctggagtgccagataggttcagtggcagcgggtcagggacagatttcacactgaaaatcagccgggtggaggctgaggatgttggggtttattactgcatgcaaggtatacaccttcct

SEQ ID NO: 473 - protein, 100 aa, synthetic construct
  REGION 1..100: note = IGKV2-29*03 light chain variable region sequence
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLP

SEQ ID NO: 474 - DNA, 69 bp, synthetic construct
  misc_feature 1..69: note = IGKV2-29*03 light chain variable framework region 1 (FWR1) acidsequence
  source 1..69: mol_type = other DNA
gatattgtgatgacccagactccactctctctgtccgtcacccctggacagccggcctccatctcctgc

SEQ ID NO: 475 - protein, 23 aa, synthetic construct
  REGION 1..23: note = IGKV2-29*03 light chain variable framework region 1 (FWR1) acidsequence
DIVMTQTPLSLSVTPGQPASISC

SEQ ID NO: 476 - DNA, 49 bp, synthetic construct
  misc_feature 1..49: note = IGKV2-29*03 light chain variable complementarity determiningregions 1 (CDR1) sequence
  source 1..49: mol_type = other DNA
aagtctagtcagagcctcctgcatagtgatggaaagacctatttsgtat

SEQ ID NO: 477 - protein, 16 aa, synthetic construct
  REGION 1..16: note = IGKV2-29*03 light chain variable complementarity determiningregions 1 (CDR1) sequence
KSSQSLLHSDGKTYLY

SEQ ID NO: 478 - DNA, 45 bp, synthetic construct
  misc_feature 1..45: note = IGKV2-29*03 light chain variable framework region 2 (FWR2)sequence
  source 1..45: mol_type = other DNA
tggtacctgcagaagccaggccagtctccacagctcctgatctat

SEQ ID NO: 479 - protein, 15 aa, synthetic construct
  REGION 1..15: note = IGKV2-29*03 light chain variable framework region 2 (FWR2)sequence
WYLQKPGQSPQLLIY

SEQ ID NO: 480 - DNA, 18 bp, synthetic construct
  misc_feature 1..18: note = IGKV2-29*03 light chain variable complementarity determiningregions 2 (CDR2) sequence
  source 1..18: mol_type = other DNA
gaagtttccagccggttc

SEQ ID NO: 481 - protein, 7 aa, synthetic construct
  REGION 1..7: note = IGKV2-29*03 light chain variable complementarity determiningregions 2 (CDR2) sequence
EVSSRFS

SEQ ID NO: 482 - DNA, 96 bp, synthetic construct
  misc_feature 1..96: note = IGKV2-29*03 light chain variable framework region 3 (FWR3)sequence
  source 1..96: mol_type = other DNA
ggagtgccagataggttcagtggcagcgggtcagggacagatttcacactgaaaatcagccgggtggaggctgaggatgttggggtttattactgc

SEQ ID NO: 483 - protein, 32 aa, synthetic construct
  REGION 1..32: note = IGKV2-29*03 light chain variable framework region 3 (FWR3)sequence
GVPDRFSGSGSGTDFTLKISRVEAEDVGVYYC

SEQ ID NO: 484 - DNA, 21 bp, synthetic construct
  misc_feature 1..21: note = IGKV2-29*03 light chain variable complementarity determiningregions3 (CDR3) sequence
  source 1..21: mol_type = other DNA
atgcaaggtatacaccttcct

SEQ ID NO: 485 - protein, 7 aa, synthetic construct
  REGION 1..7: note = IGKV2-29*03 light chain variable complementarity determiningregions3 (CDR3) sequence
MQGIHLP

SEQ ID NO: 486 - DNA, 342 bp, synthetic construct
  misc_feature 1..342: note = Humanized C3 light chain variable region sequence
  source 1..342: mol_type = other DNA
gatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaact

SEQ ID NO: 487 - protein, 114 aa, synthetic construct
  REGION 1..114: note = Humanized C3 light chain variable region sequence
DIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRT

SEQ ID NO: 488 - DNA, 69 bp, synthetic construct; identical to SEQ ID NO: 474
  misc_feature 1..69: note = Humanized C3 light chain variable framework region 1 (FWR1) acidsequence
  source 1..69: mol_type = other DNA

SEQ ID NO: 489 - protein, 23 aa, synthetic construct
  REGION 1..23: note = Humanized C3 light chain variable framework region 1 (FWR1) acidsequence
DIVMTQTPLSLSVTPGQPASISC

SEQ ID NO: 490 - DNA, 47 bp, synthetic construct
  misc_feature 1..47: note = Humanized C3 light chain variable complementarity determiningregions 1 (CDR1) sequence
  source 1..47: mol_type = other DNA
ggtctagtcagaccattgtccatagtaatggaaacacctatttggag

SEQ ID NO: 491 - protein, 16 aa, synthetic construct
  REGION 1..16: note = Humanized C3 light chain variable complementarity determiningregions 1 (CDR1) sequence
RSSQTIVHSNGNTYLE

SEQ ID NO: 492 - DNA, 45 bp, synthetic construct; identical to SEQ ID NO: 478
  misc_feature 1..45: note = Humanized C3 light chain variable framework region 2 (FWR2) acidsequence
  source 1..45: mol_type = other DNA

SEQ ID NO: 493 - protein, 15 aa, synthetic construct
  REGION 1..15: note = Humanized C3 light chain variable framework region 2 (FWR2) acidsequence
WYLQKPGQSPQLLIY

SEQ ID NO: 494 - DNA, 21 bp, synthetic construct
  misc_feature 1..21: note = Humanized C3 light chain variable complementarity determiningregions 2 (CDR2) sequence
  source 1..21: mol_type = other DNA
aaggtttccaaccggttctct

SEQ ID NO: 495 - protein, 7 aa, synthetic construct
  REGION 1..7: note = Humanized C3 light chain variable complementarity determiningregions 2 (CDR2) sequence
KVSNRFS

SEQ ID NO: 496 - DNA, 96 bp, synthetic construct; identical to SEQ ID NO: 482
  misc_feature 1..96: note = Humanized C3 light chain variable framework region 3 (FWR3) acidsequence
  source 1..96: mol_type = other DNA

SEQ ID NO: 497 - protein, 32 aa, synthetic construct; identical to SEQ ID NO: 483
  REGION 1..32: note = Humanized C3 light chain variable framework region 3 (FWR3) acidsequence

SEQ ID NO: 498 - DNA, 27 bp, synthetic construct
  misc_feature 1..27: note = Humanized C3 light chain variable complementarity determiningregions 3 (CDR3) sequence
  source 1..27: mol_type = other DNA
ttccaaggtagccacgtgcctttcacc

SEQ ID NO: 499 - protein, 9 aa, synthetic construct
  REGION 1..9: note = Humanized C3 light chain variable complementarity determiningregions 3 (CDR3) sequence
FQGSHVPFT

SEQ ID NO: 500 - DNA, 666 bp, synthetic construct
  misc_feature 1..666: note = Humanized C3 lambda light chainsequence
  source 1..666: mol_type = other DNA
gatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaactggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagtaa

SEQ ID NO: 501 - protein, 220 aa, synthetic construct
  REGION 1..220: note = Humanized C3 lambda light chainsequence
DIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRTGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 502 - DNA, 666 bp, synthetic construct
  misc_feature 1..666: note = Humanized C3 Kappa light chain
  source 1..666: mol_type = other DNA
gatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaactacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaa

SEQ ID NO: 503 - protein, 220 aa, synthetic construct
  REGION 1..220: note = Humanized C3 Kappa light chain
DIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRTTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 504 - DNA, 815 bp, synthetic construct
  misc_feature 1..815: note = Humanized C3 Kappa light gBLOCK sequence
  source 1..815: mol_type = other DNA
agctggctaggtaagcttggtaccgagctcggatccacgccaccatggagacagacacactcctgctatgggtactgctgctctgggttccaggttccactggtgacgatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaactacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaagtttaaacccgctgatcagcctcgactgtgccttctagttgc

SEQ ID NO: 505 - DNA, 321 bp, synthetic construct
  misc_feature 1..321: note = Mouse C8 heavy chain variable region sequence
  source 1..321: mol_type = other DNA
gaagtgatggtcgtggaaagcggcggtggtctggtaaagccggggggatcccttaagctttcttgcgccgcatccgggttcacgttctccggctatgccatgtcctgggtccgacagactcccgaaaagcgcttggaatgggtggccactatctcctccggggggacgtacatctactaccccgacagtgtgaaaggaagatttacaatatctcgcgacaacgcaaaaaataccttgtatcttcaaatgagctccctgcggtcagaggacactgccatgtactattgcgcccgcctgggcggcgacaattactatgagtat

SEQ ID NO: 506 - protein, 107 aa, synthetic construct
  REGION 1..107: note = Mouse C8 heavy chain variable region sequence
EVMVVESGGGLVKPGGSLKLSCAASGFTFSGYAMSWVRQTPEKRLEWVATISSGGTYIYYPDSVKGRFTISRDNAKNTLYLQMSSLRSEDTAMYYCARLGGDNYYEY

SEQ ID NO: 507 - DNA, 15 bp, synthetic construct
  misc_feature 1..15: note = Mouse C8 heavy chain variable complementarity determining region1 (CDR1) sequence
  source 1..15: mol_type = other DNA
ggctatgccatgtcc

SEQ ID NO: 508 - protein, 5 aa, synthetic construct
  REGION 1..5: note = Mouse C8 heavy chain variable complementarity determining region1 (CDR1) sequence
GYAMS

SEQ ID NO: 509 - DNA, 51 bp, synthetic construct
  misc_feature 1..51: note = Mouse C8 heavy chain variable complementarity determining region2 (CDR2) sequence
  source 1..51: mol_type = other DNA
actatctcctccggggggacgtacatctactaccccgacagtgtgaaagga

SEQ ID NO: 510 - protein, 17 aa, synthetic construct
  REGION 1..17: note = Mouse C8 heavy chain variable complementarity determining region2 (CDR2) sequence
TISSGGTYIYYPDSVKG

SEQ ID NO: 511 - DNA, 27 bp, synthetic construct
  misc_feature 1..27: note = Mouse C8 heavy chain variable complementarity determining region3 (CDR3) sequence
  source 1..27: mol_type = other DNA
ctgggcggcgacaattactatgagtat

SEQ ID NO: 512 - protein, 9 aa, synthetic construct
  REGION 1..9: note = Mouse C8 heavy chain variable complementarity determining region3 (CDR3) sequence
LGGDNYYEY

SEQ ID NO: 513 - DNA, 292 bp, synthetic construct
  misc_feature 1..292: note = IGHV3-21*04 heavy chain variable region sequence
  source 1..292: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggccgtgtattactgtgcga

SEQ ID NO: 514 - protein, 98 aa, synthetic construct; identical to SEQ ID NO: 27
  REGION 1..98: note = IGHV3-21*04 heavy chain variable region sequence

SEQ ID NO: 515 - DNA, 90 bp, synthetic construct; identical to SEQ ID NO: 28
  misc_feature 1..90: note = IGHV3-21*04 heavy chain variable framework region 1 (FWR1)sequence
  source 1..90: mol_type = other DNA

SEQ ID NO: 516 - protein, 30 aa, synthetic construct; identical to SEQ ID NO: 29
  REGION 1..30: note = IGHV3-21*04 heavy chain variable framework region 1 (FWR1)sequence

SEQ ID NO: 517 - DNA, 15 bp, synthetic construct
  misc_feature 1..15: note = IGHV3-21*04 heavy chain variable complementarity determiningregions 1 (CDR1) sequence
  source 1..15: mol_type = other DNA
agctatagcatgaac

SEQ ID NO: 518 - protein, 5 aa, synthetic construct
  REGION 1..5: note = IGHV3-21*04 heavy chain variable complementarity determiningregions 1 (CDR1) sequence
SYSMN

SEQ ID NO: 519 - DNA, 39 bp, synthetic construct
  misc_feature 1..39: note = IGHV3-21*04 heavy chain variable framework region 2 (FWR2)sequence
  source 1..39: mol_type = other DNA
tgggtccgccaggctccagggaaggggctggagtgggtc

SEQ ID NO: 520 - protein, 13 aa, synthetic construct
  REGION 1..13: note = IGHV3-21*04 heavy chain variable framework region 2 (FWR2)sequence
WVRQAPGKGLEWV

SEQ ID NO: 521 - DNA, 54 bp, synthetic construct
  misc_feature 1..54: note = IGHV3-21*04 heavy chain variable complementarity determiningregions 2 (CDR2) sequence
  source 1..54: mol_type = other DNA
tcatccattagtagtagtagtagttacatatactacgcagactcagtgaagggc

SEQ ID NO: 522 - protein, 18 aa, synthetic construct
  REGION 1..18: note = IGHV3-21*04 heavy chain variable complementarity determiningregions 2 (CDR2) sequence
SSISSSSSYIYYADSVKG

SEQ ID NO: 523 - DNA, 94 bp, synthetic construct
  misc_feature 1..94: note = IGHV3-21*04 heavy chain variable framework region 3 (FWR3)sequence
  source 1..94: mol_type = other DNA
cgattcaccatctccagagacaacgccaagaactcactgtatctgcaaatgaacagcctgagagccgaggacacggccgtgtattactgtgcga

SEQ ID NO: 524 - protein, 32 aa, synthetic construct; identical to SEQ ID NO: 37
  REGION 1..32: note = IGHV3-21*04 heavy chain variable framework region 3 (FWR3)sequence

SEQ ID NO: 525 - DNA, 354 bp, synthetic construct
  misc_feature 1..354: note = Humanized C8 heavy chain variable region sequence
  source 1..354: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccctgactcagtgaagggccgattcaccatctccagagacaacgccaagaactcactgtatctgcaaatgaacagcctgagagccgaggacacggccgtgtattactgtgcgagactgggcggcgataactattatgaatattggggcaaagggaccacggtcaccgtctcctcc

SEQ ID NO: 526 - protein, 118 aa, synthetic construct
  REGION 1..118: note = Humanized C8 heavy chain variable region sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSS

SEQ ID NO: 527 - DNA, 90 bp, synthetic construct; identical to SEQ ID NO: 28
  misc_feature 1..90: note = Humanized C8 heavy chain variable framework region 1 (FWR1)sequence
  source 1..90: mol_type = other DNA

SEQ ID NO: 528 - protein, 30 aa, synthetic construct; identical to SEQ ID NO: 29
  REGION 1..30: note = Humanized C8 heavy chain variable framework region 1 (FWR1)sequence

SEQ ID NO: 529 - DNA, 15 bp, synthetic construct
  misc_feature 1..15: note = Humanized C8 heavy chain variable complementarity determiningregion 1 (CDR1) sequence
  source 1..15: mol_type = other DNA
ggctatgccatgagc

SEQ ID NO: 530 - protein, 5 aa, synthetic construct
  REGION 1..5: note = Humanized C8 heavy chain variable complementarity determiningregion 1 (CDR1) sequence
GYAMS

SEQ ID NO: 531 - DNA, 42 bp, synthetic construct; identical to SEQ ID NO: 32
  misc_feature 1..42: note = Humanized C8 heavy chain variable framework region 2 (FWR2)sequence
  source 1..42: mol_type = other DNA

SEQ ID NO: 532 - protein, 14 aa, synthetic construct
  REGION 1..14: note = Humanized C8 heavy chain variable framework region 2 (FWR2)sequence
WVRQAPGKGLEWVS

SEQ ID NO: 533 - DNA, 51 bp, synthetic construct
  misc_feature 1..51: note = Humanized C8 heavy chain variable complementarity determiningregion 2 (CDR2) sequence
  source 1..51: mol_type = other DNA
accattagtagtggcggaacctacatatactaccctgactcagtgaagggc

SEQ ID NO: 534 - protein, 17 aa, synthetic construct
  REGION 1..17: note = Humanized C8 heavy chain variable complementarity determiningregion 2 (CDR2) sequence
TISSGGTYIYYPDSVKG

SEQ ID NO: 535 - DNA, 96 bp, synthetic construct; identical to SEQ ID NO: 142
  misc_feature 1..96: note = Humanized C8 heavy chain variable framework region 3 (FWR3)sequence
  source 1..96: mol_type = other DNA

SEQ ID NO: 536 - protein, 32 aa, synthetic construct; identical to SEQ ID NO: 37
  REGION 1..32: note = Humanized C8 heavy chain variable framework region 3 (FWR3)sequence

SEQ ID NO: 537 - DNA, 27 bp, synthetic construct
  misc_feature 1..27: note = Humanized C8 heavy chain variable complementarity determiningregion 3 (CDR3) sequence
  source 1..27: mol_type = other DNA
ctgggcggcgataactattatgaatat

SEQ ID NO: 538 - protein, 9 aa, synthetic construct
  REGION 1..9: note = Humanized C8 heavy chain variable complementarity determiningregion 3 (CDR3) sequence
LGGDNYYEY

SEQ ID NO: 539 - DNA, 1350 bp, synthetic construct
  misc_feature 1..1350: note = Humanized C8 IgG1 heavy chain sequence
  source 1..1350: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccctgactcagtgaagggccgattcaccatctccagagacaacgccaagaactcactgtatctgcaaatgaacagcctgagagccgaggacacggccgtgtattactgtgcgagactgggcggcgataactattatgaatattggggcaaagggaccacggtcaccgtctcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 540 - protein, 448 aa, synthetic construct
  REGION 1..448: note = Humanized C8 IgG1 heavy chain sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 541 - DNA, 1338 bp, synthetic construct
  misc_feature 1..1338: note = Humanized C8 IgG2 heavy chain sequence
  source 1..1338: mol_type = other DNA
gaggtgcagctggtggagtctgggggaggcctggtcaagcctggggggtccctgagactctcctgtgcagcctctggattcaccttcagtggctatgccatgagctgggtccgccaggctccagggaaggggctggagtgggtctcaaccattagtagtggcggaacctacatatactaccctgactcagtgaagggccgattcaccatctccagagacaacgccaagaactcactgtatctgcaaatgaacagcctgagagccgaggacacggccgtgtattactgtgcgagactgggcggcgataactattatgaatattggggcaaagggaccacggtcaccgtctcctccgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagtaa

SEQ ID NO: 542 - protein, 444 aa, synthetic construct
  REGION 1..444: note = Humanized C8 IgG2 heavy chain sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 543 - DNA, 306 bp, synthetic construct
  misc_feature 1..306: note = Mouse C8 light chain variable region sequence
  source 1..306: mol_type = other DNA
gacatcgtcattacgcagacccctgccagtcttgccgtttctctgggccagagggccactatcagttacagggcgagtaagtctgtgagtaccagcggctatagttacatgcattggaaccagcagaaaccgggacagccaccacgcctgcttatttatctggtgtctaatcttgagtccggggtgcccgccaggttcagcggcagcggctctgggaccgacttcacactcaacattcatccagtggaagaagaggacgctgctacatactactgtcaacacattcgggaactgaccaggagtgaa

SEQ ID NO: 544 - protein, 102 aa, synthetic construct
  REGION 1..102: note = Mouse C8 light chain variable region sequence
DIVITQTPASLAVSLGQRATISYRASKSVSTSGYSYMHWNQQKPGQPPRLLIYLVSNLESGVPARFSGSGSGTDFTLNIHPVEEEDAATYYCQHIRELTRSE

SEQ ID NO: 545 - DNA, 45 bp, synthetic construct
  misc_feature 1..45: note = Mouse C8 light chain variable complementarity determining region1 (CDR1) sequence
  source 1..45: mol_type = other DNA
agggcgagtaagtctgtgagtaccagcggctatagttacatgcat

SEQ ID NO: 546 - protein, 15 aa, synthetic construct
  REGION 1..15: note = Mouse C8 light chain variable complementarity determining region1 (CDR1) sequence
RASKSVSTSGYSYMH

SEQ ID NO: 547 - DNA, 21 bp, synthetic construct
  misc_feature 1..21: note = Mouse C8 light chain variable complementarity determining region2 (CDR2) sequence
  source 1..21: mol_type = other DNA
ctggtgtctaatcttgagtcc

SEQ ID NO: 548 - protein, 7 aa, synthetic construct
  REGION 1..7: note = Mouse C8 light chain variable complementarity determining region2 (CDR2) sequence
LVSNLES

SEQ ID NO: 549 - DNA, 30 bp, synthetic construct
  misc_feature 1..30: note = Mouse C8 light chain variable complementarity determining region3 (CDR3) sequence
  source 1..30: mol_type = other DNA
caacacattcgggaactgaccaggagtgaa

SEQ ID NO: 550 - protein, 10 aa, synthetic construct
  REGION 1..10: note = Mouse C8 light chain variable complementarity determining region3 (CDR3) sequence
QHIRELTRSE

SEQ ID NO: 551 - DNA, 303 bp, synthetic construct
  misc_feature 1..303: note = NCBI germline z00023 light chain variable region sequence
  source 1..303: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcct

SEQ ID NO: 552 - protein, 101 aa, synthetic construct
  REGION 1..101: note = NCBI germline z00023 light chain variable region sequence
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 553 - DNA, 69 bp, synthetic construct
  misc_feature 1..69: note = NCBI germline z00023 light chain variable framework region 1(FWR1) acid sequence
  source 1..69: mol_type = other DNA
gacatcgtgatgacccagtctccagactccctggctgtgtctctgggcgagagggccaccatcaactgc

SEQ ID NO: 554 - protein, 23 aa, synthetic construct
  REGION 1..23: note = NCBI germline z00023 light chain variable framework region 1(FWR1) acid sequence
DIVMTQSPDSLAVSLGERATINC

SEQ ID NO: 555 - DNA, 51 bp, synthetic construct
  misc_feature 1..51: note = NCBI germline z00023 light chain variable complementaritydetermining regions 1 (CDR1) sequence
  source 1..51: mol_type = other DNA
aagtccagccagagtgttttatacagctccaacaataagaactacttagct

SEQ ID NO: 556 - protein, 17 aa, synthetic construct
  REGION 1..17: note = NCBI germline z00023 light chain variable complementaritydetermining regions 1 (CDR1) sequence
KSSQSVLYSSNNKNYLA

SEQ ID NO: 557 - DNA, 45 bp, synthetic construct
  misc_feature 1..45: note = NCBI germline z00023 light chain variable framework region 2(FWR2) sequence
  source 1..45: mol_type = other DNA
tggtaccagcagaaaccaggacagcctcctaagctgctcatttac

SEQ ID NO: 558 - protein, 15 aa, synthetic construct
  REGION 1..15: note = NCBI germline z00023 light chain variable framework region 2(FWR2) sequence
WYQQKPGQPPKLLIY

SEQ ID NO: 559 - DNA, 21 bp, synthetic construct
  misc_feature 1..21: note = NCBI germline z00023 light chain variable complementaritydetermining regions 2 (CDR2) sequence
  source 1..21: mol_type = other DNA
tgggcatctacccgggaatcc

SEQ ID NO: 560 - protein, 7 aa, synthetic construct
  REGION 1..7: note = NCBI germline z00023 light chain variable complementaritydetermining regions 2 (CDR2) sequence
WASTRES

SEQ ID NO: 561 - DNA, 96 bp, synthetic construct
  misc_feature 1..96: note = NCBI germline z00023 light chain variable framework region 3(FWR3) sequence
  source 1..96: mol_type = other DNA
ggggtccctgaccgattcagtggcagcgggtctgggacagatttcactctcaccatcagcagcctgcaggctgaagatgtggcagtttattactgt

SEQ ID NO: 562 - protein, 32 aa, synthetic construct
  REGION 1..32: note = NCBI germline z00023 light chain variable framework region 3(FWR3) sequence
GVPDRFSGSGSGTDFTLTISSLQAEDVAVYYC

SEQ ID NO: 563 - DNA, 21 bp, synthetic construct
  misc_feature 1..21: note = NCBI germline z00023 light chain variable complementaritydetermining regions3 (CDR3) sequence
  source 1..21: mol_type = other DNA
cagcaatattatagtactcct

SEQ ID NO: 564 - protein, 7 aa, synthetic construct
  REGION 1..7: note = NCBI germline z00023 light chain variable complementaritydetermining regions3 (CDR3) sequence
QQYYSTP

SEQ ID NO: 565 - DNA, 342 bp, synthetic construct
  misc_feature 1..342: note = Humanized C8 light chain variable region sequence
  source 1..342: mol_type = other DNA
gacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaact

SEQ ID NO: 566 - protein, 114 aa, synthetic construct
  REGION 1..114: note = Humanized C8 light chain variable region sequence
DIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRT

SEQ ID NO: 567 - DNA, 69 bp, synthetic construct; identical to SEQ ID NO: 553
  misc_feature 1..69: note = Humanized C8 light chain variable framework region 1 (FWR1)sequence
  source 1..69: mol_type = other DNA

SEQ ID NO: 568 - protein, 23 aa, synthetic construct
  REGION 1..23: note = Humanized C8 light chain variable framework region 1 (FWR1)sequence
DIVMTQSPDSLAVSLGERATINC

SEQ ID NO: 569 - DNA, 42 bp, synthetic construct
  misc_feature 1..42: note = Humanized C8 light chain variable complementarity determiningregion 1 (CDR1) sequence
  source 1..42: mol_type = other DNA
agggccagcaagagtgttagcaccagcggctacagctacatg

SEQ ID NO: 570 - protein, 14 aa, synthetic construct
  REGION 1..14: note = Humanized C8 light chain variable complementarity determiningregion 1 (CDR1) sequence
RASKSVSTSGYSYM

SEQ ID NO: 571 - DNA, 48 bp, synthetic construct
  misc_feature 1..48: note = Humanized C8 light chain variable framework region 2 (FWR2)sequence
  source 1..48: mol_type = other DNA
cactggtaccagcagaaaccaggacagcctcctaagctgctcatttac

SEQ ID NO: 572 - protein, 16 aa, synthetic construct
  REGION 1..16: note = Humanized C8 light chain variable framework region 2 (FWR2)sequence
HWYQQKPGQPPKLLIY

SEQ ID NO: 573 - DNA, 21 bp, synthetic construct
  misc_feature 1..21: note = Humanized C8 light chain variable complementarity determiningregion 2 (CDR2) sequence
  source 1..21: mol_type = other DNA
ctggtgtctaacctggaatcc

SEQ ID NO: 574 - protein, 7 aa, synthetic construct
  REGION 1..7: note = Humanized C8 light chain variable complementarity determiningregion 2 (CDR2) sequence
LVSNLES

SEQ ID NO: 575 - DNA, 96 bp, synthetic construct; identical to SEQ ID NO: 561
  misc_feature 1..96: note = Humanized C8 light chain variable framework region 3 (FWR3)sequence
  source 1..96: mol_type = other DNA

SEQ ID NO: 576 - protein, 32 aa, synthetic construct; identical to SEQ ID NO: 562
  REGION 1..32: note = Humanized C8 light chain variable framework region 3 (FWR3)sequence

SEQ ID NO: 577 - DNA, 30 bp, synthetic construct; identical to SEQ ID NO: 549
  misc_feature 1..30: note = Humanized C8 light chain variable complementarity determiningregion 3 (CDR3) sequence
  source 1..30: mol_type = other DNA

SEQ ID NO: 578 - protein, 10 aa, synthetic construct
  REGION 1..10: note = Humanized C8 light chain variable complementarity determiningregion 3 (CDR3) sequence
QHIRELTRSE

SEQ ID NO: 579 - DNA, 666 bp, synthetic construct
  misc_feature 1..666: note = Humanized C8 Lambda light chain sequence
  source 1..666: mol_type = other DNA
gacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaactggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagtaa

SEQ ID NO: 580 - protein, 220 aa, synthetic construct
  REGION 1..220: note = Humanized C8 Lambda light chain sequence
DIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRTGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 581 - DNA, 666 bp, synthetic construct
  misc_feature 1..666: note = Humanized C8 Kappa light chain sequence
  source 1..666: mol_type = other DNA
gacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaactacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaa

SEQ ID NO: 582 - protein, 220 aa, synthetic construct
  REGION 1..220: note = Humanized C8 Kappa light chain sequence
DIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRTTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 583 - DNA, 815 bp, synthetic construct
  misc_feature 1..815: note = Humanized C8 Kappa light chain gBLOCk sequence
  source 1..815: mol_type = other DNA
agctggctaggtaagcttggtaccgagctcggatccacgccaccatggagacagacacactcctgctatgggtactgctgctctgggttccaggttccactggtgacgacatcgtgatgacccagtctccagactccctggctgtgtctctgggcgagagggccaccatcaactgcagggccagcaagagtgttagcaccagcggctacagctacatgcactggtaccagcagaaaccaggacagcctcctaagctgctcatttacctggtgtctaacctggaatccggggtccctgaccgattcagtggcagcgggtctgggacagatttcactctcaccatcagcagcctgcaggctgaagatgtggcagtttattactgtcaacacattcgggaactgaccaggagtgaattcggcggagggaccaaggtggagatcaaacgaactacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtaagtttaaacccgctgatcagcctcgactgtgccttctagttgc

SEQ ID NO: 584 - DNA, 942 bp, synthetic construct
  misc_feature 1..942: note = CAR-T E6 CD8 sequence
  source 1..942: mol_type = other DNA
gaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgctgataa

SEQ ID NO: 585 - protein, 312 aa, synthetic construct
  REGION 1..312: note = CAR-T E6 CD8 sequence
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYC

SEQ ID NO: 586 - DNA, 960 bp, synthetic construct
  misc_feature 1..960: note = CAR-T C2 CD8 sequence
  source 1..960: mol_type = other DNA
gaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgctgataa

SEQ ID NO: 587 - protein, 318 aa, synthetic construct
  REGION 1..318: note = CAR-T C2 CD8 sequence
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYC

SEQ ID NO: 588 - DNA, 339 bp, synthetic construct
  misc_feature 1..339: note = CD8/4-1BB sequence
  source 1..339: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgtgataa

SEQ ID NO: 589 - protein, 111 aa, synthetic construct
  REGION 1..111: note = CD8/4-1BB sequence
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 590 - DNA, 336 bp, synthetic construct
  misc_feature 1..336: note = CD8/CD28 sequence
  source 1..336: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtcctgataa

SEQ ID NO: 591 - protein, 110 aa, synthetic construct
  REGION 1..110: note = CD8/CD28 sequence
TTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 592 - DNA, 549 bp, synthetic construct
  misc_feature 1..549: note = CD8/CD3z sequence
  source 1..549: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 593 - protein, 181 aa, synthetic construct
  REGION 1..181: note = CD8/CD3z sequence
TTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 594 - DNA, 672 bp, synthetic construct
  misc_feature 1..672: note = CD8/CD28/CD3z sequence
  source 1..672: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtcccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 595 - protein, 222 aa, synthetic construct
  REGION 1..222: note = CD8/CD28/CD3z sequence
TTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 596 - DNA, 675 bp, synthetic construct
  misc_feature 1..675: note = CD8/4-1BB/CD3z sequence
  source 1..675: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 597 - protein, 223 aa, synthetic construct
  REGION 1..223: note = CD8/4-1BB/CD3z sequence
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 598 - DNA, 798 bp, synthetic construct
  misc_feature 1..798: note = CD8/CD28/4-1BB/CD3z sequence
  source 1..798: mol_type = other DNA
acgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtccaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 599 - protein, 264 aa, synthetic construct
  REGION 1..264: note = CD8/CD28/4-1BB/CD3z sequence
TTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 600 - DNA, 1482 bp, synthetic construct
  misc_feature 1..1482: note = CAR-T C3 4-1BB/CD3z sequence
  source 1..1482: mol_type = other DNA
atggccctgcccgtgaccgctttgctgctccccctggcgctgctgctgcacgccgccaggccacaggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcggcggtggcggatccggcggtggcggatccggcggtggcggatccgatattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaactacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 601 - protein, 471 aa, synthetic construct
  REGION 1..471: note = CAR-T C3 4-1BB/CD3z sequence
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSGGGGSGGGGSGGGGSDIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 602 - DNA, 567 bp, synthetic construct
  misc_feature 1..567: note = C3 CAR gBLOCK 1 sequence
  source 1..567: mol_type = other DNA
atccacgctgttttgacctccatagaagattctagagctagctgtagagcttggtaccgagggccaccatggccctgcccgtgaccgctttgctgctccccctggcgctgctgctgcacgccgccaggccacaggttcagctggtgcagtctggagctgaggtgaagaagcctggggcctcagtgaaggtctcctgcaaggcttctggttacacctttaccgactacgccatgaactgggtgcgacaggcccctggacaagggcttgagtggatgggagtgatcagcaccttcagcggtaacacaaacttcaaccagaagttcaagggcagagtcaccatgaccacagacacatccacgagcacagcctacatggagctgaggagcctgagatctgacgacacggccgtgtattactgtgcgagaagcgactactacggcccatacttcgactactggggccagggcaccaccctgaccgtgtccagcggcggtggcggatccggcggtggcggatccggcggtggcggatccgatattgtgatgacccagactccactctctctgt

SEQ ID NO: 603 - DNA, 509 bp, synthetic construct
  misc_feature 1..509: note = C3 CAR gBLOCK 2 sequence
  source 1..509: mol_type = other DNA
tattgtgatgacccagactccactctctctgtccgtcacccctggacagccggcctccatctcctgcaggtctagtcagaccattgtccatagtaatggaaacacctatttggagtggtacctgcagaagccaggccagtctccacagctcctgatctataaggtttccaaccggttctctggagtgccagataggttcagtggcagcgggtcagggacagatttcacactgaaaatcagccgggtggaggctgaggatgttggggtttattactgcttccaaggtagccacgtgcctttcaccttcggcggagggaccaaggtggagatcaaacgaactacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtg

SEQ ID NO: 604 - DNA, 455 bp, synthetic construct
  misc_feature 1..455: note = E6 scFV gBLOCK 1 sequence
  source 1..455: mol_type = other DNA
tgctctgggttccaggttccactggtgacgcggcccagccggccgaggtgcagctggtggagtctgggggaggcctggtcaagcctggggggtccctgagactctcctgtgcagcctctggattcaccttcagtaggtatggcatgagctgggtccgccaggctccagggaagaggctggagtgggtctcaaccattagtggcggaggcacctacatatactacccagactcagtgaagggccgattcaccatctccagagacaacgccaagaacaccctgtatctgcaaatgaacagcctgagagccgaggacacggctgtgtattactgtaccagagataactatggccgcaactatgattatggcatggattattggggccagggcaccctggtgaccgtgagcagcggcggtggcggatccggcggtggcggatccggcggtggcggatcc

SEQ ID NO: 605 - DNA, 432 bp, synthetic construct
  misc_feature 1..432: note = E6 scFV gBLOCK 2 sequence
  source 1..432: mol_type = other DNA
ggcggtggcggatccggcggtggcggatccggcggtggcggatccgaaattgtgttgacacagtctccagccaccctgtctttgtctccaggggaaagagccaccctcacctgcagcgccaccagcagtgttagctacatccactggtaccaacagaggcctggccagagccccaggctcctcatctatagcacctccaacctggccagcggcatcccagccaggttcagtggcagtgggtctgggagcgactacactctcaccatcagcagcctagagcctgaagattttgcagtttattactgtcagcagcgtagcagctcccctttcacctttggcagcggcaccaaagtggaaattaaaaccggtcatcatcaccatcaccactgataagtttaaacccgctgatcagcctcgactgtgccttctagt

SEQ ID NO: 606 - DNA, 1359 bp, synthetic construct
  misc_feature 1..1359: note = CAR-T C2 CD3z sequence
  source 1..1359: mol_type = other DNA
atggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 607 - protein, 451 aa, synthetic construct
  REGION 1..451: note = CAR-T C2 CD3z sequence
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 608 - DNA, 1482 bp, synthetic construct
  misc_feature 1..1482: note = CAR-T C2 CD28/CD3z sequence
  source 1..1482: mol_type = other DNA
atggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtcccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 609 - protein, 492 aa, synthetic construct
  REGION 1..492: note = CAR-T C2 CD28/CD3z sequence
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 610 - DNA, 1485 bp, synthetic construct
  misc_feature 1..1485: note = CAR-T C2 4-1BB/CD3z sequence
  source 1..1485: mol_type = other DNA
atggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcaaaaggggccgcaaaaaactcctttacatttttaagcagccttttatgaggccagtacagacgactcaagaggaagacgggtgctcatgccgctttcctgaggaggaggaaggagggtgcgaactgcgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 611 - protein, 493 aa, synthetic construct
  REGION 1..493: note = CAR-T C2 4-1BB/CD3z sequence
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 612 - DNA, 1470 bp, synthetic construct
  misc_feature 1..1470: note = CAR-T C2 OX40/CD3z sequence
  source 1..1470: mol_type = other DNA
atggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgccggagggaccagaggctgccccccgatgcccacaagccccctgggggaggcagtttccggacccccatccaagaggagcaggccgacgcccactccaccctggccaagatccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 613 - protein, 488 aa, synthetic construct
  REGION 1..488: note = CAR-T C2 OX40/CD3z sequence
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 614 - DNA, 1593 bp, synthetic construct
  misc_feature 1..1593: note = CAR-T C2 CD28/OX40/CD3z sequence
  source 1..1593: mol_type = other DNA
atggccttgccagtgacggccctgctgctgccattggctcttctgttgcacgctgccaggcctgaagtgcagctcgtagagagtggcgggggactggtgaagcccggtggaagcctcagactcagttgcgccgcctcaggtttcactttttcaggttacgccatgtcctgggtaagacaggcaccggggaaaggactcgagtgggtgtctactatcagctcaggaggcacttatatatattatcctgactctgtaaaaggccgatttacgatttctcgcgacaatgcaaagaactccctctacctccaaatgaacagtcttagggcagaagacactgctgtatactattgtgcacgcctcggcggcgacaactactacgagtactttgacgtgtgggggaaagggactaccgtgacagtttcaagcggaggaggtggctcaggtggaggcgggtcaggggggggaggaagtgatattgtgctcacacaatccccagcctccctggctgtgtctcccggccaacgcgctacaattacatgtcgggcctccaaaagcgtgagcaccagcggctacagctacatgcactggtatcaacagaaaccaggacaaccccccaaactgttgatttatctcgcttcaaacttggagtccggcgtgcctgcgcgcttttcagggagtgggagcggcacagattttacgctgactatcaaccccgtagaagcaaacgatacagcgaattattattgtcaacattcccgggaactcccctttacgttcggcgggggcacaaaggtcgaaattaagagaaccacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtcccggagggaccagaggctgccccccgatgcccacaagccccctgggggaggcagtttccggacccccatccaagaggagcaggccgacgcccactccaccctggccaagatccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 615 - protein, 529 aa, synthetic construct
  REGION 1..529: note = CAR-T C2 CD28/OX40/CD3z sequence
MALPVTALLLPLALLLHAARPEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 616 - DNA, 1452 bp, synthetic construct
  misc_feature 1..1452: note = CAR-T E6 OX40/CD3z sequence
  source 1..1452: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgccggagggaccagaggctgccccccgatgcccacaagccccctgggggaggcagtttccggacccccatccaagaggagcaggccgacgcccactccaccctggccaagatccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 617 - protein, 482 aa, synthetic construct
  REGION 1..482: note = CAR-T E6 OX40/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 618 - DNA, 1575 bp, synthetic construct
  misc_feature 1..1575: note = CAR-T E6 CD28/OX40/CD3z sequence
  source 1..1575: mol_type = other DNA
atggccctgcccgtgaccgctttgctgctccccctggcgctgctgctgcacgccgccaggccagaggtccagctggttgagagtggcggtgggctggttaagcctggcggctccctgcggctgagctgcgccgcgagtggatttactttcagccgatatgggatgagttgggtgcggcaagctcccgggaagaggctggaatgggtctcaacaatctccggggggggcacttacatctattaccccgactcagtcaaggggagatttaccatttcacgagacaacgctaagaataccctgtatttgcagatgaattctctgagagcagaggacacagctgtttactattgtacccgcgacaactatggcaggaactacgactacggtatggactattggggacaagggacattggttacagtgagcagtggcggcgggggcagcggaggaggaggcagcggtggggggggcagcgagatagtgctcacgcagtcacccgcgactctcagtctctcacctggggaacgagctaccctgacgtgctctgctacctcctcagtgtcatatattcactggtatcagcaacggcccgggcagtcccctagattgctcatttatagtacctctaatctggcctcaggtatccctgcacgattttctggatctggttcaggttctgattacaccctcactatctctagcctggagcctgaagactttgccgtttattactgccagcagaggtctagctccccattcacctttgggagtgggaccaaggttgaaattaaaacgacaaccccggcccccagaccaccaacgccagcccccaccatcgccagccaacccctgtctctgagaccagaagcctgtaggcctgccgccggtggagctgtgcacacaagaggactggatttcgcctgtgatatctacatttgggccccgctcgcaggcacatgtggagtgctcctcctctccctggtgattaccctgtactgcagaagcaagcggtctcggctcctgcattctgattacatgaacatgaccccaagaagaccaggccccaccaggaaacattaccagccctacgctccgccacgcgacttcgctgcctaccggtcccggagggaccagaggctgccccccgatgcccacaagccccctgggggaggcagtttccggacccccatccaagaggagcaggccgacgcccactccaccctggccaagatccgcgttaagttctcccgatcagccgacgcgcctgcttacaagcagggccagaaccaactgtacaacgagctgaatctcggtagacgggaagagtacgacgtgttggacaaacggagaggccgcgacccagaaatgggcggcaagcctcgcaggaaaaacccccaggagggactgtacaatgagttgcagaaagataagatggcagaagcttatagcgagatcggaatgaagggggaaaggagacgagggaaaggacacgacggcctttatcagggcctgtccacagcaacaaaagatacgtatgacgccctccatatgcaggcacttccaccacggtgataa

SEQ ID NO: 619 - protein, 523 aa, synthetic construct
  REGION 1..523: note = CAR-T E6 CD28/OX40/CD3z sequence
MALPVTALLLPLALLLHAARPEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 620 - protein, 43 aa, synthetic construct
  REGION 1..43: note = MUC1 FRAGMENT
SNIKFRPGSVVVQLTLAFREGTINVHDVETQFNQYKTEAASRY

SEQ ID NO: 621 - protein, 35 aa, synthetic construct
  REGION 1..35: note = MUC1 FRAGMENT
SVVVQLTLAFREGTINVHDVETQFNQYKTEAASRY

SEQ ID NO: 622 - protein, 24 aa, synthetic construct
  REGION 1..24: note = Cytoplasmic MUC1 truncated segment
VQLTLAFREGTINVHDVETQFNQY

SEQ ID NO: 623 - protein, 24 aa, synthetic construct
  REGION 1..24: note = Cytoplasmic MUC1 truncated segment
SNIKFRPGSVVVQLTLAFREGTIN

SEQ ID NO: 624 - DNA, 28 bp, synthetic construct
  misc_feature 1..28: note = Primer
  source 1..28: mol_type = other DNA
attctaagcttgggccaccatggaactg

SEQ ID NO: 625 - DNA, 41 bp, synthetic construct
  misc_feature 1..41: note = Primer
  source 1..41: mol_type = other DNA
tctagagtttaaacttactatttacccggagacagggagag

SEQ ID NO: 626 - DNA, 41 bp, synthetic construct
  misc_feature 1..41: note = Primer
  source 1..41: mol_type = other DNA
agtatggcccagccggccgaggtgcagctggtggagtctgg

SEQ ID NO: 627 - DNA, 26 bp, synthetic construct
  misc_feature 1..26: note = Primer
  source 1..26: mol_type = other DNA
tagaaggcacagtcgaggctgatcag

SEQ ID NO: 628 - DNA, 27 bp, synthetic construct
  misc_feature 1..27: note = Primer
  source 1..27: mol_type = other DNA
attctaagcttgggccaccatggaagc

SEQ ID NO: 629 - DNA, 40 bp, synthetic construct
  misc_feature 1..40: note = Primer
  source 1..40: mol_type = other DNA
tctagagtttaaacttactaacactctcccctgttgaagc

SEQ ID NO: 630 - DNA, 43 bp, synthetic construct
  misc_feature 1..43: note = Primer
  source 1..43: mol_type = other DNA
agtatggcccagccggccgaaattgtgttgacacagtctccag

SEQ ID NO: 631 - DNA, 26 bp, synthetic construct
  misc_feature 1..26: note = Primer
  source 1..26: mol_type = other DNA
tagaaggcacagtcgaggctgatcag

SEQ ID NO: 632 - DNA, 33 bp, synthetic construct
  misc_feature 1..33: note = Primer
  source 1..33: mol_type = other DNA
actgtcatatggaggtgcagctggtggagtctg

SEQ ID NO: 633 - DNA, 37 bp, synthetic construct
  misc_feature 1..37: note = Primer
  source 1..37: mol_type = other DNA
actgtctcgagtttaatttccactttggtgccgctgc

SEQ ID NO: 634 - DNA, 33 bp, synthetic construct; identical to SEQ ID NO: 632
  misc_feature 1..33: note = Primer
  source 1..33: mol_type = other DNA

SEQ ID NO: 635 - DNA, 37 bp, synthetic construct
  misc_feature 1..37: note = Primer
  source 1..37: mol_type = other DNA
actgtaccggttttaatttccactttggtgccgctgc

SEQ ID NO: 636 - DNA, 29 bp, synthetic construct
  misc_feature 1..29: note = Primer
  source 1..29: mol_type = other DNA
cttcttcctcaggagcaagctcaccgtgg

SEQ ID NO: 637 - DNA, 18 bp, synthetic construct
  misc_feature 1..18: note = Primer
  source 1..18: mol_type = other DNA
gagccgtcggagtccagc

SEQ ID NO: 638 - DNA, 18 bp, synthetic construct
  misc_feature 1..18: note = Primer
  source 1..18: mol_type = other DNA
gcacctgaactcctgggg

SEQ ID NO: 639 - DNA, 22 bp, synthetic construct
  misc_feature 1..22: note = Primer
  source 1..22: mol_type = other DNA
tttaatttccactttggtgccg

SEQ ID NO: 640 - DNA, 32 bp, synthetic construct
  misc_feature 1..32: note = Primer
  source 1..32: mol_type = other DNA
cgcggctagcttaagcttggtaccgagggcca

SEQ ID NO: 641 - DNA, 36 bp, synthetic construct
  misc_feature 1..36: note = Primer
  source 1..36: mol_type = other DNA
cgcggcggccgcctgatcagcgggtttaaacttatc

SEQ ID NO: 642 - protein, 35 aa, synthetic construct
  REGION 1..35: note = PSMGFR N-10
QFNQYKTEAASRYNLTISDVSVSDVPFPFSAQSGA

SEQ ID NO: 643 - protein, 35 aa, synthetic construct
  REGION 1..35: note = PSMGFR C-10
GTINVHDVETQFNQYKTEAASRYNLTISDVSVSDV